(19) United States
(12) Patent Application Publication
(10) Pub. No.: US 2023/0263883 A1
(43) Pub. Date: Aug. 24, 2023

(54) Severe Acute Respiratory Syndrome Coronavirus 2 (SARS CoV-2) Peptide Epitopes

(71) Applicant: The Brigham and Women`s Hospital, Inc., Boston, MA (US)

(72) Inventors: Stephen J. Elledge, Brookline, MA (US); Ellen Shrock, Boston, MA (US); Eric Fujimura, Boston, MA (US)

(21) Appl. No.: 18/014,947

(22) PCT Filed: Jul. 8, 2021
(86) PCT No.: PCT/US21/40920

§ 371 Date: Jan. 6, 2023

Related U.S. Application Data
(60) Provisional application No. 63/049,359, filed on Jul. 8, 2020.
(60) Provisional application No. 63/083,607, filed on Sep. 25, 2020.

(51) Int. Cl. A61K 39/215; C07K 16/10; A61K 49/00; G01N 33/569

(52) U.S. Cl. CPC A61K 39/215; C07K 16/10; A61K 49/0004; G01N 33/56983; G01N 2333/165; G01N 2469/20

(57) ABSTRACT

Peptide epitopes identified in subjects infected with severe acute respiratory syndrome coronavirus 2 (SARS-CoV-2) and methods of use thereof for diagnosing, determining prognosis, and treating Coronavirus Disease 2019 (COVID-19), and developing prophylactic or therapeutic vaccines against SARS-CoV-2.

### CLAIM OF PRIORITY

This application claims the benefit of U.S. Provisional Patent Application Serial Nos. 63/049,359, filed on Jul. 8, 2020, and 63/083,607, filed on Sep. 25, 2020. The entire contents of the foregoing are hereby incorporated by reference.

### FEDERALLY SPONSORED RESEARCH OR DEVELOPMENT

This invention was made with Government support under Grant No. All 18633 awarded by the National Institutes of Health. The Government has certain rights in the invention.

### TECHNICAL FIELD

Described herein are peptide epitopes identified in subjects infected with severe acute respiratory syndrome coronavirus 2 (SARS-CoV-2) and methods of use thereof for diagnosing, determining prognosis, and treating Coronavirus Disease 2019 (COVID-19), and developing prophylactic or therapeutic vaccines against SARS-CoV-2.

### BACKGROUND

Coronaviruses comprise a large family of enveloped, positive-sense single-stranded RNA viruses that cause diseases in birds and mammals (1). Among the strains that infect humans are the alpha-coronaviruses HCoV-229E and HCoV-NL63 and the beta-coronaviruses HCoV-OC43 and HCoV-HKU1, which cause common colds (FIG. 1A). Three additional beta-coronavirus species result in much more severe infections in humans: Severe Acute Respiratory Syndrome Coronavirus (SARS-CoV) was responsible for an outbreak in Asia in 2003 which resulted in ˜8000 infections and over 800 deaths (2); Middle East Respiratory Syndrome Coronavirus (MERS-CoV), which emerged in 2012 and resulted in −2500 infections and over 800 deaths (3); and SARS-CoV-2, a novel coronavirus that emerged in late 2019 in Asia and quickly spread throughout the globe (4). As of early June 2020, SARS-CoV-2 had caused over 9 million confirmed infections and was responsible for over 475,000 deaths (5).

### SUMMARY

As described herein, VirScan (see PCT/US2018/036663) was used to map a total of 3071 SARS-CoV-2 epitopes, including 813 unique epitopes, with unprecedented resolution. Kinetics of induction and variation in epitope selection were observed over time in recently-infected individuals. A machine learning model was developed, trained on VirScan data to detect SARS-CoV-2 exposure history with very high sensitivity and specificity. VirScan identified public epitopes that are specific to SARS-CoV-2, and we employed these in a rapid Luminex assay to distinguish recently-infected COVID-19 patients from controls. Finally, VirScan enabled us to examine the history of previous viral infections and to determine correlates of COVID-19 outcomes.

Described herein are high throughput anti-SARS-CoV-2 antibody detection methodologies, e.g., the exemplary COVID-19 Luminex assay, which facilitate accurate analyses of seroprevalence. The identification of binding sites of anti-SARS-CoV-2 antibodies provides a stepping stone to the isolation and functional dissection of both neutralizing antibodies and antibodies that might exacerbate patient outcomes through antibody-dependent enhancement (ADE). Finally, the data showed that H COVID-19 patients exhibited a higher incidence of prior infection with CMV and HSV-1 but had lower levels of antibodies to most common viruses, compared to the NH cohorts.

Thus, provided herein are methods for detecting the presence of antibodies that bind to SARS-CoV-2 in a sample. The methods include providing a sample comprising or suspected of comprising antibodies that bind to SARS-CoV-2; contacting the sample with one, two, or more, e.g., 1, 2, 3, 4, 5, 8, 10, 12, 15, 20, 25, 30, 50, 75, 80, 100, 150, 200, 250, 300, 350, 400, 500, 600, 700, 800, or more, peptides comprising 4 or more consecutive amino acids from a SARS-CoV-2 epitope sequence shown herein, e.g., in Table 1, Table 3, and/or Table 4 or SEQ ID NOs:13-1170, under conditions sufficient for binding of antibodies in the sample to the peptides; and detecting binding of antibodies in the sample to the peptides.

In some embodiments, the sample is from a subject, optionally a subject who is known or suspected of being infected with SARS-CoV-2. In some embodiments, the methods include identifying a subject who has antibodies that bind to SARS-CoV-2 as having been infected with SARS-CoV-2. In some embodiments, the methods further include administering a treatment for SARS-CoV-2 to the subject or monitoring the subject for later health consequences of infection with SARS-CoV-2. In some embodiments, the subject is a human subject. In some embodiments, the sample comprises whole blood, serum, saliva or plasma.

In some embodiments, the peptides comprise a detectable moiety, are conjugated to a bead, or are conjugated to a surface. In some embodiments, the detectable moiety is a fluorescent label. In some embodiments, the surface is a multiwell plate or glass coverslip. In some embodiments, the beads are magnetic.

In some embodiments, detecting comprises performing an immunoassay, multiplex immunoassay, protein-fragment complementation assay (PCA), or single molecule array.

Also provided herein are compositions or kits comprising one, two, or a plurality of antigenic peptides comprising 4 or more consecutive amino acids from epitope sequences shown herein, e.g., in Table 1, 3, or 4 or SEQ ID NOs:13-1170, e.g., from one of SEQ ID NOs: 1036-1050.

In some embodiments, at least one of the peptides comprises a detectable moiety, is conjugated to a bead, or is conjugated to a surface. In some embodiments, the detectable moiety is a fluorescent label. In some embodiments, the surface is a multiwell plate or glass coverslip. In some embodiments, the beads are magnetic. In some embodiments, the composition comprises a pharmaceutically acceptable carrier and optionally an adjuvant.

Also provided are the compositions for use in a method of treating or reducing risk of an infection with SARS-CoV-2 in a subject.

Further provided are methods of treating or reducing risk or severity of an infection with SARS-CoV-2 in a subject, the methods comprising administering a therapeutically of prophylactically effective amount of a composition as described herein, comprising one, two, or a plurality of antigenic peptides comprising 4 or more consecutive amino acids from epitope sequences shown herein, e.g., in Table 1, 3, or 4 or SEQ ID NOs:13-1170, e.g., from one of SEQ ID NOs: 1036-1050.

Additionally, provided are methods of generating an antibody to SARS-CoV-2, the method comprising administering the compositions, and optionally an adjuvant, to a mammal, and isolating antibodies from the mammal that bind to SARS-CoV-2.

In addition, provided herein are methods for identifying antibodies that bind to neutralizing or non-neutralizing epitopes of SARS-CoV-2. The methods include providing a sample comprising an antibody obtained, preferably cloned, from a human who has had a SARS-CoV-2 infection; contacting the antibody with peptides comprising one or more, e.g., 1, 2, 3, 4, 5, 8, 10, 12, 15, 20, 25, 30, 50, 75, 80, 100, 150, 200, 250, 300, 350, 400, 500, 600, 700, 800, or more, peptides comprising at least 4, e.g., 5, 6, 7, 8, 9, 10, 11, 12, 13, 14, 15, 16, 17, 18, 19, 20 or more, consecutive amino acids from a SARS-CoV-2 epitope sequence shown herein, e.g., in Table 1, Table 3, and/or Table 4 or SEQ ID NOs:13-1170, wherein: (i) the peptides comprise non-neutralizing epitopes as shown herein, e.g., from one of SEQ ID NOs: 333-1035 or 1051-1155, and the contacting is performed under conditions to allow binding of the antibody on B cells to the peptides; and identifying the antibody as non-neutralizing if it binds to a peptide that comprises a non-neutralizing epitope; or (ii) the peptides comprise neutralizing epitopes shown herein, e.g., from one of SEQ ID NOs: 1036-1050, and the contacting is performed under conditions to allow binding of the antibody on B cells to the peptides; and identifying the antibody as neutralizing if it binds to a peptide that comprises a neutralizing epitope.

In some embodiments, the methods further include cloning one or more antibodies, wherein cloning the antibodies comprises providing a sample of B cells from a human who has had a SARS-CoV-2 infection; contacting the B cells with peptides including one, two, or more of the epitope sequences shown herein, e.g., in Table 1, Table 3, and/or Table 4, optionally one of one of SEQ ID NOs: 1036-1050; cloning and sequencing B cells encoding antibodies specific for one or more of the epitope sequences; and optionally testing these antibodies for neutralizing activity or Fc-mediated effector function (e.g., antibody-dependent cellular cytotoxicity, complement-dependent cytotoxicity, and antibody-dependent cellular phagocytosis).

In some embodiments, the methods further include formulating the optimized population of antibodies into a pharmaceutical composition by mixing the antibodies with a pharmaceutically acceptable carrier, e.g., to reduce or prevent the evolution of antibodies that are immunodominant but not protective.

In some embodiments, the methods further include administering a therapeutically effective amount of the pharmaceutical composition to a subject in need thereof.

In some embodiments, the methods further include cloning one or more antibodies identified as non-neutralizing into a pharmaceutical composition.

In some embodiments, the methods further include formulating the optimized population of antibodies into a pharmaceutical composition by mixing the antibodies with one or more of a pharmaceutically acceptable carrier, an adjuvant, and/or a SARS-CoV-2 vaccine comprising a SARS-CoV-2 protein, peptide, or nucleic acid encoding a SARS-CoV-2 protein or peptide.

In some embodiments, the methods further include administering a prophylactically effective amount of the pharmaceutical composition to a subject in need thereof.

Also provided herein are methods for selecting a vaccine composition for use in eliciting a prophylactic response to SARS-CoV-2 in a subject. The methods include administering a composition comprising a SARS-CoV-2 protein, peptide, or nucleic acid encoding a SARS-CoV-2 protein or peptide, to a test subject in an amount sufficient to elicit an immune response; obtaining a sample comprising antibodies obtained from the subject; contacting the sample with one or more, e.g., 1, 2, 3, 4, 5, 8, 10, 12, 15, 20, 25, 30, 50, 75, 80, 100, 150, 200, 250, 300, 350, 400, 500, 600, 700, 800, or more, peptides comprising at least 4, e.g., 5, 6, 7, 8, 9, 10, 11, 12, 13, 14, 15, 16, 17, 18, 19, 20 or more, consecutive amino acids from a SARS-CoV-2 epitope sequence shown herein, e.g., in Table 1, Table 3, and/or Table 4 or SEQ ID NOs:13-1170, under conditions to allow binding of the antibody to the peptides; and detecting binding of antibodies in the sample to the peptides, wherein: (i) the composition of the vaccine excludes one or more epitopes that elicit non-protective antibodies; or (ii) the composition of the vaccine comprises epitopes that elicit protective (neutralizing) antibodies shown herein, e.g., one of SEQ ID NOs: 1036-1050; and selecting a vaccine composition that elicits neutralizing antibodies.

In some embodiments, the vaccine composition comprises one or more mutations in a non-neutralizing epitope.

Also provided are compositions comprising a SARS-CoV-2 protein, peptide, or nucleic acid encoding a SARS-CoV-2 protein or peptide, wherein the SARS-CoV-2 protein, peptide, or nucleic acid encoding a SARS-CoV-2 protein or peptide comprises a mutation in a non-neutralizing epitope sequences shown herein, e.g., in Table 3 or 4, and a pharmaceutically acceptable carrier, and optionally an adjuvant, and the use thereof in eliciting a prophylactic response in a subject.

Further, provided herein are methods for generating an antibody to SARS-CoV-2, the method comprising administering the compositions to a subject.

Additionally provided are methods for treating or reducing risk or severity of an infection with SARS-CoV-2 in a subject, the method comprising administering a therapeutically or prophylactically effective amount of the compositions to the subject. Also provided are kits comprising a composition as described herein, e.g., for use in a method of detecting the presence of antibodies that bind to SARS-CoV-2 in a sample, e.g., to diagnose a subject with COVID-19.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-E. VirScan detects the humoral response to SARS-CoV-2 in sera from COVID-19 patients

(A) Phylogeny tree of 50 coronavirus sequences (13) constructed using MEGA X (14, 15). The scale bar indicates the estimated number of base substitutions per site (16). Coronaviruses included in the updated VirScan library are indicated.
(B) Schematic representation of the ORFs encoded by the SARS-CoV-2 genome (12, 17).
(C) Overview of the VirScan procedure (7-10). The coronavirus oligonucleotide library includes 56-mer peptides tiling every 28 amino acids across the proteomes of 10 coronavirus strains, and 20-mer peptides tiling every 5 amino acids across the SARS-CoV-2 proteome. Oligonucleotides were cloned into a T7 bacteriophage display vector and packaged into phage particles displaying the encoded peptides on their surface. The phage library was mixed with sera containing antibodies that bind to their cognate epitopes on the phage surface; bound phage were isolated by immunoprecipitation (IP) with either anti-IgG- or anti-IgA-coated magnetic beads. Lastly, PCR amplification and Illumina sequencing from the DNA of the bound phage revealed the peptides targeted by the serum antibodies.
(D) Detection of antibodies targeting coronavirus epitopes by VirScan. Heatmaps depict the humoral response from COVID-19 patients (n=232) and pre-COVID-19 era control samples (n=190). Each column represents a sample from a unique individual. The color intensity indicates the number of 56-mer peptides from the indicated coronaviruses significantly enriched by IgG antibodies in the serum sample.
(E) Boxplots illustrate the number of peptide hits from the indicated coronaviruses in COVID-19 patients and pre-COVID-19 era controls. The box indicates the interquartile range, with a line at the median. The whiskers represent 1.5 times the interquartile range.

FIGS. 2A-C. Overall landscape of SARS-CoV-2 protein recognition in COVID-19 patient versus control sera.

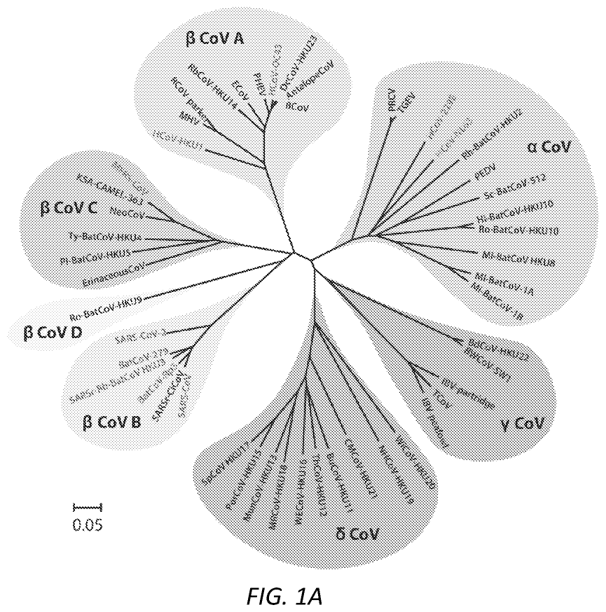

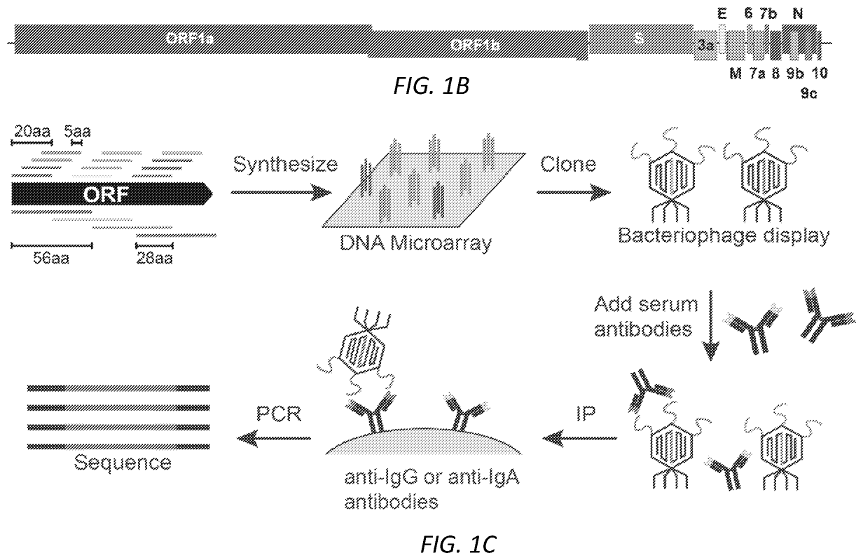

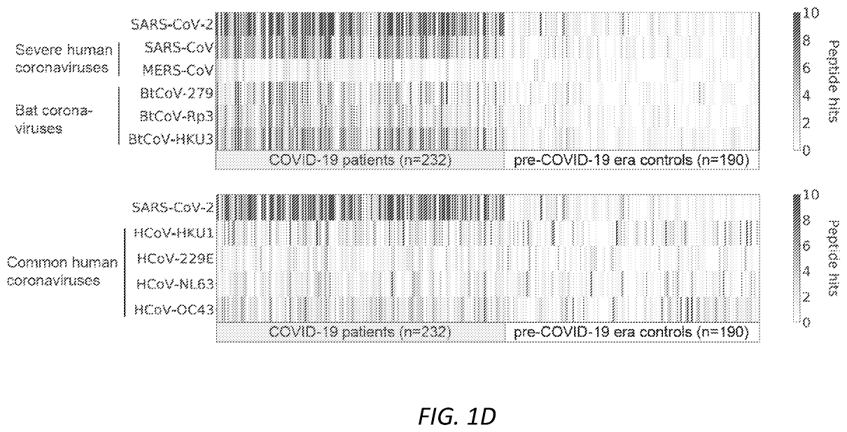

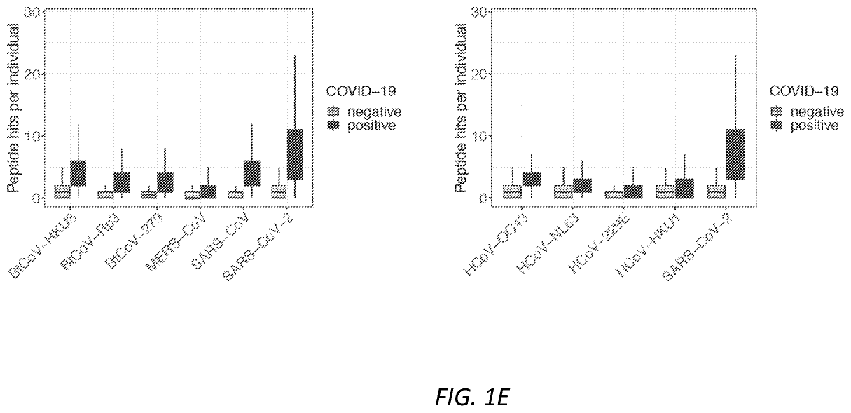

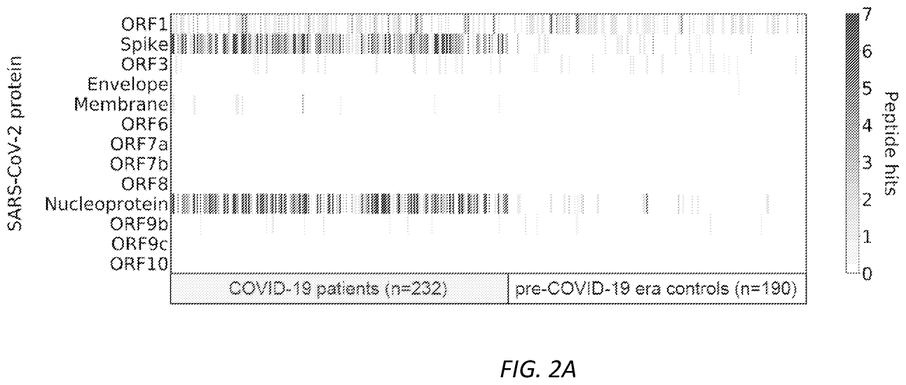

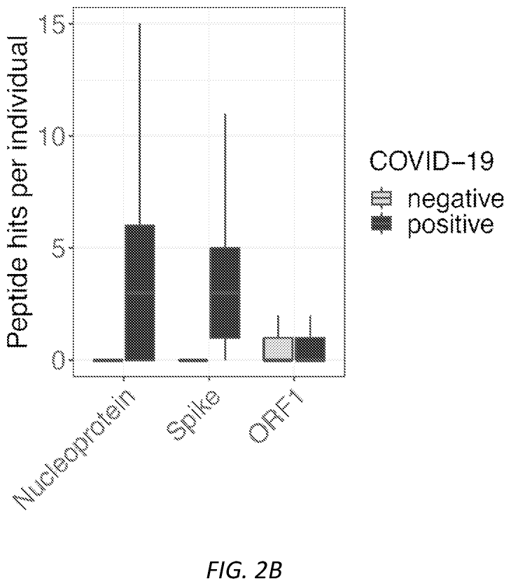

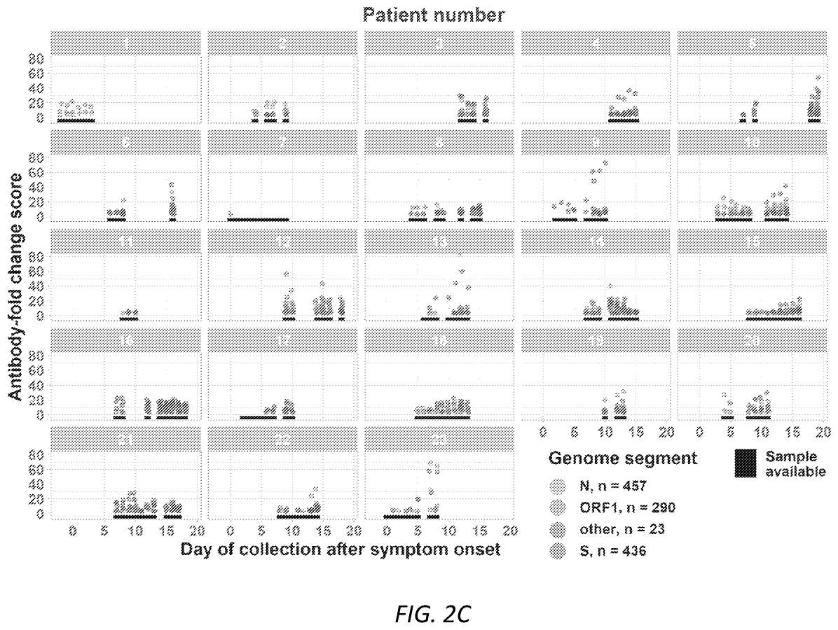

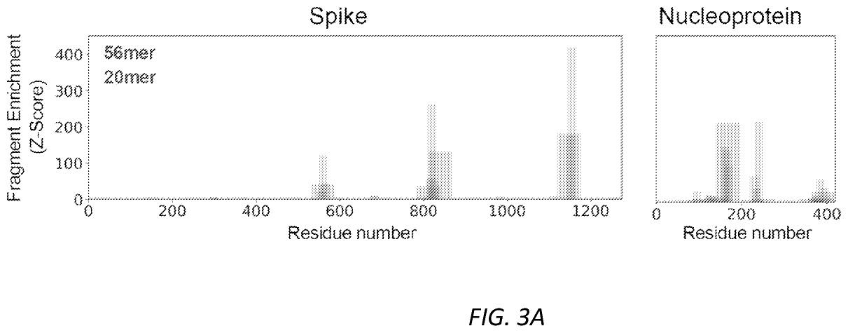

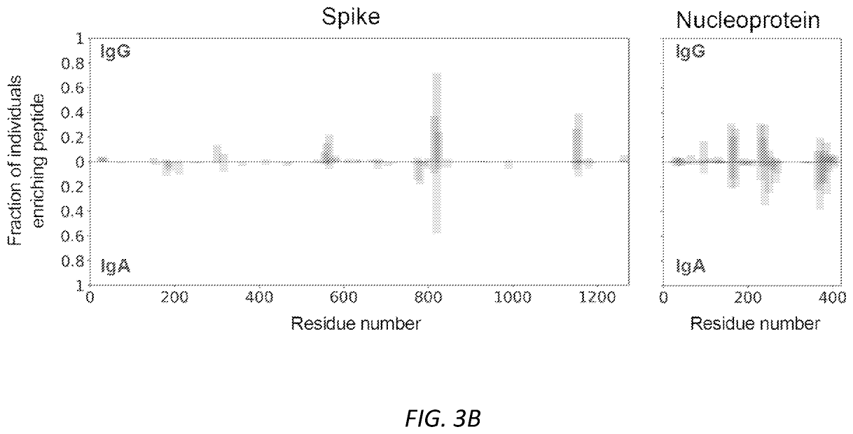

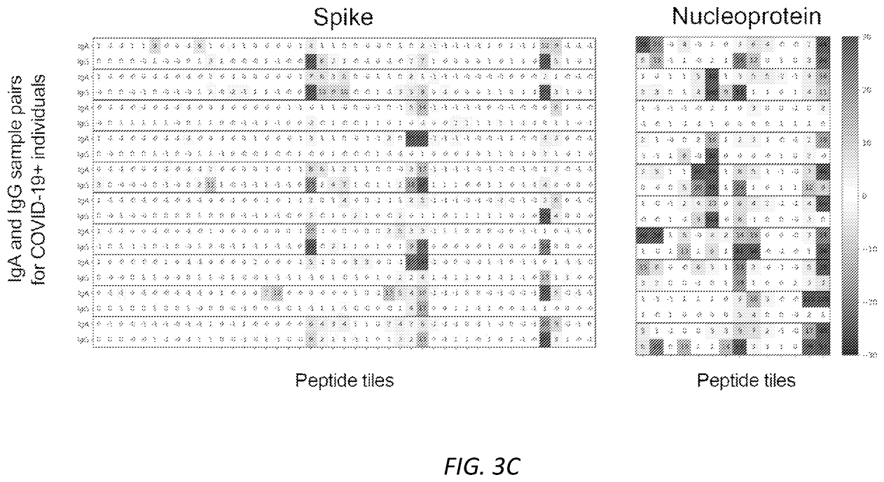

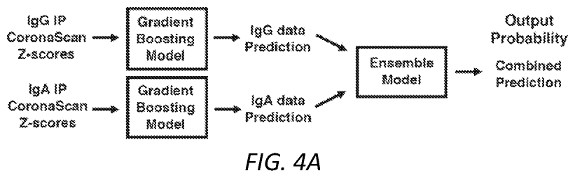

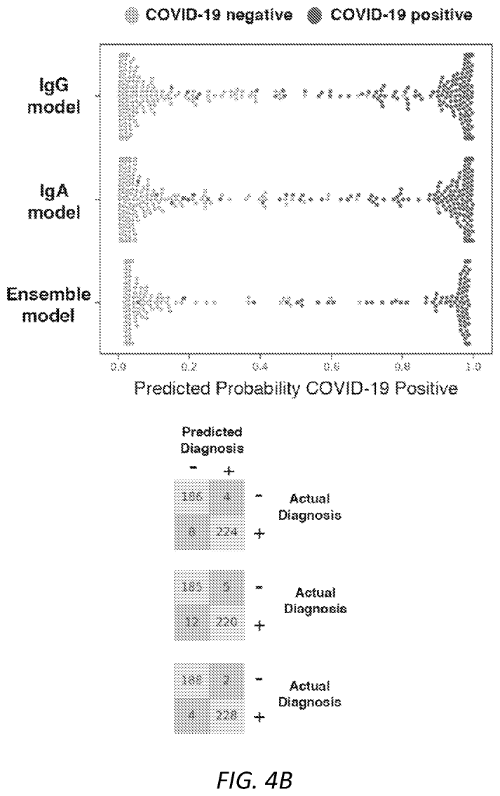

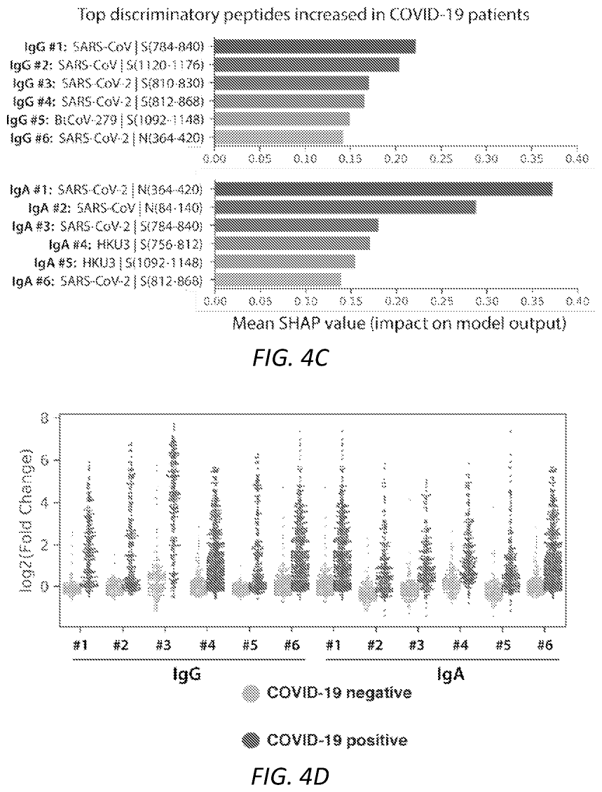

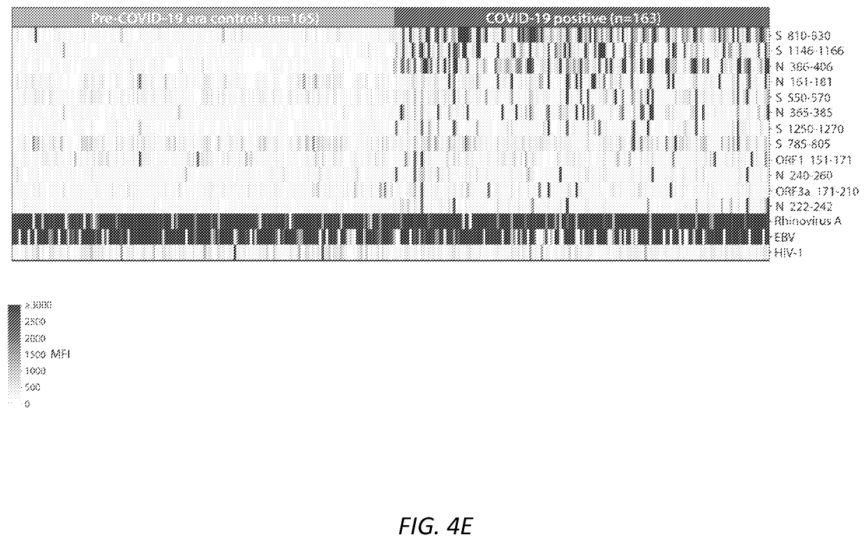

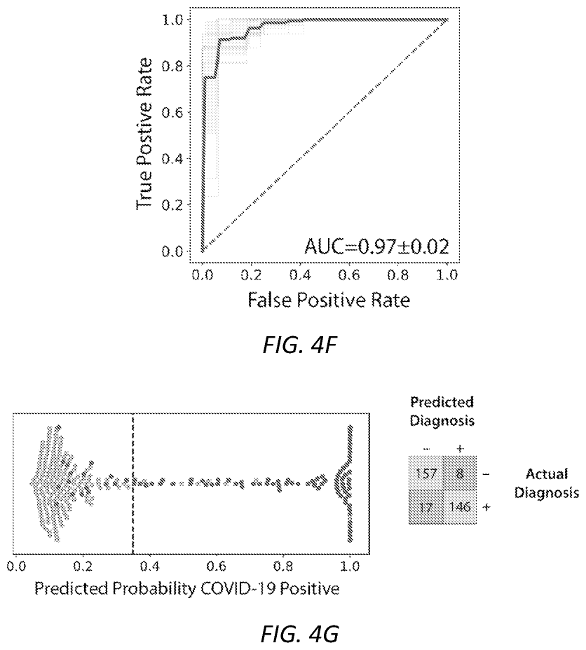

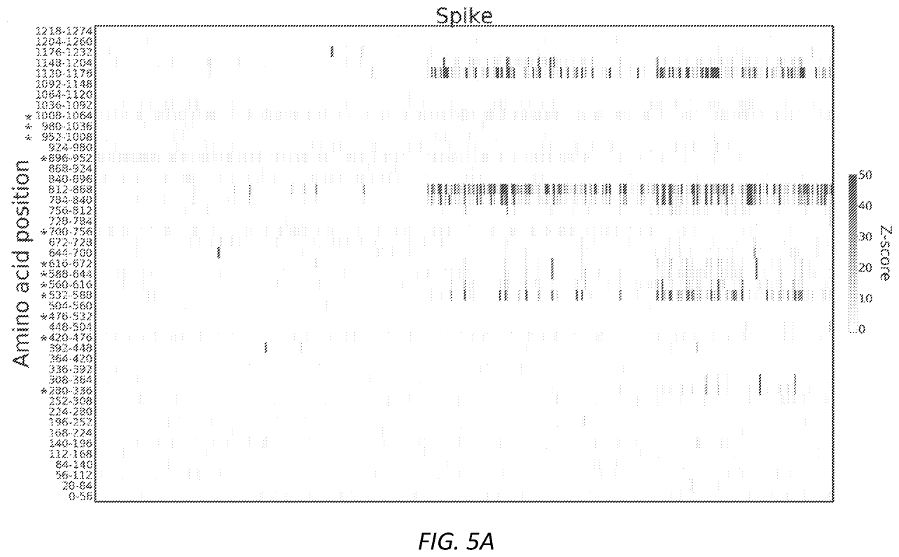

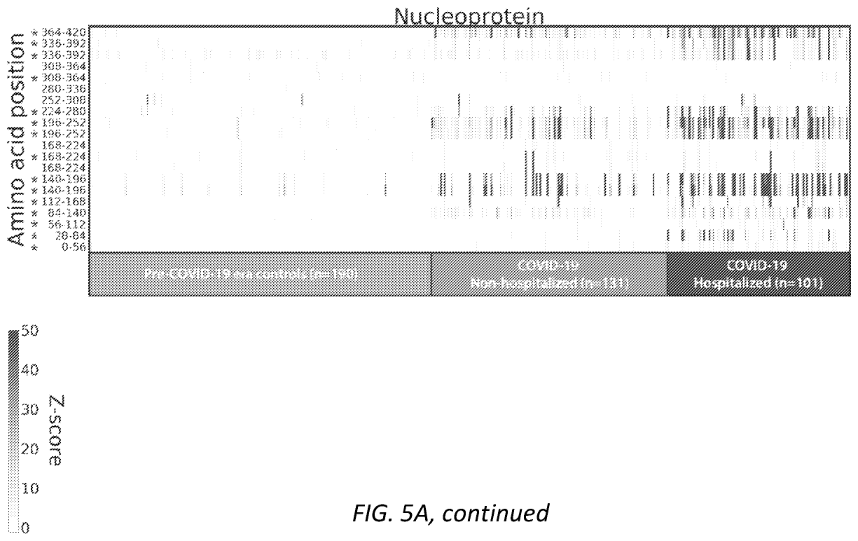

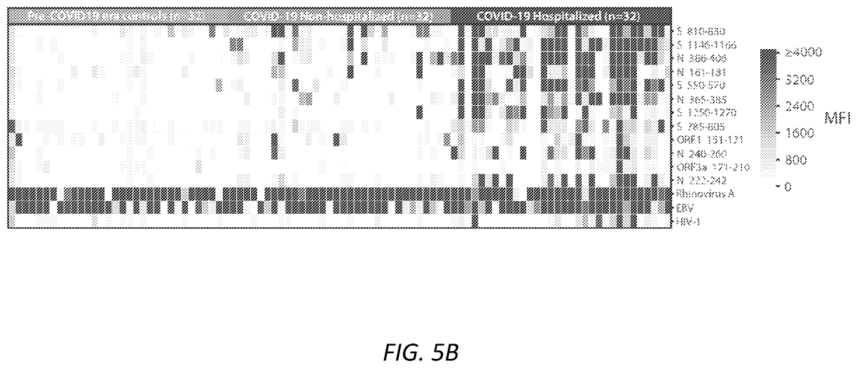

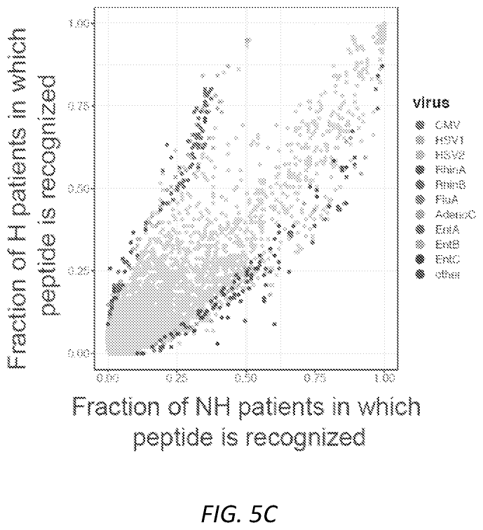

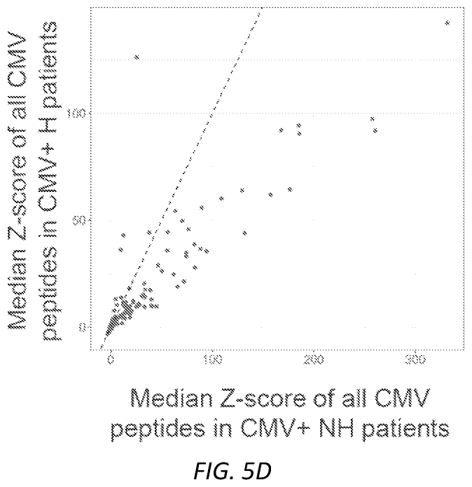

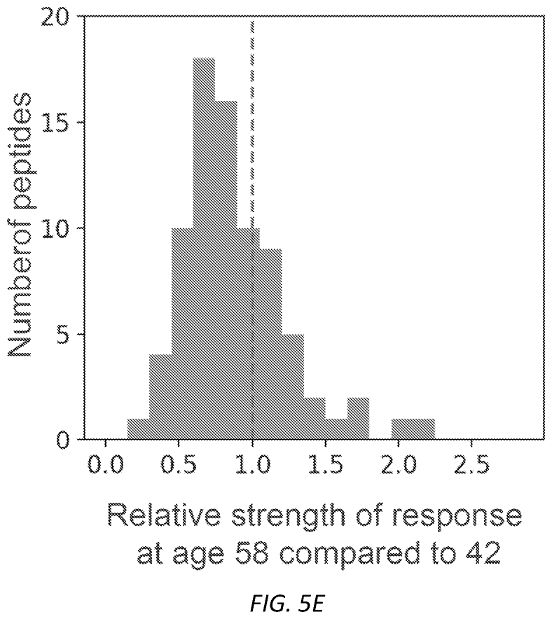

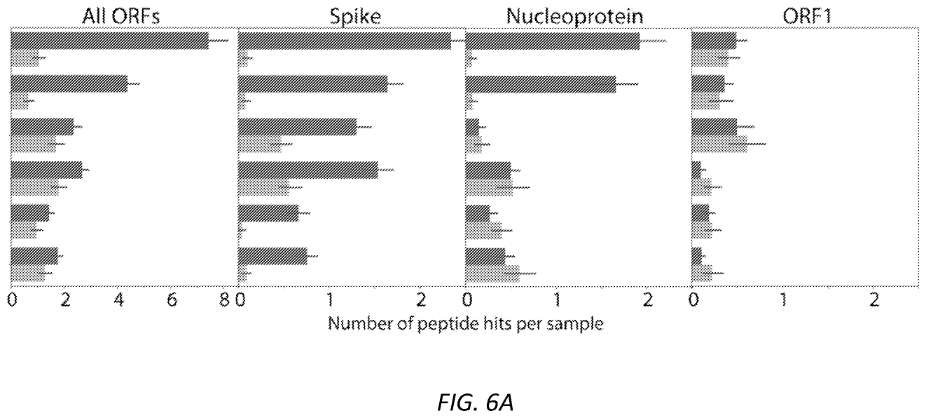

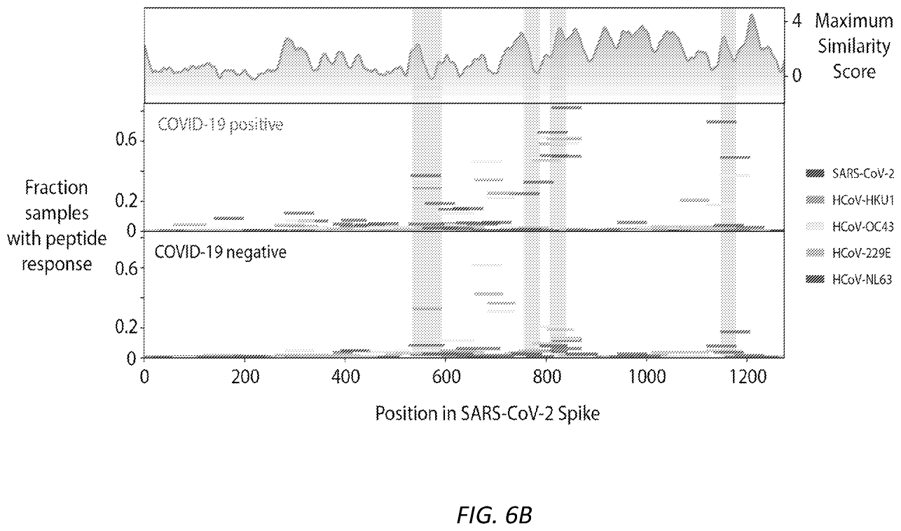

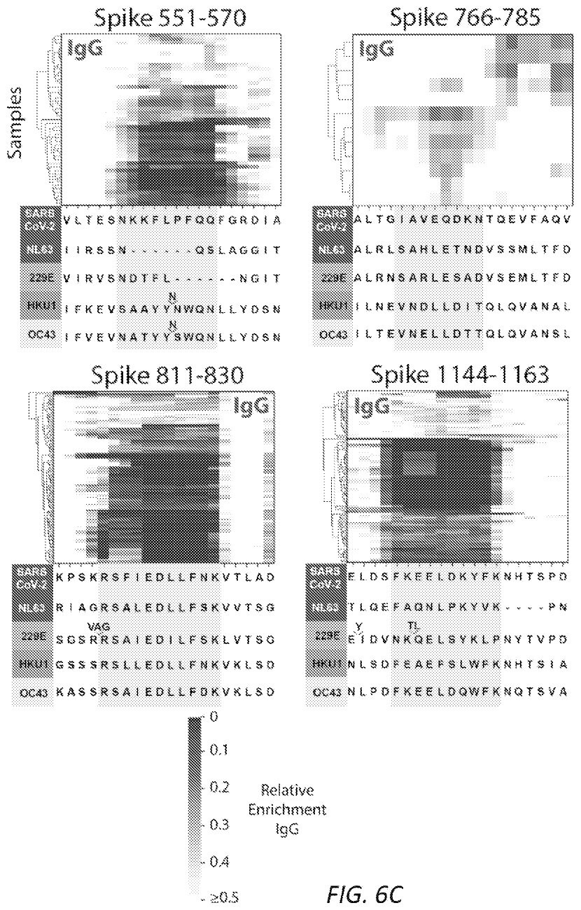

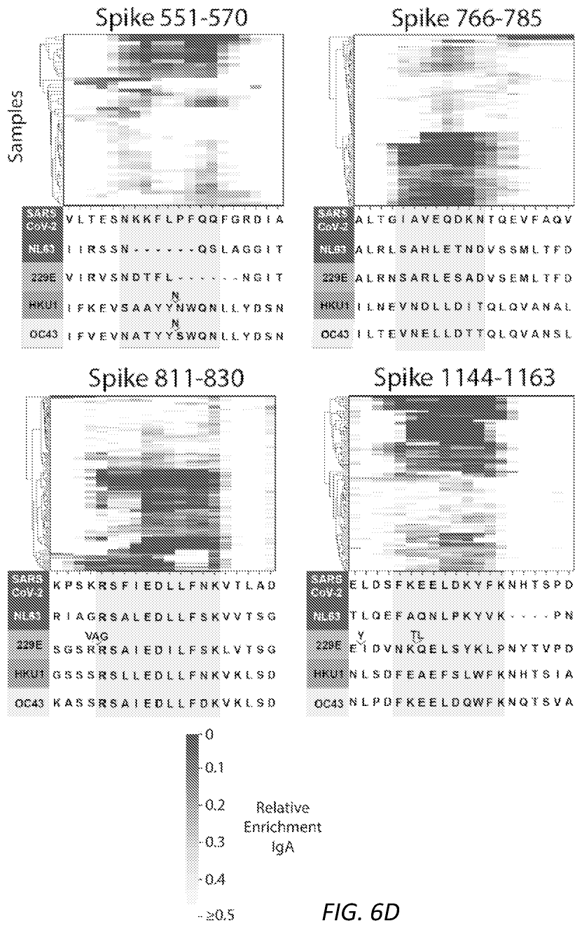

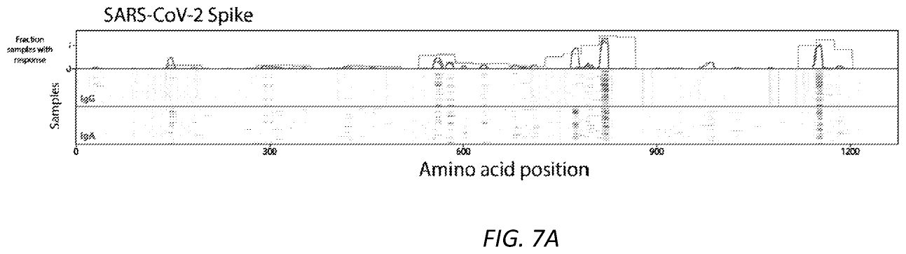

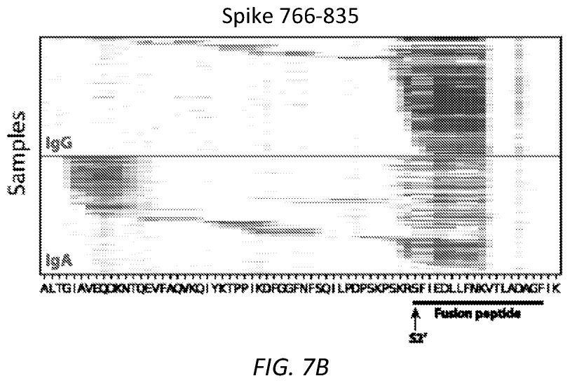

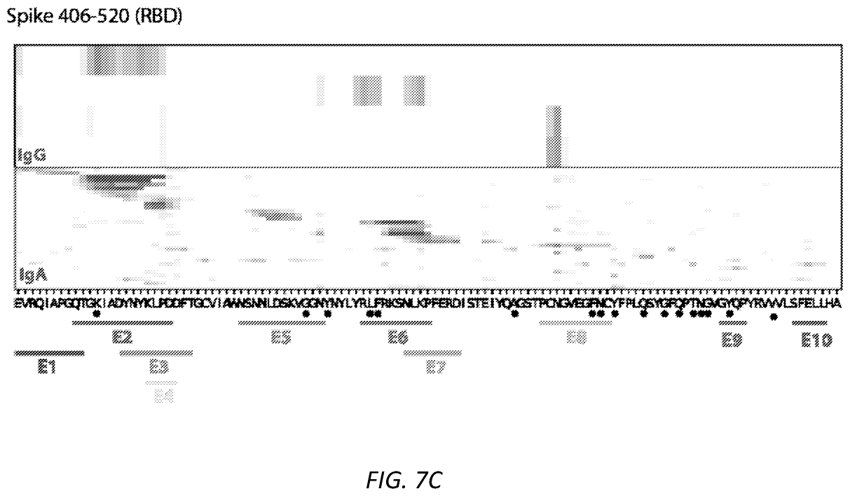

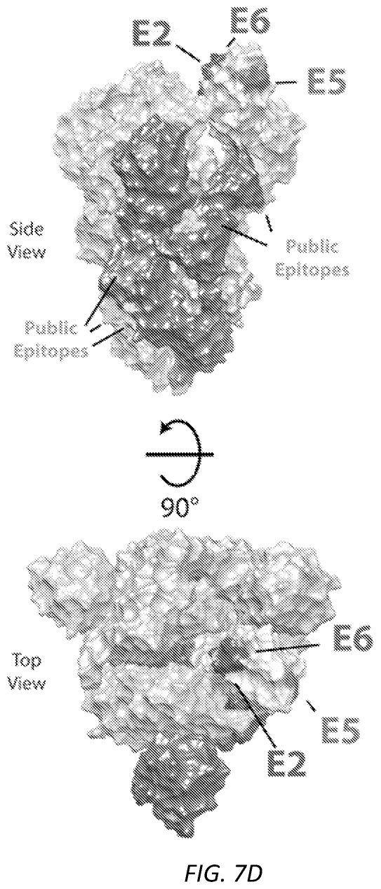

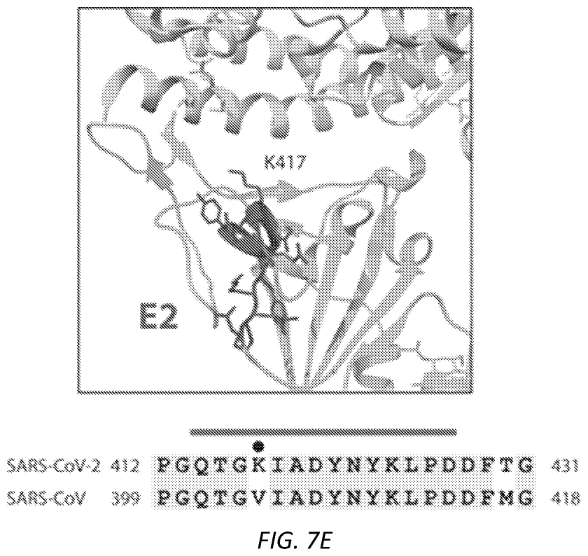

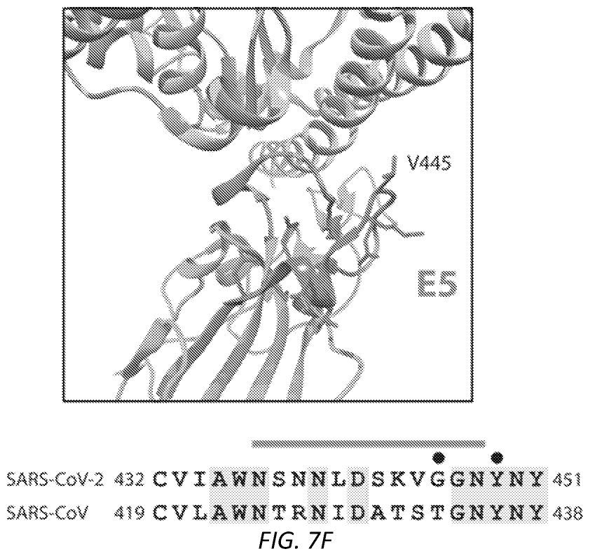

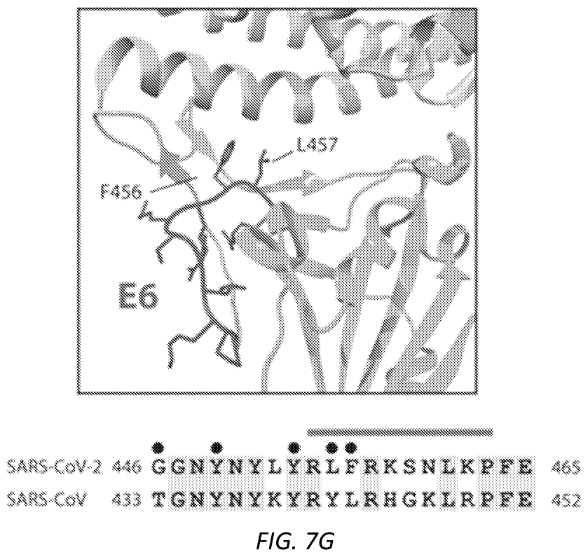

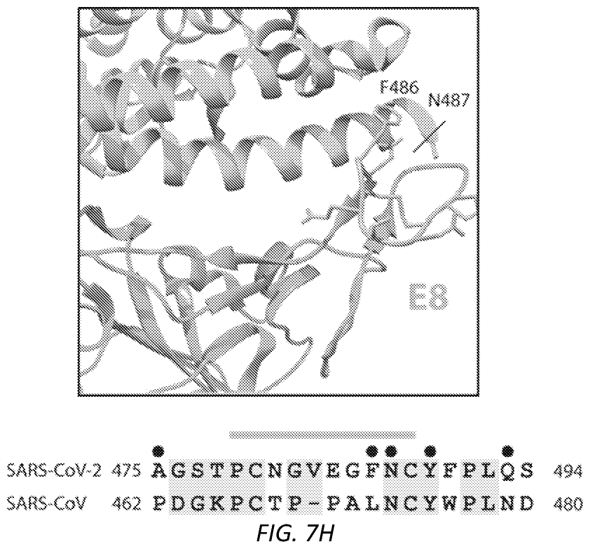

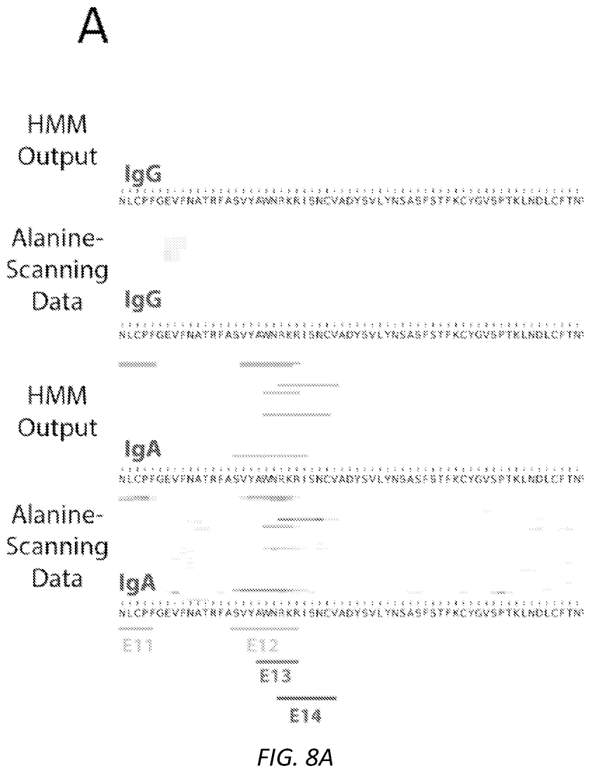

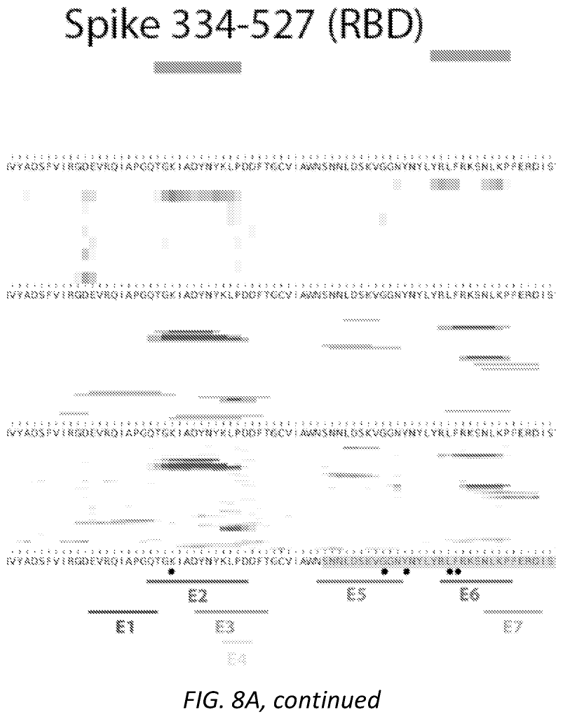

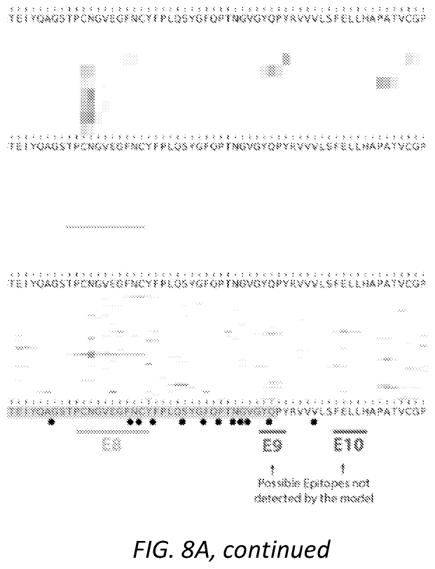

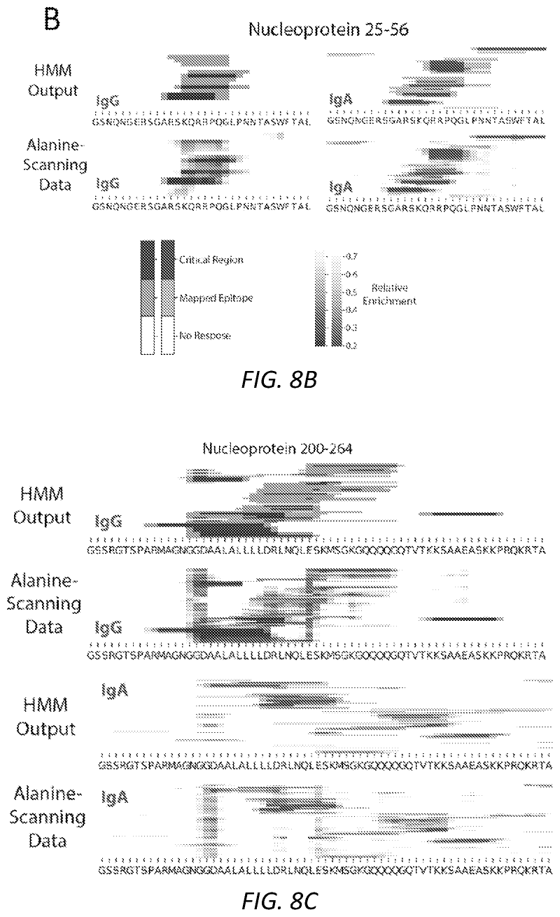

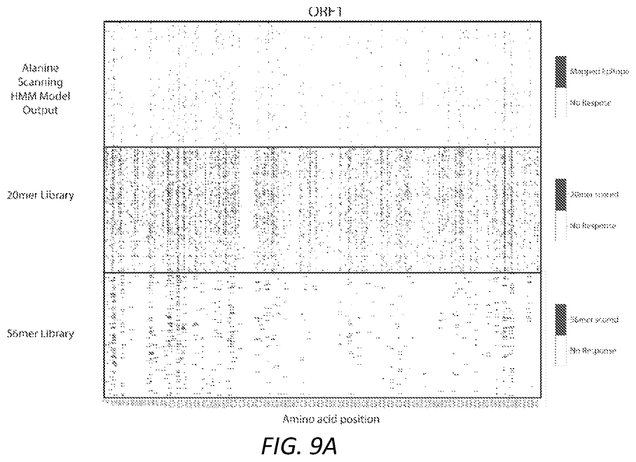

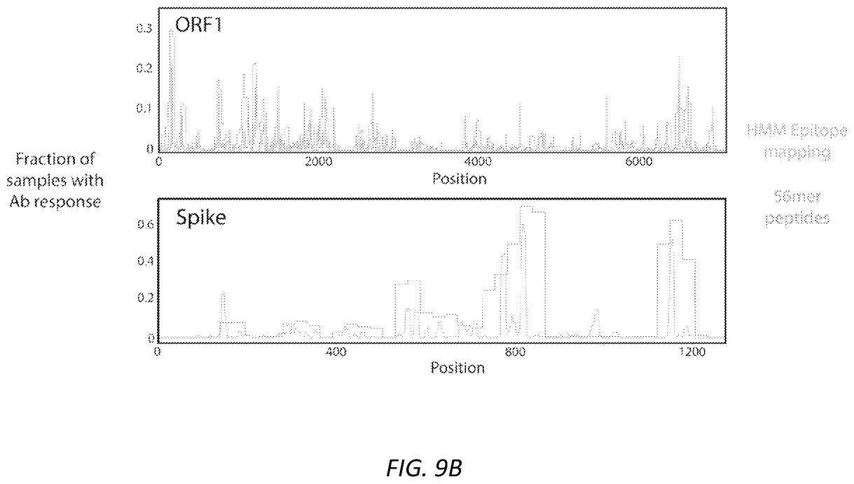

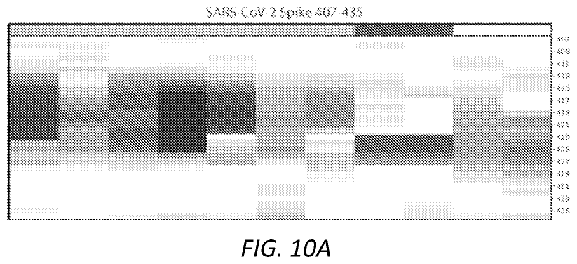

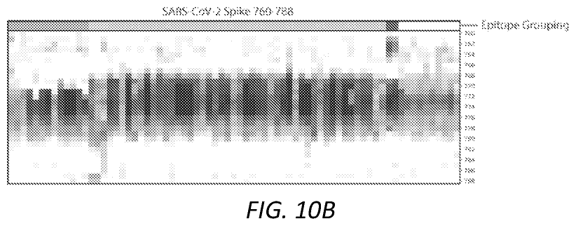

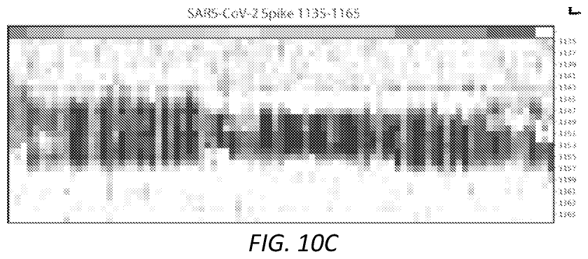

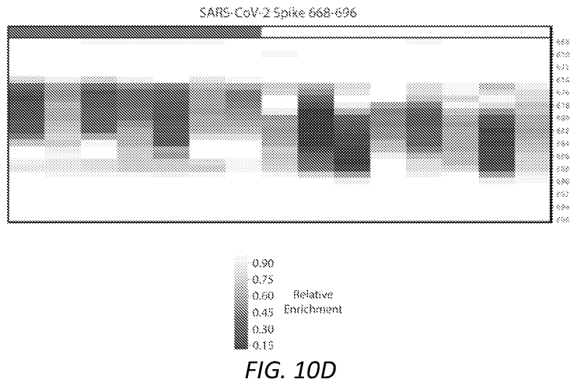

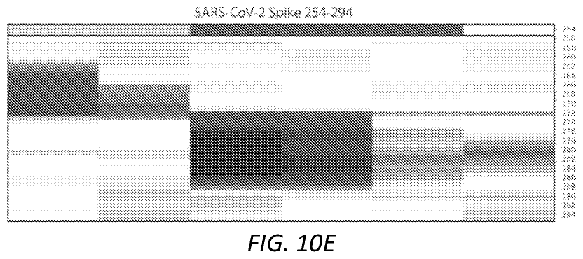

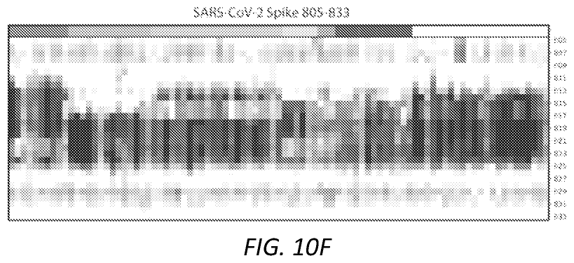

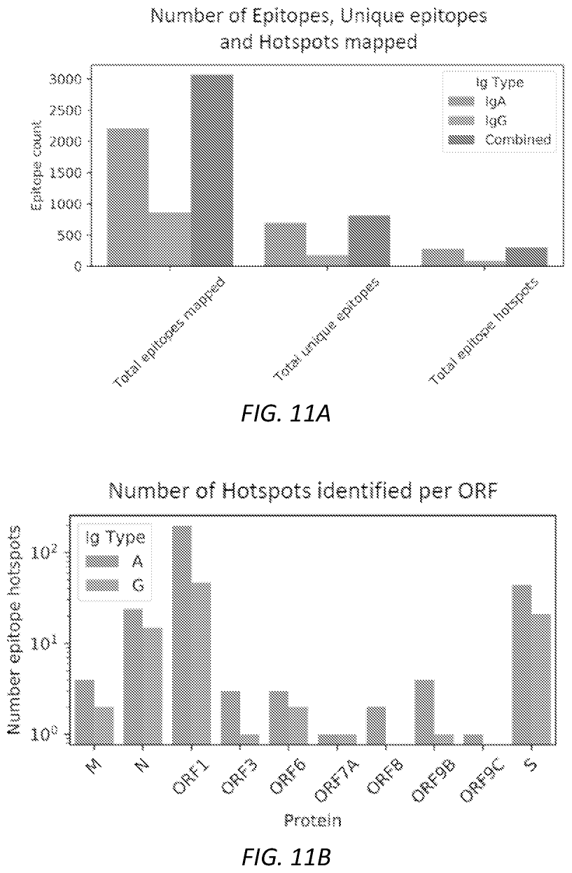

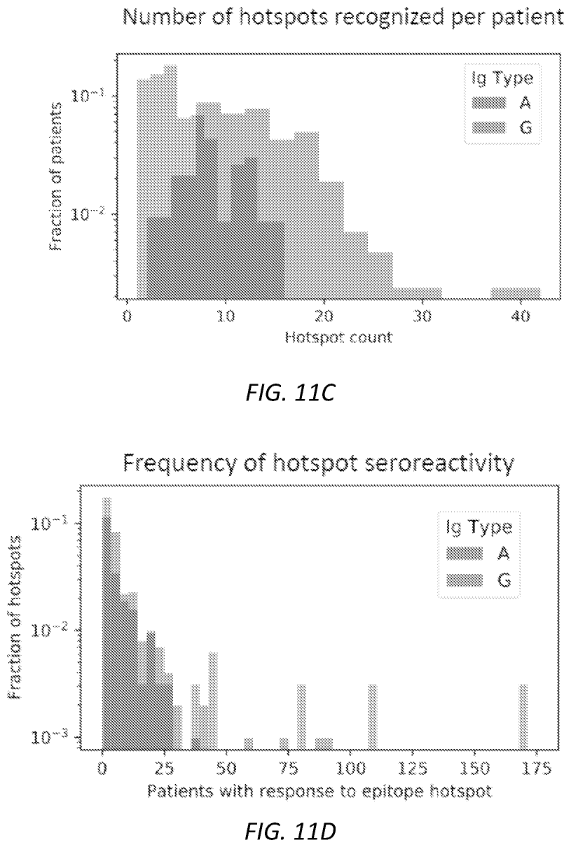

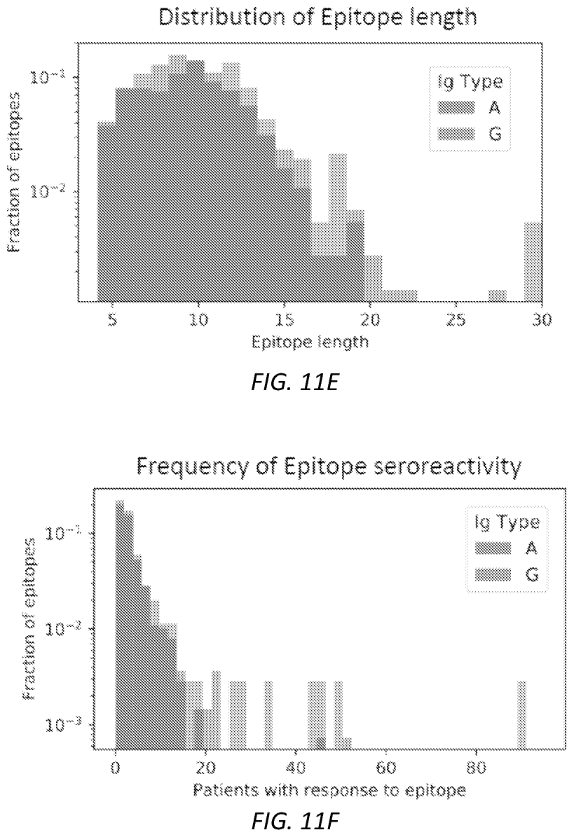

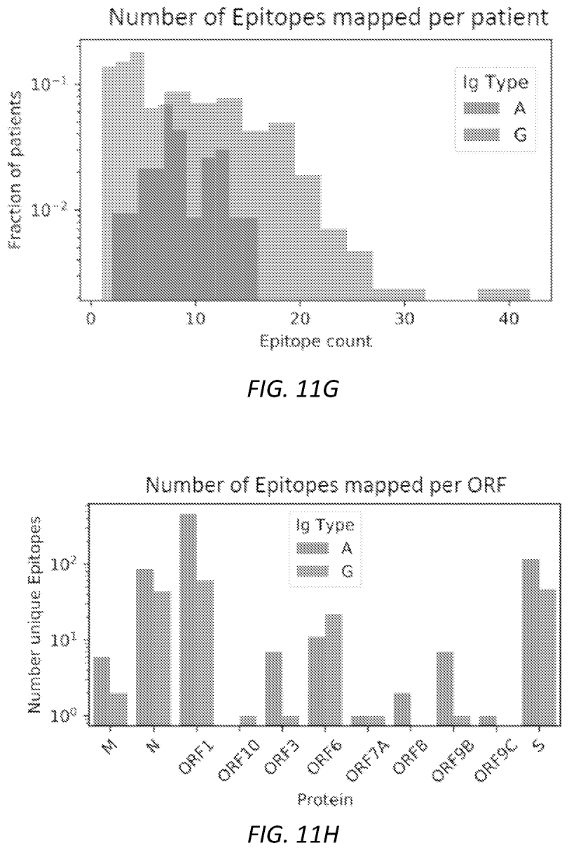

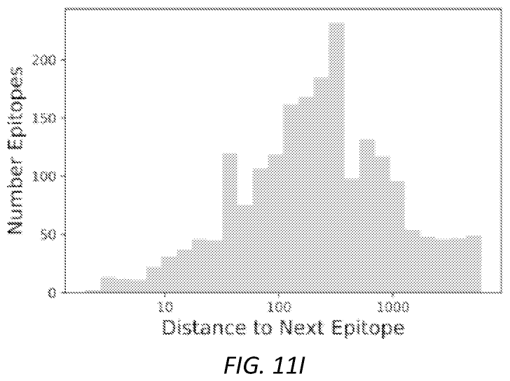

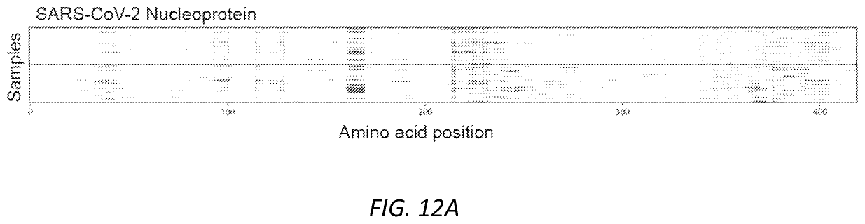

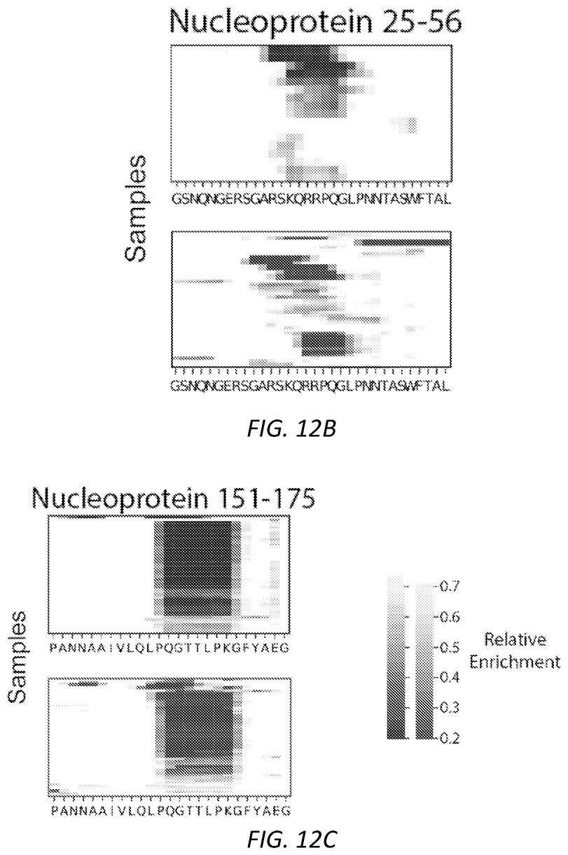

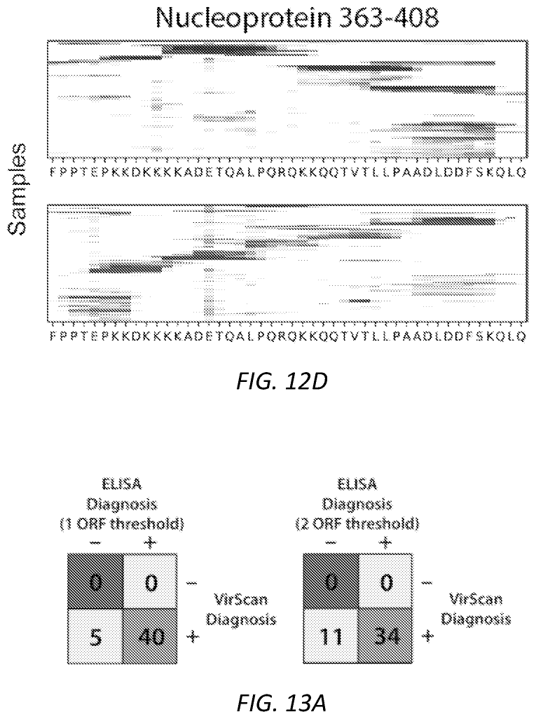

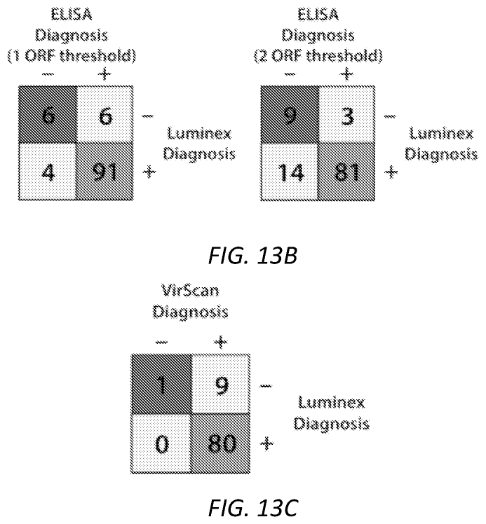

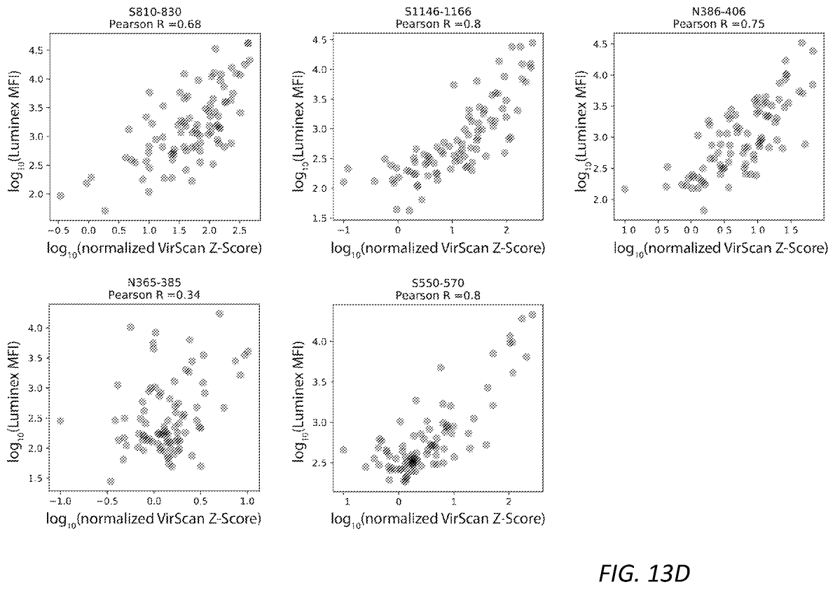

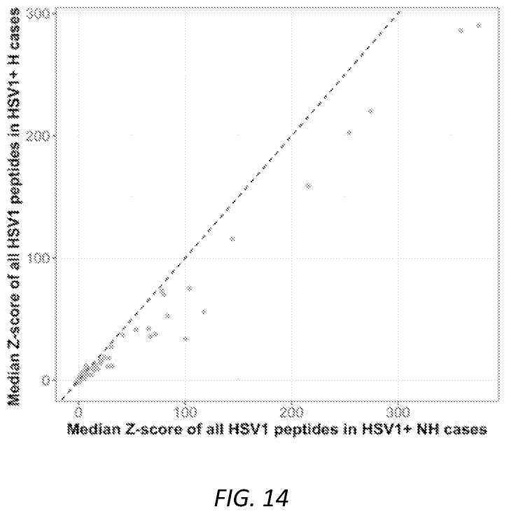

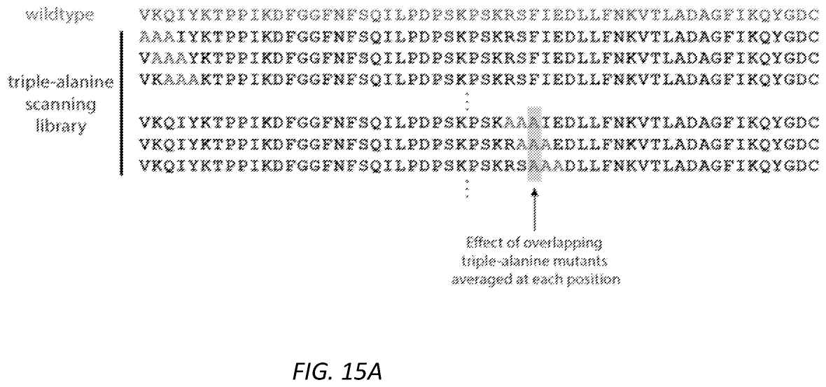

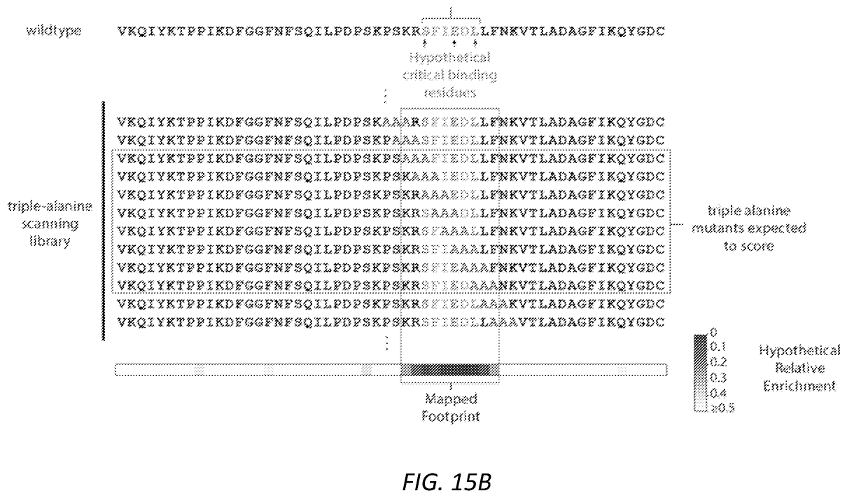

(A) Antibodies targeting SARS-CoV-2 proteins. Each column represents a unique patient sample and each row represents a SARS-CoV-2 protein. The color intensity in each cell of the heatmap indicates the number of 56-mer peptides as in FIG. 1D.
(B) Boxplots as in FIG. 1E illustrate the number of peptide hits from each of the indicated SARS-CoV-2 proteins detected in the IgG antibody response of COVID-19 patients and controls.
(C) Longitudinal analysis of the antibody response to SARS-CoV-2 for 23 patients with confirmed COVID-19. Days on which a sample was available for analysis are indicated with a black line. Each point represents the maximum antibody fold-change score per SARS-CoV-2 peptide in each sample, colored by protein target.

FIGS. 3A-C. IgG and IgA recognition of immunodominant regions in SARS-CoV-2 spike and nucleoprotein.

(A)Example response to S and N proteins from a single COVID-19 patient. The y-axis indicates the strength of enrichment (Z-Score, see methods) of each 56-mer or 20-mer peptide recognized by the IgG antibodies present in the serum sample.
(B) Common responses to S and N proteins across COVID-19 patients. The y-axis indicates the fraction of COVID-19 patient samples (n=348) enriching each 20-mer peptide with either IgG (top panel) or IgA (bottom panel) antibodies.
(C) Comparison of the IgA and IgG responses in individual COVID-19 patients. Each set of two rows represent the IgG and IgA antibody specificities of a single patient, with ten representative COVID-19 patients displayed. Numeric values indicate the degree of enrichment (Z-Score) of each peptide tiling across the S and N proteins.

FIGS. 4A-G. Machine learning models trained on VirScan data discriminate COVID-19-positive and negative individuals with very high sensitivity and specificity.

(A) Gradient boosting machine learning models were trained on IgG and IgA VirScan data from 232 COVID-19 patients and 190 pre-COVID-19 era controls. Separate models were created for the IgG and IgA data, and then a third model (Ensemble) was trained to combine the outputs of the first two.
(B) The plot shows the predicted probability that each sample is positive for COVID-19; true COVID-19 positive samples are shown as darker grey dots, and true COVID-19 negative samples are shown as lighter grey dots. The corresponding confusion matrix for each model is shown below.
(C-D) SHAP analysis to identify the most discriminatory peptides informing the models in (B). The chart in (C) summarizes the relative importance of the most discriminatory peptides increased among COVID-19 patients identified by the IgG and IgA gradient boosting models. The enrichment (log 2(Fold Change) of the normalized read counts in the sample IP versus in no-serum control reactions) of each of these peptides across all samples is shown in (D).
(E) Luminex assay using highly discriminatory SARS-CoV-2 peptides identifies IgG antibody responses in COVID-19 patients but rarely in pre-COVID-19 era controls. Each column represents a COVID-19 individual (n=163) or pre-COVID-19 era control (n=165); each row is a SARS-CoV-2-specific peptide. Peptides containing public epitopes from Rhinovirus A, EBV, and HIV-1 served as positive and negative controls. The color-scale indicates the median fluorescent intensity (MFI) signals after background subtraction.
(F) Receiver operating characteristic (ROC) curve for the Luminex assay predicting SARS-CoV-2 infection history, evaluated by 10× cross-validation. The light grey lines indicate the ROC curve for each test set, the dark line indicates the average, the grey region reflects±1std. dev. The average area under the curve (AUC) is shown.
(G) Left, the predicted probability that each sample is positive for COVID-19 by the Luminex model as in (B). The dashed line indicates the model threshold. Right, confusion matrix for the Luminex model.

FIGS. 5A-E. Correlates of COVID-19 disease severity.

(A) Differential recognition of peptides from SARS-CoV-2 nucleoprotein and spike between COVID-19 non-hospitalized patients (n=131), hospitalized patients (n=101), and pre-COVID-19 era negative controls. Each column represents a unique patient and each row represents a peptide tile; tiles are labelled by amino acid start and end position and may be duplicated for intervals for which amino acid sequence diversity are represented in the library. Color intensity represents the degree of enrichment (Z-score) of each peptide in IgG samples. Peptides exhibiting a significant increase in recognition by sera from hospitalized versus non-hospitalized patients are indicated with an asterisk, Kolmogorov-Smirnov test, Bonferroni-corrected p-value thresholds of 0.001 for S and 0.0025 for N).
(B) SARS-CoV-2 Luminex assay identifies stronger IgG responses in hospitalized COVID-19 patients than in non-hospitalized COVID-19 patients. Each column represents either a non-hospitalized (n=32) or hospitalized (n=32) COVID-19+ patient or a pre-COVID-19 era control (n=32); each row represents a peptide in the Luminex assay. The color-scale indicates the median fluorescent intensity (MFI) signals after background subtraction.
(C) All peptides in the VirScan library are plotted by the fraction of non-hospitalized (x-axis) and hospitalized COVID-19 patient IgG samples (y-axis) in which they are recognized. A Z-score threshold of 3.5 was used as an enrichment cutoff to count a peptide as positive. Peptides that exhibit statistically significant associations with hospitalization status are colored by virus of origin (Fisher's exact test, Bonferroni-corrected p-value threshold of 8.52×10-7). All peptides that do not exhibit significant association with hospitalization status are shown in grey. The significant peptides shown are collapsed for high sequence identity.
(D) All peptides derived from CMV present in the VirScan library are plotted by median Z-score for the non-hospitalized (x-axis) and hospitalized COVID-19 patients (y-axis). The line y=x is shown as a dotted line.
(E) Reduced recognition of mild-associated antigens with age. The histogram shows the relative recognition in healthy donors at age 58 compared to age 42 for each unique antigen that was more strongly recognized by antibodies in non-hospitalized than hospitalized COVID-19 patients.

FIGS. 6A-D. Cross-reactive epitopes among human coronaviruses.

(A)Bar graphs depicting the average number of 56-mer peptides derived from SARS-CoV-2, SARS-CoV, and each of the 4 common HCoVs that are significantly enriched per sample (IgG IP). Error bars represent the 95% confidence interval.
(B) Analysis of cross-reactive epitopes for HCoV S proteins. The upper plot shows the similarity of each region of the SARS-CoV-2 S protein to the corresponding region in the four common HCoVs (see Methods). The frequency of peptide recognition is shown in the bottom two plots. Peptides from each virus are indicated by the colored lines: the length of each line along the x-axis indicates the corresponding region of the SARS-CoV-2 S protein covered by each peptide according to a pairwise protein alignment, and the height of each line corresponds to the fraction of samples in which that peptide scored in either the IgG or IgA IPs. The epitopes mapped in (C) and (D) are highlighted in pink.
(C,D) Mapping of recurrently recognized SARS-CoV-2 S IgG (C) and IgA (D) epitopes by triple-alanine scanning mutagenesis. Each plot represents a 20 amino acid region of the SARS-CoV-2 S protein within the regions highlighted in (B). Each column of the heatmap corresponds to an amino acid position, and each row represents a sample. The color intensity indicates the average enrichment of 56-mer peptides containing an alanine mutation at that site relative to the median enrichment of all mutants of that 56-mer in each sample. COVID-19 patients with a minimum relative enrichment below 0.6 in the specified window are shown. The amino acid sequence across each region of SARS-CoV-2 S, as well as an alignment of the corresponding sequences in the common HCoVs, is shown below each heatmap. Shown are

S 551-570:
(SEQ ID NO: 1156)
VLTESNKKFLPFQQFGRDIA;
S 766-785:
(SEQ ID NO: 1157)
ALTGIAVEQDKNTQEVFAQV;
S 811-830:
(SEQ ID NO: 1158)
KPSKRSFIEDLLFNKVTLAD;
S1144-1163:
(SEQ ID NO: 1159)
LDSFKEELDKYFKNHTSPD.

FIGS. 7A-H: High-resolution mapping of SARS-CoV-2 epitopes.

(A)Mapping of antibody epitopes in the SARS-CoV-2 S protein using triple-alanine scanning mutagenesis. Each column of the heatmap corresponds to an amino acid position, and each row represents a COVID-19+ patient. The color intensity indicates the average enrichment of three triple-alanine mutant 56-mer peptides containing an alanine mutation at that site, relative to the median enrichment of all mutants of that 56-mer. The upper panel shows the fraction of samples that recognized each region of S as mapped by the IgA 56mer versus the IgA and IgG triple-alanine scanning.
(B-C) Detailed plot of triple-alanine scanning mutagenesis in (A) to show the epitope complexity within two regions: S 766-835 (B) and S 406-520 (C). The amino acid sequence at each position is shown on the x-axis. In (B), the fusion peptide and predicted S2′ cleavage site are indicated below the sequence (27, 28); in (C) the unique epitopes identified by the HMM and clustering algorithms are depicted by colored bars. The black dots correspond to ACE2 contact residues in the crystal structure of the RBD receptor complex (6MOJ) (29). Epitopes in regions E9 and E10 were not picked up by the HMM classifier because of their short length; however, these regions scored in multiple samples and correspond to accessible regions in the crystal structure, suggesting they may be true epitopes. Shown are

S 766-835:
(SEQ ID NO: 1160)
ALTGIAVEQDKNTQEVFAQVKQIYK
TPPIKDFGGFNFSQILPDPSKPSKRSFIEDLLFNKVTLADAGFIK;
S406-520:
(SEQ ID NO: 1161)
EVRQIAPGQTGKIADYNYKLPDDFTGCVIAWNSNNLDSKVGGNYNYLYRL
FRKSNLKPFERDISTEIYQAGSTPCNGVEGFNCYFPLQSYGFQPTNGVGY
QPYRVVVLSFELLHA.

(D) Cryo-electron microscopy (cryo-EM) structure of the partially-open SARS-CoV-2 spike trimer (6VSB) (30) highlighting the locations of the antibody epitopes mapped by triple-alanine scanning mutagenesis. The three spike monomers are depicted for the two closed and single open-conformation monomers respectively. The RBD of the open monomer is show in light grey. Three of the RBD epitopes from (C) that overlap ACE2 contact residues and are resolved in the cryo-EM structure (E2, E5, E6) are highlighted. The locations of additional public epitopes that were mapped in at least 10 samples across the IgG and IgA experiments are depicted.
(E-H) The locations of four of the epitope footprints mapped in (C) are shown in relation to the RBD-ACE2 binding interface. The upper image for each figure shows the structure (6MOJ) of SARS-2-CoV-2 RBD in complex with ACE2 (cyan). The E2, E5, E6 and E8 epitopes are highlighted. Below each image is the sequence alignment of the regions of the SARS-CoV-2 and the SARS-CoV S proteins encompassing each epitope. The bars indicate each epitope, the black dots indicate residues that directly interact with ACE2 in the crystal structure, and the shaded residues indicate conservation between SARS-CoV-2 and SARS-CoV. Shown are

S 412-431:
(SEQ ID NO: 1162)
PGQTGKIADYNYKLPD DFTG;
S 432-451:
(SEQ ID NO: 1163)
CVIAWNSNNLDSKVGGNYNY;
S 446-465:
(SEQ ID NO: 1164)
GGNYNYLYRLFRKSNLKPFE;
S 475-494:
(SEQ ID NO: 1165)
AGSTPCNGVEGFNCYFPLQS.

FIGS. 8A-C. Identification of antibody epitopes using a Hidden-Markov model (HMM).

(A) Alanine scanning mutagenesis data and the corresponding epitopes mapped in the HMM output for the full-length SARS-CoV-2 spike RBD (S334-528). Each column of the heatmap corresponds to an amino acid position, and each row represents a COVID-19+ sample. The second and fourth heatmaps from the top show the alanine-scanning data. The color intensity indicates the average enrichment of triple-alanine mutant 56-mer peptides containing an alanine mutation at that site, relative to the median enrichment of all mutants of that 56-mer in each sample. The first and third plots show the output of the HMM classification. Each position is classified as “no response”, “mapped epitope”, or “mapped critical region”. The top two heatmaps show the data for the IgG IPs; the bottom shows the data for the IgA IPs. Data is shown for samples with a minimum relative enrichment of 0.6 in the window. The row order is the same for each of the heatmaps. Unique epitopes mapped by the hierarchical clustering are shown below the sequence. Epitopes 9 and 10 were not identified by the HMM but the fact that these regions score in multiple samples and are located in surface exposed regions of the RBD structure suggest that they may be true epitopes. Black dots indicate residues that contact ACE2 in the crystal structure of the receptor-bound RBD (6V0J). Shown is

S 334-527
(SEQ ID NO: 1166)
NLCPFGEVFNATRFASVYAWNRKRISNCVADYSVLYNSASFSTFKCYGVS
PTKLNDLCFTNVYADSFVIRGDEVRQIAPGQTGKIADYNYKLPDDFTGCV
IAWNSNNLDSKVGGNYNYLYRLFRKSNLKPFERDISTEIYQAGSTPCNGV
EGFNCYFPLQSYGFQPTNGVGYQPYRVVVLSFELLHAPATVCGP.

(B-C) Results of the HMM classification and the corresponding alanine scanning data as in (A) for SARS-CoV-2 N25-56

(B; shown is N 25-56:
GSNQNGERSGARSKQRRPQGLPNNTASWFTAL; SEQ ID NO: 1167)
and
N 200-265 (C; shown is N200-264:
GSSRGTSPARMAGNGGDAALALLLLDRLNQLESKMSGKGQQQ
QGQTVTKKSAAEASKKPRQKRTA; SEQ ID NO: 1168).

FIGS. 9A-B. Concordance of positions of epitopes identified with triple-alanine scanning mutagenesis and with 56-mer and 20-mer peptide libraries.

(A) Comparison of the positions of epitopes mapped by the HMM classifier (using the triple-alanine mutagenesis data as input) and the positions of the 20-mer and 56-mer peptides enriched in COVID-19 patient samples. For each plot, the y-axis shows different IgA serum samples and the x-axis shows the amino acid position along ORF1. Each heatmap is on a binary scale. In the top heatmap, the dark color indicates epitopes mapped to each location along the length of ORF1 for each serum sample. The second and third plots show the positions of 20-mer and 56-mer peptides, respectively, that scored with a Z-score>3.5 for each sample.
(B) Fraction of COVID-19 patient IgA samples that recognize each position in ORF1 (top) and S (bottom) as mapped by the 56mer library and the HMM classifier.

FIGS. 10A-F. Clustering antibody footprints to identify unique epitopes.

(A-F) Heatmaps showing the alanine-scanning profile of epitopes within specific hotspot clusters. IgA epitopes identified by the HMM classifier were clustered based on their start and stop positions into “hotspot” clusters that represent overlapping sets of related antibody footprints. Each heatmap in (A-F) shows the alanine-scanning data for epitopes that clustered into a particular hotspot. The y-axis shows the amino acid position in the SARS-CoV-2 Spike protein. Independent samples are depicted along the x-axis. The color intensity represents the relative enrichment for each residue, as in FIG. 8A. The epitopes were further clustered to identify the number of unique epitopes within each hotspot. The results of the hierarchical clustering are shown in the color-bar along the top of each plot. Each color represents a single “unique epitope” cluster.

FIGS. 11A-I. Summary statistics of SARS-CoV-2 epitopes. Epitopes mapped using the HMM classifier on alanine-scanning data from IgA, IgG or combined IPs from sera of 169 COVID-19 positive patients.

(A) The total number of epitopes, unique epitopes, and hotspots mapped for IgG, IgA, and IgG plus IgA (combined) samples.
(B) Number of hotspots mapped in each SARS-CoV-2 ORF; only ORFs with at least one hotspot are shown.
(C)Number of hotspots recognized per patient.
(D)Distribution of the number of patients that recognized each hotspot among the 169 COVID-19+ samples analyzed.
(E) Length distribution of the unique epitopes. Epitopes smaller than 5 amino acids were not considered in the analysis.
(F) Distribution of the number of patients that recognized each unique epitope among the 169 COVID-19+ samples analyzed.
(G)Distribution of the number of epitopes mapped per patient.
(H)Distribution of the number of epitopes mapped per ORF.
(I) Distribution of the linear amino acid distance between epitopes within each protein. This was calculated using the combined IgG and IgA data for each of the 169 COVID-19 patient samples.

FIGS. 12A-D. Mapping epitopes in the SARS-CoV-2 nucleoprotein (N) using triple-alanine scanning mutagenesis.

(A) Alanine scanning mutagenesis to map antibody epitopes in the SARS-CoV-2 N protein. Each column of the heatmap corresponds to an amino acid position, and each row represents a COVID-19-positive sample. The color intensity indicates the average enrichment of triple-alanine mutant 56-mer peptides containing an alanine mutation at that site, relative to the median enrichment of all mutants of that 56-mer in each sample. The top heatmap show shows the data for the IgG IPs; the bottom heatmap shows the data for IgA IPs.
(B-D) Detailed plot of alanine-scanning in (A) to show the epitope complexity within specified regions of the SARS-CoV-2 N protein

(B: N 25-56,
(SEQ ID NO: 1167)
GSNQNGERSGARSKQRRPQGLPNNTASWFTAL;
C: N151-175,
SEQ ID NO: 1169
PANNAAIVLQLPQGTTLPKGFYAEG,;
D: N 363-408,
(SEQ ID NO: 1170)
FPPTEPKKDKKKKADETQALPQRQKKQQTVTLLPAADLDDFSKQLQ)

for COVID19-positive samples with a minimum relative-enrichment below 0.55 in the specified window. The x-axis shows the amino acid sequence at each position.

FIGS. 13A-D. Comparison of VirScan, Luminex, and ELISA SARS-CoV-2 serological assays.

(A) Number of samples classified as positive for SARS-CoV-2 infection among the set of COVID-19 positive sera run on both the VirScan and the ELISA assays (n=45). The left panel shows the ELISA samples that scored above the 99% specificity threshold for at least one of the three single-antigen ELISAs (N, S, RBD). The right panel shows samples that scored for at least 2 of the three ELISAs. (B) Number of samples classified as positive for SARS-CoV-2 infection among the set of COVID-19 positive sera run on both the Luminex and the ELISA assays (n=107) as in (A). (C) Number of samples classified as positive for SARS-CoV-2 infection among the set of COVID-19 positive sera run on both VirScan and the Luminex assays (n=90). (D) Scatterplots showing the correlation between SARS-CoV-2 peptide seroreactivity in the VirScan and Luminex assays among the COVID-19 positive samples run on both assays (n=90). The y-axis shows the log-transformed Luminex MFI values. The x-axis shows the log of normalized VirScan Z-scores. The peptide N365-385 did not score well in VirScan, leading to a relatively weak correlation; however, the overlapping peptide N360-380 performed better in VirScan and showed greater correlation with the Luminex data (R=0.64).

FIG. 14. HSV-1 recognition in non-hospitalized vs hospitalized COVID-19 patient groups.

All HSV-1 peptides in the VirScan library are plotted by median Z-score for the non-hospitalized (x-axis) and hospitalized COVID-19 patients (y-axis). The line y=x is shown as a dotted line.

FIGS. 15A-B. Design and usage of the triple-alanine scanning mutagenesis library.

(A) The design of the triple-alanine scanning mutagenesis library. For each wildtype 56-mer peptide we designed a set of mutant peptides containing three consecutive alanine mutations. In the first mutant the first three amino acids were mutated to alanine, and for each consecutive mutant peptide the starting position of the alanine mutations was moved one residue toward the C-terminus. This is repeated along the entire length of the 56mer. The complete triple-alanine scanning library contains peptides encoding triple alanine substitutions tiling across the entire length of every wildtype SARS-CoV-2 56mer. The relative enrichment at each position was calculated as the mean of the three peptides containing a mutation at that position (indicated in grey). Shown are SEQ ID NOs. 1171-1177, in order.
(B). Antibody footprint mapping by triple-alanine scanning. A hypothetical antibody epitope and its hypothetical critical antibody binding residues are shown. The top sequence shows the wild-type 56mer, the sequences in the middle represent the set of triple-alanine mutant peptides tiling across the region containing the hypothetical epitope. The mutant peptides expected to score with reduced relative enrichments based on this hypothetical epitope are indicated. The heatmap on the bottom depicts hypothetical relative enrichment values for this 56mer given the indicated epitope. Because each mutant peptide encodes three consecutive alanine substitutions, the antibody footprint mapped according to the relative enrichment values (bottom) begins two residues prior to the first critical binding residue and ends two residues after the last critical residue. Shown are SEQ ID NOs. 1171 and 1178-1189, in order

### DETAILED DESCRIPTION

The clinical course of Coronavirus Disease 19 (COVID-19)—the disease resulting from SARS-CoV-2 infection—is notable for its extreme variability: while some individuals remain entirely asymptomatic, others experience fever, anosmia, diarrhea, severe respiratory distress, pneumonia, cardiac arrhythmia, blood clotting disorders, liver and kidney distress, enhanced cytokine release and, in a small percentage of cases, death (6). Understanding the factors influencing this spectrum of outcomes is therefore an intense area of research. Disease severity is correlated with advanced age, sex, ethnicity, socio-economic status, and co-morbidities including diabetes, cardiovascular disease, chronic lung disease, obesity, and reduced immune function (6). Additional relevant factors are likely to include the inoculum of virus at infection, the individual's genetic background and viral exposure history. The complex interplay of these elements also determines how individuals respond to therapies aimed at mitigating disease severity. One of the key aspects of human physiology that integrates many of these components is the functionality of the immune system. The immune system is the primary defense against the virus. The outcome of any individual's encounter with the virus is thus dependent on the functionality of the immune system, which depends on a number of factors including genetics, stress, age and the history of prior exposures. Detailed knowledge of the immune response to SARS-CoV-2 could improve our understanding of diverse outcomes and inform the development of improved diagnostics vaccines, and antibody-based therapies.

The first SARS-CoV-2 infection was first reported from Wuhan, China, in December 2019. The genome of the virus has been determined. The genome comprises or flab encoding or flab polyproteins, genes encoding structural proteins including surface (S), envelope (E), membrane (M), and nucleocapsid N proteins, and 6 accessory proteins, encoded by ORF3a, ORF6, ORF7a, ORF7b, and ORF8 genes (Khailany et al., Gene Rep. 2020 June; 19: 100682; Wang et al., J Med Virol. 2020 June; 92(6):667-674. Epub 2020 Mar 20); genomic information is available at the NCBI Severe acute respiratory syndrome coronavirus 2 database (nhc.gov.cn/jkj/s7915/202001/e4e2d5e6f01147e0a8df3f6701d49f33.shtml) and NGDC Genome Warehouse (bigd.big.ac.cn/gwh/).

Here we describe a detailed analysis of the humoral response in COVID-19 patients using VirScan, a programmable phage-display immunoprecipitation and sequencing (PhIP-Seq) technology we developed previously to explore antiviral antibody responses across the human virome (7-9). Cohorts of COVID-19 patients, pre-COVID-19 era negative controls, and longitudinal samples from COVID-19 patients over the course of infection enabled us to characterize SARS-CoV-2-specific antibodies as well as cross-reacting antibodies. These cross-reacting antibodies can confound serological diagnosis of COVID-19. VirScan can also identify virus-specific epitopes that allow one to discriminate between different coronavirus infections. We developed a machine learning model trained on VirScan data that detects SARS-CoV-2 exposure history with extremely high sensitivity and specificity, and we employed the most differentially-recognized SARS-CoV-2 peptides between COVID-19+ patients and pre-COVID-19 era controls in a Luminex assay to produce a fast and reliable diagnostic. We compared the anti-SARS-CoV-2 antibody response and virome-wide exposure history in COVID-19 patients who did or did not require hospitalization in order to identify correlates of disease severity. Finally, we used alanine-scanning mutagenesis coupled with VirScan to map epitopes across the SARS-CoV-2 proteome to single amino acid resolution; over a dozen of these epitopes are located in the receptor binding (RBD) of the spike, and 10 of these are located on the receptor binding motif (RBM) that directly contacts ACE2 and are likely targets of neutralizing antibodies.

Using VirScan, we were able to map a total of over 3,000 SARS-CoV-2 epitopes, including 813 unique epitopes, with unprecedented resolution. Further, we were able to investigate their cross-reactivity with other human and bat coronavirus epitopes.

### Identification of SARS-CoV-2 Epitopes Recognized by COVID Patients

Antibody profiling of sera from 232 COVID-19 patients and 190 pre-COVID-19 era controls revealed robust antibody recognition of peptides encoded by SARS-CoV-2 among COVID-19 patients compared with controls. These were primarily directed against the S and N proteins, with significant cross-reactivity to SARS-CoV, and milder cross-reactivity with the more distantly related MERS-CoV and the seasonal Human coronaviruses (HCoVs). Cross-reactive responses to SARS-CoV-2 ORF1 were frequently detected in pre-COVID-19 era controls, suggesting that these result from antibodies induced by other pathogens.

Examination of the response at the epitope level revealed the existence of public epitopes targeted by many COVID-19 patients. Using a combination of both 56-mer and 20-mer peptide tiles, together with the alanine scanning mutagenesis library, we mapped epitopes within SARS-CoV-2 at unprecedented resolution.

At the population level, most SARS-CoV-2 epitopes were recognized by both IgA and IgG antibodies. We found individuals often exhibited a “checkerboard” pattern, utilizing either IgG or IgA antibodies against a given epitope. This suggests that a given IgM clone often evolves into either an IgG or an IgA antibody, potentially influenced by local signals, and that, within an individual, there may often be a largely monoclonal response to a given epitope.

Examination of the humoral response to SARS-CoV-2 at the epitope level using the triple-alanine scanning mutagenesis library revealed 145 epitopes in S, 116 in N, and 562 across the remainder of the SARS-CoV-2 proteome (FIGS. 11A-H). These epitopes ranged from private to highly public, with one public epitope cluster being recognized by 79% of COVID-19 patients (the S 811-830 region, see FIG. 6 C/D third panel from the left). Triple-alanine scanning mutagenesis showed highly conserved antibody footprints for some epitope clusters and diverse antibody footprints for others, indicating varying levels of conservation at the antibody-epitope interface among individuals (FIGS. 10A-F). Peptides containing public epitopes could be used to isolate and clone antibodies from B-cells bearing antigen-specific BCRs. If these antibodies are found to lack protective effects or have deleterious effects, these regions, e.g. S 811-830, could be mutated in future vaccines to divert the immunological response to other regions of S that might have more protective effects. Epitopes also varied in cross-reactivity, which can be explained by the presence or absence of sequence conservation between seasonal HCoVs and SARS-CoV-2 at these regions. Antibodies against several conserved epitopes in HCoVs seemed to be anamnestically boosted in COVID-19 patients. Altogether these data help explain why many serological assays for SARS-CoV-2 produce false positives, and should be taken as a cautionary note for those trying to develop such assays.

### Methods of Diagnosis: SARS-CoV-2 Signature Peptides for Detecting Seroconversion

Using machine learning models trained on VirScan data, we developed a classifier that predicts SARS-CoV-2 exposure history with 99% sensitivity and 98% specificity. We identified peptides frequently and specifically recognized by COVID-19 patients and used these to create a Luminex assay that predicted SARS-CoV-2 exposure with 90% sensitivity and 95% specificity. Remarkably, the Luminex assay only required three peptides to obtain performance comparable to full antigen ELISAs. This highlights the utility of VirScan-based serological profiling in the development of rapid and efficient diagnostic assays based on public epitopes.

The compositions and methods described herein can also be used to detect the presence of antibodies in a sample from a subject to determine whether a subject has been infected with SARS-CoV-2; the presence of antibodies that bind the epitopes indicates that the subject has had an infection with SARS-CoV-2. Thus provided herein are methods and kits for use in determining whether a subject has, or has had, SARS-CoV-2.

The methods can include providing a sample from a subject, e.g., a sample comprising whole blood, serum, saliva or plasma, that comprises antibodies from a subject. In some embodiments, the subject is suspected to have, or to have been exposed to, SARS-CoV-2. In some embodiments, the subject is a mammal, e.g., a human or non-human veterinary subject, e.g., a cat, dog, ferret, Syrian hamster, tiger, lion, mink, bat, or pangolin.

The sample is contacted with one or more, e.g., 1, 2, 3, 4, 5, 8, 10, 12, 15, 20, 25, 30, 50, 75, 80, 100, 150, 200, 250, 300, 350, 400, 500, 600, 700, 800, or more, peptides comprising at least 4, e.g., 5, 6, 7, 8, 9, 10, 11, 12, 13, 14, 15, 16, 17, 18, 19, 20 or more, consecutive amino acids from a SARS-CoV-2 epitope sequence shown herein, e.g., in Table 1, Table 3, and/or Table 4 or SEQ ID NOs:13-1170, and binding of antibodies in the sample to the peptides (e.g., formation of antibody-epitope complexes) is detected. The presence of antibodies bound to the peptides indicates the presence of the virus in the subject. Preferably, the peptides are at least 4, e.g., 5, 6, 7, 8, 9, 10, 11, 12, 13, 14, 15, 16, 17, 18, 19, 20, up to 20, 25, 30, 35, 40, 45, 50, 55, 60, 65, 70, 75, 80, 85, 90, 95, or 100 amino acids long, with each number being an endpoint for a range of sizes.

The methods can include a purification step, in which un-bound epitope peptides, un-bound antibodies, or both, are removed from the sample, or in which bound complexes are isolated from the sample, before detection is performed.

Detection of binding of antibodies to the epitopes can be performed using methods known in the art. In some embodiments, multiplex immunoassays are used, e.g., assays in which the peptides are immobilized on beads (e.g., Luminex (e.g., x eAP@ Assay) Abcam's FirePlex®, or Cytometric Bead Array (CBA) from BD Biosciences) or on a surface (e.g., RayBiotech's Quantibody® glass chip-based array), wherein each species of peptide (i.e., a species is a set of peptides that all share the same sequence) is individually identifiable, e.g., each peptide species is associated with a different label. See, e.g., Fu et al., Clin Chem. 2010 February;56(2):314-8. In some embodiments, split enzymes reconstitution or protein-fragment complementation assays (PCAs) (e.g., as described in Shekhawat and Ghosh, Curr Opin Chem Biol. 2011 December; 15(6): 789-797; Sierecki, ACS Cent. Sci. 2019, 5, 11, 1744-1746; Jones et al., ACS Cent. Sci. 2019, 5, 11, 1768-1776; Li et al., J. Proteome Res. 2019, 18, 8, 2987-2998) or single molecule detection methods (e.g., single molecule array (SIMOA) can be used (Mora et al., AAPS J. 2014 November; 16(6): 1175-1184; Costa et al., PLoS One. 2018; 13(3): e0193670; Chang et al., J Immunol Methods. 2012 Apr. 30; 378(1-2): 102-115; Libre et al., J Vis Exp. 2018; (136): 57421).

In some embodiments, the methods can include quantitating a level of antibodies in a sample, e.g., by detecting a level of antibody/epitope complexes formed.

In some embodiments, the presence and/or level of antibodies that bind to one or more peptide epitopes is comparable to or above the presence and/or level of binding in the disease reference, and the subject has one or more symptoms associated with COVID-19, then the subject has COVID-19 (i.e., a positive result) or had it in the past. In some embodiments, the subject has no overt signs or symptoms of COVID-19, but the presence and/or level of binding to one or more of the peptide epitopes is comparable to or above the presence and/or level of binding in the disease reference, then the subject has or had COVID-19 (i.e., a positive result). In some embodiments, once it has been determined that a person has COVID-19, then a treatment, e.g., as known in the art or as described herein, can be administered.

The methods can also include contacting the samples with peptide epitopes specific for other pathogens, e.g., other viruses, e.g., Severe acute respiratory syndrome coronavirus (SARS-CoV, identified in 2003); cytomegalovirus (CMV); Rhinoviruses A and/or B; Influenza A and/or B, Enteroviruses A, B and/or C; HIV-1, Epstein-Barr virus (EBV), cytomegalovirus (CMV), and Herpes Simplex Virus 1 (HSV-1), or other Human coronaviruses (HCoVs) (e.g., MERS, SARS and other coronaviruses, including alphacoronaviruses (HCoV-229E and HCoV-NL63) and betacoronaviruses (HCoV-HKU1, HCoV-OC43, MERS-CoV, SARS-CoV, SARS-CoV-2)). See, e.g., U.S. Pat. No. 10,768,181. In some embodiments, epitope mapping is performed for the HCoVs to identify HCoV specific epitopes, and these are integrated into the methods described herein to reduce false positives, i.e., some response to SARS-CoV-2 peptides and a very strong response to peptides from another HCoV indicates the presence of an active high-titer response to the HCoV and that the SARS-CoV-2 response is a cross-reaction (and therefore a false positive for SARS-CoV-2).

In these methods, a single sample can be used to detect infection with a plurality of viruses.

In some embodiments, the reference level is the limit of detection of the assay, wherein detection of any level of antibodies that bind to one or more peptide epitopes is considered a positive result. In some embodiments, a reference value is chosen. Suitable reference values can be determined using methods known in the art, e.g., using standard clinical trial methodology and statistical analysis. The reference values can have any relevant form. In some cases, the reference comprises a predetermined value for a meaningful level of binding, e.g., a control reference level that represents a normal level of antibodies, e.g., a level in a subject who was previously exposed to a different coronavirus, and/or a disease reference that represents a level of binding associated with infection, e.g., a level in a subject who has or had a SARS-CoV-2 infection. In some embodiments, the reference value is a combined score that integrates antibody binding to multiple epitopes, determined using a machine learning model.

The predetermined level can be a single cut-off (threshold) value, such as a median or mean, or a level that defines the boundaries of an upper or lower quartile, tertile, or other segment of a clinical trial population that is determined to be statistically different from the other segments. It can be a range of cut-off (or threshold) values, such as a confidence interval. It can be established based upon comparative groups, such as where association with risk of developing disease or presence of disease in one defined group is a fold higher, or lower, (e.g., approximately 2-fold, 4-fold, 8-fold, 16-fold or more) than the risk or presence of disease in another defined group. It can be a range, for example, where a population of subjects (e.g., control subjects) is divided equally (or unequally) into groups, such as a low-risk group, a medium-risk group and a high-risk group, or into quartiles, the lowest quartile being subjects with the lowest risk and the highest quartile being subjects with the highest risk, or into n-quantiles (i.e., n regularly spaced intervals) the lowest of the n-quantiles being subjects with the lowest risk and the highest of the n-quantiles being subjects with the highest risk.

In some embodiments, the predetermined level is a level or occurrence in the same subject, e.g., at a different time point, e.g., an earlier time point.

Subjects associated with predetermined values are typically referred to as reference subjects. For example, in some embodiments, a control reference subject does not have COVID-19 and/or has not been exposed to COVID-19.

A disease reference subject is one who has (or has had) COVID-19.

Thus, in some cases the level of antibody binding to an epitope described herein in a subject being less than or equal to a reference level of binding is indicative of a clinical status (e.g., indicative of absence of infection). In other cases the level of binding in a subject being greater than or equal to the reference level of binding is indicative of the presence of infection or a past infection. In some embodiments, the amount by which the level in the subject is the less than the reference level is sufficient to distinguish a subject from a control subject, and optionally is a statistically significantly less than the level in a control subject. In cases where the level of binding in a subject being equal to the reference level of binding, the “being equal” refers to being approximately equal (e.g., not statistically different).

The predetermined value can depend upon the particular population of subjects (e.g., human subjects) selected. Accordingly, the predetermined values selected may take into account the category (e.g., sex, age, health, risk, presence of other diseases) in which a subject (e.g., human subject) falls. Appropriate ranges and categories can be selected with no more than routine experimentation by those of ordinary skill in the art.

In characterizing likelihood, or risk, numerous predetermined values can be established.

In some embodiments, once a subject has been diagnosed with COVID-19 using a method described herein, a treatment can be administered. Treatments for COVID-19 are known in the art and include quarantining the subject, administration of an antiviral medication (e.g., remdesivir, Favipiravir, MK-4482; Lopinavir and ritonavir); Recombinant ACE-2; Ivermectin; Oleandrin; bradykinin signaling blockers (e.g., icatibant, ecallantide lanadelumab); vasopressors; Vitamin D; steroids (e.g., Dexamethasone); Cytokine Inhibitors; Convalescent plasma/antibodies; Interferons; ventilation/respiratory support devices; Anticoagulants. Alternatively, if the active infection is past but it is found that infections can predispose an individual with other ailments such as heart or kidney disease or a predisposition for future strokes, they could be monitored more closely for those diseases later in their lives.

### Correlates of Severity in COVID-19 Patients

An important goal is to uncover serological elements that either correlate with, or predict the severity of, COVID-19 disease. To this end, we compared cohorts of COVID-19 patients who had (H) or had not (NH) required hospitalization. Using both VirScan and the COVID-19 Luminex assay, we noticed a striking and somewhat counterintuitive increase in recognition of peptides derived from the SARS-CoV-2 S and N proteins among the H group, with more extensive epitope spreading. Whether this is a cause or a consequence of severe disease is not clear. Individuals whose innate and adaptive immune responses are not able to quell the infection early may experience a higher viral antigen load, a prolonged period of antibody evolution and epitope spreading. Consequently, these patients might develop stronger and broader antibody responses to SARS-CoV-2 and could be more likely to have hyperinflammatory reactions such as cytokine storms that increase the probability of hospitalization. We noticed that hospitalized males had stronger antibody responses to SARS-CoV-2 than hospitalized females. This may indicate that males in this group are less able to control the virus soon after infection and is consistent with reported differences in disease outcomes for males and females. The presence of antibodies that bind to these epitopes (in the SARS-CoV-2 nucleoprotein) can be used to identify subjects who are likely to have a more severe response.

VirScan also allowed us to examine viral exposure history, which revealed two striking correlations. First, the seroprevalence of CMV and HSV-1 was much greater in the H group compared to the NH group. The demographic differences in our relatively small cohort of H versus NH COVID-19 patients make it impossible for us to determine with certainty if CMV or HSV-1 infection impacts disease outcome or is simply associated with other covariates such as age, race and socioeconomic status. While CMV prevalence does slightly increase with age after 40 (31), its prevalence also differs greatly among ethnic and socioeconomic groups (32). CMV is a herpes virus that exhibits latency within the host and is known to have a profound impact on the immune system; it can skew the naive T-cell repertoire (33), decrease T and B cell function (34), and is associated with higher systemic levels of inflammatory mediators (35, 36). CMV latency also results in inversion of CD4+ and CD8+ T-cell numbers, poor proliferation response of T-cells, low B cell numbers, and has been associated with increased mortality of people over 65 years of age (37). CMV's effects on the immune system could potentially impact the response to SARS-CoV-2 infection in an older population. The effects of CMV on the immune system could impact COVID-19 outcomes.

The second striking correlation we observed was a significant decrease in the levels of antibodies targeting ubiquitous viruses such as Rhinoviruses, Enteroviruses, and Influenza viruses, in COVID-19 H patients compared with NH patients. When we examined only the CMV+ or HSV-1+ individuals in the two groups, we found that the strength of the antibody response to CMV and HSV-1 peptides was also reduced in the H group. We examined the effects of age on viral antibody levels in a pre-COVID-19 era cohort and found a diminution with age in the antibody response against viral peptides differentially recognized between the H and NH groups, consistent with previous studies on the effects of aging on the immune system (38). This inferred reduced immunity during aging could impact the severity of COVID-19 outcomes. Thus, the presence of decreased levels antibodies to CMV and/or HSV-1 epitopes can be used to identify subjects who are likely to have a more severe response.

In correlative analyses such as these, it is difficult to draw strong conclusions about causality given the demographical differences in the NH vs H groups. The NH group is younger, has a higher percentage of Caucasian individuals, and has more females (average age 42, 66% female) versus H (average age 58, 42% female). This is consistent with the well-documented age, race and sex differences among the more severely affected individuals (25, 26). However, even if age and other demographic factors are covariates, the reduction in immune function with age and CMV status described here could still impact severity of infection.

### Methods of Improving Vaccines

The present methods can include identifying public and/or immunodominant epitopes that are the targets of non-protective antibodies and generating vaccines in which these epitopes are disrupted or removed, or delivering vaccines together with antibodies against these epitopes, with the goal of reducing the production of non-protective antibodies against these epitopes and boosting the production of more protective antibodies.

As demonstrated herein, certain epitopes are more likely to be associated with neutralizing antibodies, while others may be more likely to generate immunodominant non-neutralising antibodies. It is believed that the epitopes within the receptor binding domain (see Table 4, SEQ ID NOs. 1036-1050, are believed to be associated with neutralizing antibodies. Thus, provided herein are methods for generating vaccines that are less likely to generate antibodies that bind to non-neutralizing epitopes. The methods can include administering to a mammal, e.g., a rodent (e.g., rat or mouse), rabbit, ferret, hamster, or a human or non-human primate, a composition comprising a mutated version of SARS-CoV-2 proteins, or nucleic acids encoding mutated versions of SARS-CoV-2 proteins, wherein the mutated versions comprises one or more mutations that disrupt one or more non-neutralizing epitopes as described herein, allowing sufficient time for an immune response to occur in the mammal, and obtaining antibodies from the mammal, then screening the antibodies and identifying mutant viral proteins that produce higher titers of neutralizing antibodies, but produce fewer, or do not produce any, antibodies to non-neutralizing epitopes. These methods can be used to identify and select mutations that reduce the generation of antibodies to non-neutralizing epitopes. In some embodiments, the methods are used to reduce the possibility of inducing or increasing risk of post-viral syndrome in subjects vaccinated with an antibody vaccine.

Also provided herein are mutated versions of SARS-CoV-2 proteins, or nucleic acids encoding mutated versions of SARS-CoV-2 proteins, wherein the mutations remove one or more of the non-neutralizing epitopes described herein. The mutated nucleic acids or proteins can be used to generate vaccine compositions, wherein administration of the composition would result in generation of antibodies to neutralizing epitopes but with fewer antibodies to non-neutralizing epitopes.

Also provided herein are methods that can be used for identifying those antibodies that are most likely to induce a protective immune response. The methods include providing a sample comprising (or expected to comprise) antibodies to SARS-CoV-2 from a subject who has been administered a vaccine to SARS-CoV-2; contacting the sample with one or more peptides as described herein, and detecting binding of the sample to the peptides. Vaccines that produce antibodies that bind to epitopes associated with neutralizing antibodies are likely to induce a protective response, and can be selected for further development, while vaccines that produce an antibody response to non-neutralizing epitopes, or to both neutralizing and non-neutralizing epitiopes, may be less desirable.

The present methods can also include isolating and identifying protective and non-protective antibodies from SARS-CoV-2 patient samples. The methods can include providing a sample including B cells or antibodies to SARS-CoV-2, e.g., obtained from a human subject infected with SARS-CoV-2; contacting the sample with one or more peptides described herein, and isolating B cells or antibodies that bind these peptides. The antibodies may then be tested for protective function via neutralizing activity or Fc-mediated effector function.

The methods can further include formulating the antibodies that bind neutralizing epitopes for administration as a therapeutic, and administering the antibodies that bind neutralizing epitopes to a subject, e.g., a subject who has or is at risk of contracting an infection with SARS-CoV-2. In some embodiments, the methods include detecting binding to antibodies that bind non-neutralizing epitopes, and optionally removing antibodies that bind non-neutralizing epitopes. The methods can also include isolating antibodies that bind to the non-neutralizing epitopes, and adding those antibodies to non-neutralizing epitopes to a vaccine, such that the non-neutralizing epitopes are covered (not accessible), and thus can be eliminated from the response because they are covered and are not capable of eliciting an antibody response.

Also provided herein are methods for generating antibodies to SARS-CoV-2. Methods for making suitable antibodies are known in the art. One or more of the peptides listed in Tables 1, 3, and/or 4, e.g., SEQ ID NO: 1036-1050, can be used as an immunogen, or can be used to identify antibodies made with other immunogens, e.g., cells, membrane preparations, and the like, e.g., E rosette positive purified normal human peripheral T cells, as described in U.S. Pat. Nos. 4,361,549 and 4,654,210.

Methods for making monoclonal antibodies are known in the art. Basically, the process involves obtaining antibody-secreting immune cells (lymphocytes) from the spleen of a mammal (e.g., mouse) that has been previously immunized with the antigen of interest (e.g., a neutralizing epitope antigen) either in vivo or in vitro. The antibody-secreting lymphocytes are then fused with myeloma cells or transformed cells that are capable of replicating indefinitely in cell culture, thereby producing an immortal, immunoglobulin-secreting cell line. The resulting fused cells, or hybridomas, are cultured, and the resulting colonies screened for the production of the desired monoclonal antibodies. Colonies producing such antibodies are cloned, and grown either in vivo or in vitro to produce large quantities of antibody. A description of the theoretical basis and practical methodology of fusing such cells is set forth in Kohler and Milstein, Nature 256:495 (1975), which is hereby incorporated by reference.

Mammalian lymphocytes are immunized by in vivo immunization of the animal (e.g., a mouse) with a neutralizing epitope antigen. Such immunizations are repeated as necessary at intervals of up to several weeks to obtain a sufficient titer of antibodies. Following the last antigen boost, the animals are sacrificed and spleen cells removed.

Fusion with mammalian myeloma cells or other fusion partners capable of replicating indefinitely in cell culture is effected by known techniques, for example, using polyethylene glycol (“PEG”) or other fusing agents (See Milstein and Kohler, Eur. J. Immunol. 6:511 (1976), which is hereby incorporated by reference). This immortal cell line, which is preferably murine, but can also be derived from cells of other mammalian species, including but not limited to rats and humans, is selected to be deficient in enzymes necessary for the utilization of certain nutrients, to be capable of rapid growth, and to have good fusion capability. Many such cell lines are known to those skilled in the art, and others are regularly described.

Procedures for raising polyclonal antibodies are also known. Typically, such antibodies can be raised by administering the protein or polypeptide of the present invention subcutaneously to New Zealand white rabbits that have first been bled to obtain pre-immune serum. The antigens can be injected at a total volume of 100:1 per site at six different sites. Each injected material will contain synthetic surfactant adjuvant pluronic polyols, or pulverized acrylamide gel containing the protein or polypeptide after SDS-polyacrylamide gel electrophoresis. The rabbits are then bled two weeks after the first injection and periodically boosted with the same antigen three times every six weeks. A sample of serum is then collected 10 days after each boost. Polyclonal antibodies are then recovered from the serum by affinity chromatography using the corresponding antigen to capture the antibody. Ultimately, the rabbits are euthanized, e.g., with pentobarbital 150 mg/Kg IV. This and other procedures for raising polyclonal antibodies are disclosed in E. Harlow, et. al., editors, Antibodies: A Laboratory Manual (1988).

In addition to utilizing whole antibodies, the invention encompasses the use of binding portions of such antibodies. Such binding portions include Fab fragments, F(ab′)₂ fragments, and Fv fragments. These antibody fragments can be made by conventional procedures, such as proteolytic fragmentation procedures, as described in J. Goding, Monoclonal Antibodies: Principles and Practice, pp. 98-118 (N.Y. Academic Press 1983).

The antibody can also be a single chain antibody. A single-chain antibody (scFV) can be engineered (see, for example, Colcher et al., Ann. N. Y. Acad. Sci. 880:263-80 (1999); and Reiter, Clin. Cancer Res. 2:245-52 (1996)). The single chain antibody can be dimerized or multimerized to generate multivalent antibodies having specificities for different epitopes of the same target protein. In some embodiments, the antibody is monovalent, e.g., as described in Abbs et al., Ther. Immunol. 1(6):325-31 (1994), incorporated herein by reference.

### Prophylactic and Therapeutic Compositions

Also provided herein are compositions for use in eliciting a protective immune response to SARS-CoV-2 comprising one or more peptides as described herein that bind to a neutralizing epitope. The compositions can also include an adjuvant to increase T cell response. For example, nanoparticles that enhance T cell response can be included, e.g., as described in Stano et al., Vaccine (2012) 30:7541-6 and Swaminathan et al., Vaccine (2016) 34:110-9. See also Panagioti et al., Front. Immunol., 16 Feb. 2018; doi.org/10.3389/fimmu.2018.00276. Alternatively or in addition, an adjuvant comprising poly-ICLC (carboxymethylcellulose, polyinosinic-polycytidylic acid, and 25 poly-L-lysine double-stranded RNA), Imiquimod, Resiquimod (R-848), CpG oligodeoxynuceotides and formulations (IC31, QB10), AS04 (aluminium salt formulated with 3-O-desacyl-4′-monophosphoryl lipid A (MPL)), ASO1 (MPL and the saponin QS-21), MPLA, STING agonists, other TLR agonists, Candida albicans Skin Test Antigen (Candin), GM-CSF, Fms-like tyrosine kinase-3 ligand (Flt3L), 30 and/or IFA (Incomplete Freund's adjuvant) can also be used. See, e.g., Coffman et al., Immunity. 2010 Oct. 29; 33(4): 492-503. See, e.g., WO2006071896.

These compositions can be administered in a therapeutically effective amount to subjects who have, or in a prophylactically effective amount to subjects who are at risk of developing, an infection with SARS-CoV-2. In some embodiments, the methods include administering two or more doses of the composition (e.g., an initial dose and a booster dose), e.g., 1, 2, 3, 4, 5, 6, 7, or 8, 12, 18, 24, or 52 weeks apart. In some embodiments, the methods include administering annual doses of the compositions, e.g., a prophylactically effective amount.

The present compositions can be used prophylactically to induce anti-SARS-CoV-2 immunity, or therapeutically to treat a SARS-CoV-2 infection in a subject. The methods include administering one or more doses of the vaccine compositions described herein to a subject, e.g., a subject in need thereof.

A therapeutically effective amount as used herein is an amount sufficient to reduce one or more symptoms of a SARS-CoV-2 infection in a subject, or to reduce the length of time that the subject is infected or is symptomatic. A prophylactically effective amount as used herein is an amount sufficient to reduce risk of a subject developing a SARS-CoV-2 infection, or reduce the risk that the subject will experience severe morbidity or mortality associated with a SARS-CoV-2 infection.

Dosage, toxicity and therapeutic efficacy of the therapeutic compositions can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions that exhibit high therapeutic indices are preferred. While compositions that exhibit toxic side effects may be used, care should be taken to minimize and reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compositions used in the methods described herein, the therapeutically effective dose can be estimated initially from cell culture assays. A dose may be formulated in animal models. Such information can be used to more accurately determine useful doses in humans.

Alternatively or in addition, peptides described herein as associated with a neutralizing response can be used to generate antibodies, e.g., for use in vaccines for inducing a protective response or for use in treating subjects. These methods include immunizing an animal, e.g., a mouse, rat, rabbit, guinea pig, goat, sheep, llama, or camel, with an amount of the peptides sufficient to induce an immune response. The antibodies can be isolated from the animals using known methods and formulated for administration as a therapeutic or prophylactic treatment as described herein. The antibodies can optionally be humanized or otherwise rendered less immunogenic before administration.

### Kits and Compositions

Also provided herein are kits and compositions comprising one or more of the peptides described herein. The peptides can be, e.g., labeled and/or conjugated to beads or surfaces for use in a method of screening as described herein. Beads useful in the present methods and compositions include magnetic beads, polystyrene beads, and agarose beads. Methods of conjugating the peptides to a bead or a surface are known and can include conjugations via carboxy, aldehyde, azide, or alkyne groups; avidin/streptavidin binding; or protein A/G binding. Exemplary beads include Luminex MAGPLEX Microspheres (carboxylated polystyrene micro-particles dyed into spectrally distinct sets) and DYNABEADS magnetic beads. Surfaces useful in the present methods and compositions include columns, culture dishes, assay plates such as multiwell assay plates, and coverslips, e.g., glass coverslips.

Also provided are pharmaceutical compositions, which typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

Pharmaceutical compositions are typically formulated to be compatible with its intended route of administration. Examples of routes of administration include parenteral, e.g., intravenous, intradermal, subcutaneous, intratumoral, intramuscular or subcutaneous administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, intramuscular, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

### EXAMPLES

The invention is further described in the following examples, which do not limit the scope of the invention described in the claims.

### Materials and Methods

The following materials and methods were used in the Examples below.

Sources of serum used in this study

Cohort 1

Plasma samples were from volunteers recruited at Brigham and Women's Hospital who had recovered from a confirmed case of Corona Virus Infectious Disease 19 (COVID-19). All volunteers had a PCR-confirmed diagnosis of COVID-19 prior to being admitted to the study. Volunteers were invited to donate specimens after recovering from their illness and were required to be symptom free for a minimum of 7 days. Participants provided verbal and/or written informed consent and provided blood specimens for analysis. Clinical data including date of initial symptom onset, symptom type, date of diagnosis, date of symptom cessation, and severity of symptoms was recorded for all participants, as were results of COVID-19 molecular testing. Participation in these studies was voluntary and the study protocols have been approved by the respective Institutional Review Boards.

Cohort 2

Serum samples were provided by collaborators from University of Washington in patients with PCR-confirmed COVID-19 cases while admitted to the hospital. Residual clinical blood specimens were used, as well as patients who were actively enrolled into a prospective study of COVID-19 infection. Clinical data, including symptom duration and comorbidities were extracted from the medical records and from participant-completed questionnaires. All study procedures have been approved by the University of Washington Institutional Review Board.

Cohort 3

Plasma samples were provided by collaborators from Ragon Institute of MGH, MIT and Harvard and Massachusetts General Hospital from study participants in three settings: 1) PCR-confirmed COVID-19 cases while admitted to the hospital; 2) PCR-confirmed SARS-CoV-2 infected cases seen in an ambulatory setting; 2) PCR-confirmed COVID-19 cases in their convalescent stage. All study participants provided verbal and/or written informed consent. Basic data on days since symptom onset were recorded for all participants as were results of COVID-19 molecular testing. Participation in these studies was voluntary and the study protocols have been approved by the Partners Institutional Review Board.

Cohort 4

Patients were enrolled in the Emergency Department (ED) in Massachusetts General Hospital from 3/15/2020 to 4/15/2020 in Boston at the during the peak of the COVID-19 surge, with an institutional IRB-approved waiver of informed consent. These included patients 18 years or older with a clinical concern for COVID-19 upon ED arrival, and with acute respiratory distress with at least one of the following: 1) tachypnea≥22 breaths per minute, 2) oxygen saturation≤92% on room air, 3) a requirement for supplemental oxygen, or 4) positive-pressure ventilation. A blood sample was obtained in a 10 mL EDTA tube concurrent with the initial clinical blood draw in the ED. Day 3 and day 7 blood draws were obtained if the patient was still hospitalized at those times. Clinical course was followed to 28 days post-enrollment, or until hospital discharge if that occurred after 28 days.

Enrolled subjects who were SARS-CoV-2 positive were categorized into four outcome groups: 1) Requiring mechanical ventilation with subsequent death, 2) Requiring mechanical ventilation and recovered, 3) Requiring hospitalization on supplemental oxygen but not requiring mechanical ventilation, and 4) Discharge from ED and not subsequently readmitted with supplemental oxygen. Those who were SARS-CoV-2 negative were categorized as Controls.

Demographic, past medical and clinical data were collected and summarized for each outcome group, using medians with interquartile ranges and proportions with 95% confidence intervals, where appropriate.

Cohorts 5, 6

Longitudinal Hopkins Cohort: Remnant serum specimens were collected longitudinally from PCR confirmed COVID-19 patients seen at Johns Hopkins Hospital. Samples were de-identified prior to analysis, with linked time since onset of symptom information. Specimens were obtained and utilized in accordance with an approved IRB protocol.

Cohorts 7-8

Cohorts 7-8 were previously published (9, 10).

Cohort 9

Plasma samples were collected from consented participants of the Partner's Biobank program at BWH during the period from July to August 2016 from 37 female and 51 male individuals with ages ranging from 18 to 85 years old. Plasma was harvested after a 10 minutes 1200xg ficoll density centrifugation from blood that was diluted 1:1 in phosphate buffered saline. Samples were frozen at −30 C in 1 mL aliquots. All samples were collected with Partners Institutional Review Board (IRB) approval.

### Blood Sample Collection Methods

For cohorts 1-3: Blood samples were collected into EDTA (Ethylenediamine Tetraacetic Acid) tubes and spun for 15 minutes at 2600 rpm according to standard protocol. Plasma was aliquoted into 1.5 ml cryovials and stored in −80° C. until analyzed. Only de-identified plasma aliquots including metadata (e.g., days since symptom onset, severity of illness, hospitalization, ICU status, survival) were shared for this study. When appropriate for non-convelescent samples plasma/serum was also heat inactivated at 56° C. for 60 minutes, and stored at ≤20C until analyzed.

For cohort 4: Blood samples were collected in EDTA tubes, and processed no more than 3 hours post blood draw in a Biosafety Level 2+ laboratory on site. Whole blood was diluted with room temperature RPMI medium in a 1:2 ratio to facilitate cell separation for other analyses using the SepMate PBMC isolation tubes (STEMCELL) containing 16 ml of Ficoll (GE Healthcare). Diluted whole blood was centrifuged at 1200 rcf for 20 minutes at 20C. After centrifugation, plasma (5 mL) was pipetted into 15 mL conical tubes and placed on ice during PBMC separation procedures. Plasma was then centrifuged at 1000 rcf for 5 min at 4C, pipetted in 1.5 mL aliquots into 3 cryovials (4.5 mL total), and stored at −80C. For the current study samples (200 uL) were first randomly allocated onto a 96 well plate based on disease outcome grouping.

### Design and Cloning of the Public Epitope Tiling and Alanine Scanning Library

Multiple VirScan libraries were constructed and each peptide was encoded in two distinct ways so there were distinguishable duplicate peptides for each fragment described below. We created ˜200 nt oligos encoding peptide sequences 56 AAs in length, tiled with 28-amino acid overlap through the proteomes of all coronaviruses known to infect humans including HCov-NL63, HCoV-229E, HCoV-OC43, HCoV-HKU1, SARS-CoV, MERS and SARS-CoV-2 as well as three closely related bat viruses: BatCoV-Rp3, BatCoV-HKU3 and BatCoV-279. For SARS-CoV-2 we included a number of coding variants available in early sequencing of the viruses. For SARS-CoV-2 we additionally made a 20 AA tiling library with 15-AA overlap. Additionally, for SARS-CoV-2 we made triple-mutant sequences scanning through all 56-mer peptides. Non-alanine AAs were mutated to alanine, and alanines were mutated to glycine. We reverse-translated the peptide sequences into DNA sequences that were codon-optimized for expression in Escherichia coli, that lacked restriction sites used in downstream cloning steps (EcoRI and XhoI), and that were unique in the 50 nt at the 5′ end to allow for unambiguous mapping of the sequencing results. Then we added adapter sequences to the 5′ and 3′ ends to form the final oligonucleotide sequences. Adapter sequences were added to the 5′ and 3′ ends to facilitate downstream PCR and cloning steps. Different adapters were added to each sub-library so that they could be amplified separately. The resulting sequences were synthesized on a releasable DNA microarray (Agilent). We PCR-amplified the DNA oligo library with the primers shown below, digested the product with EcoRI and XhoI, and cloned it into the EcoRI/SalI site of the T7FNS2 vector (Larman et al., 2011). We packaged the resultant library into T7 bacteriophage using the T7 Select Packaging Kit (EMD Millipore) and amplified the library according to the manufacturer's protocol.

### Primers Used for Analysis of the Different Libraries Employed.

SEQ ID NO:

CoV 56-mer Library

| | | |
|---|---|---|
| 5′ Adapter: | 5′- GAATTCGGAGCGGT -3′ | 1 |
| 3′ Adapter: | 5′- CACTGCACTCGAGA -3′ | 2 |
| Forward Primer: | 5′- AATGATACGGCGTGAATTCGGAGCGGT -3′ | 3 |
| Reverse primer: | 5′- CAAGCAGAAGACGTCTCGAGTGCAGTG -3′ | 4 |

Alanine scanning library

| | | |
|---|---|---|
| 5′ Adapter: | 5′- GAATTCCGCTGCGT -3′ | 5 |
| 3′ Adapter: | 5′- CAGGGAAGAGCTCG -3′ | 6 |
| Forward Primer: | 5′- AATGATACGGCGGGAATTCCGCTGCGT -3′ | 7 |
| Reverse primer: | 5′- CAAGCAGAAGACTCGAGCTCTTCCCTG -3′ | 8 |

20 mer SARS-COV-2 Library

| | | |
|---|---|---|
| 5′ Adapter: | 5′- GAATTCCGCTGCGT -3′ | 9 |
| 3′ Adapter: | 5′- GTACTATACCTACGGAAGGCTCG -3′ | 10 |
| Forward Primer: | 5′- AATGATACGGCGGGAATTCCGCTGCGT -3′ | 11 |
| Reverse primer: | 5′- TATCTCGCATAGCGCATATACTCGAGCCTT | 12 |
| | CCGTAGGTATAGTAC -3′ | |

### Phage Immunoprecipitation and Sequencing

We performed phage immunoprecipitation and sequencing as described previously or with slight modifications (9). For the IgA and IgG chain isotype-specific immunoprecipitations, we substituted magnetic protein A and protein G Dynabeads (Invitrogen) with 6 μg Mouse Anti-Human IgG Fc-BIOT (Southern Biotech) or 4 μg Goat Anti-Human IgA-BIOT (Southern Biotech) antibodies. We added these antibodies to the phage and serum mixture and incubated the reactions overnight a 4° C. Next, we added 25 μL or 20 μL of Pierce Streptavidin Magnetic Beads (Thermo-Fisher) to the IgG or IgA reactions, respectively, and incubated the reactions for 4 h at room temperature, then continued with the washing steps and the remainder of the protocol, as previously described (9).

### Gradient Boosting Machine Learning Algorithm

Gradient boosting classifier models were generated using the XGBoost algorithm. Classifier models were trained to discriminate either COVID-19+ and COVID-19—patients (n=232 and n=190 respectively) or severe disease and mild disease (n=101 hospitalized patients and n=131 non-hospitalized patients). Two models were generated in each case, one using the Z-scores for each VirScan peptide from the IgG immunoprecipitation as input features, and the other using the Z-scores for each VirScan peptide from the IgA immunoprecipitation as input features. Additionally, a third logistic regression classifier was trained on the output probabilities from the IgG and IgA models to generate a combined prediction. The performance of each of the three model was assessed using a 20-fold cross-validation procedure, whereby predictions for each 5% of the data points were generated from a model trained on the remaining 95%. The SHAP package was used to identify the top discriminatory peptide features from each of the XGBoost models.

### High-Resolution Epitope Identification and Clustering

To generate a single-amino acid resolution map of SARS-CoV-2 antibody epitopes triple-alanine scanning data from each 56-mer peptide were aggregated across each protein. For each position in the 56-mer, the relative enrichment for each amino-acid was calculated as the mean fold-change of the three mutant peptides containing an alanine-mutation at that location relative to the median fold-change of all alanine mutants for the 56-mer. Overlapping 56-mers were combined by taking the minimum value at each shared position to account for the possibility that an epitope is disrupted in one of the tiles by the peptide junction. To map epitopes from the alanine-scanning data for each sample we used the HMMlearn python package to develop a three-state Hidden-Markov model (HMM) assuming a gaussian distribution of relative-enrichment emissions for each state. Mapped epitopes smaller than 5 amino acids were removed from the subsequent analysis. Next, we performed two-step hierarchical clustering procedure to identify the number of unique epitopes. First, for each protein all of the epitopes identified across the 169 COVID-19+ patients were clustered based on the start and stop locations predicted by the HMM classifier to generate a set of positional clusters we refer to as hotspots. Next, to identify unique epitopes within each hotspot we performed an additional step of hierarchical clustering on the samples with epitopes within each hotspot based on the alanine-scanning relative-enrichment values within the hotspot region (FIGS. 10A-F). The total number of unique epitopes for each protein was taken as the number of distinct epitope groupings following clustering on both epitope location and the motif of relative-enrichments values within the hotspot region.

### Similarity-Score Calculation

Pairwise alignments were generated for the S protein of SARS-CoV-2 and each of the four common HCoVs. Similarity scores were calculated separately for a 21-amino acid window centered at each position of the SARS-CoV-2 S protein. The mean similarity score between SARS-CoV-2 and the corresponding sequence of the other HCoV was calculated for each window using the BLOSUM62 substitution matrix with a gap opening and extending penalty of −10 and −1 respectively. The maximum similarity was score was calculated as the maximum value among the pairwise-similarity scores between SARS-CoV-2 and each of the four common HCoVs for the sliding window centered at each position.

### Luminex Multiplex Peptide Epitope Serology Assays

Multiplexed SARS-CoV-2 peptide epitope assays were built using the peptides listed in Table 1. Peptides were synthesized by the Ragon Core Facility with a Proparglyglycine (Pra, X) (Fmok-Pra-OH) moiety in the amino terminus to facilitate crosslinking to Luminex beads using a “click” chemistry strategy as described (39). In brief, Luminex beads were first functionalized with amine-PEG4-azide and then reacted with the peptides to generate 20 different Luminex beads with attached peptides. Luminex bead-based serology assays were performed in 96-well U-bottom polypropylene plates using PBS+0.1% bovine serum albumin as the assay buffer. Bead washes were done using PBS+0.05% Triton X-100 by incubation for 1 minute on a strong magnetic plate (Millipore-Sigma, Burlington, Mass.). All assay incubation times were 20 minutes. In the first step, beads were incubated with 20 uL of plasma samples (1:300 dilution). Samples used for the classifier were diluted 1:100, samples used to compare disease severity were diluted 1:300. After a wash step, peptide bound IgA or IgG detection was performed by adding 40 μL of biotin-labeled anti-IgA or IgG antibodies at 0.1 μg/ml (Southern Biotechnology, Birmingham, Ala.). Bound IgA or IgG was detected by adding 40 μL of phycoerythrin (PE)-labeled streptavidin (0.2 μg/ml) (Biolegend, San Diego, Calif.). Assay plates were analyzed on a Luminex FLEXMAP 3D instrument (Luminex Corporation, Austin, Tex.) to generate median fluorescence intensity (MFI) values to quantify peptide-specific IgA or IgG levels.

### ELISA Serology Assays

ELISAs were performed separately using the SARS-CoV-2 N protein, S protein, or the S receptor-binding domain (RBD). 96-well plates were coated with antigen overnight. The plates were then blocked in PBS+3% BSA. After washing with PBS+0.05% Tween-20, the plasma sample were diluted 1:100, added to the plates and incubated overnight at 4° C. Following incubation, the plates were washed 3× with PBS+0.05% Tween-20. The bound IgG was detected by adding anti-Human IgG-alkaline phosphatase (Southern Biotech, Birmingham, Ala.) and incubating for 90 minutes at room temperature. The plates were washed an additional three times after which p-nitrophenyl phosphate solution (1.6 mg/mL in 0.1 M glycine, 1 mM ZnCl_{2, 1} mM MgCl₂, pH 10.4) was added to each well and allowed to develop for 2 hours. Bound IgG was quantified by measuring the OD405, and the reported values were calculated as the fold change over the pre-COVID-19 controls.

### Example 1. Development of a VirScan Library Targeting Human Coronaviruses

Our existing VirScan phage-display platform is based on an oligonucleotide library encoding 56-amino acid (56-mer) peptides tiling every 28 amino acids across the proteomes of all known pathogenic human viruses (˜400 species and strains) plus many bacterial proteins (10). In order to interrogate the serological response to SARS-CoV-2 and other human coronaviruses (HCoVs), we supplemented this library with additional oligonucleotides encoding peptides that span the proteome of SARS-CoV-2 itself, plus the proteomes of the six human coronaviruses and the three bat coronaviruses that are most closely related to SARS-CoV-2 (FIG. 1A) (11,12). These additional oligonucleotides were composed of three sub-libraries. Sublibrary 1 encoded a 56 amino acid peptide library that tiles every 28 amino acids through each of the open reading frames (ORFs) expressed by the 10 coronavirus species (FIG. 1B); sublibrary 2 encoded 20 amino acid peptides tiling every 5 amino acids across the SARS-CoV-2 proteome, thereby permitting more precise delineation of epitopes; and sublibrary 3 comprised triple alanine scanning mutants of the 56-mer peptides tiling across the SARS-CoV-2 proteome, enabling epitopes to be mapped at amino acid resolution.

We used VirScan (FIG. 1C) to profile the antibody repertoires of 8 cohorts of individuals from multiple locations including Baltimore, Md., Boston, Mass. and Seattle, Wash. These cohorts comprised individuals enrolled in prospective studies of COVID-19 infection, patients with active COVID-19 receiving treatment either in hospital or outpatient settings, and convalescent patients who had recovered from COVID-19. Some of the cohorts included longitudinal samples, with patients tracked for several weeks following an initial sample taken at either the time of symptom onset or point of hospital entry. (For simplicity, we will refer to all individuals who have experienced COVID-19 as COVID-19 patients.) Our cohorts also included a diverse set of control sera collected prior to the outbreak of COVID-19; to facilitate the identification of epitopes specific to other HCoVs, these included a cohort of young children experiencing HCoV infections for the first time. In total, we analyzed approximately 2,000 individual samples in duplicate for IgG and IgA antibodies, assessing 200 million potential antibody peptide interactions.

### Example 2. Detection of SARS-CoV-2 Seropositivity with VirScan

To measure immune responses to SARS-CoV-2, we compared VirScan profiles of serum samples from COVID-19 patients to those of controls obtained before the emergence of SARS-CoV-2 in 2019. These pre-COVID-19 era controls facilitate identification of (1) SARS-CoV-2 peptides encoding epitopes specific to COVID-19 patients and (2) SARS-CoV-2 peptides encoding epitopes that are cross-reactive with antibodies developed in response to the ubiquitous common-cold HCoVs. Sera from COVID-19 patients exhibited much more SARS-CoV-2 reactivity compared to pre-COVID-19 era controls (FIGS. 1D-E). Some cross-reactivity towards SARS-CoV-2 peptides was observed in the pre-COVID-19 era samples, but this was expected since nearly everyone has been exposed to HCoVs (18).

COVID-19 patient sera also showed significant levels of cross-reactivity with the other highly pathogenic HCoVs, SARS-CoV and MERS-CoV, although less was observed against the more distantly-related MERS-CoV. Extensive cross-reactivity was also observed against peptides derived from the three bat coronaviruses that share the greatest sequence identity with SARS-CoV-2 (FIGS. 1A, 1D-E) (11). We know that these represent cross-reactivities as, given the low prevalence and circumscribed geographical location of SARS-CoV and MERS-CoV, none of the individuals in this study are likely to have encountered these viruses.

COVID-19 patient sera also exhibited a significantly higher level of reactivity to seasonal HCoV peptides compared to pre-COVID-19 era controls (FIGS. 1D-E). This could be due to the elicitation of novel antibodies that cross-react, or to an anamnestic response boosting B cell memory against HCoVs. The converse is not always true: many pre-COVID-19 era samples exhibit strong recognition of seasonal HCoV peptides but little or no recognition of SARS-CoV-2 peptides (FIG. 1D), although one caveat may be that the concentrations of antibodies against seasonal HCoVs may be below the threshold of detection in the pre-COVID-19 era samples.

### Example 3. Coronavirus Proteins Targeted by Antibodies in COVID-19 Patients

Analysis of SARS-CoV-2 proteins targeted by COVID-19 patient antibodies revealed that the primary responses to SARS-CoV-2 are reactive with peptides derived from spike (S) and nucleoprotein (N) (FIGS. 2A-B). These two proteins exhibit significant differential recognition by sera from COVID-19 patients versus pre-COVID-19 era controls, indicating that their recognition is a result of antibody responses to SARS-CoV-2. Third-most frequently recognized is the replicase polyprotein ORF1, but, unlike S and N, ORF1 is recognized to a similar extent by sera from COVID-19 patients and pre-COVID-19 era controls. This suggests that recognition of SARS-CoV-2 ORF1 is a result of cross-reactions from antibodies elicited by exposure to other pathogens, possibly HCoVs. Antibody responses to peptides from membrane glycoprotein (M), ORF3 and ORF9b were occasionally detected in COVID-19 patients.

We also analyzed longitudinal samples from 23 COVID-19 patients. Most patients displayed an antibody response to peptides derived from the S or N in the second week after symptom onset, with many displaying an antibody response by the end of the first week (FIG. 2C). The relative strength and onset of the antibody response to the S and N differed dramatically between individuals, and the initial immune response showed no preference for the S or N. The signal intensity of antibodies recognizing SARS-CoV-2 ORF1 epitopes did not increase over time, further suggesting that ORF1 antibodies likely represent a preexisting cross-reactive response.

### Example 4. Identification of Immunogenic Regions of SARS-CoV-2 Proteins

To more precisely define the immunogenic regions of the SARS-CoV-2 proteome, we examined the specific 56-mer and 20-mer peptides that were detected by VirScan in COVID-19 patients compared to pre-COVID-19 era controls. An example IgG response from a single patient to the SARS-CoV-2 S and N is shown in FIG. 3A. We observed strong concordance between the viral regions enriched by the 56-mer and 20-mer fragments, demonstrating the robustness of VirScan. In many cases we observed recognition of overlapping 56-mer peptides, indicating an epitope in the common region.

Next, we compared the protein regions recognized by IgG and IgA across COVID-19 patients (FIG. 3B). We identified four regions each in the S and N that are recurrently targeted by antibodies from >15% of COVID-19 patients, with additional regions recognized less frequently. Overall, IgG and IgA recognize the same protein regions with similar frequencies across the population. However, when IgG and IgA responses were compared within individuals, we observed considerable divergence (FIG. 3C): many epitopes were recognized by only IgG, only IgA, or both IgG and IgA within an individual patient. Together, these data suggest that patients raise distinct IgG and IgA antibody responses to SARS-CoV-2, but the regions targeted are largely shared at a population level.

### Example 5. Machine Learning Guides the Design of a Luminex Assay for Rapid COVID-19 Diagnosis

To predict SARS-CoV-2 exposure history from VirScan data, we developed a gradient-boosting algorithm (XGBoost) that integrated both IgG and IgA data and predicted current or past COVID-19 disease with 99.1% sensitivity and 98.4% specificity (FIGS. 4A-B). Interrogating the model using Shapley Additive exPlanations (SHAP), a method to compute the contribution of each feature of the data to the predictive model (20), we identified peptides from SARS-CoV-2 S and N plus homologous peptides from SARS-CoV and BatCoV-HKU-3 and BatCoV-279 that were highly predictive of SARS-CoV-2 exposure (FIGS. 4C-D).

We leveraged these insights to develop a simple, rapid Luminex-based diagnostic for COVID-19. We chose 12 SARS-CoV-2 peptides predicted by VirScan data and the machine-learning model to be highly indicative of SARS-CoV-2 exposure history (Table 1). These SARS-CoV-2 peptides, plus two positive control peptides from Rhinovirus A and Epstein-Barr virus (EBV) that are recognized in over 80% of seropositive individuals by VirScan (9), and a negative control peptide from HIV-1, were coupled to Luminex beads (39). We tested 163 COVID-19 patient samples and 165 pre-COVID-19 era controls for IgG reactivity to the Luminex panel. We detected clear responses to SARS-CoV-2 peptides in COVID-19 patient samples but rarely in the pre-COVID-19 era controls (FIG. 4E). Using the Luminex data, we developed a logistic regression model that predicted COVID-19 infection history with 89.6% sensitivity and 95.2% specificity (AUC=0.97) (FIGS. 4F-G). A subset of the COVID-19 positive samples (n=107) were also examined using an in-house ELISA using three SARS-CoV-2 antigens: N, S, and the S receptor-binding domain (RBD). Considering a sample positive if it scored above the 99% specificity threshold on any one of the three ELISA antigens, we determined that the sensitivity of the Luminex assay for this subset (88.8%) was similar to that of the ELISA (90.7%) (FIGS. 13A-D). Among samples run on all three assays, VirScan significantly out-performed both the Luminex and ELISAs (FIG. 13A and C). Remarkably, our optimal model integrated only 3 SARS-CoV-2 peptides which were also the most discriminatory 20-mers in the VirScan data: N 386-406, S 810-830, and S 1146-1166. IgG responses in the COVID-19 patients were highly correlated between the Luminex and VirScan assays, providing orthogonal validation of the VirScan data and supporting the prevalence of SARS-CoV-2-induced humoral responses to these regions of the S and N (FIG. 13D).

TABLE 1
Peptide sequences used for the LUMINEX assay

| Species | Protein | Start | End | Sequence | # | Notes |
|---|---|---|---|---|---|---|
| SARS-COV2 | ORF1 | 151 | 171 | XKKSFDLGDELGTDPYEDFQENWNTKH | 13 | SARS-COV2- |
| | | | | | | specific |
| SARS-COV2 | ORF3 | 171 | 210 | XKKGDGTTSPISEHDYQIGGYTEKWESGV | 1190 | SARS-COV2- |
| | | | | KDCVVLHS | | specific |
| SARS-COV2 | N | 161 | 181 | XKKNNAAIVLQLPQGTTLPKGFYAEGS | 14 | SARS-COV2- |
| | | | | | | specific |
| SARS-COV2 | S | 200 | 220 | XKKIDGYFKIYSKHTPINLVRDLPQGF | 15 | SARS-COV2- |
| | | | | | | specific |
| SARS-COV2 | N | 222 | 242 | XKKLLLLDRLNQLESKMSGKGQQQQGQ | 16 | SARS-COV2- |
| | | | | | | specific |
| SARS-COV2 | N | 240 | 260 | XKKGQQQQGQTVTKKSAAEASKKPRQ | 17 | SARS-COV2- |
| | | | | | | specific |
| SARS-COV2 | N | 365 | 385 | XKKDAYKTFPPTEPKKDKKKKADETQA | 18 | SARS-COV2- |
| | | | | | | specific |
| SARS-COV2 | N | 386 | 406 | XKKLPQRQKKQQTVTLLPAADLDDFSK | 19 | SARS-COV2- |
| | | | | | | specific |
| SARS-COV2 | S | 550 | 570 | XKKTGTGVLTESNKKFLPFQQFGRDIA | 20 | SARS-COV2- |
| | | | | | | specific |
| SARS-COV2 | S | 681 | 706 | XKKRRARSVASQSIIAYTMSLGAENSVA | 21 | SARS-COV2- |
| | | | | | | specific |
| SARS-COV2 | S | 765 | 785 | XKKQLNRALTGIAVEQDKNTQEVFAQV | 22 | SARS-COV2- |
| | | | | | | specific |
| SARS-COV2 | S | 785 | 805 | XKKFAQVKQIYKTPPIKDFGGFNFSQI | 23 | SARS-COV2- |
| | | | | | | specific |
| SARS-COV2 | S | 810 | 830 | XKKPDPSKPSKRSFIEDLLFNKVTLAD | 24 | SARS-COV2- |
| | | | | | | specific |
| SARS-COV2 | S | 1146 | 1166 | XKKYDPLQPELDSFKEELDKYFKNHTS | 25 | SARS-COV2- |
| | | | | | | specific |
| SARS-COV2 | S | 1250 | 1270 | XKKCCSCGSCCKFDEDDSEPVLKGVKL | 26 | SARS-COV2- |
| | | | | | | specific |
| Rhinovirus A | | | | XKKNPIENYVDEVLNEVLVVPNINSSHP | 27 | positive ctl* |
| Human | | | | XKKPPPGRRPFFHPVAEADYFEYHQEGG | 28 | positive ctl ** |
| Herpesvirus | | | | | | |
| 4 | | | | | | |
| HIV-1 | | | | XKKQDNSDIKVVPRRKAKIIRDYGKQMA | 29 | negative |
| | | | | | | ctl *** |

# SEQ ID NO:
*Rhinovirus A public epitope; ** Human Herpesvirus 4_public epitope; *** HIV-1_public epitope
X a propargylgylcine amino acid. The propargylgylcine and lysine residues were added onto the beginning of the peptide to allow for coupling to the bead, so the epitope sequences do not include the XKK.

### Example 6. Differential Antibody Responses to Common Viruses in Hospitalized and Non-Hospitalized Patients

We next considered whether differences in the antibody response to SARS-CoV-2 or to other viruses might be associated with the severity of COVID-19 disease. We grouped the COVID-19 patients into two subsets: those who required hospitalization (n=101), and those who did not (n=131). We compared the responses to peptides derived from the SARS-CoV-2 S and N proteins between the hospitalized (H) and non-hospitalized (NH) groups, and found that the H group exhibited stronger and broader antibody responses to S and N peptides that might be due to epitope spreading (FIG. 5A). We then analyzed 32 NH COVID-19 samples, 32 H COVID-19 samples, and 32 pre-COVID-19 era negative controls with the Luminex assay, and similarly observed that the H group had stronger and broader antibody responses to SARS-CoV-2-specific peptides compared with the NH group (FIG. 5B).

VirScan also offers the opportunity to examine the history of previous viral infections and to determine correlates of COVID-19 outcomes. For example, prior viral exposure could provide some protection if cross-reactive neutralizing antibodies or T cell responses are stimulated upon exposure to SARS-CoV-2 (21, 22). Alternatively, cross-reactive antibodies to viral surface proteins could increase the risk of severe disease due to antibody-dependent enhancement (ADE), as has been observed for SARS-CoV (23, 24). Furthermore, exposure to certain viruses could impact the response to SARS-CoV-2 by altering the immune system. To examine these possibilities, we analyzed the virome-wide VirScan data and found that overall, the NH patients exhibited greater responses to individual peptides from common viruses such as Rhinoviruses, Influenza viruses, and Enteroviruses, while the H patients displayed greater responses to peptides from cytomegalovirus (CMV) and Herpes Simplex Virus 1 (HSV-1) (FIG. 5C). These observations may be influenced by demographic differences in the NH and H cohorts as described below.

We sought to understand whether the differential reactivity to CMV and HSV-1 between the H and NH patients was due to differences in the strength of antibody responses or the prevalence of infection (these viruses are common, but not ubiquitous as are Rhinoviruses, Enteroviruses and Influenza viruses). Using VirScan data, we found that the H group had a higher incidence of both CMV and HSV-1 infection: 82.2% (83/101) of the H group were positive for CMV versus 37.4% (49/131) of the NH group, while 92.1% (93/101) of the H group were positive for HSV-1 versus 45.8% (60/131) of the NH group. To examine the relative strength of the antibody responses, we considered only CIV or HSV-1 seropositive individuals from the NH and H groups: the antibody response to both CMV (FIG. 5D) and HSV-1 (FIG. 14) was stronger among the NH individuals. Thus, the differing seroprevalence of CIV and HSV-1 in the NH versus H groups likely explain the results shown in FIG. 5C. We conclude that antibody responses to nearly all viruses, except SARS-CoV-2, were weaker in the H patients compared to the NH patients.

These striking differences led us to examine potential demographic covariates between the NH and H groups. We found that age, sex, and race were all significantly associated with COVID-19 severity, as has been reported (25, 26). Higher age, male sex, and non-white ethnicity groups were significantly overrepresented in the H group compared with the NH group. Furthermore, hospitalized males exhibited stronger responses to N than hospitalized females while non-hospitalized males and females did not exhibit differential responses to any SARS-CoV-2 proteins. Advanced age is a dominant risk factor for severe COVID-19 and is correlated with reduced immune function (38). In light of the age difference between the H (median age 58) and NH (median age 42) patients in our cohort, we reasoned that the antigens recognized more strongly in the NH group might reflect more general age-associated changes in humoral immunity. To test this hypothesis, we examined VirScan data for a cohort of 648 healthy, pre-pandemic donors. We characterized the recognition of each NH-associated peptide in subsets of the healthy donors representing different age groups and observed a general decline in recognition with age, including a median 19% reduction in recognition from age 42 to 58 (FIG. 5E). These data suggest that age-related changes to the immune system may in part explain the observation of weaker antibody responses to most viruses in the H group. While correlative and potentially influenced by other demographic differences between the NH and H cohorts, the broad age-related diminution in immune system activity we observed could be an important aspect of the increased severity in the H group.

### Example 7. Cross Reactivity of SARS-CoV-2 Epitopes

We returned to the question of epitope cross-reactivity, this time examining antibody responses to the triple-alanine scanning library. For each 56-mer peptide spanning the SARS-CoV-2 proteome, this library contained a collection of scanning mutants: the first mutant peptide encoded 3 alanines instead of the first 3 residues, the second mutant peptide contained the 3 alanines moved one residue downstream, and so on (FIGS. 14A-B). Antibodies that recognize the wild-type 56-mer peptide will not recognize mutant versions of the peptide containing alanine substitutions at critical residues; thus, the location of the linear epitope can be deduced by looking for “antibody footprints”, indicated by stretches of alanine mutants missing from the pool of immunoprecipitated phage. The first and last triple-alanine mutations to interfere with binding are expected to start two amino acids before the first residue essential for the antibody binding, and end two amino acids after the last.

With respect to cross-reactivity, IgG from COVID-19 patients recognized more 56-mer peptides from the common HCoVs HKU1, OC43, 299E, and NL63, than IgG from pre-COVID-19 era controls. This difference is primarily driven by a dramatic increase in recognition of S peptides from the HCoVs and is likely a result of cross-reactivity of antibodies developed during SARS-CoV-2 infection (FIG. 6A).

We mapped the position of all HCoV S peptides that display increased recognition in COVID-19 patient samples onto the SARS-CoV-2 S protein. This revealed four immunodominant regions recognized by >25% of COVID-19 patients (FIG. 6B). Comparing the frequency of peptide recognition between the COVID-19 patients and pre-COVID-19 era controls showed that two of these immunogenic regions in SARS-CoV-2 S are likely strongly cross-reactive with homologous regions of other HCoVs, as the frequency of recognition of the HCoV peptides at these regions rises in COVID-19 patients. For instance, peptides from all four seasonal HCoVs that span the region corresponding to residues 811-830 of SARS-CoV-2 S are frequently recognized by COVID-19 patients but much less so by pre-COVID-19 era controls, suggesting that this recognition is a result of antibodies developed or boosted in response to SARS-CoV-2 infection. Using triple-alanine scanning mutagenesis (FIGS. 14A-B), we mapped the antibody footprints in this region to an 11 amino acid stretch that is highly conserved between SARS-CoV-2 and all four common HCoVs, which explains the cross-reactivity (FIGS. 6C-D). Similarly, both SARS-CoV-2 and HCoV-OC43 peptides corresponding to S 1144-1163 were recognized much more frequently by COVID-19 patients than pre-COVID-19 era controls, and triple-alanine-scanning mutagenesis confirmed that the antibody footprints are located within a 10 amino acid stretch conserved between SARS-CoV-2 and HCoV-OC43 but not the other HCoVs. In contrast, the epitope sequences around S 551-570 and S 766-785 are not conserved between SARS-CoV-2 and the seasonal HCoVs, and indeed these epitopes are not cross-reactive. One HCoV-HKU1 peptide spanning S 551-570 scores in both COVID-19 patients and pre-COVID-19 era control samples; however, its frequency of detection is not further boosted in COVID-19 patients, suggesting the antibody responsible for boosting the SARS-CoV-2 S 551-570 peptide is distinct from the antibody recognizing the HCoV-HKU1 peptide, consistent with differences in sequence at this location (FIG. 6C).

Interestingly, we detected antibody responses to SARS-CoV-2 S 811-830 in 79% of COVID-19 patients, but we also saw responses to the corresponding peptides from OC43 and 229E in ˜20% of the pre-COVID-19 era controls and these responses seem to cross-react with SARS-CoV-2. It is possible that some patients have pre-existing antibodies to this region that cross-react and are expanded during SARS-CoV-2 infection. This might explain the remarkably high prevalence of antibody responses to this epitope, and suggests that anamnestic responses to seasonal coronaviruses may influence the antibody response to SARS-CoV-2. Interestingly, this region is located directly after the predicted S2′ cleavage site for SARS-CoV-2 and overlaps the fusion peptide. A recent study showed that adding an excess of the fusion peptide reduced neutralization, implying that an antibody that binds the fusion peptide might contribute to neutralization by interfering with membrane fusion (27, 29). Given the frequency of seroreactivity toward this epitope in COVID-19 patients, it will be important to determine if the antibodies recognizing this epitope are neutralizing in future studies.

### Example 8. High Resolution Epitope Mapping Reveals Hundreds of Distinct SARS-CoV-2 Epitopes, Including Likely Epitopes of Neutralizing Antibodies

We also used the triple-alanine scanning mutagenesis library to map antibody footprints across the entire SARS-CoV-2 proteome (FIG. 7, FIGS. 12A-D and Tables 3-4). We used a Hidden Markov Model (HMM) to analyze the mutagenesis data and detect antibody footprints. By integrating signals across stretches of consecutive residues, the HMM successfully distinguished antibody footprints from random noise and thus detected regions containing epitopes with improved sensitivity and with far greater resolution than was possible with the 56-mer peptide data alone (see Methods) (FIGS. 8A-C, FIGS. 9A-B). We performed hierarchical clustering on the antibody footprints identified by the HMM to determine the number of distinct epitopes (here defined as unique antibody footprints) that we detected across the SARS-CoV-2 proteome (FIGS. 10A-F). Overall, we identified 3103 antibody footprints across 169 COVID-19 patient samples and mapped 823 distinct epitopes (Table 4). These epitopes are not evenly distributed along the proteins but rather fall into 303 epitope clusters, each of which contains multiple overlapping epitopes (FIGS. 10A-F, Table 3). For example, across the 89 IgA samples that recognized the epitope cluster from S 1135-1165, we identified 9 epitopes that overlap but have distinct triple-alanine scanning profiles that indicate unique antibody-epitope interactions (FIG. 10C). Individual epitopes are recognized at a wide range of frequencies in the COVID-19 patients. The average COVID-19 patient sample contained antibodies to ˜18 distinct linear epitopes (FIGS. 11A-I), although this is likely an underestimate of the total epitope count per person as VirScan does not efficiently detect antibodies recognizing discontinuous (conformational) epitopes (although such antibodies may retain some affinity to linear peptides comprising the epitope).

The SARS-CoV-2 epitope landscape includes regions recognized by a large fraction of COVID-19 patients (public epitopes) and regions recognized by one or a few individuals (private epitopes). For example, we mapped 6 distinct epitopes in the region spanning N 151-175 (FIG. 12C). One of these epitopes was recognized by nearly one-third of the COVID-19 patients, while the rest were detected by less than 2% of the COVID-19 patients. Similarly, the region spanning S 766-835 contained over 20 distinct epitopes, including the highly public epitope cluster near S815 and the public epitope cluster near S770 that is preferentially recognized by IgA (FIG. 7B). This epitope cluster was identified by 43% of COVID-19 patient IgA samples but only 4% of COVID-19 patient IgG samples. In another example, we detected at least 20 distinct epitopes within a stretch of just 46 residues in N 363-408, 10 of which were specific to IgA and 2 of which were specific to IgG (FIG. 12D).

We also mapped at least 12 distinct epitopes in the SARS-CoV-2 RBD, including 5 in the receptor binding motif (RBM) that binds ACE2, the human receptor for SARS-CoV-2, and 5 that are directly adjacent to ACE2 binding sites (FIGS. 7C-D, FIG. 8A). For example, S 414-427 (labeled E2 in FIGS. 7A-H) spans residue K417 in the RBD; K417 makes a direct contact with the human ACE2 protein in structures of ACE2 bound to the RBD. Thus, antibodies recognizing E2 are likely to block ACE2 binding and have neutralizing activity (FIG. 7E). Epitope S 454-463 (labeled E6 in FIGS. 7A-H) also overlaps ACE2 contact residues and partially overlaps the binding site of the neutralizing antibody CB6, suggesting that antibodies recognizing this epitope also have neutralizing potential (28-30) (FIG. 7G). Several other epitopes also span or are adjacent to critical residues contacted by ACE2 (FIGS. 7F and H). Thus, our data reveal some of the likely binding sites for neutralizing antibodies.

Table 3 presents 303 peptide epitope clusters, and Table 4 presents 823 epitopes with their peptide sequences, with an indication of whether the peptide is believed to be the receptor binding domain (RBD) (True/False).

TABLE 3
SARS-COV-2_Epitope_Sequences

| Epitope_cluster_ | | | | | |
|---|---|---|---|---|---|
| id | Protein | Start | End | Epitope_cluster_sequence | SEQ ID NO: |
| 1_ORF3 | ORF3 | 161 | 192 | SVTSSIVITSGDGTTSPISEHDYQIGGYTE | 30 |
| | | | | K | |
| 3_ORF3 | ORF3 | 256 | 275 | NPVMEPIYDEPTTTTSVPL | 31 |
| 2_ORF3 | ORF3 | 230 | 250 | FIYNKIVDEPEEHVQIHTID | 32 |
| 3_M | M | 153 | 168 | HHLGRCDIKDLPKEI | 33 |
| 1_M | M | 1 | 11 | ADSNGTITVE | 34 |
| 2_M | M | 199 | 222 | RIGNYKLNTDHSSSSDNIALLVQ | 35 |
| 4_M | M | 177 | 196 | YYKLGASQRVAGDSGFAAY | 36 |
| 2_ORF6 | ORF6 | 10 | 29 | IAEILLIIMRTFKVSIWNL | 37 |
| 1_ORF6 | ORF6 | 1 | 12 | FHLVDFQVTIA | 38 |
| 3_ORF6 | ORF6 | 33 | 56 | NLIIKNLSKSLTENKYSQLDEEQ | 39 |
| 4_ORF6 | ORF6 | 50 | 61 | QLDEEQPMEID | 40 |
| 5_ORF6 | ORF6 | 40 | 61 | SKSLTENKYSQLDEEQPMEID | 41 |
| 1_ORF7A | ORF7A | 40 | 52 | EGNSPFHPLADN | 42 |
| 1_ORF8 | ORF8 | 62 | 70 | DEAGSKSP | 43 |
| 2_ORF8 | ORF8 | 30 | 39 | YVVDDPCPI | 44 |
| 3_ORF8 | ORF8 | 115 | 121 | VVLDFI | 45 |
| 8_N | N | 35 | 57 | RSKQRRPQGLPNNTASWFTALT | 46 |
| 4_N | N | 391 | 407 | VTLLPAADLDDFSKQL | 47 |
| 14_N | N | 182 | 198 | SSRSSSRSRNSSRNST | 48 |
| 22_N | N | 235 | 257 | GKGQQQQGQTVTKKSAAEASKK | 49 |
| 15_N | N | 158 | 174 | LQLPQGTTLPKGFYAE | 50 |
| 19_N | N | 218 | 235 | LALLLLDRLNQLESKMS | 51 |
| 25_N | N | 267 | 279 | YNVTQAFGRRGP | 52 |
| 13_N | N | 84 | 111 | GYYRRATRRIRGGDGKMKDLSPRWYFY | 53 |
| 26_N | N | 253 | 274 | ASKKPRQKRTATKAYNVTQAF | 54 |
| 3_N | N | 361 | 377 | TFPPTEPKKDKKKKAD | 55 |
| 23_N | N | 281 | 300 | TQGNFGDQELIRQGTDYKH | 56 |
| 7_N | N | 370 | 389 | DKKKKADETQALPQRQKKQ | 57 |
| 5_N | N | 395 | 419 | PAADLDDFSKQLQQSMSSADSTQA | 58 |
| 2_N | N | 351 | 370 | LLNKHIDAYKTFPPTEPKK | 59 |
| 1_N | N | 338 | 361 | LDDKDPNFKDQVILLNKHIDAYK | 60 |
| 10_N | N | 32 | 46 | SGARSKQRRPQGLP | |
| 9_N | N | 23 | 31 | TGSNQNGE | 62 |
| 12_N | N | 59 | 78 | GKEDLKFPRGQGVPINTNS | 63 |
| 16_N | N | 141 | 157 | PKDHIGTRNPANNAAI | 64 |
| 20_N | N | 213 | 251 | GGDAALALLLLDRLNQLESKMSGKGQQ | 65 |
| | | | | QQGQTVTKKSA | |
| 6_N | N | 376 | 398 | DETQALPQRQKKQQTVTLLPAA | 66 |
| 11_N | N | 109 | 145 | FYYLGTGPEAGLPYGANKDGIIWVATEG | 67 |
| | | | | ALNTPKDH | |
| 21_N | N | 243 | 258 | QTVTKKSAAEASKKP | 68 |
| 18_N | N | 201 | 228 | SRGTSPARMAGNGGDAALALLLLDRLN | |
| 24_N | N | 311 | 323 | SAFFGMSRIGME | 70 |
| 17_N | N | 213 | 233 | GGDAALALLLLDRLNQLESK | 71 |
| 4_ORF9B | ORF9B | 83 | 89 | TEELPD | 72 |
| 2_ORF9B | ORF9B | 50 | 81 | PLSLNMARKTLNSLEDKAFQLTPIAVQM | 73 |
| | | | | TKL | |
| 3_ORF9B | ORF9B | 42 | 51 | PIILRLGSP | 74 |
| 1_ORF9B | ORF9B | 1 | 11 | DPKISEMHPA | 75 |
| 1_ORF9C | ORF9C | 14 | 24 | QKASTQKGAE | 76 |
| 49_S | S | 766 | 782 | LTGIAVEQDKNTQEVF | 77 |
| 45_S | S | 811 | 827 | PSKRSFIEDLLFNKVT | 78 |
| 23_S | S | 1141 | 1164 | QPELDSFKEELDKYFKNHTSPDV | 79 |
| 42_S | S | 972 | 996 | ISSVLNDILSRLDKVEAEVQIDRL | 80 |
| 10_S | S | 413 | 430 | QTGKIADYNYKLPDDFT | 81 |
| 11_S | S | 437 | 448 | SNNLDSKVGGN | 82 |
| 34_S | S | 685 | 708 | SVASQSIIAYTMSLGAENSVAYS | 83 |
| 44_S | S | 798 | 826 | GFNFSQILPDPSKPSKRSFIEDLLFNKV | 84 |
| 40_S | S | 1014 | 1036 | AAEIRASANLAATKMSECVLGQ | 85 |
| 37_S | S | 917 | 942 | ENQKLIANQFNSAIGKIQDSLSSTA | 86 |
| 12_S | S | 452 | 467 | YRLFRKSNLKPFERD | 87 |
| 19_S | S | 260 | 289 | GAAAYYVGYLQPRTFLLKYNENGTITDA | 88 |
| | | | | V | |
| 17_S | S | 299 | 311 | KCTLKSFTVEKG | 89 |
| 27_S | S | 547 | 559 | GTGVLTESNKKF | 90 |
| 38_S | S | 899 | 907 | MQMAYRFN | 91 |
| 30_S | S | 571 | 588 | TTDAVRDPQTLEILDIT | 92 |
| 5_S | S | 135 | 155 | CNDPFLGVYYHKNNKSWMES | 93 |
| 35_S | S | 674 | 690 | QTQTNSPRRARSVASQ | 94 |
| 25_S | S | 1177 | 1199 | NIQKEIDRLNEVAKNLNESLID | 95 |
| 46_S | S | 790 | 803 | TPPIKDFGGFNFS | 96 |
| 16_S | S | 307 | 324 | VEKGIYQTSNFRVQPTE | 97 |
| 14_S | S | 326 | 338 | VRFPNITNLCPF | 98 |
| 28_S | S | 553 | 570 | ESNKKELPFQQFGRDIA | 99 |
| 18_S | S | 286 | 305 | DAVDCALDPLSETKCTLKS | 100 |
| 31_S | S | 620 | 642 | PVAIHADQLTPTWRVYSTGSNV | 101 |
| 36_S | S | 650 | 671 | IGAEHVNNSYECDIPIGAGIC | 102 |
| 47_S | S | 786 | 800 | QIYKTPPIKDFGGF | 103 |
| 7_S | S | 86 | 94 | NDGVYFAS | 104 |
| 32_S | S | 701 | 718 | ENSVAYSNNSIAIPTNF | 105 |
| 29_S | S | 598 | 607 | TPGTNTSNQ | 106 |
| 9_S | S | 403 | 417 | GDEVRQIAPGQTGK | 107 |
| 43_S | S | 841 | 868 | GDIAARDLICAQKFNGLTVLPPLLTDE | 108 |
| 48_S | S | 771 | 791 | VEQDKNTQEVFAQVKQIYKT | 109 |
| 24_S | S | 1161 | 1176 | PDVDLGDISGINASV | 110 |
| 22_S | S | 1051 | 1060 | FPQSAPHGV | 111 |
| 13_S | S | 477 | 488 | TPCNGVEGFNC | 112 |
| 33_S | S | 731 | 737 | TKTSVD | 113 |
| 20_S | S | 1091 | 1113 | EGVFVSNGTHWFVTQRNFYEPQ | 114 |
| 4_S | S | 177 | 196 | DLEGKQGNFKNLREFVFKN | 115 |
| 2_S | 5 | 227 | 240 | DLPIGINITRFQT | 116 |
| 8_S | S | 115 | 123 | SLLIVNNA | 117 |
| 26_S | S | 535 | 547 | NKCVNFNFNGLT | 118 |
| 41_S | S | 965 | 989 | LSSNFGAISSVLNDILSRLDKVEA | 119 |
| 15_S | S | 348 | 362 | SVYAWNRKRISNCV | 120 |
| 3_S | S | 195 | 207 | NIDGYFKIYSKH | 121 |
| 6_S | S | 160 | 166 | SSANNC | 122 |
| 39_S | S | 877 | 884 | LAGTITS | 123 |
| 21_S | S | 1072 | 1083 | KNFTTAPAICH | 124 |
| 1_S | S | 25 | 42 | PAYTNSFTRGVYYPDKV | 125 |
| 154_ORF1 | ORF1 | 244 | 273 | SEKSYELQTPFEIKLAKKFDTFNGECPNF | 126 |
| 165_ORF1 | ORF1 | 766 | 780 | EQPTSEAVEAPLVG | 127 |
| 177_ORF1 | ORF1 | 1592 | 1608 | QFGPTYLDGADVTKIK | 128 |
| 108_ORF1 | ORF1 | 1888 | 1914 | CTEIDPKLDNYYKKDNSYFTEQPIDL | 129 |
| 94_ORF1 | ORF1 | 2187 | 2206 | SRIKASMPTTIAKNTVKSV | 130 |
| 133_ORF1 | ORF1 | 2670 | 2685 | NNYMLTYNKVENMTP | 131 |
| 103_ORF1 | ORF1 | 1815 | 1826 | LKHGTFTCASE | 132 |
| 112_ORF1 | ORF1 | 1932 | 1951 | DNIKFADDLNQLTGYKKPA | 133 |
| 113_ORF1 | ORF1 | 1973 | 1992 | HYTPSFKKGAKLLHKPIVW | 134 |
| 117_ORF1 | ORF1 | 3230 | 3240 | CCHLAKALND | 135 |
| 25_ORF1 | ORF1 | 3824 | 3841 | SQGLLPPKNSIDAFKLN | 136 |
| 48_ORF1 | ORF1 | 5702 | 5727 | ATNYDLSVVNARLRAKHYVYIGDPA | 137 |
| 81_ORF1 | ORF1 | 6505 | 6528 | AFELWAKRNIKPVPEVKILNNLG | 138 |
| 86_ORF1 | ORF1 | 6588 | 6603 | ARNGVLITEGSVKGL | 139 |
| 65_ORF1 | ORF1 | 6926 | 6944 | SDMYDPKTKNVTKENDSK | 140 |
| 166_ORF1 | ORF1 | 747 | 768 | PTEVLTEEVVLKTGDLQPLEQ | 141 |
| 170_ORF1 | ORF1 | 880 | 891 | KTLQPVSELLT | 142 |
| 192_ORF1 | ORF1 | 1035 | 1050 | DNVYIKNADIVEEAK | 143 |
| 190_ORF1 | ORF1 | 1112 | 1134 | HCLHVVGPNVNKGEDIQLLKSA | 144 |
| 99_ORF1 | ORF1 | 2093 | 2121 | DLMAAYVDNSSLTIKKPNELSRVLGLKT | 145 |
| 128_ORF1 | ORF1 | 2463 | 2486 | FISDEVARDLSLOFKRPINPTDQ | 146 |
| 35_ORF1 | ORF1 | 4118 | 4140 | SPNLAWPLIVTALRANSAVKLQ | 147 |
| 43_ORF1 | ORF1 | 5592 | 5609 | YQKVGMQKYSTLQGPPG | 148 |
| 51_ORF1 | ORF1 | 5826 | 5835 | NPAWRKAVF | 149 |
| 85_ORF1 | ORF1 | 6548 | 6576 | STIGVCSMTDIAKKPTETICAPLTVFFD | 150 |
| 161_ORF1 | ORF1 | 112 | 132 | EIPVAYRKVLLRKNGNKGAG | 151 |
| 164_ORF1 | ORF1 | 152 | 167 | PYEDFQENWNTKHSS | 152 |
| 191_ORF1 | ORF1 | 1066 | 1090 | HGGGVAGALNKATNNAMQVESDDY | 153 |
| 201_ORF1 | ORF1 | 1205 | 1223 | IPKEEVKPFITESKPSVE | 154 |
| 180_ORF1 | ORF1 | 1480 | 1502 | DAVTAYNGYLTSSSKTPEEHFI | 155 |
| 100_ORF1 | ORF1 | 2065 | 2095 | VKTTEVVGDIILKPANNSLKITEEVGHTDL | 156 |
| 134_ORF1 | ORF1 | 2689 | 2708 | ACIDCSARHINAQVAKSHN | 157 |
| 30_ORF1 | ORF1 | 4507 | 4531 | RQRLTKYTMADLVYALRHFDEGNC | 158 |
| 6_ORF1 | ORF1 | 5178 | 5192 | YYQNNVFMSEAKCW | 159 |
| 74_ORF1 | ORF1 | 6204 | 6230 | LAVHECFVKRVDWTIEYPIIGDELKI | 160 |
| 76_ORF1 | ORF1 | 6236 | 6254 | VQHMVVKAALLADKFPVL | 161 |
| 91_ORF1 | ORF1 | 6684 | 6689 | EHIVY | 162 |
| 148_ORF1 | ORF1 | 447 | 471 | DNLLEILQKEKVNINIVGDFKLNE | 163 |
| 205_ORF1 | ORF1 | 1291 | 1324 | QEGVLTAVVIPTKKAGGTTEMLAKALRK | 164 |
| | | | | VPTDN | |
| 139_ORF1 | ORF1 | 2719 | 2741 | SLSEQLRKQIRSAAKKNNLPFK | 165 |
| 80_ORF1 | ORF1 | 6495 | 6512 | ENKTTLPVNVAFELWAK | 166 |
| 171_ORF1 | ORF1 | 803 | 819 | PNMMVTNNTFTLKGGA | 167 |
| 92_ORF1 | ORF1 | 2135 | 2150 | DTIANYAKPFLNKVV | 168 |
| 138_ORF1 | ORF1 | 2754 | 2767 | TTKIALKGGKIVN | 169 |
| 116_ORF1 | ORF1 | 3258 | 3273 | SAVLQSGFRKMAFPS | 170 |
| 19_ORF1 | ORF1 | 3973 | 3992 | EVVLKKLKKSLNVAKSEFD | 171 |
| 26_ORF1 | ORF1 | 4651 | 4661 | DLTKPYIKWD | 172 |
| 90_ORF1 | ORF1 | 6651 | 6662 | LQEFKPRSQME | 173 |
| 193_ORF1 | ORF1 | 1047 | 1068 | EAKKVKPTVVVNAANVYLKHG | 174 |
| 129_ORF1 | ORF1 | 2490 | 2512 | VDSVTVKNGSIHLYFDKAGQKT | 175 |
| 159_ORF1 | ORF1 | 28 | 41 | RGFGDSVEEVLSE | 176 |
| 203_ORF1 | ORF1 | 1279 | 1294 | KKDAPYIVGDVVQEG | 177 |
| 53_ORF1 | ORF1 | 5792 | 5817 | AQCFKMFYKGVITHDVSSAINRPQI | 178 |
| 144_ORF1 | ORF1 | 349 | 355 | TKEGAT | 179 |
| 197_ORF1 | ORF1 | 1178 | 1196 | DKNLYDKLVSSFLEMKSE | 180 |
| 105_ORF1 | ORF1 | 1765 | 1784 | EAVMYMGTLSYEQFKKGVQ | 181 |
| 132_ORF1 | ORF1 | 2902 | 2909 | IEYTDFA | 182 |
| 21_ORF1 | ORF1 | 3950 | 3977 | LPSYAAFATAQEAYEQAVANGDSEVVL | 183 |
| 88_ORF1 | ORF1 | 6619 | 6638 | IGEAVKTQFNYYKKVDGVV | 184 |
| 157_ORF1 | ORF1 | 68 | 80 | VFIKRSDARTAP | 185 |
| 158_ORF1 | ORF1 | 91 | 102 | LEGIQYGRSGE | 186 |
| 163_ORF1 | ORF1 | 143 | 157 | DLGDELGTDPYEDF | 187 |
| 188_ORF1 | ORF1 | 1125 | 1158 | EDIQLLKSAYENFNQHEVLLAPLLSAGIFG | 188 |
| | | | | ADP | |
| 200_ORF1 | ORF1 | 1216 | 1233 | ESKPSVEQRKQDDKKIK | 189 |
| 101_ORF1 | ORF1 | 1795 | 1818 | YLVQQESPFVMMSAPPAQYELKH | 190 |
| 152_ORF1 | ORF1 | 280 | 295 | IKTIQPRVEKKKLDG | 191 |
| 143_ORF1 | ORF1 | 364 | 378 | VVKIYCPACHNSEV | 192 |
| 173_ORF1 | ORF1 | 843 | 859 | ELDERIDKVLNEKCSA | 193 |
| 136_ORF1 | ORF1 | 2605 | 2629 | MEKLKTLVATAEAELAKNVSLDNV | 194 |
| 28_ORF1 | ORF1 | 4416 | 4434 | GTSTDVVYRAFDIYNDKV | 195 |
| 11_ORF1 | ORF1 | 4879 | 4893 | INANQVIVNNLDKS | 196 |
| 8_ORF1 | ORF1 | 4932 | 4947 | QMNLKYAISAKNRAR | 197 |
| 87_ORF1 | ORF1 | 6598 | 6620 | SVKGLQPSVGPKQASLNGVTLI | 198 |
| 64_ORF1 | ORF1 | 6915 | 6938 | VHTANKWDLIISDMYDPKTKNVT | 199 |
| 114_ORF1 | ORF1 | 1987 | 2005 | KPIVWHVNNATNKATYKP | 200 |
| 55_ORF1 | ORF1 | 5770 | 5785 | EIVDTVSALVYDNKL | 201 |
| 151_ORF1 | ORF1 | 310 | 325 | ECNQMCLSTLMKCDH | 202 |
| 62_ORF1 | ORF1 | 5868 | 5877 | IFTQTTETA | 203 |
| 115_ORF1 | ORF1 | 3157 | 3178 | SNYLKRRVVFNGVSFSTFEEA | 204 |
| 2_ORF1 | ORF1 | 5240 | 5259 | KTDGTLMIERFVSLAIDAY | 205 |
| 1_ORF1 | ORF1 | 5265 | 5276 | NQEYADVFHLY | 206 |
| 189_ORF1 | ORF1 | 1095 | 1114 | PLKVGGSCVLSGHNLAKHC | 207 |
| 162_ORF1 | ORF1 | 166 | 181 | SGVTRELMRELNGGA | 208 |
| 131_ORF1 | ORF1 | 2916 | 2937 | AECTIFKDASGKPVPYCYDTN | 209 |
| 38_ORF1 | ORF1 | 4338 | 4352 | PKGFCDLKGKYVQI | 210 |
| 106_ORF1 | ORF1 | 1690 | 1705 | NPPALQDAYYRARAG | 211 |
| 96_ORF1 | ORF1 | 2048 | 2068 | EEVVENPTIQKDVLECNVKT | 212 |
| 72_ORF1 | ORF1 | 6355 | 6374 | FDKSAFVNLKQLPFFYYSD | 213 |
| 71_ORF1 | ORF1 | 6379 | 6396 | HGKQVVSDIDYVPLKSA | 214 |
| 204_ORF1 | ORF1 | 1268 | 1283 | TLVSDIDITFLKKDA | 215 |
| 182_ORF1 | ORF1 | 1526 | 1540 | FLKRGDKSVYYTSN | 216 |
| 44_ORF1 | ORF1 | 5432 | 5442 | IATCDWTNAG | 217 |
| 187_ORF1 | ORF1 | 1403 | 1418 | RKYKGIKIQEGVVDY | 218 |
| 142_ORF1 | ORF1 | 2836 | 2849 | WFSQRGGSYTNDK | 219 |
| 47_ORF1 | ORF1 | 5494 | 5518 | KPRPPLNRNYVFTGYRVTKNSKVQ | 220 |
| 23_ORF1 | ORF1 | 3723 | 3728 | GNALD | 221 |
| 118_ORF1 | ORF1 | 3197 | 3218 | LLPLTQYNRYLALYNKYKYFS | 222 |
| 109_ORF1 | ORF1 | 1867 | 1893 | YKENSYTTTIKPVTYKLDGVVCTEID | 223 |
| 24_ORF1 | ORF1 | 3840 | 3860 | NIKLLGVGGKPCIKVATVQS | 224 |
| 3_ORF1 | ORF1 | 5087 | 5108 | ICQAVTANVNALLSTDGNKIA | 225 |
| 49_ORF1 | ORF1 | 5724 | 5751 | DPAQLPAPRTLLTKGTLEPEYFNSVCR | 226 |
| 149_ORF1 | ORF1 | 479 | 508 | FSASTSAFVETVKGLDYKAFKQIVESCGN | 227 |
| 137_ORF1 | ORF1 | 2581 | 2590 | DSAEVAVKM | 228 |
| 125_ORF1 | ORF1 | 2522 | 2534 | NLDNLRANNTKG | 229 |
| 123_ORF1 | ORF1 | 3350 | 3371 | KLKVDTANPKTPKYKFVRIQP | 230 |
| 10_ORF1 | ORF1 | 4903 | 4926 | ARLYYDSMSYEDQDALFAYTKRN | 231 |
| 98_ORF1 | ORF1 | 2007 | 2029 | WCIRCLWSTKPVETSNSFDVLK | 232 |
| 20_ORF1 | ORF1 | 3933 | 3952 | MLDNRATLQAIASEFSSLP | 233 |
| 198_ORF1 | ORF1 | 1202 | 1214 | IAEIPKEEVKPF | 234 |
| 186_ORF1 | ORF1 | 1390 | 1400 | VCVETKAIVS | 235 |
| 68_ORF1 | ORF1 | 6863 | 6883 | RVIHFGAGSDKGVAPGTAVL | 236 |
| 160_ORF1 | ORF1 | 1 | 12 | ESLVPGFNEKT | 237 |
| 179_ORF1 | ORF1 | 1544 | 1567 | HLDGEVITFDNLKTLLSLREVRT | 238 |
| 69_ORF1 | ORF1 | 6821 | 6837 | KCDLQNYGDSATLPKG | 239 |
| 168_ORF1 | ORF1 | 723 | 741 | REETGLLMPLKAPKEIIF | 240 |
| 102_ORF1 | ORF1 | 1832 | 1843 | CGHYKHITSKE | 241 |
| 13_ORF1 | ORF1 | 4747 | 4764 | NQDVNLHSSRLSFKELL | 242 |
| 46_ORF1 | ORF1 | 5518 | 5543 | IGEYTFEKGDYGDAVVYRGTTTYKL | 243 |
| 77_ORF1 | ORF1 | 6266 | 6295 | PQADVEWKFYDAQPCSDKAYKIEELFYS | 244 |
| | | | | Y | |
| 83_ORF1 | ORF1 | 6722 | 6730 | MDSTVKNY | 245 |
| 127_ORF1 | ORF1 | 2546 | 2568 | SKCEESSAKSASVYYSQLMCQP | 246 |
| 181_ORF1 | ORF1 | 1515 | 1524 | YSGQSTQLG | 247 |
| 54_ORF1 | ORF1 | 5780 | 5794 | YDNKLKAHKDKSAQ | 248 |
| 172_ORF1 | ORF1 | 787 | 806 | LMLLEIKDTEKYCALAPNM | 249 |
| 104_ORF1 | ORF1 | 1752 | 1764 | KTCGQQQTTLKG | 250 |
| 126_ORF1 | ORF1 | 2540 | 2548 | IVFDGKSK | 251 |
| 119_ORF1 | ORF1 | 3531 | 3549 | KELLQNGMNGRTILGSAL | 252 |
| 150_ORF1 | ORF1 | 504 | 529 | SCGNFKVTKGKAKKGAWNIGEQKSI | 253 |
| 206_ORF1 | ORF1 | 1311 | 1330 | MLAKALRKVPTDNYITTYP | 254 |
| 97_ORF1 | ORF1 | 2026 | 2043 | VLKSEDAQGMDNLACED | 255 |
| 89_ORF1 | ORF1 | 6631 | 6653 | KKVDGVVQQLPETYFTQSRNLQ | 256 |
| 61_ORF1 | ORF1 | 5910 | 5934 | FTSLEIPRRNVATLQAENVTGLFK | 257 |
| 153_ORF1 | ORF1 | 285 | 309 | PRVEKKKLDGFMGRIRSVYPVASP | 258 |
| 184_ORF1 | ORF1 | 1455 | 1481 | GLNLEEAARYMRSLKVPATVSVSSPD | 259 |
| 196_ORF1 | ORF1 | 944 | 970 | TQYEYGTEDDYQGKPLEFGATSAALQ | 260 |
| 199_ORF1 | ORF1 | 1191 | 1212 | EMKSEKQVEQKIAEIPKEEVK | 261 |
| 58_ORF1 | ORF1 | 6053 | 6073 | NNTDFSRVSAKPPPGDQFKH | 262 |
| 79_ORF1 | ORF1 | 6454 | 6464 | ENVAFNVVNK | 263 |
| 84_ORF1 | ORF1 | 6706 | 6720 | AKRFKESPFELEDF | 264 |
| 37_ORF1 | ORF1 | 4074 | 4081 | PDYNTYK | 265 |
| 31_ORF1 | ORF1 | 4449 | 4469 | EKDEDDNLIDSYFVVKRHTF | 266 |
| 183_ORF1 | ORF1 | 1432 | 1443 | SLINTLNDLNE | 267 |
| 175_ORF1 | ORF1 | 1638 | 1654 | DPSFLGRYMSALNHTK | 268 |
| 41_ORF1 | ORF1 | 4230 | 4239 | IKGLNNLNR | 269 |
| 14_ORF1 | ORF1 | 4791 | 4821 | ALTNNVAFQTVKPGNFNKDFYDFAVSK | 270 |
| | | | | GFF | |
| 155_ORF1 | ORF1 | 209 | 226 | KASCTLSEQLDFIDTKR | 271 |
| 7_ORF1 | ORF1 | 5161 | 5183 | YASQGLVASIKNFKSVLYYQNN | 272 |
| 167_ORF1 | ORF1 | 703 | 719 | NLGETFVTHSKGLYRK | 273 |
| 174_ORF1 | ORF1 | 823 | 841 | TFGDDTVIEVQGYKSVNI | 274 |
| 110_ORF1 | ORF1 | 1848 | 1871 | DGALLTKSSEYKGPITDVFYKEN | 275 |
| 95_ORF1 | ORF1 | 2255 | 2261 | NLGMPS | 276 |
| 140_ORF1 | ORF1 | 2802 | 2815 | SSEIIGYKAIDGG | 277 |
| 120_ORF1 | ORF1 | 3477 | 3485 | GDRWFLNR | 278 |
| 18_ORF1 | ORF1 | 4035 | 4047 | MLRKLDNDALNN | 279 |
| 42_ORF1 | ORF1 | 4257 | 4263 | TEVPAN | 280 |
| 39_ORF1 | ORF1 | 4315 | 4321 | MDQESF | 281 |
| 4_ORF1 | ORF1 | 5131 | 5137 | DFVNEF | 282 |
| 56_ORF1 | ORF1 | 6102 | 6118 | SDRVVFVLWAHGFELT | 283 |
| 82_ORF1 | ORF1 | 6740 | 6755 | KCVCSVIDLLLDDFV | 284 |
| 52_ORF1 | ORF1 | 5833 | 5849 | VFISPYNSQNAVASKI | 285 |
| 15_ORF1 | ORF1 | 4821 | 4836 | KEGSSVELKHFFFAQ | 286 |
| 185_ORF1 | ORF1 | 1356 | 1367 | PSIISNEKQEI | 287 |
| 59_ORF1 | ORF1 | 6016 | 6045 | EGCHATREAVGTNLPLQLGFSTGVNLVA | 288 |
| | | | | V | |
| 202_ORF1 | ORF1 | 1255 | 1265 | YIDINGNLHP | 289 |
| 135_ORF1 | ORF1 | 2635 | 2658 | AARQGFVDSDVETKDVVECLKLS | 290 |
| 147_ORF1 | ORF1 | 463 | 480 | VGDFKLNEEIAIILASF | 291 |
| 107_ORF1 | ORF1 | 1716 | 1747 | YCNKTVGELGDVRETMSYLFQHANLDS | 292 |
| | | | | CKRV | |
| 145_ORF1 | ORF1 | 393 | 401 | KTILRKGG | 293 |
| 32_ORF1 | ORF1 | 4474 | 4506 | EETIYNLLKDCPAVAKHDFFKFRIDGDM | 294 |
| | | | | VPHI | |
| 78_ORF1 | ORF1 | 6465 | 6493 | HFDGQQGEVPVSIINNTVYTKVDGVDVE | 295 |
| 34_ORF1 | ORF1 | 4153 | 4159 | CAAGTT | 296 |
| 146_ORF1 | ORF1 | 423 | 431 | VPRASANI | 297 |
| 60_ORF1 | ORF1 | 5929 | 5947 | TGLFKDCSKVITGLHPTQ | 298 |
| 121_ORF1 | ORF1 | 3392 | 3407 | MRPNFTIKGSFLNGS | 299 |
| 178_ORF1 | ORF1 | 1571 | 1580 | TTVDNINLH | 300 |
| 40_ORF1 | ORF1 | 4274 | 4304 | DAAKAYKDYLASGGQPITNCVKMLCTHT | 301 |
| | | | | GT | |
| 33_ORF1 | ORF1 | 4180 | 4198 | VLALLSDLQDLKWARFPK | 302 |
| 194_ORF1 | ORF1 | 995 | 1017 | DNQTTTIQTIVEVQPQLEMELT | 303 |
| 130_ORF1 | ORF1 | 2946 | 2965 | SLRPDTRYVLMDGSIIQFP | 304 |
| 27_ORF1 | ORF1 | 4599 | 4612 | DNQDLNGNWYDFG | 305 |
| 12_ORF1 | ORF1 | 4896 | 4907 | PFNKWGKARLY | 306 |
| 156_ORF1 | ORF1 | 194 | 210 | GYPLECIKDLLARAGK | 307 |
| 207_ORF1 | ORF1 | 1332 | 1341 | GLNGYTVEE | 308 |
| 195_ORF1 | ORF1 | 928 | 936 | EDEEEGDC | 309 |
| 17_ORF1 | ORF1 | 4010 | 4022 | QMYKQARSEDKR | 310 |
| 57_ORF1 | ORF1 | 6135 | 6144 | DRRATCFST | 311 |
| 73_ORF1 | ORF1 | 6338 | 6347 | CDGGSLYVN | 312 |
| 29_ORF1 | ORF1 | 4398 | 4413 | FLNRVCGVSAARLTP | 313 |
| 66_ORF1 | ORF1 | 6975 | 6980 | ADLYK | 314 |
| 169_ORF1 | ORF1 | 658 | 669 | IVGGQIVTCAK | 315 |
| 122_ORF1 | ORF1 | 3426 | 3441 | HMELPTGVHAGTDLE | 316 |
| 22_ORF1 | ORF1 | 3897 | 3905 | ILLAKDTT | 317 |
| 5_ORF1 | ORF1 | 5198 | 5222 | KGPHEFCSQHTMLVKQGDDYVYLP | 318 |
| 67_ORF1 | ORF1 | 6891 | 6905 | LLVDSDLNDFVSDA | 319 |
| 63_ORF1 | ORF1 | 5893 | 5908 | VGILCIMSDRDLYDK | 320 |
| 124_ORF1 | ORF1 | 3320 | 3331 | LIRKSNHNFLV | 321 |
| 111_ORF1 | ORF1 | 1916 | 1930 | NQPYPNASFDNFKF | 322 |
| 176_ORF1 | ORF1 | 1654 | 1673 | KWKYPQVNGLTSIKWADNN | 323 |
| 36_ORF1 | ORF1 | 4102 | 4113 | DADSKIVQLSE | 324 |
| 141_ORF1 | ORF1 | 2818 | 2832 | DIASTDTCFANKHA | 325 |
| 50_ORF1 | ORF1 | 5667 | 5683 | DKFKVNSTLEQYVFCT | 326 |
| 45_ORF1 | ORF1 | 5459 | 5470 | ETLKATEETFK | 327 |
| 93_ORF1 | ORF1 | 2163 | 2170 | VCTNYMP | 328 |
| 16_ORF1 | ORF1 | 4847 | 4856 | YRYNLPTMC | 329 |
| 75_ORF1 | ORF1 | 6177 | 6183 | LQSNHD | 330 |
| 70_ORF1 | ORF1 | 6790 | 6798 | ETFYPKLQ | 331 |
| 9_ORF1 | ORF1 | 4959 | 4969 | NRQFHQKLLK | 332 |

TABLE 4
Epitopes and Associated Peptide_Sequences

| Epitope_id | Start | End | Protein | Epitope_Sequence | # | RBD |
|---|---|---|---|---|---|---|
| 1_1.0_M | 1 | 11 | M | ADSNGTITVE | 333 | FALSE |
| 3_1.0_M | 153 | 165 | M | HHLGRCDIKDLP | 334 | FALSE |
| 3_2.0_M | 157 | 168 | M | RCDIKDLPKEI | 335 | FALSE |
| 4_1.0_M | 177 | 191 | M | YYKLGASQRVAGDS | 336 | FALSE |
| 4_2.0_M | 188 | 196 | M | GDSGFAAY | 337 | FALSE |
| 2_1.0_M | 199 | 210 | M | RIGNYKLNTDH | 338 | FALSE |
| 2_2.0_M | 206 | 221 | M | NTDHSSSSDNIALLVQ | 339 | FALSE |
| 2_3.0_M | 207 | 221 | M | TDHSSSSDNIALLVQ | 340 | FALSE |
| 9_1.0_N | 23 | 31 | N | TGSNQNGE | 341 | FALSE |
| 10_2.0_N | 32 | 41 | N | SGARSKQRR | 342 | FALSE |
| 10_3.0_N | 33 | 44 | N | GARSKQRRPQG | 343 | FALSE |
| 10_1.0_N | 35 | 46 | N | RSKQRRPQGLP | 344 | FALSE |
| 8_3.0_N | 35 | 48 | N | RSKQRRPQGLPNN | 345 | FALSE |
| 8_4.0_N | 38 | 48 | N | QRRPQGLPNN | 346 | FALSE |
| 8_2.0_N | 41 | 49 | N | PQGLPNNT | 347 | FALSE |
| 8_1.0_N | 45 | 57 | N | PNNTASWFTALT | 348 | FALSE |
| 12_2.0_N | 59 | 67 | N | GKEDLKFP | 349 | FALSE |
| 12_1.0_N | 71 | 78 | N | VPINTNS | 350 | FALSE |
| 13_1.0_N | 84 | 94 | N | GYYRRATRRI | 351 | FALSE |
| 13_2.0_N | 90 | 103 | N | TRRIRGGDGKMKD | 352 | FALSE |
| 13_3.0_N | 91 | 102 | N | RRIRGGDGKMK | 353 | FALSE |
| 13_7.0_N | 91 | 105 | N | RRIRGGDGKMKDLS | 354 | FALSE |
| 13_5.0_N | 92 | 104 | N | RIRGGDGKMKDL | 355 | FALSE |
| 13_4.0_N | 93 | 104 | N | IRGGDGKMKDL | 356 | FALSE |
| 13_6.0_N | 95 | 105 | N | GGDGKMKDLS | 357 | FALSE |
| 13_8.0_N | 101 | 111 | N | KDLSPRWYFY | 358 | FALSE |
| 11_2.0_N | 109 | 132 | N | FYYLGTGPEAGLPYGANKDGIIW | 359 | FALSE |
| 11_1.0_N | 114 | 129 | N | TGPEAGLPYGANKDG | 360 | FALSE |
| 11_5.0_N | 121 | 132 | N | PYGANKDGIIW | 361 | FALSE |
| 11_4.0_N | 126 | 132 | N | KDGIIW | 362 | FALSE |
| 11_3.0_N | 126 | 145 | N | KDGIIWVATEGALNTPKDH | 363 | FALSE |
| 16_2.0_N | 141 | 152 | N | PKDHIGTRNPA | 364 | FALSE |
| 16_1.0_N | 151 | 157 | N | ANNAAI | 365 | FALSE |
| 15_2.0_N | 158 | 171 | N | LQLPQGTTLPKGF | 366 | FALSE |
| 15_1.0_N | 159 | 168 | N | QLPQGTTLP | 367 | FALSE |
| 15_4.0_N | 159 | 174 | N | QLPQGTTLPKGFYAE | 368 | FALSE |
| 15_3.0_N | 160 | 174 | N | LPQGTTLPKGFYAE | 369 | FALSE |
| 14_1.0_N | 182 | 193 | N | SSRSSSRSRNS | 370 | FALSE |
| 14_2.0_N | 186 | 197 | N | SSRSRNSSRNS | 371 | FALSE |
| 14_3.0_N | 186 | 197 | N | SSRSRNSSRNS | 372 | FALSE |
| 14_4.0_N | 191 | 198 | N | NSSRNST | 373 | FALSE |
| 18_5.0_N | 201 | 209 | N | SRGTSPAR | 374 | FALSE |
| 18_1.0_N | 207 | 226 | N | ARMAGNGGDAALALLLLDR | 375 | FALSE |
| 18_6.0_N | 210 | 220 | N | AGNGGDAALA | 376 | FALSE |
| 18_7.0_N | 212 | 222 | N | NGGDAALALL | 377 | FALSE |
| 18_4.0_N | 213 | 218 | N | GGDAA | 378 | FALSE |
| 18_8.0_N | 213 | 226 | N | GGDAALALLLLDR | 379 | FALSE |
| 18_2.0_N | 213 | 227 | N | GGDAALALLLLDRL | 380 | FALSE |
| 17_1.0_N | 213 | 232 | N | GGDAALALLLLDRLNQLES | 381 | FALSE |
| 17_2.0_N | 213 | 233 | N | GGDAALALLLLDRLNQLESK | 382 | FALSE |
| 20_5.0_N | 213 | 243 | N | GGDAALALLLLDRLNQLESKMSGKGQQQQG | 383 | FALSE |
| 18_3.0_N | 214 | 228 | N | GDAALALLLLDRLN | 384 | FALSE |
| 19_1.0_N | 218 | 232 | N | LALLLLDRLNQLES | 385 | FALSE |
| 19_3.0_N | 220 | 233 | N | LLLLDRLNQLESK | 386 | FALSE |
| 19_7.0_N | 220 | 235 | N | LLLLDRLNQLESKMS | 387 | FALSE |
| 19_2.0_N | 221 | 232 | N | LLLDRLNQLES | 388 | FALSE |
| 20_8.0_N | 221 | 243 | N | LLLDRLNQLESKMSGKGQQQQG | 389 | FALSE |
| 19_6.0_N | 222 | 235 | N | LLDRLNQLESKMS | 390 | FALSE |
| 20_6.0_N | 223 | 242 | N | LDRLNQLESKMSGKGQQQQ | 391 | FALSE |
| 20_9.0_N | 223 | 243 | N | LDRLNQLESKMSGKGQQQQG | 392 | FALSE |
| 19_5.0_N | 224 | 234 | N | DRLNQLESKM | 393 | FALSE |
| 19_4.0_N | 224 | 235 | N | DRLNQLESKMS | 394 | FALSE |
| 20_10.0N | 224 | 251 | N | DRLNQLESKMSGKGQQQQGQTVTKKSA | 395 | FALSE |
| 20_4.0_N | 229 | 242 | N | LESKMSGKGQQQQ | 396 | FALSE |
| 20_1.0_N | 229 | 243 | N | LESKMSGKGQQQQG | 397 | FALSE |
| 20_3.0_N | 230 | 242 | N | ESKMSGKGQQQQ | 398 | FALSE |
| 20_2.0_N | 230 | 244 | N | ESKMSGKGQQQQGQ | 399 | FALSE |
| 20_7.0_N | 231 | 238 | N | SKMSGKG | 400 | FALSE |
| 22_5.0_N | 235 | 247 | N | GKGQQQQGQTVT | 401 | FALSE |
| 22_4.0_N | 238 | 251 | N | QQQQGQTVTKKSA | 402 | FALSE |
| 22_1.0_N | 238 | 257 | N | QQQQGQTVTKKSAAEASKK | 403 | FALSE |
| 22_2.0_N | 239 | 254 | N | QQQGQTVTKKSAAEA | 404 | FALSE |
| 22_3.0_N | 241 | 250 | N | QGQTVTKKS | 405 | FALSE |
| 21_3.0_N | 243 | 258 | N | QTVTKKSAAEASKKP | 406 | FALSE |
| 21_2.0_N | 245 | 258 | N | VTKKSAAEASKKP | 407 | FALSE |
| 21_1.0_N | 248 | 258 | N | KSAAEASKKP | 408 | FALSE |
| 26_1.0_N | 253 | 263 | N | ASKKPRQKRT | 409 | FALSE |
| 26_2.0_N | 255 | 266 | N | KKPRQKRTATK | 410 | FALSE |
| 26_5.0_N | 258 | 269 | N | RQKRTATKAYN | 411 | FALSE |
| 26_3.0_N | 259 | 274 | N | QKRTATKAYNVTQAF | 412 | FALSE |
| 26_4.0_N | 260 | 265 | N | KRTAT | 413 | FALSE |
| 26_6.0_N | 262 | 267 | N | TATKA | 414 | FALSE |
| 26_7.0_N | 264 | 272 | N | TKAYNVTQ | 415 | FALSE |
| 25_1.0_N | 267 | 278 | N | YNVTQAFGRRG | 416 | FALSE |
| 25_2.0_N | 267 | 279 | N | YNVTQAFGRRGP | 417 | FALSE |
| 23_1.0_N | 281 | 289 | N | TQGNFGDQ | 418 | FALSE |
| 23_2.0_N | 291 | 300 | N | IRQGTDYKH | 419 | FALSE |
| 24_1.0_N | 311 | 323 | N | SAFFGMSRIGME | 420 | FALSE |
| 1_1.0_N | 338 | 350 | N | LDDKDPNFKDQV | 421 | FALSE |
| 1_2.0_N | 338 | 361 | N | LDDKDPNFKDQVILLNKHIDAYK | 422 | FALSE |
| 1_3.0_N | 344 | 357 | N | NFKDQVILLNKHI | 423 | FALSE |
| 1_4.0_N | 347 | 355 | N | DQVILLNK | 424 | FALSE |
| 2_1.0_N | 351 | 361 | N | LLNKHIDAYK | 425 | FALSE |
| 2_2.0_N | 353 | 361 | N | NKHIDAYK | 426 | FALSE |
| 2_4.0_N | 354 | 370 | N | KHIDAYKTFPPTEPKK | 427 | FALSE |
| 2_3.0_N | 357 | 369 | N | DAYKTFPPTEPK | 428 | FALSE |
| 3_1.0_N | 361 | 372 | N | TFPPTEPKKDK | 429 | FALSE |
| 3_3.0_N | 362 | 370 | N | FPPTEPKK | 430 | FALSE |
| 3_2.0_N | 364 | 371 | N | PTEPKKD | 431 | FALSE |
| 3_4.0_N | 364 | 374 | N | PTEPKKDKKK | 432 | FALSE |
| 3_6.0_N | 366 | 375 | N | EPKKDKKKK | 433 | FALSE |
| 3_5.0_N | 366 | 377 | N | EPKKDKKKKAD | 434 | FALSE |
| 7_5.0_N | 370 | 388 | N | DKKKKADETQALPQRQKK | 435 | FALSE |
| 7_6.0_N | 372 | 383 | N | KKKADETQALP | 436 | FALSE |
| 7_4.0_N | 373 | 389 | N | KKADETQALPQRQKKQ | 437 | FALSE |
| 7_7.0_N | 376 | 381 | N | DETQA | 438 | FALSE |
| 7_3.0_N | 376 | 388 | N | DETQALPQRQKK | 439 | FALSE |
| 7_1.0_N | 376 | 389 | N | DETQALPQRQKKQ | 440 | FALSE |
| 6_7.0_N | 376 | 398 | N | DETQALPQRQKKQQTVTLLPAA | 441 | FALSE |
| 7_2.0_N | 377 | 388 | N | ETQALPQRQKK | 442 | FALSE |
| 6_1.0_N | 381 | 394 | N | LPQRQKKQQTVTL | 443 | FALSE |
| 6_4.0_N | 385 | 398 | N | QKKQQTVTLLPAA | 444 | FALSE |
| 6_6.0_N | 386 | 394 | N | KKQQTVTL | 445 | FALSE |
| 6_5.0_N | 386 | 396 | N | KKQQTVTLLP | 446 | FALSE |
| 6_2.0_N | 390 | 395 | N | TVTLL | 447 | FALSE |
| 6_3.0_N | 390 | 396 | N | TVTLLP | 448 | FALSE |
| 4_3.0_N | 391 | 405 | N | VTLLPAADLDDFSK | 449 | FALSE |
| 4_1.0_N | 391 | 406 | N | VTLLPAADLDDFSKQ | 450 | FALSE |
| 4_4.0_N | 393 | 406 | N | LLPAADLDDFSKQ | 451 | FALSE |
| 4_2.0_N | 395 | 407 | N | PAADLDDFSKQL | 452 | FALSE |
| 5_1.0_N | 395 | 411 | N | PAADLDDFSKQLQQSM | 453 | FALSE |
| 5_3.0_N | 400 | 416 | N | DDFSKQLQQSMSSADS | 454 | FALSE |
| 5_2.0_N | 403 | 415 | N | SKQLQQSMSSAD | 455 | FALSE |
| 5_4.0_N | 414 | 418 | N | DSTQA | 456 | FALSE |
| 160_1.0_ORF1 | 1 | 10 | ORF1 | ESLVPGFNE | 457 | FALSE |
| 160_2.0_ORF1 | 3 | 12 | ORF1 | LVPGFNEKT | 458 | FALSE |
| 159_1.0_ORF1 | 28 | 41 | ORF1 | RGFGDSVEEVLSE | 459 | FALSE |
| 157_1.0_ORF1 | 68 | 80 | ORF1 | VFIKRSDARTAP | 460 | FALSE |
| 158_1.0_ORF1 | 91 | 102 | ORF1 | LEGIQYGRSGE | 461 | FALSE |
| 161_3.0_ORF1 | 112 | 121 | ORF1 | EIPVAYRKV | 462 | FALSE |
| 161_1.0_ORF1 | 114 | 128 | ORF1 | PVAYRKVLLRKNGN | 463 | FALSE |
| 161_2.0_ORF1 | 118 | 123 | ORF1 | RKVLL | 464 | FALSE |
| 161_5.0_ORF1 | 119 | 130 | ORF1 | KVLLRKNGNKG | 465 | FALSE |
| 161_4.0_ORF1 | 122 | 132 | ORF1 | LRKNGNKGAG | 466 | FALSE |
| 163_1.0_ORF1 | 143 | 151 | ORF1 | DLGDELGT | 467 | FALSE |
| 163_2.0_ORF1 | 147 | 154 | ORF1 | ELGTDPY | 468 | FALSE |
| 163_3.0_ORF1 | 152 | 157 | ORF1 | PYEDF | 469 | FALSE |
| 164_3.0_ORF1 | 152 | 166 | ORF1 | PYEDFQENWNTKHS | 470 | FALSE |
| 164_2.0_ORF1 | 152 | 166 | ORF1 | PYEDFQENWNTKHS | 471 | FALSE |
| 164_1.0_ORF1 | 156 | 165 | ORF1 | FQENWNTKH | 472 | FALSE |
| 164_4.0_ORF1 | 161 | 167 | ORF1 | NTKHSS | 473 | FALSE |
| 162_1.0_ORF1 | 166 | 173 | ORF1 | SGVTREL | 474 | FALSE |
| 162_2.0_ORF1 | 170 | 181 | ORF1 | RELMRELNGGA | 475 | FALSE |
| 162_4.0_ORF1 | 171 | 177 | ORF1 | ELMREL | 476 | FALSE |
| 162_3.0_ORF1 | 172 | 178 | ORF1 | LMRELN | 477 | FALSE |
| 156_1.0_ORF1 | 194 | 202 | ORF1 | GYPLECIK | 478 | FALSE |
| 156_2.0_ORF1 | 202 | 210 | ORF1 | DLLARAGK | 479 | FALSE |
| 155_1.0_ORF1 | 209 | 226 | ORF1 | KASCTLSEQLDFIDTKR | 480 | FALSE |
| 154_4.0_ORF1 | 244 | 249 | ORF1 | SEKSY | 481 | FALSE |
| 154_1.0_ORF1 | 252 | 261 | ORF1 | TPFEIKLAK | 482 | FALSE |
| 154_2.0_ORF1 | 252 | 265 | ORF1 | TPFEIKLAKKFDT | 483 | FALSE |
| 154_3.0_ORF1 | 260 | 273 | ORF1 | KKFDTFNGECPNF | 484 | FALSE |
| 152_2.0_ORF1 | 280 | 291 | ORF1 | IKTIQPRVEKK | 485 | FALSE |
| 152_3.0_ORF1 | 282 | 293 | ORF1 | TIQPRVEKKKL | 486 | FALSE |
| 152_1.0_ORF1 | 282 | 295 | ORF1 | TIQPRVEKKKLDG | 487 | FALSE |
| 153_1.0_ORF1 | 285 | 302 | ORF1 | PRVEKKKLDGFMGRIRS | 488 | FALSE |
| 153_3.0_ORF1 | 290 | 297 | ORF1 | KKLDGFM | 489 | FALSE |
| 153_2.0_ORF1 | 298 | 309 | ORF1 | RIRSVYPVASP | 490 | FALSE |
| 151_1.0_ORF1 | 310 | 325 | ORF1 | ECNQMCLSTLMKCDH | 491 | FALSE |
| 144_1.0_ORF1 | 349 | 355 | ORF1 | TKEGAT | 492 | FALSE |
| 143_1.0_ORF1 | 364 | 376 | ORF1 | VVKIYCPACHNS | 493 | FALSE |
| 143_2.0_ORF1 | 369 | 378 | ORF1 | CPACHNSEV | 494 | FALSE |
| 145_1.0_ORF1 | 393 | 401 | ORF1 | KTILRKGG | 495 | FALSE |
| 146_1.0_ORF1 | 423 | 431 | ORF1 | VPRASANI | 496 | FALSE |
| 148_1.0_ORF1 | 447 | 458 | ORF1 | DNLLEILQKEK | 497 | FALSE |
| 148_2.0_ORF1 | 455 | 471 | ORF1 | KEKVNINIVGDFKLNE | 498 | FALSE |
| 147_3.0_ORF1 | 463 | 471 | ORF1 | VGDFKLNE | 499 | FALSE |
| 147_1.0_ORF1 | 467 | 473 | ORF1 | KLNEEI | 500 | FALSE |
| 147_2.0_ORF1 | 469 | 475 | ORF1 | NEEIAI | 501 | FALSE |
| 147_4.0_ORF1 | 471 | 480 | ORF1 | EIAIILASF | 502 | FALSE |
| 149_6.0_ORF1 | 479 | 495 | ORF1 | FSASTSAFVETVKGLD | 503 | FALSE |
| 149_1.0_ORF1 | 489 | 508 | ORF1 | TVKGLDYKAFKQIVESCGN | 504 | FALSE |
| 149_5.0_ORF1 | 490 | 498 | ORF1 | VKGLDYKA | 505 | FALSE |
| 149_3.0_ORF1 | 491 | 500 | ORF1 | KGLDYKAFK | 506 | FALSE |
| 149_4.0_ORF1 | 493 | 508 | ORF1 | LDYKAFKQIVESCGN | 507 | FALSE |
| 149_2.0_ORF1 | 494 | 500 | ORF1 | DYKAFK | 508 | FALSE |
| 150_2.0_ORF1 | 504 | 515 | ORF1 | SCGNFKVTKGK | 509 | FALSE |
| 150_1.0_ORF1 | 511 | 517 | ORF1 | TKGKAK | 510 | FALSE |
| 150_4.0_ORF1 | 516 | 526 | ORF1 | KKGAWNIGEQ | 511 | FALSE |
| 150_3.0_ORF1 | 522 | 529 | ORF1 | IGEQKSI | 512 | FALSE |
| 169_1.0_ORF1 | 658 | 669 | ORF1 | IVGGQIVTCAK | 513 | FALSE |
| 167_1.0_ORF1 | 703 | 714 | ORF1 | NLGETFVTHSK | 514 | FALSE |
| 167_2.0_ORF1 | 705 | 719 | ORF1 | GETFVTHSKGLYRK | 515 | FALSE |
| 168_2.0_ORF1 | 723 | 738 | ORF1 | REETGLLMPLKAPKE | 516 | FALSE |
| 168_1.0_ORF1 | 730 | 741 | ORF1 | MPLKAPKEIIF | 517 | FALSE |
| 166_1.0_ORF1 | 747 | 760 | ORF1 | PTEVLTEEVVLKT | 518 | FALSE |
| 166_3.0_ORF1 | 748 | 766 | ORF1 | TEVLTEEVVLKTGDLQPL | 519 | FALSE |
| 166_2.0_ORF1 | 752 | 768 | ORF1 | TEEVVLKTGDLQPLEQ | 520 | FALSE |
| 166_4.0_ORF1 | 753 | 762 | ORF1 | EEVVLKTGD | 521 | FALSE |
| 165_1.0_ORF1 | 766 | 776 | ORF1 | EQPTSEAVEA | 522 | FALSE |
| 165_2.0_ORF1 | 769 | 780 | ORF1 | TSEAVEAPLVG | 523 | FALSE |
| 172_2.0_ORF1 | 787 | 796 | ORF1 | LMLLEIKDT | 524 | FALSE |
| 172_1.0_ORF1 | 790 | 799 | ORF1 | LEIKDTEKY | 525 | FALSE |
| 172_3.0_ORF1 | 794 | 806 | ORF1 | DTEKYCALAPNM | 526 | FALSE |
| 171_2.0_ORF1 | 803 | 817 | ORF1 | PNMMVTNNTFTLKG | 527 | FALSE |
| 171_1.0_ORF1 | 809 | 819 | ORF1 | NNTFTLKGGA | 528 | FALSE |
| 174_2.0_ORF1 | 823 | 830 | ORF1 | TFGDDTV | 529 | FALSE |
| 174_1.0_ORF1 | 826 | 838 | ORF1 | DDTVIEVQGYKS | 530 | FALSE |
| 174_3.0_ORF1 | 832 | 841 | ORF1 | VQGYKSVNI | 531 | FALSE |
| 173_2.0_ORF1 | 843 | 853 | ORF1 | ELDERIDKVL | 532 | FALSE |
| 173_1.0_ORF1 | 843 | 859 | ORF1 | ELDERIDKVLNEKCSA | 533 | FALSE |
| 170_2.0_ORF1 | 880 | 886 | ORF1 | KTLQPV | 534 | FALSE |
| 170_1.0_ORF1 | 881 | 891 | ORF1 | TLQPVSELLT | 535 | FALSE |
| 170_3.0_ORF1 | 882 | 888 | ORF1 | LQPVSE | 536 | FALSE |
| 195_1.0_ORF1 | 928 | 936 | ORF1 | EDEEEGDC | 537 | FALSE |
| 196_1.0_ORF1 | 944 | 959 | ORF1 | TQYEYGTEDDYQGKP | 538 | FALSE |
| 196_2.0_ORF1 | 957 | 970 | ORF1 | KPLEFGATSAALQ | 539 | FALSE |
| 194_2.0_ORF1 | 995 | 1003 | ORF1 | DNQTTTIQ | 540 | FALSE |
| 194_3.0_ORF1 | 999 | 1005 | ORF1 | TTIQTI | 541 | FALSE |
| 194_1.0_ORF1 | 1004 | 1017 | ORF1 | IVEVQPQLEMELT | 542 | FALSE |
| 192_3.0_ORF1 | 1035 | 1042 | ORF1 | DNVYIKN | 543 | FALSE |
| 192_2.0_ORF1 | 1035 | 1046 | ORF1 | DNVYIKNADIV | 544 | FALSE |
| 192_1.0_ORF1 | 1035 | 1050 | ORF1 | DNVYIKNADIVEEAK | 545 | FALSE |
| 193_2.0_ORF1 | 1047 | 1056 | ORF1 | EAKKVKPTV | 546 | FALSE |
| 193_3.0_ORF1 | 1048 | 1062 | ORF1 | AKKVKPTVVVNAAN | 547 | FALSE |
| 193_1.0_ORF1 | 1054 | 1068 | ORF1 | TVVVNAANVYLKHG | 548 | FALSE |
| 191_1.0_ORF1 | 1066 | 1079 | ORF1 | HGGGVAGALNKAT | 549 | FALSE |
| 191_2.0_ORF1 | 1072 | 1082 | ORF1 | GALNKATNNA | 550 | FALSE |
| 191_4.0_ORF1 | 1075 | 1084 | ORF1 | NKATNNAMQ | 551 | FALSE |
| 191_3.0_ORF1 | 1075 | 1090 | ORF1 | NKATNNAMQVESDDY | 552 | FALSE |
| 189_2.0_ORF1 | 1095 | 1107 | ORF1 | PLKVGGSCVLSG | 553 | FALSE |
| 189_1.0_ORF1 | 1106 | 1114 | ORF1 | GHNLAKHC | 554 | FALSE |
| 190_1.0_ORF1 | 1112 | 1127 | ORF1 | HCLHVVGPNVNKGED | 555 | FALSE |
| 190_2.0_ORF1 | 1118 | 1130 | ORF1 | GPNVNKGEDIQL | 556 | FALSE |
| 190_3.0_ORF1 | 1122 | 1134 | ORF1 | NKGEDIQLLKSA | 557 | FALSE |
| 188_1.0_ORF1 | 1125 | 1145 | ORF1 | EDIQLLKSAYENFNQHEVLL | 558 | FALSE |
| 188_2.0_ORF1 | 1136 | 1145 | ORF1 | NFNQHEVLL | 559 | FALSE |
| 188_3.0_ORF1 | 1147 | 1158 | ORF1 | LLSAGIFGADP | 560 | FALSE |
| 197_2.0_ORF1 | 1178 | 1187 | ORF1 | DKNLYDKLV | 561 | FALSE |
| 197_3.0_ORF1 | 1180 | 1196 | ORF1 | NLYDKLVSSFLEMKSE | 562 | FALSE |
| 197_1.0_ORF1 | 1181 | 1196 | ORF1 | LYDKLVSSFLEMKSE | 563 | FALSE |
| 199_1.0_ORF1 | 1191 | 1202 | ORF1 | EMKSEKQVEQK | 564 | FALSE |
| 199_2.0_ORF1 | 1193 | 1205 | ORF1 | KSEKQVEQKIAE | 565 | FALSE |
| 199_3.0_ORF1 | 1195 | 1200 | ORF1 | EKQVE | 566 | FALSE |
| 199_4.0_ORF1 | 1198 | 1207 | ORF1 | VEQKIAEIP | 567 | FALSE |
| 199_5.0_ORF1 | 1198 | 1212 | ORF1 | VEQKIAEIPKEEVK | 568 | FALSE |
| 198_2.0_ORF1 | 1202 | 1214 | ORF1 | IAEIPKEEVKPF | 569 | FALSE |
| 201_3.0_ORF1 | 1205 | 1219 | ORF1 | IPKEEVKPFITESK | 570 | FALSE |
| 198_1.0_ORF1 | 1208 | 1214 | ORF1 | EEVKPF | 571 | FALSE |
| 201_4.0_ORF1 | 1208 | 1222 | ORF1 | EEVKPFITESKPSV | 572 | FALSE |
| 201_2.0_ORF1 | 1208 | 1222 | ORF1 | EEVKPFITESKPSV | 573 | FALSE |
| 201_1.0_ORF1 | 1216 | 1223 | ORF1 | ESKPSVE | 574 | FALSE |
| 200_4.0_ORF1 | 1216 | 1229 | ORF1 | ESKPSVEQRKQDD | 575 | FALSE |
| 200_2.0_ORF1 | 1217 | 1233 | ORF1 | SKPSVEQRKQDDKKIK | 576 | FALSE |
| 200_3.0_ORF1 | 1218 | 1229 | ORF1 | KPSVEQRKQDD | 577 | FALSE |
| 200_1.0_ORF1 | 1224 | 1230 | ORF1 | RKQDDK | 578 | FALSE |
| 202_1.0_ORF1 | 1255 | 1265 | ORF1 | YIDINGNLHP | 579 | FALSE |
| 204_2.0_ORF1 | 1268 | 1277 | ORF1 | TLVSDIDIT | 580 | FALSE |
| 204_1.0_ORF1 | 1272 | 1283 | ORF1 | DIDITFLKKDA | 581 | FALSE |
| 203_1.0_ORF1 | 1279 | 1294 | ORF1 | KKDAPYIVGDVVQEG | 582 | FALSE |
| 205_1.0_ORF1 | 1291 | 1305 | ORF1 | QEGVLTAVVIPTKK | 583 | FALSE |
| 205_2.0_ORF1 | 1300 | 1305 | ORF1 | IPTKK | 584 | FALSE |
| 205_4.0_ORF1 | 1301 | 1312 | ORF1 | PTKKAGGTTEM | 585 | FALSE |
| 205_3.0_ORF1 | 1301 | 1324 | ORF1 | PTKKAGGTTEMLAKALRKVPTDN | 586 | FALSE |
| 205_6.0_ORF1 | 1302 | 1313 | ORF1 | TKKAGGTTEML | 587 | FALSE |
| 205_7.0_ORF1 | 1306 | 1313 | ORF1 | GGTTEML | 588 | FALSE |
| 205_8.0_ORF1 | 1306 | 1315 | ORF1 | GGTTEMLAK | 589 | FALSE |
| 205_5.0_ORF1 | 1308 | 1318 | ORF1 | TTEMLAKALR | 590 | FALSE |
| 206_1.0_ORF1 | 1311 | 1323 | ORF1 | MLAKALRKVPTD | 591 | FALSE |
| 206_3.0_ORF1 | 1315 | 1327 | ORF1 | ALRKVPTDNYIT | 592 | FALSE |
| 206_2.0_ORF1 | 1317 | 1325 | ORF1 | RKVPTDNY | 593 | FALSE |
| 206_4.0_ORF1 | 1317 | 1330 | ORF1 | RKVPTDNYITTYP | 594 | FALSE |
| 207_1.0_ORF1 | 1332 | 1341 | ORF1 | GLNGYTVEE | 595 | FALSE |
| 185_2.0_ORF1 | 1356 | 1366 | ORF1 | PSIISNEKQE | 596 | FALSE |
| 185_1.0_ORF1 | 1360 | 1367 | ORF1 | SNEKQEI | 597 | FALSE |
| 186_1.0_ORF1 | 1390 | 1400 | ORF1 | VCVETKAIVS | 598 | FALSE |
| 187_2.0_ORF1 | 1403 | 1414 | ORF1 | RKYKGIKIQEG | 599 | FALSE |
| 187_1.0_ORF1 | 1408 | 1418 | ORF1 | IKIQEGVVDY | 600 | FALSE |
| 183_1.0_ORF1 | 1432 | 1443 | ORF1 | SLINTLNDLNE | 601 | FALSE |
| 184_1.0_ORF1 | 1455 | 1471 | ORF1 | GLNLEEAARYMRSLKV | 602 | FALSE |
| 184_2.0_ORF1 | 1463 | 1481 | ORF1 | RYMRSLKVPATVSVSSPD | 603 | FALSE |
| 180_3.0_ORF1 | 1480 | 1497 | ORF1 | DAVTAYNGYLTSSSKTP | 604 | FALSE |
| 180_4.0_ORF1 | 1486 | 1499 | ORF1 | NGYLTSSSKTPEE | 605 | FALSE |
| 180_1.0_ORF1 | 1487 | 1500 | ORF1 | GYLTSSSKTPEEH | 606 | FALSE |
| 180_2.0_ORF1 | 1492 | 1502 | ORF1 | SSKTPEEHFI | 607 | FALSE |
| 181_1.0_ORF1 | 1515 | 1520 | ORF1 | YSGQS | 608 | FALSE |
| 181_2.0_ORF1 | 1515 | 1524 | ORF1 | YSGQSTQLG | 609 | FALSE |
| 182_1.0_ORF1 | 1526 | 1536 | ORF1 | FLKRGDKSVY | 610 | FALSE |
| 182_2.0_ORF1 | 1529 | 1540 | ORF1 | RGDKSVYYTSN | 611 | FALSE |
| 179_3.0_ORF1 | 1544 | 1550 | ORF1 | HLDGEV | 612 | FALSE |
| 179_2.0_ORF1 | 1558 | 1565 | ORF1 | LLSLREV | 613 | FALSE |
| 179_1.0_ORF1 | 1561 | 1567 | ORF1 | LREVRT | 614 | FALSE |
| 178_1.0_ORF1 | 1571 | 1580 | ORF1 | TTVDNINLH | 615 | FALSE |
| 177_3.0_ORF1 | 1592 | 1603 | ORF1 | QFGPTYLDGAD | 616 | FALSE |
| 177_2.0_ORF1 | 1599 | 1608 | ORF1 | DGADVTKIK | 617 | FALSE |
| 177_1.0_ORF1 | 1602 | 1608 | ORF1 | DVTKIK | 618 | FALSE |
| 175_2.0_ORF1 | 1638 | 1649 | ORF1 | DPSFLGRYMSA | 619 | FALSE |
| 175_1.0_ORF1 | 1644 | 1654 | ORF1 | RYMSALNHTK | 620 | FALSE |
| 176_2.0_ORF1 | 1654 | 1659 | ORF1 | KWKYP | 621 | FALSE |
| 176_1.0_ORF1 | 1657 | 1673 | ORF1 | YPQVNGLTSIKWADNN | 622 | FALSE |
| 106_1.0_ORF1 | 1690 | 1705 | ORF1 | NPPALQDAYYRARAG | 623 | FALSE |
| 106_2.0_ORF1 | 1694 | 1705 | ORF1 | LQDAYYRARAG | 624 | FALSE |
| 107_5.0_ORF1 | 1716 | 1725 | ORF1 | YCNKTVGEL | 625 | FALSE |
| 107_3.0_ORF1 | 1726 | 1732 | ORF1 | DVRETM | 626 | FALSE |
| 107_2.0_ORF1 | 1728 | 1733 | ORF1 | RETMS | 627 | FALSE |
| 107_1.0_ORF1 | 1729 | 1736 | ORF1 | ETMSYLF | 628 | FALSE |
| 107_4.0_ORF1 | 1739 | 1747 | ORF1 | NLDSCKRV | 629 | FALSE |
| 104_1.0_ORF1 | 1752 | 1764 | ORF1 | KTCGQQQTTLKG | 630 | FALSE |
| 105_2.0_ORF1 | 1765 | 1781 | ORF1 | EAVMYMGTLSYEQFKK | 631 | FALSE |
| 105_1.0_ORF1 | 1772 | 1784 | ORF1 | TLSYEQFKKGVQ | 632 | FALSE |
| 101_1.0_ORF1 | 1795 | 1804 | ORF1 | YLVQQESPF | 633 | FALSE |
| 101_2.0_ORF1 | 1797 | 1810 | ORF1 | VQQESPFVMMSAP | 634 | FALSE |
| 101_3.0_ORF1 | 1799 | 1818 | ORF1 | QESPFVMMSAPPAQYELKH | 635 | FALSE |
| 103_1.0_ORF1 | 1815 | 1826 | ORF1 | LKHGTFTCASE | 636 | FALSE |
| 102_1.0_ORF1 | 1832 | 1843 | ORF1 | CGHYKHITSKE | 637 | FALSE |
| 110_4.0_ORF1 | 1848 | 1856 | ORF1 | DGALLTKS | 638 | FALSE |
| 110_3.0_ORF1 | 1853 | 1865 | ORF1 | TKSSEYKGPITD | 639 | FALSE |
| 110_1.0_ORF1 | 1855 | 1870 | ORF1 | SSEYKGPITDVFYKE | 640 | FALSE |
| 110_2.0_ORF1 | 1858 | 1871 | ORF1 | YKGPITDVFYKEN | 641 | FALSE |
| 109_4.0_ORF1 | 1867 | 1880 | ORF1 | YKENSYTTTIKPV | 642 | FALSE |
| 109_3.0_ORF1 | 1874 | 1879 | ORF1 | TTIKP | 643 | FALSE |
| 109_5.0_ORF1 | 1876 | 1883 | ORF1 | IKPVTYK | 644 | FALSE |
| 109_6.0_ORF1 | 1878 | 1885 | ORF1 | PVTYKLD | 645 | FALSE |
| 109_1.0_ORF1 | 1881 | 1888 | ORF1 | YKLDGVV | 646 | FALSE |
| 109_2.0_ORF1 | 1881 | 1893 | ORF1 | YKLDGVVCTEID | 647 | FALSE |
| 108_1.0_ORF1 | 1888 | 1901 | ORF1 | CTEIDPKLDNYYK | 648 | FALSE |
| 108_2.0_ORF1 | 1890 | 1902 | ORF1 | EIDPKLDNYYKK | 649 | FALSE |
| 108_3.0_ORF1 | 1892 | 1903 | ORF1 | DPKLDNYYKKD | 650 | FALSE |
| 108_4.0_ORF1 | 1893 | 1898 | ORF1 | PKLDN | 651 | FALSE |
| 108_5.0_ORF1 | 1897 | 1910 | ORF1 | NYYKKDNSYFTEQ | 652 | FALSE |
| 108_6.0_ORF1 | 1909 | 1914 | ORF1 | QPIDL | 653 | FALSE |
| 111_2.0_ORF1 | 1916 | 1926 | ORF1 | NQPYPNASFD | 654 | FALSE |
| 111_1.0_ORF1 | 1922 | 1930 | ORF1 | ASFDNFKF | 655 | FALSE |
| 112_1.0_ORF1 | 1932 | 1942 | ORF1 | DNIKFADDLN | 656 | FALSE |
| 112_2.0_ORF1 | 1933 | 1947 | ORF1 | NIKFADDLNQLTGY | 657 | FALSE |
| 112_3.0_ORF1 | 1939 | 1949 | ORF1 | DLNQLTGYKK | 658 | FALSE |
| 112_4.0_ORF1 | 1942 | 1951 | ORF1 | QLTGYKKPA | 659 | FALSE |
| 113_3.0_ORF1 | 1973 | 1992 | ORF1 | HYTPSFKKGAKLLHKPIVW | 660 | FALSE |
| 113_2.0_ORF1 | 1974 | 1985 | ORF1 | YTPSFKKGAKL | 661 | FALSE |
| 113_1.0_ORF1 | 1978 | 1988 | ORF1 | FKKGAKLLHK | 662 | FALSE |
| 114_2.0_ORF1 | 1987 | 2001 | ORF1 | KPIVWHVNNATNKA | 663 | FALSE |
| 114_1.0_ORF1 | 1996 | 2005 | ORF1 | ATNKATYKP | 664 | FALSE |
| 98_3.0_ORF1 | 2007 | 2019 | ORF1 | WCIRCLWSTKPV | 665 | FALSE |
| 98_1.0_ORF1 | 2016 | 2023 | ORF1 | KPVETSN | 666 | FALSE |
| 98_2.0_ORF1 | 2019 | 2029 | ORF1 | ETSNSFDVLK | 667 | FALSE |
| 97_1.0_ORF1 | 2026 | 2036 | ORF1 | VLKSEDAQGM | 668 | FALSE |
| 97_2.0_ORF1 | 2027 | 2043 | ORF1 | LKSEDAQGMDNLACED | 669 | FALSE |
| 96_3.0_ORF1 | 2048 | 2058 | ORF1 | EEVVENPTIQ | 670 | FALSE |
| 96_2.0_ORF1 | 2049 | 2060 | ORF1 | EVVENPTIQKD | 671 | FALSE |
| 96_1.0_ORF1 | 2056 | 2068 | ORF1 | IQKDVLECNVKT | 672 | FALSE |
| 100_6.0_ORF1 | 2065 | 2080 | ORF1 | VKTTEVVGDIILKPA | 673 | FALSE |
| 100_7.0_ORF1 | 2071 | 2081 | ORF1 | VGDIILKPAN | 674 | FALSE |
| 100_2.0_ORF1 | 2073 | 2088 | ORF1 | DIILKPANNSLKITE | 675 | FALSE |
| 100_1.0_ORF1 | 2075 | 2087 | ORF1 | ILKPANNSLKIT | 676 | FALSE |
| 100_3.0_ORF1 | 2075 | 2089 | ORF1 | ILKPANNSLKITEE | 677 | FALSE |
| 100_5.0_ORF1 | 2082 | 2092 | ORF1 | SLKITEEVGH | 678 | FALSE |
| 100_4.0_ORF1 | 2087 | 2095 | ORF1 | EEVGHTDL | 679 | FALSE |
| 99_4.0_ORF1 | 2093 | 2105 | ORF1 | DLMAAYVDNSSL | 680 | FALSE |
| 99_3.0_ORF1 | 2099 | 2111 | ORF1 | VDNSSLTIKKPN | 681 | FALSE |
| 99_1.0_ORF1 | 2106 | 2115 | ORF1 | IKKPNELSR | 682 | FALSE |
| 99_2.0_ORF1 | 2110 | 2121 | ORF1 | NELSRVLGLKT | 683 | FALSE |
| 92_2.0_ORF1 | 2135 | 2144 | ORF1 | DTIANYAKP | 684 | FALSE |
| 92_1.0_ORF1 | 2140 | 2150 | ORF1 | YAKPFLNKVV | 685 | FALSE |
| 93_1.0_ORF1 | 2163 | 2170 | ORF1 | VCTNYMP | 686 | FALSE |
| 94_1.0_ORF1 | 2187 | 2192 | ORF1 | SRIKA | 687 | FALSE |
| 94_3.0_ORF1 | 2188 | 2199 | ORF1 | RIKASMPTTIA | 688 | FALSE |
| 94_5.0_ORF1 | 2189 | 2199 | ORF1 | IKASMPTTIA | 689 | FALSE |
| 94_4.0_ORF1 | 2189 | 2202 | ORF1 | IKASMPTTIAKNT | 690 | FALSE |
| 94_2.0_ORF1 | 2193 | 2206 | ORF1 | MPTTIAKNTVKSV | 691 | FALSE |
| 95_1.0_ORF1 | 2255 | 2261 | ORF1 | NLGMPS | 692 | FALSE |
| 128_3.0_ORF1 | 2463 | 2475 | ORF1 | FISDEVARDLSL | 693 | FALSE |
| 128_2.0_ORF1 | 2466 | 2478 | ORF1 | DEVARDLSLQFK | 694 | FALSE |
| 128_1.0_ORF1 | 2476 | 2486 | ORF1 | FKRPINPTDQ | 695 | FALSE |
| 129_4.0_ORF1 | 2490 | 2499 | ORF1 | VDSVTVKNG | 696 | FALSE |
| 129_3.0_ORF1 | 2495 | 2501 | ORF1 | VKNGSI | 697 | FALSE |
| 129_2.0_ORF1 | 2495 | 2506 | ORF1 | VKNGSIHLYFD | 698 | FALSE |
| 129_1.0_ORF1 | 2504 | 2512 | ORF1 | FDKAGQKT | 699 | FALSE |
| 125_2.0_ORF1 | 2522 | 2531 | ORF1 | NLDNLRANN | 700 | FALSE |
| 125_1.0_ORF1 | 2524 | 2534 | ORF1 | DNLRANNTKG | 701 | FALSE |
| 126_1.0_ORF1 | 2540 | 2548 | ORF1 | IVFDGKSK | 702 | FALSE |
| 127_1.0_ORF1 | 2546 | 2568 | ORF1 | SKCEESSAKSASVYYSQLMCQP | 703 | FALSE |
| 127_2.0_ORF1 | 2551 | 2562 | ORF1 | SSAKSASVYYS | 704 | FALSE |
| 137_1.0_ORF1 | 2581 | 2590 | ORF1 | DSAEVAVKM | 705 | FALSE |
| 136_2.0_ORF1 | 2605 | 2611 | ORF1 | MEKLKT | 706 | FALSE |
| 136_1.0_ORF1 | 2620 | 2629 | ORF1 | AKNVSLDNV | 707 | FALSE |
| 135_2.0_ORF1 | 2635 | 2641 | ORF1 | AARQGF | 708 | FALSE |
| 135_1.0_ORF1 | 2637 | 2651 | ORF1 | RQGFVDSDVETKDV | 709 | FALSE |
| 135_3.0_ORF1 | 2646 | 2658 | ORF1 | ETKDVVECLKLS | 710 | FALSE |
| 133_1.0_ORF1 | 2670 | 2682 | ORF1 | NNYMLTYNKVEN | 711 | FALSE |
| 133_2.0_ORF1 | 2670 | 2685 | ORF1 | NNYMLTYNKVENMTP | 712 | FALSE |
| 134_1.0_ORF1 | 2689 | 2707 | ORF1 | ACIDCSARHINAQVAKSH | 713 | FALSE |
| 134_2.0_ORF1 | 2703 | 2708 | ORF1 | AKSHN | 714 | FALSE |
| 139_3.0_ORF1 | 2719 | 2731 | ORF1 | SLSEQLRKQIRS | 715 | FALSE |
| 139_4.0_ORF1 | 2719 | 2734 | ORF1 | SLSEQLRKQIRSAAK | 716 | FALSE |
| 139_2.0_ORF1 | 2721 | 2737 | ORF1 | SEQLRKQIRSAAKKNN | 717 | FALSE |
| 139_1.0_ORF1 | 2733 | 2741 | ORF1 | KKNNLPFK | 718 | FALSE |
| 138_1.0_ORF1 | 2754 | 2767 | ORF1 | TTKIALKGGKIVN | 719 | FALSE |
| 140_1.0_ORF1 | 2802 | 2810 | ORF1 | SSEIIGYK | 720 | FALSE |
| 140_2.0_ORF1 | 2803 | 2815 | ORF1 | SEIIGYKAIDGG | 721 | FALSE |
| 141_1.0_ORF1 | 2818 | 2832 | ORF1 | DIASTDTCFANKHA | 722 | FALSE |
| 142_1.0_ORF1 | 2836 | 2849 | ORF1 | WFSQRGGSYTNDK | 723 | FALSE |
| 132_1.0_ORF1 | 2902 | 2909 | ORF1 | IEYTDFA | 724 | FALSE |
| 131_1.0_ORF1 | 2916 | 2937 | ORF1 | AECTIFKDASGKPVPYCYDTN | 725 | FALSE |
| 131_2.0_ORF1 | 2918 | 2931 | ORF1 | CTIFKDASGKPVP | 726 | FALSE |
| 130_1.0_ORF1 | 2946 | 2965 | ORF1 | SLRPDTRYVLMDGSIIQFP | 727 | FALSE |
| 130_2.0_ORF1 | 2948 | 2956 | ORF1 | RPDTRYVL | 728 | FALSE |
| 115_3.0_ORF1 | 3157 | 3165 | ORF1 | SNYLKRRV | 729 | FALSE |
| 115_5.0_ORF1 | 3160 | 3169 | ORF1 | LKRRVVFNG | 730 | FALSE |
| 115_4.0_ORF1 | 3160 | 3178 | ORF1 | LKRRVVFNGVSFSTFEEA | 731 | FALSE |
| 115_1.0_ORF1 | 3170 | 3175 | ORF1 | SFSTF | 732 | FALSE |
| 115_2.0_ORF1 | 3171 | 3176 | ORF1 | FSTFE | 733 | FALSE |
| 118_2.0_ORF1 | 3197 | 3214 | ORF1 | LLPLTQYNRYLALYNKY | 734 | FALSE |
| 118_1.0_ORF1 | 3201 | 3218 | ORF1 | TQYNRYLALYNKYKYFS | 735 | FALSE |
| 117_1.0_ORF1 | 3230 | 3240 | ORF1 | CCHLAKALND | 736 | FALSE |
| 117_2.0_ORF1 | 3234 | 3240 | ORF1 | AKALND | 737 | FALSE |
| 116_3.0_ORF1 | 3258 | 3265 | ORF1 | SAVLQSG | 738 | FALSE |
| 116_2.0_ORF1 | 3262 | 3269 | ORF1 | QSGFRKM | 739 | FALSE |
| 116_1.0_ORF1 | 3264 | 3273 | ORF1 | GFRKMAFPS | 740 | FALSE |
| 124_1.0_ORF1 | 3320 | 3331 | ORF1 | LIRKSNHNFLV | 741 | FALSE |
| 123_1.0_ORF1 | 3350 | 3359 | ORF1 | KLKVDTANP | 742 | FALSE |
| 123_2.0_ORF1 | 3355 | 3363 | ORF1 | TANPKTPK | 743 | FALSE |
| 123_3.0_ORF1 | 3363 | 3371 | ORF1 | YKFVRIQP | 744 | FALSE |
| 121_2.0_ORF1 | 3392 | 3401_1 | ORF1 | MRPNFTIKG | 745 | FALSE |
| 121_1.0_ORF1 | 3399 | 3407 | ORF1 | KGSFLNGS | 746 | FALSE |
| 122_1.0_ORF1 | 3426 | 3441 | ORF1 | HMELPTGVHAGTDLE | 747 | FALSE |
| 120_1.0_ORF1 | 3477 | 3485 | ORF1 | GDRWFLNR | 748 | FALSE |
| 119_4.0_ORF1 | 3531 | 3537 | ORF1 | KELLQN | 749 | FALSE |
| 119_2.0_ORF1 | 3534 | 3541 | ORF1 | LQNGMNG | 750 | FALSE |
| 119_1.0_ORF1 | 3536 | 3542 | ORF1 | NGMNGR | 751 | FALSE |
| 119_3.0_ORF1 | 3541 | 3549 | ORF1 | RTILGSAL | 752 | FALSE |
| 23_1.0_ORF1 | 3723 | 3728 | ORF1 | GNALD | 753 | FALSE |
| 25_1.0_ORF1 | 3824 | 3841 | ORF1 | SQGLLPPKNSIDAFKLN | 754 | FALSE |
| 25_2.0_ORF1 | 3829 | 3839 | ORF1 | PPKNSIDAFK | 755 | FALSE |
| 25_3.0_ORF1 | 3831 | 3837 | ORF1 | KNSIDA | 756 | FALSE |
| 24_1.0_ORF1 | 3840 | 3855 | ORF1 | NIKLLGVGGKPCIKV | 757 | FALSE |
| 24_3.0_ORF1 | 3845 | 3860 | ORF1 | GVGGKPCIKVATVQS | 758 | FALSE |
| 24_2.0_ORF1 | 3848 | 3857 | ORF1 | GKPCIKVAT | 759 | FALSE |
| 22_1.0_ORF1 | 3897 | 3905 | ORF1 | ILLAKDTT | 760 | FALSE |
| 20_2.0_ORF1 | 3933 | 3940 | ORF1 | MLDNRAT | 761 | FALSE |
| 20_1.0_ORF1 | 3935 | 3952 | ORF1 | DNRATLQAIASEFSSLP | 762 | FALSE |
| 21_2.0_ORF1 | 3950 | 3969 | ORF1 | LPSYAAFATAQEAYEQAVA | 763 | FALSE |
| 21_1.0_ORF1 | 3955 | 3967 | ORF1 | AFATAQEAYEQA | 764 | FALSE |
| 21_3.0_ORF1 | 3958 | 3977 | ORF1 | TAQEAYEQAVANGDSEVVL | 765 | FALSE |
| 21_4.0_ORF1 | 3961 | 3967 | ORF1 | EAYEQA | 766 | FALSE |
| 19_3.0_ORF1 | 3973 | 3985 | ORF1 | EVVLKKLKKSLN | 767 | FALSE |
| 19_1.0_ORF1 | 3979 | 3987 | ORF1 | LKKSLNVA | 768 | FALSE |
| 19_2.0_ORF1 | 3979 | 3992 | ORF1 | LKKSLNVAKSEFD | 769 | FALSE |
| 19_4.0_ORF1 | 3986 | 3992 | ORF1 | AKSEFD | 770 | FALSE |
| 17_1.0_ORF1 | 4010 | 4022 | ORF1 | QMYKQARSEDKR | 771 | FALSE |
| 18_1.0_ORF1 | 4035 | 4047 | ORF1 | MLRKLDNDALNN | 772 | FALSE |
| 37_1.0_ORF1 | 4074 | 4081 | ORF1 | PDYNTYK | 773 | FALSE |
| 36_1.0_ORF1 | 4102 | 4113 | ORF1 | DADSKIVQLSE | 774 | FALSE |
| 35_2.0_ORF1 | 4118 | 4125 | ORF1 | SPNLAWP | 775 | FALSE |
| 35_1.0_ORF1 | 4118 | 4129 | ORF1 | SPNLAWPLIVT | 776 | FALSE |
| 35_3.0_ORF1 | 4129 | 4140 | ORF1 | ALRANSAVKLQ | 777 | FALSE |
| 34_1.0_ORF1 | 4153 | 4159 | ORF1 | CAAGTT | 778 | FALSE |
| 33_1.0_ORF1 | 4180 | 4195 | ORF1 | VLALLSDLQDLKWAR | 779 | FALSE |
| 33_2.0_ORF1 | 4188 | 4198 | ORF1 | QDLKWARFPK | 780 | FALSE |
| 41_1.0_ORF1 | 4230 | 4239 | ORF1 | IKGLNNLNR | 781 | FALSE |
| 42_1.0_ORF1 | 4257 | 4263 | ORF1 | TEVPAN | 782 | FALSE |
| 40_2.0_ORF1 | 4274 | 4293 | ORF1 | DAAKAYKDYLASGGQPITN | 783 | FALSE |
| 40_1.0_ORF1 | 4277 | 4290 | ORF1 | KAYKDYLASGGQP | 784 | FALSE |
| 40_3.0_ORF1 | 4292 | 4304 | ORF1 | NCVKMLCTHTGT | 785 | FALSE |
| 39_1.0_ORF1 | 4315 | 4321 | ORF1 | MDQESF | 786 | FALSE |
| 38_1.0_ORF1 | 4338 | 4352 | ORF1 | PKGFCDLKGKYVQI | 787 | FALSE |
| 29_1.0_ORF1 | 4398 | 4413 | ORF1 | FLNRVCGVSAARLTP | 788 | FALSE |
| 28_1.0_ORF1 | 4416 | 4434 | ORF1 | GTSTDVVYRAFDIYNDKV | 789 | FALSE |
| 31_2.0_ORF1 | 4449 | 4459 | ORF1 | EKDEDDNLID | 790 | FALSE |
| 31_1.0_ORF1 | 4458 | 4469 | ORF1 | DSYFVVKRHTF | 791 | FALSE |
| 32_1.0_ORF1 | 4474 | 4487 | ORF1 | EETIYNLLKDCPA | 792 | FALSE |
| 32_2.0_ORF1 | 4493 | 4506 | ORF1 | FKFRIDGDMVPHI | 793 | FALSE |
| 30_3.0_ORF1 | 4507 | 4521 | ORF1 | RQRLTKYTMADLVY | 794 | FALSE |
| 30_2.0_ORF1 | 4510 | 4519 | ORF1 | LTKYTMADL | 795 | FALSE |
| 30_1.0_ORF1 | 4512 | 4531 | ORF1 | KYTMADLVYALRHFDEGNC | 796 | FALSE |
| 27_1.0_ORF1 | 4599 | 4605 | ORF1 | DNQDLN | 797 | FALSE |
| 27_2.0_ORF1 | 4603 | 4612 | ORF1 | LNGNWYDFG | 798 | FALSE |
| 26_2.0_ORF1 | 4651 | 4657 | ORF1 | DLTKPY | 799 | FALSE |
| 26_1.0_ORF1 | 4651 | 4661 | ORF1 | DLTKPYIKWD | 800 | FALSE |
| 13_1.0_ORF1 | 4747 | 4763 | ORF1 | NQDVNLHSSRLSFKEL | 801 | FALSE |
| 13_2.0_ORF1 | 4755 | 4764 | ORF1 | SRLSFKELL | 802 | FALSE |
| 14_5.0_ORF1 | 4791 | 4797 | ORF1 | ALTNNV | 803 | FALSE |
| 14_4.0_ORF1 | 4793 | 4807 | ORF1 | TNNVAFQTVKPGNF | 804 | FALSE |
| 14_3.0_ORF1 | 4801 | 4810 | ORF1 | VKPGNFNKD | 805 | FALSE |
| 14_1.0_ORF1 | 4803 | 4816 | ORF1 | PGNFNKDFYDFAV | 806 | FALSE |
| 14_2.0_ORF1 | 4806 | 4821 | ORF1 | FNKDFYDFAVSKGFF | 807 | FALSE |
| 15_1.0_ORF1 | 4821 | 4830 | ORF1 | KEGSSVELK | 808 | FALSE |
| 15_2.0_ORF1 | 4826 | 4836 | ORF1 | VELKHFFFAQ | 809 | FALSE |
| 16_1.0_ORF1 | 4847 | 4856 | ORF1 | YRYNLPTMC | 810 | FALSE |
| 11_1.0_ORF1 | 4879 | 4893 | ORF1 | INANQVIVNNLDKS | 811 | FALSE |
| 12_1.0_ORF1 | 4896 | 4902 | ORF1 | PFNKWG | 812 | FALSE |
| 12_2.0_ORF1 | 4902 | 4907 | ORF1 | KARLY | 813 | FALSE |
| 10_3.0_ORF1 | 4903 | 4916 | ORF1 | ARLYYDSMSYEDQ | 814 | FALSE |
| 10_2.0_ORF1 | 4906 | 4920 | ORF1 | YYDSMSYEDQDALF | 815 | FALSE |
| 10_1.0_ORF1 | 4913 | 4926 | ORF1 | EDQDALFAYTKRN | 816 | FALSE |
| 8_2.0_ORF1 | 4932 | 4941 | ORF1 | QMNLKYAIS | 817 | FALSE |
| 8_1.0_ORF1 | 4934 | 4947 | ORF1 | NLKYAISAKNRAR | 818 | FALSE |
| 8_3.0_ORF1 | 4939 | 4947 | ORF1 | ISAKNRAR | 819 | FALSE |
| 9_1.0_ORF1 | 4959 | 4969 | ORF1 | NRQFHQKLLK | 820 | FALSE |
| 3_2.0_ORF1 | 5087 | 5099 | ORF1 | ICQAVTANVNAL | 821 | FALSE |
| 3_1.0_ORF1 | 5100 | 5108 | ORF1 | STDGNKIA | 822 | FALSE |
| 4_1.0_ORF1 | 5131 | 5137 | ORF1 | DFVNEF | 823 | FALSE |
| 7_3.0_ORF1 | 5161 | 5169 | ORF1 | YASQGLVA | 824 | FALSE |
| 7_2.0_ORF1 | 5168 | 5178 | ORF1 | ASIKNFKSVL | 825 | FALSE |
| 7_1.0_ORF1 | 5171 | 5183 | ORF1 | KNFKSVLYYQNN | 826 | FALSE |
| 6_1.0_ORF1 | 5178 | 5189 | ORF1 | YYQNNVFMSEA | 827 | FALSE |
| 6_2.0_ORF1 | 5183 | 5192 | ORF1 | VFMSEAKCW | 828 | FALSE |
| 5_2.0_ORF1 | 5198 | 5204 | ORF1 | KGPHEF | 829 | FALSE |
| 5_1.0_ORF1 | 5208 | 5222 | ORF1 | TMLVKQGDDYVYLP | 830 | FALSE |
| 2_2.0_ORF1 | 5240 | 5247 | ORF1 | KTDGTLM | 831 | FALSE |
| 2_1.0_ORF1 | 5253 | 5259 | ORF1 | LAIDAY | 832 | FALSE |
| 1_1.0_ORF1 | 5265 | 5276 | ORF1 | NQEYADVFHLY | 833 | FALSE |
| 44_1.0_ORF1 | 5432 | 5442 | ORF1 | IATCDWTNAG | 834 | FALSE |
| 45_1.0_ORF1 | 5459 | 5470 | ORF1 | ETLKATEETFK | 835 | FALSE |
| 47_1.0_ORF1 | 5494 | 5508 | ORF1 | KPRPPLNRNYVFTG | 836 | FALSE |
| 47_2.0_ORF1 | 5509 | 5518 | ORF1 | RVTKNSKVQ | 837 | FALSE |
| 46_3.0_ORF1 | 5518 | 5528 | ORF1 | IGEYTFEKGD | 838 | FALSE |
| 46_1.0_ORF1 | 5525 | 5533 | ORF1 | KGDYGDAV | 839 | FALSE |
| 46_2.0_ORF1 | 5533 | 5543 | ORF1 | VYRGTTTYKL | 840 | FALSE |
| 43_3.0_ORF1 | 5592 | 5602 | ORF1 | YQKVGMQKYS | 841 | FALSE |
| 43_2.0_ORF1 | 5594 | 5609 | ORF1 | KVGMQKYSTLQGPPG | 842 | FALSE |
| 43_1.0_ORF1 | 5597 | 5609 | ORF1 | MQKYSTLQGPPG | 843 | FALSE |
| 50_1.0_ORF1 | 5667 | 5683 | ORF1 | DKFKVNSTLEQYVFCT | 844 | FALSE |
| 48_3.0_ORF1 | 5702 | 5718 | ORF1 | ATNYDLSVVNARLRAK | 845 | FALSE |
| 48_4.0_ORF1 | 5710 | 5718 | ORF1 | VNARLRAK | 846 | FALSE |
| 48_2.0_ORF1 | 5713 | 5721 | ORF1 | RLRAKHYV | 847 | FALSE |
| 48_1.0_ORF1 | 5716 | 5727 | ORF1 | AKHYVYIGDPA | 848 | FALSE |
| 49_1.0_ORF1 | 5724 | 5743 | ORF1 | DPAQLPAPRTLLTKGTLEP | 849 | FALSE |
| 49_2.0_ORF1 | 5730 | 5738 | ORF1 | APRTLLTK | 850 | FALSE |
| 49_3.0_ORF1 | 5745 | 5751 | ORF1 | FNSVCR | 851 | FALSE |
| 55_1.0_ORF1 | 5770 | 5785 | ORF1 | EIVDTVSALVYDNKL | 852 | FALSE |
| 55_2.0_ORF1 | 5771 | 5782 | ORF1 | IVDTVSALVYD | 853 | FALSE |
| 54_1.0_ORF1 | 5780 | 5794 | ORF1 | YDNKLKAHKDKSAQ | 854 | FALSE |
| 53_3.0_ORF1 | 5792 | 5801 | ORF1 | AQCFKMFYK | 855 | FALSE |
| 53_2.0_ORF1 | 5799 | 5809 | ORF1 | YKGVITHDVS | 856 | FALSE |
| 53_1.0_ORF1 | 5804 | 5817 | ORF1 | THDVSSAINRPQI | 857 | FALSE |
| 51_1.0_ORF1 | 5826 | 5835 | ORF1 | NPAWRKAVF | 858 | FALSE |
| 51_2.0_ORF1 | 5830 | 5835 | ORF1 | RKAVF | 859 | FALSE |
| 52_1.0_ORF1 | 5833 | 5843 | ORF1 | VFISPYNSQN | 860 | FALSE |
| 52_3.0_ORF1 | 5837 | 5849 | ORF1 | PYNSQNAVASKI | 861 | FALSE |
| 52_2.0_ORF1 | 5838 | 5849 | ORF1 | YNSQNAVASKI | 862 | FALSE |
| 62_1.0_ORF1 | 5868 | 5877 | ORF1 | IFTQTTETA | 863 | FALSE |
| 63_1.0_ORF1 | 5893 | 5902 | ORF1 | VGILCIMSD | 864 | FALSE |
| 63_3.0_ORF1 | 5894 | 5902 | ORF1 | GILCIMSD | 865 | FALSE |
| 63_2.0_ORF1 | 5894 | 5902 | ORF1 | GILCIMSD | 866 | FALSE |
| 63_4.0_ORF1 | 5894 | 5904 | ORF1 | GILCIMSDRD | 867 | FALSE |
| 63_5.0_ORF1 | 5896 | 5908 | ORF1 | LCIMSDRDLYDK | 868 | FALSE |
| 61_2.0_ORF1 | 5910 | 5920 | ORF1 | FTSLEIPRRN | 869 | FALSE |
| 61_4.0_ORF1 | 5914 | 5922 | ORF1 | EIPRRNVA | 870 | FALSE |
| 61_1.0_ORF1 | 5914 | 5922 | ORF1 | EIPRRNVA | 871 | FALSE |
| 61_3.0_ORF1 | 5915 | 5928 | ORF1 | IPRRNVATLQAEN | 872 | FALSE |
| 61_5.0_ORF1 | 5924 | 5934 | ORF1 | QAENVTGLFK | 873 | FALSE |
| 60_1.0_ORF1 | 5929 | 5947 | ORF1 | TGLFKDCSKVITGLHPTQ | 874 | FALSE |
| 59_2.0_ORF1 | 6016 | 6027 | ORF1 | EGCHATREAVG | 875 | FALSE |
| 59_1.0_ORF1 | 6017 | 6033 | ORF1 | GCHATREAVGTNLPLQ | 876 | FALSE |
| 59_3.0_ORF1 | 6036 | 6045 | ORF1 | STGVNLVAV | 877 | FALSE |
| 58_3.0_ORF1 | 6053 | 6066 | ORF1 | NNTDFSRVSAKPP | 878 | FALSE |
| 58_2.0_ORF1 | 6060 | 6068 | ORF1 | VSAKPPPG | 879 | FALSE |
| 58_1.0_ORF1 | 6062 | 6073 | ORF1 | AKPPPGDQFKH | 880 | FALSE |
| 56_1.0_ORF1 | 6102 | 6115 | ORF1 | SDRVVFVLWAHGF | 881 | FALSE |
| 56_2.0_ORF1 | 6109 | 6118 | ORF1 | LWAHGFELT | 882 | FALSE |
| 57_1.0_ORF1 | 6135 | 6144 | ORF1 | DRRATCFST | 883 | FALSE |
| 75_1.0_ORF1 | 6177 | 6183 | ORF1 | LQSNHD | 884 | FALSE |
| 74_2.0_ORF1 | 6204 | 6213 | ORF1 | LAVHECFVK | 885 | FALSE |
| 74_1.0_ORF1 | 6219 | 6230 | ORF1 | EYPIIGDELKI | 886 | FALSE |
| 76_2.0_ORF1 | 6236 | 6253 | ORF1 | VQHMVVKAALLADKFPV | 887 | FALSE |
| 76_1.0_ORF1 | 6247 | 6254 | ORF1 | ADKFPVL | 888 | FALSE |
| 77_2.0_ORF1 | 6266 | 6275 | ORF1 | PQADVEWKF | 889 | FALSE |
| 77_1.0_ORF1 | 6273 | 6282 | ORF1 | KFYDAQPCS | 890 | FALSE |
| 77_3.0_ORF1 | 6286 | 6295 | ORF1 | KIEELFYSY | 891 | FALSE |
| 73_1.0_ORF1 | 6338 | 6347 | ORF1 | CDGGSLYVN | 892 | FALSE |
| 72_5.0_ORF1 | 6355 | 6363 | ORF1 | FDKSAFVN | 893 | FALSE |
| 72_4.0_ORF1 | 6356 | 6368 | ORF1 | DKSAFVNLKQLP | 894 | FALSE |
| 72_3.0_ORF1 | 6361 | 6368 | ORF1 | VNLKQLP | 895 | FALSE |
| 72_2.0_ORF1 | 6362 | 6371 | ORF1 | NLKQLPFFY | 896 | FALSE |
| 72_1.0_ORF1 | 6362 | 6374 | ORF1 | NLKQLPFFYYSD | 897 | FALSE |
| 71_1.0_ORF1 | 6379 | 6391 | ORF1 | HGKQVVSDIDYV | 898 | FALSE |
| 71_2.0_ORF1 | 6387 | 6396 | ORF1 | IDYVPLKSA | 899 | FALSE |
| 79_1.0_ORF1 | 6454 | 6464 | ORF1 | ENVAFNVVNK | 900 | FALSE |
| 78_1.0_ORF1 | 6465 | 6481 | ORF1 | HFDGQQGEVPVSIINN | 901 | FALSE |
| 78_2.0_ORF1 | 6471 | 6481 | ORF1 | GEVPVSIINN | 902 | FALSE |
| 78_3.0_ORF1 | 6484 | 6493 | ORF1 | TKVDGVDVE | 903 | FALSE |
| 80_2.0_ORF1 | 6495 | 6500 | ORF1 | ENKTT | 904 | FALSE |
| 80_1.0_ORF1 | 6496 | 6512 | ORF1 | NKTTLPVNVAFELWAK | 905 | FALSE |
| 80_3.0_ORF1 | 6502 | 6512 | ORF1 | VNVAFELWAK | 906 | FALSE |
| 81_2.0_ORF1 | 6505 | 6520 | ORF1 | AFELWAKRNIKPVPE | 907 | FALSE |
| 81_3.0_ORF1 | 6509 | 6526 | ORF1 | WAKRNIKPVPEVKILNN | 908 | FALSE |
| 81_1.0_ORF1 | 6510 | 6520 | ORF1 | AKRNIKPVPE | 909 | FALSE |
| 81_6.0_ORF1 | 6511 | 6521 | ORF1 | KRNIKPVPEV | 910 | FALSE |
| 81_5.0_ORF1 | 6511 | 6526 | ORF1 | KRNIKPVPEVKILNN | 911 | FALSE |
| 81_4.0_ORF1 | 6521 | 6528 | ORF1 | KILNNLG | 912 | FALSE |
| 85_2.0_ORF1 | 6548 | 6558 | ORF1 | STIGVCSMTD | 913 | FALSE |
| 85_1.0_ORF1 | 6556 | 6565 | ORF1 | TDIAKKPTE | 914 | FALSE |
| 85_3.0_ORF1 | 6565 | 6576 | ORF1 | TICAPLTVFFD | 915 | FALSE |
| 86_1.0_ORF1 | 6588 | 6603 | ORF1 | ARNGVLITEGSVKGL | 916 | FALSE |
| 87_4.0_ORF1 | 6598 | 6605 | ORF1 | SVKGLQP | 917 | FALSE |
| 87_3.0_ORF1 | 6600 | 6612 | ORF1 | KGLQPSVGPKQA | 918 | FALSE |
| 87_1.0_ORF1 | 6608 | 6616 | ORF1 | PKQASLNG | 919 | FALSE |
| 87_2.0_ORF1 | 6608 | 6620 | ORF1 | PKQASLNGVTLI | 920 | FALSE |
| 88_5.0_ORF1 | 6619 | 6634 | ORF1 | IGEAVKTQFNYYKKV | 921 | FALSE |
| 88_1.0_ORF1 | 6620 | 6627 | ORF1 | GEAVKTQ | 922 | FALSE |
| 88_4.0_ORF1 | 6623 | 6635 | ORF1 | VKTQFNYYKKVD | 923 | FALSE |
| 88_3.0_ORF1 | 6625 | 6634 | ORF1 | TQFNYYKKV | 924 | FALSE |
| 88_2.0_ORF1 | 6625 | 6638 | ORF1 | TQFNYYKKVDGVV | 925 | FALSE |
| 89_1.0_ORF1 | 6631 | 6643 | ORF1 | KKVDGVVQQLPE | 926 | FALSE |
| 89_2.0_ORF1 | 6641 | 6653 | ORF1 | PETYFTQSRNLQ | 927 | FALSE |
| 90_1.0_ORF1 | 6651 | 6662 | ORF1 | LQEFKPRSQME | 928 | FALSE |
| 91_1.0_ORF1 | 6684 | 6689 | ORF1 | EHIVY | 929 | FALSE |
| 84_1.0_ORF1 | 6706 | 6714 | ORF1 | AKRFKESP | 930 | FALSE |
| 84_2.0_ORF1 | 6710 | 6720 | ORF1 | KESPFELEDF | 931 | FALSE |
| 83_1.0_ORF1 | 6722 | 6730 | ORF1 | MDSTVKNY | 932 | FALSE |
| 82_2.0_ORF1 | 6740 | 6746 | ORF1 | KCVCSV | 933 | FALSE |
| 82_1.0_ORF1 | 6745 | 6755 | ORF1 | VIDLLLDDFV | 934 | FALSE |
| 70_1.0_ORF1 | 6790 | 6798 | ORF1 | ETFYPKLQ | 935 | FALSE |
| 69_1.0_ORF1 | 6821 | 6837 | ORF1 | KCDLQNYGDSATLPKG | 936 | FALSE |
| 68_1.0_ORF1 | 6863 | 6875 | ORF1 | RVIHFGAGSDKG | 937 | FALSE |
| 68_2.0_ORF1 | 6875 | 6883 | ORF1 | VAPGTAVL | 938 | FALSE |
| 67_1.0_ORF1 | 6891 | 6903 | ORF1 | LLVDSDLNDFVS | 939 | FALSE |
| 67_2.0_ORF1 | 6898 | 6905 | ORF1 | NDFVSDA | 940 | FALSE |
| 64_1.0_ORF1 | 6915 | 6936 | ORF1 | VHTANKWDLIISDMYDPKTKN | 941 | FALSE |
| 64_2.0_ORF1 | 6918 | 6938 | ORF1 | ANKWDLIISDMYDPKTKNVT | 942 | FALSE |
| 64_3.0_ORF1 | 6920 | 6931 | ORF1 | KWDLIISDMYD | 943 | FALSE |
| 65_2.0_ORF1 | 6926 | 6940 | ORF1 | SDMYDPKTKNVTKE | 944 | FALSE |
| 65_1.0_ORF1 | 6926 | 6941 | ORF1 | SDMYDPKTKNVTKEN | 945 | FALSE |
| 65_3.0_ORF1 | 6932 | 6942 | ORF1 | KTKNVTKEND | 946 | FALSE |
| 65_4.0_ORF1 | 6932 | 6944 | ORF1 | KTKNVTKENDSK | 947 | FALSE |
| 65_5.0_ORF1 | 6935 | 6942 | ORF1 | NVTKEND | 948 | FALSE |
| 66_1.0_ORF1 | 6975 | 6980 | ORF1 | ADLYK | 949 | FALSE |
| 1_3.0_ORF3 | 161 | 167 | ORF3 | SVTSSI | 950 | FALSE |
| 1_1.0_ORF3 | 167 | 183 | ORF3 | VITSGDGTTSPISEHD | 951 | FALSE |
| 1_2.0_ORF3 | 183 | 192 | ORF3 | YQIGGYTEK | 952 | FALSE |
| 2_1.0_ORF3 | 230 | 239 | ORF3 | FIYNKIVDE | 953 | FALSE |
| 2_2.0_ORF3 | 236 | 250 | ORF3 | VDEPEEHVQIHTID | 954 | FALSE |
| 3_3.0_ORF3 | 256 | 261 | ORF3 | NPVME | 955 | FALSE |
| 3_2.0_ORF3 | 258 | 267 | ORF3 | VMEPIYDEP | 956 | FALSE |
| 3_1.0_ORF3 | 263 | 274 | ORF3 | YDEPTTTTSVPL | 957 | FALSE |
| 1_1.0_ORF6 | 1 | 7 | ORF6 | FHLVDF | 958 | FALSE |
| 1_2.0_ORF6 | 2 | 9 | ORF6 | HLVDFQV | 959 | FALSE |
| 1_4.0_ORF6 | 4 | 11 | ORF6 | VDFQVTI | 960 | FALSE |
| 1_3.0_ORF6 | 5 | 12 | ORF6 | DFQVTIA | 961 | FALSE |
| 1_5.0_ORF6 | 6 | 12 | ORF6 | FQVTIA | 962 | FALSE |
| 2_5.0_ORF6 | 10 | 20 | ORF6 | IAEILLIIMR | 963 | FALSE |
| 2_6.0_ORF6 | 11 | 17 | ORF6 | AEILLI | 964 | FALSE |
| 2_3.0_ORF6 | 16 | 23 | ORF6 | IIMRTFK | 965 | FALSE |
| 2_4.0_ORF6 | 16 | 26 | ORF6 | IIMRTFKVSI | 966 | FALSE |
| 2_1.0_ORF6 | 18 | 29 | ORF6 | MRTFKVSIWNL | 967 | FALSE |
| 2_2.0_ORF6 | 20 | 27 | ORF6 | TFKVSIW | 968 | FALSE |
| 3_18.0_ORF6 | 33 | 50 | ORF6 | NLIIKNLSKSLTENKYS | 969 | FALSE |
| 3_2.0_ORF6 | 33 | 53 | ORF6 | NLIIKNLSKSLTENKYSQLD | 970 | FALSE |
| 3_21.0_ORF6 | 35 | 40 | ORF6 | IIKNL | 971 | FALSE |
| 3_20.0_ORF6 | 35 | 41 | ORF6 | IIKNLS | 972 | FALSE |
| 3_9.0_ORF6 | 36 | 45 | ORF6 | IKNLSKSLT | 973 | FALSE |
| 3_14.0_ORF6 | 36 | 51 | ORF6 | IKNLSKSLTENKYSQ | 974 | FALSE |
| 3_7.0_ORF6 | 38 | 46 | ORF6 | NLSKSLTE | 975 | FALSE |
| 3_13.0_ORF6 | 38 | 50 | ORF6 | NLSKSLTENKYS | 976 | FALSE |
| 3_11.0_ORF6 | 38 | 50 | ORF6 | NLSKSLTENKYS | 977 | FALSE |
| 3_17.0_ORF6 | 38 | 50 | ORF6 | NLSKSLTENKYS | 978 | FALSE |
| 3_6.0_ORF6 | 38 | 50 | ORF6 | NLSKSLTENKYS | 979 | FALSE |
| 3_10.0_ORF6 | 38 | 56 | ORF6 | NLSKSLTENKYSQLDEEQ | 980 | FALSE |
| 3_19.0_ORF6 | 39 | 48 | ORF6 | LSKSLTENK | 981 | FALSE |
| 3_12.0_ORF6 | 39 | 53 | ORF6 | LSKSLTENKYSQLD | 982 | FALSE |
| 3_16.0_ORF6 | 40 | 50 | ORF6 | SKSLTENKYS | 983 | FALSE |
| 3_5.0_ORF6 | 40 | 55 | ORF6 | SKSLTENKYSQLDEE | 984 | FALSE |
| 5_4.0_ORF6 | 40 | 60 | ORF6 | SKSLTENKYSQLDEEQPMEID | 985 | FALSE |
| 3_8.0_ORF6 | 41 | 47 | ORF6 | KSLTEN | 986 | FALSE |
| 3_3.0_ORF6 | 41 | 51 | ORF6 | KSLTENKYSQ | 987 | FALSE |
| 3_1.0_ORF6 | 41 | 53 | ORF6 | KSLTENKYSQLD | 988 | FALSE |
| 5_1.0_ORF6 | 41 | 60 | ORF6 | KSLTENKYSQLDEEQPMEID | 989 | FALSE |
| 3_15.0_ORF6 | 42 | 50 | ORF6 | SLTENKYS | 990 | FALSE |
| 3_4.0_ORF6 | 42 | 51 | ORF6 | SLTENKYSQ | 991 | FALSE |
| 5_2.0_ORF6 | 43 | 60 | ORF6 | LTENKYSQLDEEQPMEID | 992 | FALSE |
| 5_3.0_ORF6 | 46 | 56 | ORF6 | NKYSQLDEEQ | 993 | FALSE |
| 4_5.0_ORF6 | 50 | 60 | ORF6 | QLDEEQPMEID | 994 | FALSE |
| 4_3.0_ORF6 | 52 | 60 | ORF6 | DEEQPMEID | 995 | FALSE |
| 4_4.0_ORF6 | 53 | 60 | ORF6 | EEQPMEID | 996 | FALSE |
| 4_2.0_ORF6 | 56 | 60 | ORF6 | PMEID | 997 | FALSE |
| 4_1.0_ORF6 | 56 | 60 | ORF6 | PMEID | 998 | FALSE |
| 1_1.0_ORF7A | 40 | 52 | ORF7A | EGNSPFHPLADN | 999 | FALSE |
| 2_1.0_ORF8 | 30 | 39 | ORF8 | YVVDDPCPI | 1000 | FALSE |
| 1_1.0_ORF8 | 62 | 70 | ORF8 | DEAGSKSP | 1001 | FALSE |
| 3_1.0_ORF8 | 115 | 120 | ORF8 | VVLDFI | 1002 | FALSE |
| 1_1.0_ORF9B | 1 | 11 | ORF9B | DPKISEMHPA | 1003 | FALSE |
| 3_1.0_ORF9B | 42 | 51 | ORF9B | PIILRLGSP | 1004 | FALSE |
| 2_3.0_ORF9B | 50 | 66 | ORF9B | PLSLNMARKTLNSLED | 1005 | FALSE |
| 2_2.0_ORF9B | 57 | 68 | ORF9B | RKTLNSLEDKA | 1006 | FALSE |
| 2_1.0_ORF9B | 57 | 73 | ORF9B | RKTLNSLEDKAFQLTP | 1007 | FALSE |
| 2_4.0_ORF9B | 64 | 81 | ORF9B | EDKAFQLTPIAVQMTKL | 1008 | FALSE |
| 4_1.0_ORF9B | 83 | 89 | ORF9B | TEELPD | 1009 | FALSE |
| 1_1.0_ORF9C | 14 | 24 | ORF9C | QKASTQKGAE | 1010 | FALSE |
| 1_1.0_S | 25 | 41 | S | PAYTNSFTRGVYYPDK | 1011 | FALSE |
| 1_2.0_S | 30 | 42 | S | SFTRGVYYPDKV | 1012 | FALSE |
| 7_1.0_S | 86 | 94 | S | NDGVYFAS | 1013 | FALSE |
| 8_1.0_S | 115 | 123 | S | SLLIVNNA | 1014 | FALSE |
| 5_1.0_S | 135 | 141 | S | CNDPFL | 1015 | FALSE |
| 5_2.0_S | 136 | 151 | S | NDPFLGVYYHKNNKS | 1016 | FALSE |
| 5_6.0_S | 136 | 154 | S | NDPFLGVYYHKNNKSWME | 1017 | FALSE |
| 5_4.0_S | 142 | 155 | S | VYYHKNNKSWMES | 1018 | FALSE |
| 5_8.0_S | 143 | 150 | S | YYHKNNK | 1019 | FALSE |
| 5_7.0_S | 143 | 154 | S | YYHKNNKSWME | 1020 | FALSE |
| 5_5.0_S | 143 | 155 | S | YYHKNNKSWMES | 1021 | FALSE |
| 5_3.0_S | 143 | 155 | S | YYHKNNKSWMES | 1022 | FALSE |
| 6_1.0_S | 160 | 166 | S | SSANNC | 1023 | FALSE |
| 4_1.0_S | 177 | 188 | S | DLEGKQGNFKN | 1024 | FALSE |
| 4_2.0_S | 179 | 196 | S | EGKQGNFKNLREFVFKN | 1025 | FALSE |
| 4_3.0_S | 183 | 192 | S | GNFKNLREF | 1026 | FALSE |
| 3_1.0_S | 195 | 207 | S | NIDGYFKIYSKH | 1027 | FALSE |
| 2_1.0_S | 227 | 240 | S | DLPIGINITRFQT | 1028 | FALSE |
| 19_1.0_S | 260 | 273 | S | GAAAYYVGYLQPR | 1029 | FALSE |
| 19_2.0_S | 270 | 289 | S | QPRTFLLKYNENGTITDAV | 1030 | FALSE |
| 19_3.0_S | 278 | 285 | S | YNENGTI | 1031 | FALSE |
| 18_1.0_S | 286 | 305 | S | DAVDCALDPLSETKCTLKS | 1032 | FALSE |
| 17_1.0_S | 299 | 305 | S | KCTLKS | 1033 | FALSE |
| 17_2.0_S | 300 | 311 | S | CTLKSFTVEKG | 1034 | FALSE |
| 16_1.0_S | 307 | 324 | S | VEKGIYQTSNFRVQPTE | 1035 | FALSE |
| 14_2.0_S | 326 | 333 | S | VRFPNIT | 1036 | TRUE |
| 14_1.0_S | 329 | 338 | S | PNITNLCPF | 1037 | TRUE |
| 15_1.0_S | 348 | 358 | S | SVYAWNRKRI | 1038 | TRUE |
| 15_2.0_S | 350 | 361 | S | YAWNRKRISNC | 1039 | TRUE |
| 15_3.0_S | 354 | 362 | S | RKRISNCV | 1040 | TRUE |
| 9_1.0_S | 403 | 417 | S | GDEVRQIAPGQTGK | 1041 | TRUE |
| 10_1.0_S | 413 | 427 | S | QTGKIADYNYKLPD | 1042 | TRUE |
| 10_3.0_S | 417 | 430 | S | IADYNYKLPDDFT | 1043 | TRUE |
| 10_2.0_S | 423 | 428 | S | KLPDD | 1044 | TRUE |
| 11_1.0_S | 437 | 448 | S | SNNLDSKVGGN | 1045 | TRUE |
| 12_3.0_S | 452 | 463 | S | YRLFRKSNLKP | 1046 | TRUE |
| 12_2.0_S | 454 | 463 | S | LFRKSNLKP | 1047 | TRUE |
| 12_1.0_S | 458 | 467 | S | SNLKPFERD | 1048 | TRUE |
| 13_1.0_S | 477 | 488 | S | TPCNGVEGFNC | 1049 | TRUE |
| 26_1.0_S | 535 | 547 | S | NKCVNFNFNGLT | 1050 | TRUE |
| 26_2.0_S | 541 | 547 | S | NFNGLT | 1051 | FALSE |
| 27_1.0_S | 547 | 559 | S | GTGVLTESNKKF | 1052 | FALSE |
| 27_2.0_S | 550 | 556 | S | VLTESN | 1053 | FALSE |
| 28_1.0_S | 553 | 566 | S | ESNKKFLPFQQFG | 1054 | FALSE |
| 28_6.0_S | 554 | 565 | S | SNKKFLPFQQF | 1055 | FALSE |
| 28_4.0_S | 554 | 566 | S | SNKKFLPFQQFG | 1056 | FALSE |
| 28_2.0_S | 554 | 569 | S | SNKKFLPFQQFGRDI | 1057 | FALSE |
| 28_5.0_S | 554 | 569 | S | SNKKFLPFQQFGRDI | 1058 | FALSE |
| 28_7.0_S | 555 | 570 | S | NKKFLPFQQFGRDIA | 1059 | FALSE |
| 28_3.0_S | 559 | 569 | S | LPFQQFGRDI | 1060 | FALSE |
| 30_5.0_S | 571 | 581 | S | TTDAVRDPQT | 1061 | FALSE |
| 30_3.0_S | 572 | 588 | S | TDAVRDPQTLEILDIT | 1062 | FALSE |
| 30_4.0_S | 574 | 584 | S | AVRDPQTLEI | 1063 | FALSE |
| 30_2.0_S | 574 | 585 | S | AVRDPQTLEIL | 1064 | FALSE |
| 30_1.0_S | 574 | 585 | S | AVRDPQTLEIL | 1065 | FALSE |
| 29_2.0_S | 598 | 603 | S | TPGTN | 1066 | FALSE |
| 29_1.0_S | 599 | 607 | S | PGTNTSNQ | 1067 | FALSE |
| 31_1.0_S | 620 | 636 | S | PVAIHADQLTPTWRVY | 1068 | FALSE |
| 31_2.0_S | 626 | 640 | S | DQLTPTWRVYSTGS | 1069 | FALSE |
| 31_3.0_S | 628 | 642 | S | LTPTWRVYSTGSNV | 1070 | FALSE |
| 31_4.0_S | 629 | 639 | S | TPTWRVYSTG | 1071 | FALSE |
| 31_5.0_S | 635 | 641 | S | YSTGSN | 1072 | FALSE |
| 36_1.0_S | 650 | 658 | S | IGAEHVNN | 1073 | FALSE |
| 36_2.0_S | 665 | 671 | S | IGAGIC | 1074 | FALSE |
| 35_1.0_S | 674 | 689 | S | QTQTNSPRRARSVAS | 1075 | FALSE |
| 35_2.0_S | 675 | 690 | S | TQTNSPRRARSVASQ | 1076 | FALSE |
| 34_3.0_S | 685 | 703 | S | SVASQSIIAYTMSLGAEN | 1077 | FALSE |
| 34_5.0_S | 688 | 693 | S | SQSII | 1078 | FALSE |
| 34_4.0_S | 688 | 697 | S | SQSIIAYTM | 1079 | FALSE |
| 34_1.0_S | 693 | 701 | S | AYTMSLGA | 1080 | FALSE |
| 34_2.0_S | 694 | 708 | S | YTMSLGAENSVAYS | 1081 | FALSE |
| 32_1.0_S | 701 | 718 | S | ENSVAYSNNSIAIPTNF | 1082 | FALSE |
| 32_3.0_S | 703 | 710 | S | SVAYSNN | 1083 | FALSE |
| 32_2.0_S | 708 | 715 | S | NNSIAIP | 1084 | FALSE |
| 33_1.0_S | 731 | 737 | S | TKTSVD | 1085 | FALSE |
| 49_2.0_S | 766 | 780 | S | LTGIAVEQDKNTQE | 1086 | FALSE |
| 49_5.0_S | 766 | 781 | S | LTGIAVEQDKNTQEV | 1087 | FALSE |
| 49_4.0_S | 768 | 782 | S | GIAVEQDKNTQEVF | 1088 | FALSE |
| 49_3.0_S | 769 | 780 | S | IAVEQDKNTQE | 1089 | FALSE |
| 49_1.0_S | 769 | 781 | S | IAVEQDKNTQEV | 1090 | FALSE |
| 48_1.0_S | 771 | 791 | S | VEQDKNTQEVFAQVKQIYKT | 1091 | FALSE |
| 47_2.0_S | 786 | 800 | S | QIYKTPPIKDFGGF | 1092 | FALSE |
| 47_1.0_S | 787 | 797 | S | IYKTPPIKDF | 1093 | FALSE |
| 46_1.0_S | 790 | 803 | S | TPPIKDFGGFNFS | 1094 | FALSE |
| 44_2.0_s | 798 | 825 | S | GFNFSQILPDPSKPSKRSFIEDLLFNK | 1095 | FALSE |
| 44_5.0_S | 801 | 826 | S | FSQILPDPSKPSKRSFIEDLLFNKV | 1096 | FALSE |
| 44_1.0_S | 804 | 815 | S | ILPDPSKPSKR | 1097 | FALSE |
| 44_6.0_S | 808 | 821 | S | PSKPSKRSFIEDL | 1098 | FALSE |
| 44_3.0_S | 811 | 821 | S | PSKRSFIEDL | 1099 | FALSE |
| 45_8.0_S | 811 | 826 | S | PSKRSFIEDLLFNKV | 1100 | FALSE |
| 45_9.0_S | 811 | 826 | S | PSKRSFIEDLLFNKV | 1101 | FALSE |
| 45_7.0_S | 812 | 826 | S | SKRSFIEDLLFNKV | 1102 | FALSE |
| 45_6.0_S | 812 | 827 | S | SKRSFIEDLLFNKVT | 1103 | FALSE |
| 44_4.0_S | 813 | 820 | S | KRSFIED | 1104 | FALSE |
| 45_4.0_S | 813 | 826 | S | KRSFIEDLLFNKV | 1105 | FALSE |
| 45_5.0_S | 813 | 826 | S | KRSFIEDLLFNKV | 1106 | FALSE |
| 45_2.0_S | 813 | 826 | S | KRSFIEDLLFNKV | 1107 | FALSE |
| 45_1.0_S | 814 | 827 | S | RSFIEDLLENKVT | 1108 | FALSE |
| 45_3.0_S | 816 | 825 | S | FIEDLLENK | 1109 | FALSE |
| 43_1.0_S | 841 | 856 | S | GDIAARDLICAQKFN | 1110 | FALSE |
| 43_2.0_S | 852 | 868 | S | QKFNGLTVLPPLLTDE | 1111 | FALSE |
| 39_1.0_S | 877 | 884 | S | LAGTITS | 1112 | FALSE |
| 38_1.0_S | 899 | 907 | S | MQMAYRFN | 1113 | FALSE |
| 37_1.0_S | 917 | 928 | S | ENQKLIANQFN | 1114 | FALSE |
| 37_4.0_S | 926 | 932 | S | FNSAIG | 1115 | FALSE |
| 37_2.0_S | 931 | 942 | S | GKIQDSLSSTA | 1116 | FALSE |
| 37_3.0_S | 934 | 940 | S | QDSLSS | 1117 | FALSE |
| 41_1.0_S | 965 | 989 | S | LSSNFGAISSVLNDILSRLDKVEA | 1118 | FALSE |
| 42_4.0_S | 972 | 990 | S | ISSVLNDILSRLDKVEAE | 1119 | FALSE |
| 42_3.0_S | 973 | 990 | S | SSVLNDILSRLDKVEAE | 1120 | FALSE |
| 42_5.0_S | 977 | 988 | S | NDILSRLDKVE | 1121 | FALSE |
| 42_1.0_S | 977 | 992 | S | NDILSRLDKVEAEVQ | 1122 | FALSE |
| 42_6.0_S | 978 | 990 | S | DILSRLDKVEAE | 1123 | FALSE |
| 42_2.0_S | 983 | 996 | S | LDKVEAEVQIDRL | 1124 | FALSE |
| 40_1.0_S | 1014 | 1036 | S | AAEIRASANLAATKMSECVLGQ | 1125 | FALSE |
| 40_2.0_S | 1016 | 1032 | S | EIRASANLAATKMSEC | 1126 | FALSE |
| 22_1.0_S | 1051 | 1060 | S | FPQSAPHGV | 1127 | FALSE |
| 21_1.0_S | 1072 | 1083 | S | KNFTTAPAICH | 1128 | FALSE |
| 20_1.0_S | 1091 | 1099 | S | EGVFVSNG | 1129 | FALSE |
| 20_2.0_S | 1104 | 1113 | S | TQRNFYEPQ | 1130 | FALSE |
| 23_10.0_S | 1141 | 1155 | S | QPELDSFKEELDKY | 1131 | FALSE |
| 23_13.0_S | 1141 | 1157 | S | QPELDSFKEELDKYFK | 1132 | FALSE |
| 23_3.0_S | 1141 | 1158 | S | QPELDSFKEELDKYFKN | 1133 | FALSE |
| 23_1.0_S | 1142 | 1157 | S | PELDSFKEELDKYFK | 1134 | FALSE |
| 23_7.0_S | 1143 | 1158 | S | ELDSFKEELDKYFKN | 1135 | FALSE |
| 23_4.0_S | 1143 | 1158 | S | ELDSFKEELDKYFKN | 1136 | FALSE |
| 23_6.0_S | 1143 | 1158 | S | ELDSFKEELDKYFKN | 1137 | FALSE |
| 23_2.0_S | 1145 | 1158 | S | DSFKEELDKYFKN | 1138 | FALSE |
| 23_9.0_S | 1146 | 1156 | S | SFKEELDKYF | 1139 | FALSE |
| 23_14.0_S | 1146 | 1157 | S | SFKEELDKYFK | 1140 | FALSE |
| 23_8.0_S | 1146 | 1157 | S | SFKEELDKYFK | 1141 | FALSE |
| 23_17.0_S | 1146 | 1159 | S | SFKEELDKYFKNH | 1142 | FALSE |
| 23_5.0_S | 1146 | 1161 | S | SFKEELDKYFKNHTS | 1143 | FALSE |
| 23_11.0_S | 1147 | 1157 | S | FKEELDKYFK | 1144 | FALSE |
| 23_19.0_S | 1147 | 1158 | S | FKEELDKYFKN | 1145 | FALSE |
| 23_12.0_S | 1148 | 1156 | S | KEELDKYF | 1146 | FALSE |
| 23_15.0_S | 1149 | 1164 | S | EELDKYFKNHTSPDV | 1147 | FALSE |
| 23_18.0_S | 1151 | 1157 | S | LDKYFK | 1148 | FALSE |
| 23_16.0_S | 1152 | 1160 | S | DKYFKNHT | 1149 | FALSE |
| 24_1.0_S | 1161 | 1176 | S | PDVDLGDISGINASV | 1150 | FALSE |
| 25_2.0_S | 1177 | 1190 | S | NIQKEIDRLNEVA | 1151 | FALSE |
| 25_3.0_S | 1179 | 1190 | S | QKEIDRLNEVA | 1152 | FALSE |
| 25_1.0_S | 1179 | 1191 | S | QKEIDRLNEVAK | 1153 | FALSE |
| 25_4.0_S | 1181 | 1199 | S | EIDRLNEVAKNLNESLID | 1154 | FALSE |
| 25_5.0_S | 1192 | 1197 | S | LNESL | 1155 | FALSE |

# SEQ ID NO:
RBD receptor binding domain

### REFERENCES

1. J. Cui, F. Li, Z.-L. Shi, Origin and evolution of pathogenic coronaviruses. Nat. Rev. Microbiol. 17, 181-192 (2019).
2. T. G. Ksiazek, et al., SARS Working Group, A novel coronavirus associated with severe acute respiratory syndrome. N. Engl. J. Med. 348, 1953-1966 (2003).
3. A. M. Zaki, et al., Isolation of a novel coronavirus from a man with pneumonia in Saudi Arabia. N. Engl. J. Med. 367, 1814-1820 (2012).
4. D.-G. Ahn, et al., Current Status of Epidemiology, Diagnosis, Therapeutics, and Vaccines for Novel Coronavirus Disease 2019 (COVID-19). J. Microbiol. Biotechnol. 30, 313-324 (2020).
5. coronavirus.jhu.edu/map.html
6. K. Yuki, M. Fujiogi, S. Koutsogiannaki, COVID-19 pathophysiology: A review. Clin.

Immunol. 215, 108427 (2020).
7. H. B. Larman, et al., Autoantigen discovery with a synthetic human peptidome. Nature Biotechnology. 29:535-41 (2011). doi: 10.1038/nbt.1856.
8. D. Mohan et al., PhIP-Seq characterization of serum antibodies using oligonucleotide-encoded peptidomes. Nat. Protoc. 13, 1958-1978 (2018).
9. G. J. Xu et al., Viral immunology. Comprehensive serological profiling of human populations using a synthetic human virome. Science. 348, aaa0698 (2015).
10. M. J. Mina, et al., Measles virus infection diminishes preexisting antibodies that offer protection from other pathogens. Science. 366, 599-606 (2019).
11. Protein [Internet]. Bethesda (Md.): National Library of Medicine (US), National Center for Biotechnology Information; 2004 [cited 2020-2-29]. Available from: ncbi.nlm.nih.gov/protein/
12. P. Zhou, et al. A pneumonia outbreak associated with a new coronavirus of probable bat origin. Nature. 579, 270-273 (2020).
13. J. F. W. Chan, et al., Middle East respiratory syndrome coronavirus: another zoonotic betacoronavirus causing SARS-like disease. Clin. Microbiol. Rev. 28, 465-522 (2015).
14. N. Saitou, M. Nei, The neighbor-joining method: a new method for reconstructing phylogenetic trees. Mol. Biol. Evol. 4, 406-425 (1987).
15. S. Kumar, et al., MEGA X: Molecular Evolutionary Genetics Analysis across Computing Platforms. Mol. Biol. Evol. 35, 1547-1549 (2018).
16. K. Tamura, M. Nei, S. Kumar, Prospects for inferring very large phylogenies by using the neighbor-joining method. Proc. Natl. Acad. Sci. U.S.A 101, 11030-11035 (2004).
17. D. E. Gordon, et al., A SARS-CoV-2 protein interaction map reveals targets for drug repurposing. Nature (2020), doi:10.1038/s41586-020-2286-9.
18. G. J. Gorse, et al., Prevalence of antibodies to four human coronaviruses is lower in nasal secretions than in serum. Clin. Vaccine Immunol. 17, 1875-1880 (2010).
19. X. Tian, et al., Potent binding of 2019 novel coronavirus spike protein by a SARS coronavirus-specific human monoclonal antibody. Emerg. Microbes Infect. 9, 382-385 (2020)
20. S. M. Lundberg, et al., From local explanations to global understanding with explainable AI for trees. Nature Machine Intelligence. 2, 56-67 (2020).
21. A. Grifoni, et al., Targets of T Cell Responses to SARS-CoV-2 Coronavirus in Humans with COVID-19 Disease and Unexposed Individuals. Cell (2020), doi:10.1016/j.cell.2020.05.015.
22. Nisreen M. A. et al., Severe Acute Respiratory Syndrome Coronavirus 2-Specific Antibody Responses in Coronavirus Disease 2019 Patients. Emerging Infectious Disease journal. 26 (2020), doi:10.3201/eid2607.200841.
23. Y. Wan et al., Molecular Mechanism for Antibody-Dependent Enhancement of Coronavirus Entry. J. Virol. 94 (2020), doi:10.1128/JVI.02015-19.
24. S.-F. Wang, et al., Antibody-dependent SARS coronavirus infection is mediated by antibodies against spike proteins. Biochem. Biophys. Res. Commun. 451, 208-214 (2014).
25. S. Garg, et al., Hospitalization Rates and Characteristics of Patients Hospitalized with Laboratory-Confirmed Coronavirus Disease 2019-COVID-NET, 14 States, Mar. 1-30, 2020. MMWR Morb. Mortal. Wkly. Rep. 69, 458-464 (2020).
26. M. Webb Hooper, A. M. Napoles, E. J. Perez-Stable, COVID-19 and Racial/Ethnic Disparities. JAMA (2020), doi:10.1001/jama.2020.8598.
27. C. M. Poh, et al., Two linear epitopes on the SARS-CoV-2 spike protein that elicit neutralising antibodies in COVID-19 patients. Nat. Commun. 11, 2806 (2020)
28. J. Lan, J. Ge, J. Yu, S. Shan, H. Zhou, S. Fan, Q. Zhang, X. Shi, Q. Wang, L. Zhang, X. Wang, Structure of the SARS-CoV-2 spike receptor-binding domain bound to the ACE2 receptor. Nature. 581, 215-220 (2020).
29. A. C. Walls, et al., Structure, Function, and Antigenicity of the SARS-CoV-2 Spike Glycoprotein. Cell. 181, 281-292.e6 (2020).
30. D. Wrapp, et al., Cryo-EM structure of the 2019-nCoV spike in the prefusion conformation. Science. 367, 1260-1263 (2020).
31. R. Lachmann, et al., Cytomegalovirus (CMV) seroprevalence in the adult population of Germany. PLoS One. 13, e0200267 (2018).
32. S. L. Bate, et al., Cytomegalovirus seroprevalence in the United States: the national health and nutrition examination surveys, 1988-2004. Clin. Infect. Dis. 50, 1439-1447 (2010).
33. P. Klenerman, P. R. Dunbar, CMV and the art of memory maintenance. Immunity. 29 (2008), pp. 520-522.
34. G. Pawelec, et al., Immunosenescence, suppression and tumour progression. Cancer Immunol. Immunother. 55, 981-986 (2006).
35. S. Prosch, et al., Stimulation of the human cytomegalovirus IE enhancer/promoter in HL-60 cells by TNFalpha is mediated via induction of NF-kappaB. Virology. 208, 197-206 (1995).
36. J. L. Craigen, et al., Human cytomegalovirus infection up-regulates interleukin-8 gene expression and stimulates neutrophil transendothelial migration. Immunology. 92, 138-145 (1997).
37. G. M. Savva, et al., Medical Research Council Cognitive Function and Ageing Study, Cytomegalovirus infection is associated with increased mortality in the older population. Aging Cell. 12, 381-387 (2013).
38. E. Montecino-Rodriguez et al., Causes, consequences, and reversal of immune system aging. J. Clin. Invest. 123, 958-965 (2013).
39. M. B. Coppock, D. N. Stratis-Cullum, A universal method for the functionalization of dyed magnetic microspheres with peptides. Methods. 158, 12-16 (2019).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of detecting the presence of antibodies that bind to SARS-CoV-2 in a sample, the method comprising:
providing a sample comprising or suspected of comprising antibodies that bind to SARS-CoV-2;
contacting the sample with one, two, or more, e.g., 1, 2, 3, 4, 5, 8, 10, 12, 15, 20, 25, 30, 50, 75, 80, 100, 150, 200, 250, 300, 350, 400, 500, 600, 700, 800, or more, peptides comprising 4 or more consecutive amino acids from a SARS-CoV-2 epitope sequence shown in Table 1, Table 3, and/or Table 4 or SEQ ID NOs:13-1170, under conditions sufficient for binding of antibodies in the sample to the peptides; and
detecting binding of antibodies in the sample to the peptides.

2. The method of claim 1, wherein the sample is from a subject, optionally a subject who is known or suspected of being infected with SARS-CoV-2.

3. The method of claim 2, further comprising identifying a subject who has antibodies that bind to SARS-CoV-2 as having been infected with SARS-CoV-2.

4. The method of claim 3, further comprising administering a treatment for SARS-CoV-2 to the subject or monitoring the subject for later health consequences of infection with SARS-CoV-2.

5. The method of claims 2-4, wherein the subject is a human subject.

6. The method of claims 1-5, wherein the sample comprises whole blood, serum, saliva or plasma.

7. The method of claims 1-6, wherein the peptides comprise a detectable moiety, are conjugated to a bead, or are conjugated to a surface.

8. The method of claims 1-7, wherein the detectable moiety is a fluorescent label.

9. The method of claim 7, wherein the surface is a multiwell plate or glass coverslip.

10. The method of claim 7, wherein the beads are magnetic.

11. The method of claims 1-10, wherein detecting comprises performing an immunoassay, multiplex immunoassay, protein-fragment complementation assay (PCA), or single molecule array.

12. A composition comprising one, two, or a plurality of antigenic peptides comprising 4 or more consecutive amino acids from epitope sequences shown in Table 1, 3, or 4 or SEQ ID NOs:13-1170, e.g., from one of SEQ ID NOs: 1036-1050.

13. The composition of claim 12, wherein at least one of the peptides comprises a detectable moiety, is conjugated to a bead, or is conjugated to a surface.

14. The composition of claim 13, wherein the detectable moiety is a fluorescent label.

15. The composition of claim 13, wherein the surface is a multiwell plate or glass coverslip.

16. The composition of claim 13, wherein the beads are magnetic.

17. The composition of claim 12, further comprising a pharmaceutically acceptable carrier and optionally an adjuvant.

18. The composition of claims 12 or 17, for use in a method of treating or reducing risk of an infection with SARS-CoV-2 in a subject.

19. A method of treating or reducing risk or severity of an infection with SARS-CoV-2 in a subject, the method comprising administering a therapeutically of prophylactically effective amount of the composition of claims 12 or 17.

20. A method of generating an antibody to SARS-CoV-2, the method comprising administering the composition of claims 12 or 17, and optionally an adjuvant, to a mammal, and isolating antibodies from the mammal that bind to SARS-CoV-2.

21. A method of identifying antibodies that bind to neutralizing or non-neutralizing epitopes of SARS-CoV-2, the method comprising:
providing a sample comprising an antibody obtained, preferably cloned, from a human who has had a SARS-CoV-2 infection;
contacting the antibody with peptides comprising one or more, e.g., 1, 2, 3, 4, 5, 8, 10, 12, 15, 20, 25, 30, 50, 75, 80, 100, 150, 200, 250, 300, 350, 400, 500, 600, 700, 800, or more, peptides comprising at least 4, e.g., 5, 6, 7, 8, 9, 10, 11, 12, 13, 14, 15, 16, 17, 18, 19, 20 or more, consecutive amino acids from a SARS-CoV-2 epitope sequence shown herein, e.g., in Table 1, Table 3, and/or Table 4 or SEQ ID NOs:13-1170, wherein:
(i) the peptides comprise non-neutralizing epitopes, e.g., from one of SEQ ID NOs: 333-1035 or 1051-1155, and the contacting is performed under conditions to allow binding of the antibody on B cells to the peptides; and identifying the antibody as non-neutralizing if it binds to a peptide that comprises a non-neutralizing epitope; or
(ii) the peptides comprise neutralizing epitopes, e.g., from one of SEQ ID NOs: 1036-1050, and the contacting is performed under conditions to allow binding of the antibody on B cells to the peptides; and identifying the antibody as neutralizing if it binds to a peptide that comprises a neutralizing epitope.

22. The method of claim 21, further comprising cloning one or more antibodies, wherein cloning the antibodies comprises providing a sample of B cells from a human who has had a SARS-CoV-2 infection; contacting the B cells with peptides including one, two, or more of the epitope sequences shown in Table 1, Table 3, and/or Table 4, optionally one of one of SEQ ID NOs: 1036-1050; cloning and sequencing B cells encoding antibodies specific for one or more of the epitope sequences; and optionally testing these antibodies for neutralizing activity or Fc-mediated effector function (e.g., antibody-dependent cellular cytotoxicity, complement-dependent cytotoxicity, and antibody-dependent cellular phagocytosis).

23. The method of claim 21, further comprising formulating the optimized population of antibodies into a pharmaceutical composition by mixing the antibodies with a pharmaceutically acceptable carrier.

24. The method of claim 23, further comprising administering a therapeutically effective amount of the pharmaceutical composition to a subject in need thereof.

25. The method of claim 21, further comprising cloning one or more antibodies identified as non-neutralizing into a pharmaceutical composition.

26. The method of claim 21, further comprising formulating the optimized population of antibodies into a pharmaceutical composition by mixing the antibodies with one or more of a pharmaceutically acceptable carrier, an adjuvant, and/or a SARS-CoV-2 vaccine comprising a SARS-CoV-2 protein, peptide, or nucleic acid encoding a SARS-CoV-2 protein or peptide.

27. The method of claim 26, further comprising administering a prophylactically effective amount of the pharmaceutical composition to a subject in need thereof.

28. A method of selecting a vaccine composition for use in eliciting a prophylactic response to SARS-CoV-2 in a subject, the method comprising:
administering a composition comprising a SARS-CoV-2 protein, peptide, or nucleic acid encoding a SARS-CoV-2 protein or peptide, to a test subject in an amount sufficient to elicit an immune response;
obtaining a sample comprising antibodies obtained from the subject;
contacting the sample with one or more, e.g., 1, 2, 3, 4, 5, 8, 10, 12, 15, 20, 25, 30, 50, 75, 80, 100, 150, 200, 250, 300, 350, 400, 500, 600, 700, 800, or more, peptides comprising at least 4, e.g., 5, 6, 7, 8, 9, 10, 11, 12, 13, 14, 15, 16, 17, 18, 19, 20 or more, consecutive amino acids from a SARS-CoV-2 epitope sequence shown herein, e.g., in Table 1, Table 3, and/or Table 4 or SEQ ID NOs:13-1170, under conditions to allow binding of the antibody to the peptides;
detecting binding of antibodies in the sample to the peptides, wherein:
(i) the composition of the vaccine excludes one or more epitopes that elicit non-protective antibodies; or
(ii) the composition of the vaccine comprises epitopes that elicit protective (neutralizing) antibodies, e.g., one of one of SEQ ID NOs: 1036-1050; and selecting a vaccine composition that elicits neutralizing antibodies.

29. The method of claim 28, wherein the vaccine composition comprises one or more mutations in a non-neutralizing epitope.

30. A composition comprising a SARS-CoV-2 protein, peptide, or nucleic acid encoding a SARS-CoV-2 protein or peptide, wherein the SARS-CoV-2 protein, peptide, or nucleic acid encoding a SARS-CoV-2 protein or peptide comprises a mutation in a non-neutralizing epitope sequences shown in Table 3 or 4, and a pharmaceutically acceptable carrier, and optionally an adjuvant.

31. The composition of claim 30, for use in eliciting a prophylactic response in a subject.

32. A method of generating an antibody to SARS-CoV-2, the method comprising administering the composition of claim 30 to a subject.

33. A method of treating or reducing risk or severity of an infection with SARS-CoV-2 in a subject, the method comprising administering a therapeutically or prophylactically effective amount of the composition of claim 30 to the subject.

34. A kit comprising the composition of claims 12-16, for use in a method of detecting the presence of antibodies that bind to SARS-CoV-2 in a sample.


SEQUENCE LISTING (1211 sequences)

SEQ ID NO: 1 - DNA, 14 bp, Artificial Sequence
  Description of Artificial Sequence Synthetic oligonucleotide
gaattcggagcggt

SEQ ID NO: 2 - DNA, 14 bp, Artificial Sequence
  Description of Artificial Sequence Synthetic oligonucleotide
cactgcactcgaga

SEQ ID NO: 3 - DNA, 27 bp, Artificial Sequence
  Description of Artificial Sequence Synthetic primer
aatgatacggcgtgaattcggagcggt

SEQ ID NO: 4 - DNA, 27 bp, Artificial Sequence
  Description of Artificial Sequence Synthetic primer
caagcagaagacgtctcgagtgcagtg

SEQ ID NO: 5 - DNA, 14 bp, Artificial Sequence
  Description of Artificial Sequence Synthetic oligonucleotide
gaattccgctgcgt

SEQ ID NO: 6 - DNA, 14 bp, Artificial Sequence
  Description of Artificial Sequence Synthetic oligonucleotide
cagggaagagctcg

SEQ ID NO: 7 - DNA, 27 bp, Artificial Sequence
  Description of Artificial Sequence Synthetic primer
aatgatacggcgggaattccgctgcgt

SEQ ID NO: 8 - DNA, 27 bp, Artificial Sequence
  Description of Artificial Sequence Synthetic primer
caagcagaagactcgagctcttccctg

SEQ ID NO: 9 - DNA, 14 bp, Artificial Sequence
  Description of Artificial Sequence Synthetic oligonucleotide
gaattccgctgcgt

SEQ ID NO: 10 - DNA, 23 bp, Artificial Sequence
  Description of Artificial Sequence Synthetic oligonucleotide
gtactatacctacggaaggctcg

SEQ ID NO: 11 - DNA, 27 bp, Artificial Sequence
  Description of Artificial Sequence Synthetic primer
aatgatacggcgggaattccgctgcgt

SEQ ID NO: 12 - DNA, 45 bp, Artificial Sequence
  Description of Artificial Sequence Synthetic primer
tatctcgcatagcgcatatactcgagccttccgtaggtatagtac

SEQ ID NO: 13 - protein, 27 aa, Artificial Sequence
  Description of Artificial Sequence Synthetic peptide
  MOD_RES (1)..(1): Propargylgylcine
XKKSFDLGDELGTDPYEDFQENWNTKH

SEQ ID NO: 14 - protein, 27 aa, Artificial Sequence
  Description of Artificial Sequence Synthetic peptide
  MOD_RES (1)..(1): Propargylgylcine
XKKNNAAIVLQLPQGTTLPKGFYAEGS

SEQ ID NO: 15 - protein, 27 aa, Artificial Sequence
  Description of Artificial Sequence Synthetic peptide
  MOD_RES (1)..(1): Propargylgylcine
XKKIDGYFKIYSKHTPINLVRDLPQGF

SEQ ID NO: 16 - protein, 27 aa, Artificial Sequence
  Description of Artificial Sequence Synthetic peptide
  MOD_RES (1)..(1): Propargylgylcine
XKKLLLLDRLNQLESKMSGKGQQQQGQ

SEQ ID NO: 17 - protein, 26 aa, Artificial Sequence
  Description of Artificial Sequence Synthetic peptide
  MOD_RES (1)..(1): Propargylgylcine
XKKGQQQQGQTVTKKSAAEASKKPRQ

SEQ ID NO: 18 - protein, 27 aa, Artificial Sequence
  Description of Artificial Sequence Synthetic peptide
  MOD_RES (1)..(1): Propargylgylcine
XKKDAYKTFPPTEPKKDKKKKADETQA

SEQ ID NO: 19 - protein, 27 aa, Artificial Sequence
  Description of Artificial Sequence Synthetic peptide
  MOD_RES (1)..(1): Propargylgylcine
XKKLPQRQKKQQTVTLLPAADLDDFSK

SEQ ID NO: 20 - protein, 27 aa, Artificial Sequence
  Description of Artificial Sequence Synthetic peptide
  MOD_RES (1)..(1): Propargylgylcine
XKKTGTGVLTESNKKFLPFQQFGRDIA

SEQ ID NO: 21 - protein, 28 aa, Artificial Sequence
  Description of Artificial Sequence Synthetic peptide
  MOD_RES (1)..(1): Propargylgylcine
XKKRRARSVASQSIIAYTMSLGAENSVA

SEQ ID NO: 22 - protein, 27 aa, Artificial Sequence
  Description of Artificial Sequence Synthetic peptide
  MOD_RES (1)..(1): Propargylgylcine
XKKQLNRALTGIAVEQDKNTQEVFAQV

SEQ ID NO: 23 - protein, 27 aa, Artificial Sequence
  Description of Artificial Sequence Synthetic peptide
  MOD_RES (1)..(1): Propargylgylcine
XKKFAQVKQIYKTPPIKDFGGFNFSQI

SEQ ID NO: 24 - protein, 27 aa, Artificial Sequence
  Description of Artificial Sequence Synthetic peptide
  MOD_RES (1)..(1): Propargylgylcine
XKKPDPSKPSKRSFIEDLLFNKVTLAD

SEQ ID NO: 25 - protein, 27 aa, Artificial Sequence
  Description of Artificial Sequence Synthetic peptide
  MOD_RES (1)..(1): Propargylgylcine
XKKYDPLQPELDSFKEELDKYFKNHTS

SEQ ID NO: 26 - protein, 27 aa, Artificial Sequence
  Description of Artificial Sequence Synthetic peptide
  MOD_RES (1)..(1): Propargylgylcine
XKKCCSCGSCCKFDEDDSEPVLKGVKL

SEQ ID NO: 27 - protein, 28 aa, Artificial Sequence
  Description of Artificial Sequence Synthetic peptide
  MOD_RES (1)..(1): Propargylgylcine
XKKNPIENYVDEVLNEVLVVPNINSSHP

SEQ ID NO: 28 - protein, 28 aa, Artificial Sequence
  Description of Artificial Sequence Synthetic peptide
  MOD_RES (1)..(1): Propargylgylcine
XKKPPPGRRPFFHPVAEADYFEYHQEGG

SEQ ID NO: 29 - protein, 28 aa, Artificial Sequence
  Description of Artificial Sequence Synthetic peptide
  MOD_RES (1)..(1): Propargylgylcine
XKKQDNSDIKVVPRRKAKIIRDYGKQMA

SEQ ID NO: 30 - protein, 31 aa, Severe acute respiratory syndrome coronavirus 2
SVTSSIVITSGDGTTSPISEHDYQIGGYTEK

SEQ ID NO: 31 - protein, 19 aa, Severe acute respiratory syndrome coronavirus 2
NPVMEPIYDEPTTTTSVPL

SEQ ID NO: 32 - protein, 20 aa, Severe acute respiratory syndrome coronavirus 2
FIYNKIVDEPEEHVQIHTID

SEQ ID NO: 33 - protein, 15 aa, Severe acute respiratory syndrome coronavirus 2
HHLGRCDIKDLPKEI

SEQ ID NO: 34 - protein, 10 aa, Severe acute respiratory syndrome coronavirus 2
ADSNGTITVE

SEQ ID NO: 35 - protein, 23 aa, Severe acute respiratory syndrome coronavirus 2
RIGNYKLNTDHSSSSDNIALLVQ

SEQ ID NO: 36 - protein, 19 aa, Severe acute respiratory syndrome coronavirus 2
YYKLGASQRVAGDSGFAAY

SEQ ID NO: 37 - protein, 19 aa, Severe acute respiratory syndrome coronavirus 2
IAEILLIIMRTFKVSIWNL

SEQ ID NO: 38 - protein, 11 aa, Severe acute respiratory syndrome coronavirus 2
FHLVDFQVTIA

SEQ ID NO: 39 - protein, 23 aa, Severe acute respiratory syndrome coronavirus 2
NLIIKNLSKSLTENKYSQLDEEQ

SEQ ID NO: 40 - protein, 11 aa, Severe acute respiratory syndrome coronavirus 2
QLDEEQPMEID

SEQ ID NO: 41 - protein, 21 aa, Severe acute respiratory syndrome coronavirus 2
SKSLTENKYSQLDEEQPMEID

SEQ ID NO: 42 - protein, 12 aa, Severe acute respiratory syndrome coronavirus 2
EGNSPFHPLADN

SEQ ID NO: 43 - protein, 8 aa, Severe acute respiratory syndrome coronavirus 2
DEAGSKSP

SEQ ID NO: 44 - protein, 9 aa, Severe acute respiratory syndrome coronavirus 2
YVVDDPCPI

SEQ ID NO: 45 - protein, 6 aa, Severe acute respiratory syndrome coronavirus 2
VVLDFI

SEQ ID NO: 46 - protein, 22 aa, Severe acute respiratory syndrome coronavirus 2
RSKQRRPQGLPNNTASWFTALT

SEQ ID NO: 47 - protein, 16 aa, Severe acute respiratory syndrome coronavirus 2
VTLLPAADLDDFSKQL

SEQ ID NO: 48 - protein, 16 aa, Severe acute respiratory syndrome coronavirus 2
SSRSSSRSRNSSRNST

SEQ ID NO: 49 - protein, 22 aa, Severe acute respiratory syndrome coronavirus 2
GKGQQQQGQTVTKKSAAEASKK

SEQ ID NO: 50 - protein, 16 aa, Severe acute respiratory syndrome coronavirus 2
LQLPQGTTLPKGFYAE

SEQ ID NO: 51 - protein, 17 aa, Severe acute respiratory syndrome coronavirus 2
LALLLLDRLNQLESKMS

SEQ ID NO: 52 - protein, 12 aa, Severe acute respiratory syndrome coronavirus 2
YNVTQAFGRRGP

SEQ ID NO: 53 - protein, 27 aa, Severe acute respiratory syndrome coronavirus 2
GYYRRATRRIRGGDGKMKDLSPRWYFY

SEQ ID NO: 54 - protein, 21 aa, Severe acute respiratory syndrome coronavirus 2
ASKKPRQKRTATKAYNVTQAF

SEQ ID NO: 55 - protein, 16 aa, Severe acute respiratory syndrome coronavirus 2
TFPPTEPKKDKKKKAD

SEQ ID NO: 56 - protein, 19 aa, Severe acute respiratory syndrome coronavirus 2
TQGNFGDQELIRQGTDYKH

SEQ ID NO: 57 - protein, 19 aa, Severe acute respiratory syndrome coronavirus 2
DKKKKADETQALPQRQKKQ

SEQ ID NO: 58 - protein, 24 aa, Severe acute respiratory syndrome coronavirus 2
PAADLDDFSKQLQQSMSSADSTQA

SEQ ID NO: 59 - protein, 19 aa, Severe acute respiratory syndrome coronavirus 2
LLNKHIDAYKTFPPTEPKK

SEQ ID NO: 60 - protein, 23 aa, Severe acute respiratory syndrome coronavirus 2
LDDKDPNFKDQVILLNKHIDAYK

SEQ ID NO: 61 - protein, 14 aa, Severe acute respiratory syndrome coronavirus 2
SGARSKQRRPQGLP

SEQ ID NO: 62 - protein, 8 aa, Severe acute respiratory syndrome coronavirus 2
TGSNQNGE

SEQ ID NO: 63 - protein, 19 aa, Severe acute respiratory syndrome coronavirus 2
GKEDLKFPRGQGVPINTNS

SEQ ID NO: 64 - protein, 16 aa, Severe acute respiratory syndrome coronavirus 2
PKDHIGTRNPANNAAI

SEQ ID NO: 65 - protein, 38 aa, Severe acute respiratory syndrome coronavirus 2
GGDAALALLLLDRLNQLESKMSGKGQQQQGQTVTKKSA

SEQ ID NO: 66 - protein, 22 aa, Severe acute respiratory syndrome coronavirus 2
DETQALPQRQKKQQTVTLLPAA

SEQ ID NO: 67 - protein, 36 aa, Severe acute respiratory syndrome coronavirus 2
FYYLGTGPEAGLPYGANKDGIIWVATEGALNTPKDH

SEQ ID NO: 68 - protein, 15 aa, Severe acute respiratory syndrome coronavirus 2
QTVTKKSAAEASKKP

SEQ ID NO: 69 - protein, 27 aa, Severe acute respiratory syndrome coronavirus 2
SRGTSPARMAGNGGDAALALLLLDRLN

SEQ ID NO: 70 - protein, 12 aa, Severe acute respiratory syndrome coronavirus 2
SAFFGMSRIGME

SEQ ID NO: 71 - protein, 20 aa, Severe acute respiratory syndrome coronavirus 2
GGDAALALLLLDRLNQLESK

SEQ ID NO: 72 - protein, 6 aa, Severe acute respiratory syndrome coronavirus 2
TEELPD

SEQ ID NO: 73 - protein, 31 aa, Severe acute respiratory syndrome coronavirus 2
PLSLNMARKTLNSLEDKAFQLTPIAVQMTKL

SEQ ID NO: 74 - protein, 9 aa, Severe acute respiratory syndrome coronavirus 2
PIILRLGSP

SEQ ID NO: 75 - protein, 10 aa, Severe acute respiratory syndrome coronavirus 2
DPKISEMHPA

SEQ ID NO: 76 - protein, 10 aa, Severe acute respiratory syndrome coronavirus 2
QKASTQKGAE

SEQ ID NO: 77 - protein, 16 aa, Severe acute respiratory syndrome coronavirus 2
LTGIAVEQDKNTQEVF

SEQ ID NO: 78 - protein, 16 aa, Severe acute respiratory syndrome coronavirus 2
PSKRSFIEDLLFNKVT

SEQ ID NO: 79 - protein, 23 aa, Severe acute respiratory syndrome coronavirus 2
QPELDSFKEELDKYFKNHTSPDV

SEQ ID NO: 80 - protein, 24 aa, Severe acute respiratory syndrome coronavirus 2
ISSVLNDILSRLDKVEAEVQIDRL

SEQ ID NO: 81 - protein, 17 aa, Severe acute respiratory syndrome coronavirus 2
QTGKIADYNYKLPDDFT

SEQ ID NO: 82 - protein, 11 aa, Severe acute respiratory syndrome coronavirus 2
SNNLDSKVGGN

SEQ ID NO: 83 - protein, 23 aa, Severe acute respiratory syndrome coronavirus 2
SVASQSIIAYTMSLGAENSVAYS

SEQ ID NO: 84 - protein, 28 aa, Severe acute respiratory syndrome coronavirus 2
GFNFSQILPDPSKPSKRSFIEDLLFNKV

SEQ ID NO: 85 - protein, 22 aa, Severe acute respiratory syndrome coronavirus 2
AAEIRASANLAATKMSECVLGQ

SEQ ID NO: 86 - protein, 25 aa, Severe acute respiratory syndrome coronavirus 2
ENQKLIANQFNSAIGKIQDSLSSTA

SEQ ID NO: 87 - protein, 15 aa, Severe acute respiratory syndrome coronavirus 2
YRLFRKSNLKPFERD

SEQ ID NO: 88 - protein, 29 aa, Severe acute respiratory syndrome coronavirus 2
GAAAYYVGYLQPRTFLLKYNENGTITDAV

SEQ ID NO: 89 - protein, 12 aa, Severe acute respiratory syndrome coronavirus 2
KCTLKSFTVEKG

SEQ ID NO: 90 - protein, 12 aa, Severe acute respiratory syndrome coronavirus 2
GTGVLTESNKKF

SEQ ID NO: 91 - protein, 8 aa, Severe acute respiratory syndrome coronavirus 2
MQMAYRFN

SEQ ID NO: 92 - protein, 17 aa, Severe acute respiratory syndrome coronavirus 2
TTDAVRDPQTLEILDIT

SEQ ID NO: 93 - protein, 20 aa, Severe acute respiratory syndrome coronavirus 2
CNDPFLGVYYHKNNKSWMES

SEQ ID NO: 94 - protein, 16 aa, Severe acute respiratory syndrome coronavirus 2
QTQTNSPRRARSVASQ

SEQ ID NO: 95 - protein, 22 aa, Severe acute respiratory syndrome coronavirus 2
NIQKEIDRLNEVAKNLNESLID

SEQ ID NO: 96 - protein, 13 aa, Severe acute respiratory syndrome coronavirus 2
TPPIKDFGGFNFS

SEQ ID NO: 97 - protein, 17 aa, Severe acute respiratory syndrome coronavirus 2
VEKGIYQTSNFRVQPTE

SEQ ID NO: 98 - protein, 12 aa, Severe acute respiratory syndrome coronavirus 2
VRFPNITNLCPF

SEQ ID NO: 99 - protein, 17 aa, Severe acute respiratory syndrome coronavirus 2
ESNKKFLPFQQFGRDIA

SEQ ID NO: 100 - protein, 19 aa, Severe acute respiratory syndrome coronavirus 2
DAVDCALDPLSETKCTLKS

SEQ ID NO: 101 - protein, 22 aa, Severe acute respiratory syndrome coronavirus 2
PVAIHADQLTPTWRVYSTGSNV

SEQ ID NO: 102 - protein, 21 aa, Severe acute respiratory syndrome coronavirus 2
IGAEHVNNSYECDIPIGAGIC

SEQ ID NO: 103 - protein, 14 aa, Severe acute respiratory syndrome coronavirus 2
QIYKTPPIKDFGGF

SEQ ID NO: 104 - protein, 8 aa, Severe acute respiratory syndrome coronavirus 2
NDGVYFAS

SEQ ID NO: 105 - protein, 17 aa, Severe acute respiratory syndrome coronavirus 2
ENSVAYSNNSIAIPTNF

SEQ ID NO: 106 - protein, 9 aa, Severe acute respiratory syndrome coronavirus 2
TPGTNTSNQ

SEQ ID NO: 107 - protein, 14 aa, Severe acute respiratory syndrome coronavirus 2
GDEVRQIAPGQTGK

SEQ ID NO: 108 - protein, 27 aa, Severe acute respiratory syndrome coronavirus 2
GDIAARDLICAQKFNGLTVLPPLLTDE

SEQ ID NO: 109 - protein, 20 aa, Severe acute respiratory syndrome coronavirus 2
VEQDKNTQEVFAQVKQIYKT

SEQ ID NO: 110 - protein, 15 aa, Severe acute respiratory syndrome coronavirus 2
PDVDLGDISGINASV

SEQ ID NO: 111 - protein, 9 aa, Severe acute respiratory syndrome coronavirus 2
FPQSAPHGV

SEQ ID NO: 112 - protein, 11 aa, Severe acute respiratory syndrome coronavirus 2
TPCNGVEGFNC

SEQ ID NO: 113 - protein, 6 aa, Severe acute respiratory syndrome coronavirus 2
TKTSVD

SEQ ID NO: 114 - protein, 22 aa, Severe acute respiratory syndrome coronavirus 2
EGVFVSNGTHWFVTQRNFYEPQ

SEQ ID NO: 115 - protein, 19 aa, Severe acute respiratory syndrome coronavirus 2
DLEGKQGNFKNLREFVFKN

SEQ ID NO: 116 - protein, 13 aa, Severe acute respiratory syndrome coronavirus 2
DLPIGINITRFQT

SEQ ID NO: 117 - protein, 8 aa, Severe acute respiratory syndrome coronavirus 2
SLLIVNNA

SEQ ID NO: 118 - protein, 12 aa, Severe acute respiratory syndrome coronavirus 2
NKCVNFNFNGLT

SEQ ID NO: 119 - protein, 24 aa, Severe acute respiratory syndrome coronavirus 2
LSSNFGAISSVLNDILSRLDKVEA

SEQ ID NO: 120 - protein, 14 aa, Severe acute respiratory syndrome coronavirus 2
SVYAWNRKRISNCV

SEQ ID NO: 121 - protein, 12 aa, Severe acute respiratory syndrome coronavirus 2
NIDGYFKIYSKH

SEQ ID NO: 122 - protein, 6 aa, Severe acute respiratory syndrome coronavirus 2
SSANNC

SEQ ID NO: 123 - protein, 7 aa, Severe acute respiratory syndrome coronavirus 2
LAGTITS

SEQ ID NO: 124 - protein, 11 aa, Severe acute respiratory syndrome coronavirus 2
KNFTTAPAICH

SEQ ID NO: 125 - protein, 17 aa, Severe acute respiratory syndrome coronavirus 2
PAYTNSFTRGVYYPDKV

SEQ ID NO: 126 - protein, 29 aa, Severe acute respiratory syndrome coronavirus 2
SEKSYELQTPFEIKLAKKFDTFNGECPNF

SEQ ID NO: 127 - protein, 14 aa, Severe acute respiratory syndrome coronavirus 2
EQPTSEAVEAPLVG

SEQ ID NO: 128 - protein, 16 aa, Severe acute respiratory syndrome coronavirus 2
QFGPTYLDGADVTKIK

SEQ ID NO: 129 - protein, 26 aa, Severe acute respiratory syndrome coronavirus 2
CTEIDPKLDNYYKKDNSYFTEQPIDL

SEQ ID NO: 130 - protein, 19 aa, Severe acute respiratory syndrome coronavirus 2
SRIKASMPTTIAKNTVKSV

SEQ ID NO: 131 - protein, 15 aa, Severe acute respiratory syndrome coronavirus 2
NNYMLTYNKVENMTP

SEQ ID NO: 132 - protein, 11 aa, Severe acute respiratory syndrome coronavirus 2
LKHGTFTCASE

SEQ ID NO: 133 - protein, 19 aa, Severe acute respiratory syndrome coronavirus 2
DNIKFADDLNQLTGYKKPA

SEQ ID NO: 134 - protein, 19 aa, Severe acute respiratory syndrome coronavirus 2
HYTPSFKKGAKLLHKPIVW

SEQ ID NO: 135 - protein, 10 aa, Severe acute respiratory syndrome coronavirus 2
CCHLAKALND

SEQ ID NO: 136 - protein, 17 aa, Severe acute respiratory syndrome coronavirus 2
SQGLLPPKNSIDAFKLN

SEQ ID NO: 137 - protein, 25 aa, Severe acute respiratory syndrome coronavirus 2
ATNYDLSVVNARLRAKHYVYIGDPA

SEQ ID NO: 138 - protein, 23 aa, Severe acute respiratory syndrome coronavirus 2
AFELWAKRNIKPVPEVKILNNLG

SEQ ID NO: 139 - protein, 15 aa, Severe acute respiratory syndrome coronavirus 2
ARNGVLITEGSVKGL

SEQ ID NO: 140 - protein, 18 aa, Severe acute respiratory syndrome coronavirus 2
SDMYDPKTKNVTKENDSK

SEQ ID NO: 141 - protein, 21 aa, Severe acute respiratory syndrome coronavirus 2
PTEVLTEEVVLKTGDLQPLEQ

SEQ ID NO: 142 - protein, 11 aa, Severe acute respiratory syndrome coronavirus 2
KTLQPVSELLT

SEQ ID NO: 143 - protein, 15 aa, Severe acute respiratory syndrome coronavirus 2
DNVYIKNADIVEEAK

SEQ ID NO: 144 - protein, 22 aa, Severe acute respiratory syndrome coronavirus 2
HCLHVVGPNVNKGEDIQLLKSA

SEQ ID NO: 145 - protein, 28 aa, Severe acute respiratory syndrome coronavirus 2
DLMAAYVDNSSLTIKKPNELSRVLGLKT

SEQ ID NO: 146 - protein, 23 aa, Severe acute respiratory syndrome coronavirus 2
FISDEVARDLSLQFKRPINPTDQ

SEQ ID NO: 147 - protein, 22 aa, Severe acute respiratory syndrome coronavirus 2
SPNLAWPLIVTALRANSAVKLQ

SEQ ID NO: 148 - protein, 17 aa, Severe acute respiratory syndrome coronavirus 2
YQKVGMQKYSTLQGPPG

SEQ ID NO: 149 - protein, 9 aa, Severe acute respiratory syndrome coronavirus 2
NPAWRKAVF

SEQ ID NO: 150 - protein, 28 aa, Severe acute respiratory syndrome coronavirus 2
STIGVCSMTDIAKKPTETICAPLTVFFD

SEQ ID NO: 151 - protein, 20 aa, Severe acute respiratory syndrome coronavirus 2
EIPVAYRKVLLRKNGNKGAG

SEQ ID NO: 152 - protein, 15 aa, Severe acute respiratory syndrome coronavirus 2
PYEDFQENWNTKHSS

SEQ ID NO: 153 - protein, 24 aa, Severe acute respiratory syndrome coronavirus 2
HGGGVAGALNKATNNAMQVESDDY

SEQ ID NO: 154 - protein, 18 aa, Severe acute respiratory syndrome coronavirus 2
IPKEEVKPFITESKPSVE

SEQ ID NO: 155 - protein, 22 aa, Severe acute respiratory syndrome coronavirus 2
DAVTAYNGYLTSSSKTPEEHFI

SEQ ID NO: 156 - protein, 30 aa, Severe acute respiratory syndrome coronavirus 2
VKTTEVVGDIILKPANNSLKITEEVGHTDL

SEQ ID NO: 157 - protein, 19 aa, Severe acute respiratory syndrome coronavirus 2
ACIDCSARHINAQVAKSHN

SEQ ID NO: 158 - protein, 24 aa, Severe acute respiratory syndrome coronavirus 2
RQRLTKYTMADLVYALRHFDEGNC

SEQ ID NO: 159 - protein, 14 aa, Severe acute respiratory syndrome coronavirus 2
YYQNNVFMSEAKCW

SEQ ID NO: 160 - protein, 26 aa, Severe acute respiratory syndrome coronavirus 2
LAVHECFVKRVDWTIEYPIIGDELKI

SEQ ID NO: 161 - protein, 18 aa, Severe acute respiratory syndrome coronavirus 2
VQHMVVKAALLADKFPVL

SEQ ID NO: 162 - protein, 5 aa, Severe acute respiratory syndrome coronavirus 2
EHIVY

SEQ ID NO: 163 - protein, 24 aa, Severe acute respiratory syndrome coronavirus 2
DNLLEILQKEKVNINIVGDFKLNE

SEQ ID NO: 164 - protein, 33 aa, Severe acute respiratory syndrome coronavirus 2
QEGVLTAVVIPTKKAGGTTEMLAKALRKVPTDN

SEQ ID NO: 165 - protein, 22 aa, Severe acute respiratory syndrome coronavirus 2
SLSEQLRKQIRSAAKKNNLPFK

SEQ ID NO: 166 - protein, 17 aa, Severe acute respiratory syndrome coronavirus 2
ENKTTLPVNVAFELWAK

SEQ ID NO: 167 - protein, 16 aa, Severe acute respiratory syndrome coronavirus 2
PNMMVTNNTFTLKGGA

SEQ ID NO: 168 - protein, 15 aa, Severe acute respiratory syndrome coronavirus 2
DTIANYAKPFLNKVV

SEQ ID NO: 169 - protein, 13 aa, Severe acute respiratory syndrome coronavirus 2
TTKIALKGGKIVN

SEQ ID NO: 170 - protein, 15 aa, Severe acute respiratory syndrome coronavirus 2
SAVLQSGFRKMAFPS

SEQ ID NO: 171 - protein, 19 aa, Severe acute respiratory syndrome coronavirus 2
EVVLKKLKKSLNVAKSEFD

SEQ ID NO: 172 - protein, 10 aa, Severe acute respiratory syndrome coronavirus 2
DLTKPYIKWD

SEQ ID NO: 173 - protein, 11 aa, Severe acute respiratory syndrome coronavirus 2
LQEFKPRSQME

SEQ ID NO: 174 - protein, 21 aa, Severe acute respiratory syndrome coronavirus 2
EAKKVKPTVVVNAANVYLKHG

SEQ ID NO: 175 - protein, 22 aa, Severe acute respiratory syndrome coronavirus 2
VDSVTVKNGSIHLYFDKAGQKT

SEQ ID NO: 176 - protein, 13 aa, Severe acute respiratory syndrome coronavirus 2
RGFGDSVEEVLSE

SEQ ID NO: 177 - protein, 15 aa, Severe acute respiratory syndrome coronavirus 2
KKDAPYIVGDVVQEG

SEQ ID NO: 178 - protein, 25 aa, Severe acute respiratory syndrome coronavirus 2
AQCFKMFYKGVITHDVSSAINRPQI

SEQ ID NO: 179 - protein, 6 aa, Severe acute respiratory syndrome coronavirus 2
TKEGAT

SEQ ID NO: 180 - protein, 18 aa, Severe acute respiratory syndrome coronavirus 2
DKNLYDKLVSSFLEMKSE

SEQ ID NO: 181 - protein, 19 aa, Severe acute respiratory syndrome coronavirus 2
EAVMYMGTLSYEQFKKGVQ

SEQ ID NO: 182 - protein, 7 aa, Severe acute respiratory syndrome coronavirus 2
IEYTDFA

SEQ ID NO: 183 - protein, 27 aa, Severe acute respiratory syndrome coronavirus 2
LPSYAAFATAQEAYEQAVANGDSEVVL

SEQ ID NO: 184 - protein, 19 aa, Severe acute respiratory syndrome coronavirus 2
IGEAVKTQFNYYKKVDGVV

SEQ ID NO: 185 - protein, 12 aa, Severe acute respiratory syndrome coronavirus 2
VFIKRSDARTAP

SEQ ID NO: 186 - protein, 11 aa, Severe acute respiratory syndrome coronavirus 2
LEGIQYGRSGE

SEQ ID NO: 187 - protein, 14 aa, Severe acute respiratory syndrome coronavirus 2
DLGDELGTDPYEDF

SEQ ID NO: 188 - protein, 33 aa, Severe acute respiratory syndrome coronavirus 2
EDIQLLKSAYENFNQHEVLLAPLLSAGIFGADP

SEQ ID NO: 189 - protein, 17 aa, Severe acute respiratory syndrome coronavirus 2
ESKPSVEQRKQDDKKIK

SEQ ID NO: 190 - protein, 23 aa, Severe acute respiratory syndrome coronavirus 2
YLVQQESPFVMMSAPPAQYELKH

SEQ ID NO: 191 - protein, 15 aa, Severe acute respiratory syndrome coronavirus 2
IKTIQPRVEKKKLDG

SEQ ID NO: 192 - protein, 14 aa, Severe acute respiratory syndrome coronavirus 2
VVKIYCPACHNSEV

SEQ ID NO: 193 - protein, 16 aa, Severe acute respiratory syndrome coronavirus 2
ELDERIDKVLNEKCSA

SEQ ID NO: 194 - protein, 24 aa, Severe acute respiratory syndrome coronavirus 2
MEKLKTLVATAEAELAKNVSLDNV

SEQ ID NO: 195 - protein, 18 aa, Severe acute respiratory syndrome coronavirus 2
GTSTDVVYRAFDIYNDKV

SEQ ID NO: 196 - protein, 14 aa, Severe acute respiratory syndrome coronavirus 2
INANQVIVNNLDKS

SEQ ID NO: 197 - protein, 15 aa, Severe acute respiratory syndrome coronavirus 2
QMNLKYAISAKNRAR

SEQ ID NO: 198 - protein, 22 aa, Severe acute respiratory syndrome coronavirus 2
SVKGLQPSVGPKQASLNGVTLI

SEQ ID NO: 199 - protein, 23 aa, Severe acute respiratory syndrome coronavirus 2
VHTANKWDLIISDMYDPKTKNVT

SEQ ID NO: 200 - protein, 18 aa, Severe acute respiratory syndrome coronavirus 2
KPIVWHVNNATNKATYKP

SEQ ID NO: 201 - protein, 15 aa, Severe acute respiratory syndrome coronavirus 2
EIVDTVSALVYDNKL

SEQ ID NO: 202 - protein, 15 aa, Severe acute respiratory syndrome coronavirus 2
ECNQMCLSTLMKCDH

SEQ ID NO: 203 - protein, 9 aa, Severe acute respiratory syndrome coronavirus 2
IFTQTTETA

SEQ ID NO: 204 - protein, 21 aa, Severe acute respiratory syndrome coronavirus 2
SNYLKRRVVFNGVSFSTFEEA

SEQ ID NO: 205 - protein, 19 aa, Severe acute respiratory syndrome coronavirus 2
KTDGTLMIERFVSLAIDAY

SEQ ID NO: 206 - protein, 11 aa, Severe acute respiratory syndrome coronavirus 2
NQEYADVFHLY

SEQ ID NO: 207 - protein, 19 aa, Severe acute respiratory syndrome coronavirus 2
PLKVGGSCVLSGHNLAKHC

SEQ ID NO: 208 - protein, 15 aa, Severe acute respiratory syndrome coronavirus 2
SGVTRELMRELNGGA

SEQ ID NO: 209 - protein, 21 aa, Severe acute respiratory syndrome coronavirus 2
AECTIFKDASGKPVPYCYDTN

SEQ ID NO: 210 - protein, 14 aa, Severe acute respiratory syndrome coronavirus 2
PKGFCDLKGKYVQI

SEQ ID NO: 211 - protein, 15 aa, Severe acute respiratory syndrome coronavirus 2
NPPALQDAYYRARAG

SEQ ID NO: 212 - protein, 20 aa, Severe acute respiratory syndrome coronavirus 2
EEVVENPTIQKDVLECNVKT

SEQ ID NO: 213 - protein, 19 aa, Severe acute respiratory syndrome coronavirus 2
FDKSAFVNLKQLPFFYYSD

SEQ ID NO: 214 - protein, 17 aa, Severe acute respiratory syndrome coronavirus 2
HGKQVVSDIDYVPLKSA

SEQ ID NO: 215 - protein, 15 aa, Severe acute respiratory syndrome coronavirus 2
TLVSDIDITFLKKDA

SEQ ID NO: 216 - protein, 14 aa, Severe acute respiratory syndrome coronavirus 2
FLKRGDKSVYYTSN

SEQ ID NO: 217 - protein, 10 aa, Severe acute respiratory syndrome coronavirus 2
IATCDWTNAG

SEQ ID NO: 218 - protein, 15 aa, Severe acute respiratory syndrome coronavirus 2
RKYKGIKIQEGVVDY

SEQ ID NO: 219 - protein, 13 aa, Severe acute respiratory syndrome coronavirus 2
WFSQRGGSYTNDK

SEQ ID NO: 220 - protein, 24 aa, Severe acute respiratory syndrome coronavirus 2
KPRPPLNRNYVFTGYRVTKNSKVQ

SEQ ID NO: 221 - protein, 5 aa, Severe acute respiratory syndrome coronavirus 2
GNALD

SEQ ID NO: 222 - protein, 21 aa, Severe acute respiratory syndrome coronavirus 2
LLPLTQYNRYLALYNKYKYFS

SEQ ID NO: 223 - protein, 26 aa, Severe acute respiratory syndrome coronavirus 2
YKENSYTTTIKPVTYKLDGVVCTEID

SEQ ID NO: 224 - protein, 20 aa, Severe acute respiratory syndrome coronavirus 2
NIKLLGVGGKPCIKVATVQS

SEQ ID NO: 225 - protein, 21 aa, Severe acute respiratory syndrome coronavirus 2
ICQAVTANVNALLSTDGNKIA

SEQ ID NO: 226 - protein, 27 aa, Severe acute respiratory syndrome coronavirus 2
DPAQLPAPRTLLTKGTLEPEYFNSVCR

SEQ ID NO: 227 - protein, 29 aa, Severe acute respiratory syndrome coronavirus 2
FSASTSAFVETVKGLDYKAFKQIVESCGN

SEQ ID NO: 228 - protein, 9 aa, Severe acute respiratory syndrome coronavirus 2
DSAEVAVKM

SEQ ID NO: 229 - protein, 12 aa, Severe acute respiratory syndrome coronavirus 2
NLDNLRANNTKG

SEQ ID NO: 230 - protein, 21 aa, Severe acute respiratory syndrome coronavirus 2
KLKVDTANPKTPKYKFVRIQP

SEQ ID NO: 231 - protein, 23 aa, Severe acute respiratory syndrome coronavirus 2
ARLYYDSMSYEDQDALFAYTKRN

SEQ ID NO: 232 - protein, 22 aa, Severe acute respiratory syndrome coronavirus 2
WCIRCLWSTKPVETSNSFDVLK

SEQ ID NO: 233 - protein, 19 aa, Severe acute respiratory syndrome coronavirus 2
MLDNRATLQAIASEFSSLP

SEQ ID NO: 234 - protein, 12 aa, Severe acute respiratory syndrome coronavirus 2
IAEIPKEEVKPF

SEQ ID NO: 235 - protein, 10 aa, Severe acute respiratory syndrome coronavirus 2
VCVETKAIVS

SEQ ID NO: 236 - protein, 20 aa, Severe acute respiratory syndrome coronavirus 2
RVIHFGAGSDKGVAPGTAVL

SEQ ID NO: 237 - protein, 11 aa, Severe acute respiratory syndrome coronavirus 2
ESLVPGFNEKT

SEQ ID NO: 238 - protein, 23 aa, Severe acute respiratory syndrome coronavirus 2
HLDGEVITFDNLKTLLSLREVRT

SEQ ID NO: 239 - protein, 16 aa, Severe acute respiratory syndrome coronavirus 2
KCDLQNYGDSATLPKG

SEQ ID NO: 240 - protein, 18 aa, Severe acute respiratory syndrome coronavirus 2
REETGLLMPLKAPKEIIF

SEQ ID NO: 241 - protein, 11 aa, Severe acute respiratory syndrome coronavirus 2
CGHYKHITSKE

SEQ ID NO: 242 - protein, 17 aa, Severe acute respiratory syndrome coronavirus 2
NQDVNLHSSRLSFKELL

SEQ ID NO: 243 - protein, 25 aa, Severe acute respiratory syndrome coronavirus 2
IGEYTFEKGDYGDAVVYRGTTTYKL

SEQ ID NO: 244 - protein, 29 aa, Severe acute respiratory syndrome coronavirus 2
PQADVEWKFYDAQPCSDKAYKIEELFYSY

SEQ ID NO: 245 - protein, 8 aa, Severe acute respiratory syndrome coronavirus 2
MDSTVKNY

SEQ ID NO: 246 - protein, 22 aa, Severe acute respiratory syndrome coronavirus 2
SKCEESSAKSASVYYSQLMCQP

SEQ ID NO: 247 - protein, 9 aa, Severe acute respiratory syndrome coronavirus 2
YSGQSTQLG

SEQ ID NO: 248 - protein, 14 aa, Severe acute respiratory syndrome coronavirus 2
YDNKLKAHKDKSAQ

SEQ ID NO: 249 - protein, 19 aa, Severe acute respiratory syndrome coronavirus 2
LMLLEIKDTEKYCALAPNM

SEQ ID NO: 250 - protein, 12 aa, Severe acute respiratory syndrome coronavirus 2
KTCGQQQTTLKG

SEQ ID NO: 251 - protein, 8 aa, Severe acute respiratory syndrome coronavirus 2
IVFDGKSK

SEQ ID NO: 252 - protein, 18 aa, Severe acute respiratory syndrome coronavirus 2
KELLQNGMNGRTILGSAL

SEQ ID NO: 253 - protein, 25 aa, Severe acute respiratory syndrome coronavirus 2
SCGNFKVTKGKAKKGAWNIGEQKSI

SEQ ID NO: 254 - protein, 19 aa, Severe acute respiratory syndrome coronavirus 2
MLAKALRKVPTDNYITTYP

SEQ ID NO: 255 - protein, 17 aa, Severe acute respiratory syndrome coronavirus 2
VLKSEDAQGMDNLACED

SEQ ID NO: 256 - protein, 22 aa, Severe acute respiratory syndrome coronavirus 2
KKVDGVVQQLPETYFTQSRNLQ

SEQ ID NO: 257 - protein, 24 aa, Severe acute respiratory syndrome coronavirus 2
FTSLEIPRRNVATLQAENVTGLFK

SEQ ID NO: 258 - protein, 24 aa, Severe acute respiratory syndrome coronavirus 2
PRVEKKKLDGFMGRIRSVYPVASP

SEQ ID NO: 259 - protein, 26 aa, Severe acute respiratory syndrome coronavirus 2
GLNLEEAARYMRSLKVPATVSVSSPD

SEQ ID NO: 260 - protein, 26 aa, Severe acute respiratory syndrome coronavirus 2
TQYEYGTEDDYQGKPLEFGATSAALQ

SEQ ID NO: 261 - protein, 21 aa, Severe acute respiratory syndrome coronavirus 2
EMKSEKQVEQKIAEIPKEEVK

SEQ ID NO: 262 - protein, 20 aa, Severe acute respiratory syndrome coronavirus 2
NNTDFSRVSAKPPPGDQFKH

SEQ ID NO: 263 - protein, 10 aa, Severe acute respiratory syndrome coronavirus 2
ENVAFNVVNK

SEQ ID NO: 264 - protein, 14 aa, Severe acute respiratory syndrome coronavirus 2
AKRFKESPFELEDF

SEQ ID NO: 265 - protein, 7 aa, Severe acute respiratory syndrome coronavirus 2
PDYNTYK

SEQ ID NO: 266 - protein, 20 aa, Severe acute respiratory syndrome coronavirus 2
EKDEDDNLIDSYFVVKRHTF

SEQ ID NO: 267 - protein, 11 aa, Severe acute respiratory syndrome coronavirus 2
SLINTLNDLNE

SEQ ID NO: 268 - protein, 16 aa, Severe acute respiratory syndrome coronavirus 2
DPSFLGRYMSALNHTK

SEQ ID NO: 269 - protein, 9 aa, Severe acute respiratory syndrome coronavirus 2
IKGLNNLNR

SEQ ID NO: 270 - protein, 30 aa, Severe acute respiratory syndrome coronavirus 2
ALTNNVAFQTVKPGNFNKDFYDFAVSKGFF

SEQ ID NO: 271 - protein, 17 aa, Severe acute respiratory syndrome coronavirus 2
KASCTLSEQLDFIDTKR

SEQ ID NO: 272 - protein, 22 aa, Severe acute respiratory syndrome coronavirus 2
YASQGLVASIKNFKSVLYYQNN

SEQ ID NO: 273 - protein, 16 aa, Severe acute respiratory syndrome coronavirus 2
NLGETFVTHSKGLYRK

SEQ ID NO: 274 - protein, 18 aa, Severe acute respiratory syndrome coronavirus 2
TFGDDTVIEVQGYKSVNI

SEQ ID NO: 275 - protein, 23 aa, Severe acute respiratory syndrome coronavirus 2
DGALLTKSSEYKGPITDVFYKEN

SEQ ID NO: 276 - protein, 6 aa, Severe acute respiratory syndrome coronavirus 2
NLGMPS

SEQ ID NO: 277 - protein, 13 aa, Severe acute respiratory syndrome coronavirus 2
SSEIIGYKAIDGG

SEQ ID NO: 278 - protein, 8 aa, Severe acute respiratory syndrome coronavirus 2
GDRWFLNR

SEQ ID NO: 279 - protein, 12 aa, Severe acute respiratory syndrome coronavirus 2
MLRKLDNDALNN

SEQ ID NO: 280 - protein, 6 aa, Severe acute respiratory syndrome coronavirus 2
TEVPAN

SEQ ID NO: 281 - protein, 6 aa, Severe acute respiratory syndrome coronavirus 2
MDQESF

SEQ ID NO: 282 - protein, 6 aa, Severe acute respiratory syndrome coronavirus 2
DFVNEF

SEQ ID NO: 283 - protein, 16 aa, Severe acute respiratory syndrome coronavirus 2
SDRVVFVLWAHGFELT

SEQ ID NO: 284 - protein, 15 aa, Severe acute respiratory syndrome coronavirus 2
KCVCSVIDLLLDDFV

SEQ ID NO: 285 - protein, 16 aa, Severe acute respiratory syndrome coronavirus 2
VFISPYNSQNAVASKI

SEQ ID NO: 286 - protein, 15 aa, Severe acute respiratory syndrome coronavirus 2
KEGSSVELKHFFFAQ

SEQ ID NO: 287 - protein, 11 aa, Severe acute respiratory syndrome coronavirus 2
PSIISNEKQEI

SEQ ID NO: 288 - protein, 29 aa, Severe acute respiratory syndrome coronavirus 2
EGCHATREAVGTNLPLQLGFSTGVNLVAV

SEQ ID NO: 289 - protein, 10 aa, Severe acute respiratory syndrome coronavirus 2
YIDINGNLHP

SEQ ID NO: 290 - protein, 23 aa, Severe acute respiratory syndrome coronavirus 2
AARQGFVDSDVETKDVVECLKLS

SEQ ID NO: 291 - protein, 17 aa, Severe acute respiratory syndrome coronavirus 2
VGDFKLNEEIAIILASF

SEQ ID NO: 292 - protein, 31 aa, Severe acute respiratory syndrome coronavirus 2
YCNKTVGELGDVRETMSYLFQHANLDSCKRV

SEQ ID NO: 293 - protein, 8 aa, Severe acute respiratory syndrome coronavirus 2
KTILRKGG

SEQ ID NO: 294 - protein, 32 aa, Severe acute respiratory syndrome coronavirus 2
EETIYNLLKDCPAVAKHDFFKFRIDGDMVPHI

SEQ ID NO: 295 - protein, 28 aa, Severe acute respiratory syndrome coronavirus 2
HFDGQQGEVPVSIINNTVYTKVDGVDVE

SEQ ID NO: 296 - protein, 6 aa, Severe acute respiratory syndrome coronavirus 2
CAAGTT

SEQ ID NO: 297 - protein, 8 aa, Severe acute respiratory syndrome coronavirus 2
VPRASANI

SEQ ID NO: 298 - protein, 18 aa, Severe acute respiratory syndrome coronavirus 2
TGLFKDCSKVITGLHPTQ

SEQ ID NO: 299 - protein, 15 aa, Severe acute respiratory syndrome coronavirus 2
MRPNFTIKGSFLNGS

SEQ ID NO: 300 - protein, 9 aa, Severe acute respiratory syndrome coronavirus 2
TTVDNINLH

SEQ ID NO: 301 - protein, 30 aa, Severe acute respiratory syndrome coronavirus 2
DAAKAYKDYLASGGQPITNCVKMLCTHTGT

SEQ ID NO: 302 - protein, 18 aa, Severe acute respiratory syndrome coronavirus 2
VLALLSDLQDLKWARFPK

SEQ ID NO: 303 - protein, 22 aa, Severe acute respiratory syndrome coronavirus 2
DNQTTTIQTIVEVQPQLEMELT

SEQ ID NO: 304 - protein, 19 aa, Severe acute respiratory syndrome coronavirus 2
SLRPDTRYVLMDGSIIQFP

SEQ ID NO: 305 - protein, 13 aa, Severe acute respiratory syndrome coronavirus 2
DNQDLNGNWYDFG

SEQ ID NO: 306 - protein, 11 aa, Severe acute respiratory syndrome coronavirus 2
PFNKWGKARLY

SEQ ID NO: 307 - protein, 16 aa, Severe acute respiratory syndrome coronavirus 2
GYPLECIKDLLARAGK

SEQ ID NO: 308 - protein, 9 aa, Severe acute respiratory syndrome coronavirus 2
GLNGYTVEE

SEQ ID NO: 309 - protein, 8 aa, Severe acute respiratory syndrome coronavirus 2
EDEEEGDC

SEQ ID NO: 310 - protein, 12 aa, Severe acute respiratory syndrome coronavirus 2
QMYKQARSEDKR

SEQ ID NO: 311 - protein, 9 aa, Severe acute respiratory syndrome coronavirus 2
DRRATCFST

SEQ ID NO: 312 - protein, 9 aa, Severe acute respiratory syndrome coronavirus 2
CDGGSLYVN

SEQ ID NO: 313 - protein, 15 aa, Severe acute respiratory syndrome coronavirus 2
FLNRVCGVSAARLTP

SEQ ID NO: 314 - protein, 5 aa, Severe acute respiratory syndrome coronavirus 2
ADLYK

SEQ ID NO: 315 - protein, 11 aa, Severe acute respiratory syndrome coronavirus 2
IVGGQIVTCAK

SEQ ID NO: 316 - protein, 15 aa, Severe acute respiratory syndrome coronavirus 2
HMELPTGVHAGTDLE

SEQ ID NO: 317 - protein, 8 aa, Severe acute respiratory syndrome coronavirus 2
ILLAKDTT

SEQ ID NO: 318 - protein, 24 aa, Severe acute respiratory syndrome coronavirus 2
KGPHEFCSQHTMLVKQGDDYVYLP

SEQ ID NO: 319 - protein, 14 aa, Severe acute respiratory syndrome coronavirus 2
LLVDSDLNDFVSDA

SEQ ID NO: 320 - protein, 15 aa, Severe acute respiratory syndrome coronavirus 2
VGILCIMSDRDLYDK

SEQ ID NO: 321 - protein, 11 aa, Severe acute respiratory syndrome coronavirus 2
LIRKSNHNFLV

SEQ ID NO: 322 - protein, 14 aa, Severe acute respiratory syndrome coronavirus 2
NQPYPNASFDNFKF

SEQ ID NO: 323 - protein, 19 aa, Severe acute respiratory syndrome coronavirus 2
KWKYPQVNGLTSIKWADNN

SEQ ID NO: 324 - protein, 11 aa, Severe acute respiratory syndrome coronavirus 2
DADSKIVQLSE

SEQ ID NO: 325 - protein, 14 aa, Severe acute respiratory syndrome coronavirus 2
DIASTDTCFANKHA

SEQ ID NO: 326 - protein, 16 aa, Severe acute respiratory syndrome coronavirus 2
DKFKVNSTLEQYVFCT

SEQ ID NO: 327 - protein, 11 aa, Severe acute respiratory syndrome coronavirus 2
ETLKATEETFK

SEQ ID NO: 328 - protein, 7 aa, Severe acute respiratory syndrome coronavirus 2
VCTNYMP

SEQ ID NO: 329 - protein, 9 aa, Severe acute respiratory syndrome coronavirus 2
YRYNLPTMC

SEQ ID NO: 330 - protein, 6 aa, Severe acute respiratory syndrome coronavirus 2
LQSNHD

SEQ ID NO: 331 - protein, 8 aa, Severe acute respiratory syndrome coronavirus 2
ETFYPKLQ

SEQ ID NO: 332 - protein, 10 aa, Severe acute respiratory syndrome coronavirus 2
NRQFHQKLLK

SEQ ID NO: 333 - protein, 10 aa, Severe acute respiratory syndrome coronavirus 2
ADSNGTITVE

SEQ ID NO: 334 - protein, 12 aa, Severe acute respiratory syndrome coronavirus 2
HHLGRCDIKDLP

SEQ ID NO: 335 - protein, 11 aa, Severe acute respiratory syndrome coronavirus 2
RCDIKDLPKEI

SEQ ID NO: 336 - protein, 14 aa, Severe acute respiratory syndrome coronavirus 2
YYKLGASQRVAGDS

SEQ ID NO: 337 - protein, 8 aa, Severe acute respiratory syndrome coronavirus 2
GDSGFAAY

SEQ ID NO: 338 - protein, 11 aa, Severe acute respiratory syndrome coronavirus 2
RIGNYKLNTDH

SEQ ID NO: 339 - protein, 16 aa, Severe acute respiratory syndrome coronavirus 2
NTDHSSSSDNIALLVQ

SEQ ID NO: 340 - protein, 15 aa, Severe acute respiratory syndrome coronavirus 2
TDHSSSSDNIALLVQ

SEQ ID NO: 341 - protein, 8 aa, Severe acute respiratory syndrome coronavirus 2
TGSNQNGE

SEQ ID NO: 342 - protein, 9 aa, Severe acute respiratory syndrome coronavirus 2
SGARSKQRR

SEQ ID NO: 343 - protein, 11 aa, Severe acute respiratory syndrome coronavirus 2
GARSKQRRPQG

SEQ ID NO: 344 - protein, 11 aa, Severe acute respiratory syndrome coronavirus 2
RSKQRRPQGLP

SEQ ID NO: 345 - protein, 13 aa, Severe acute respiratory syndrome coronavirus 2
RSKQRRPQGLPNN

SEQ ID NO: 346 - protein, 10 aa, Severe acute respiratory syndrome coronavirus 2
QRRPQGLPNN

SEQ ID NO: 347 - protein, 8 aa, Severe acute respiratory syndrome coronavirus 2
PQGLPNNT

SEQ ID NO: 348 - protein, 12 aa, Severe acute respiratory syndrome coronavirus 2
PNNTASWFTALT

SEQ ID NO: 349 - protein, 8 aa, Severe acute respiratory syndrome coronavirus 2
GKEDLKFP

SEQ ID NO: 350 - protein, 7 aa, Severe acute respiratory syndrome coronavirus 2
VPINTNS

SEQ ID NO: 351 - protein, 10 aa, Severe acute respiratory syndrome coronavirus 2
GYYRRATRRI

SEQ ID NO: 352 - protein, 13 aa, Severe acute respiratory syndrome coronavirus 2
TRRIRGGDGKMKD

SEQ ID NO: 353 - protein, 11 aa, Severe acute respiratory syndrome coronavirus 2
RRIRGGDGKMK

SEQ ID NO: 354 - protein, 14 aa, Severe acute respiratory syndrome coronavirus 2
RRIRGGDGKMKDLS

SEQ ID NO: 355 - protein, 12 aa, Severe acute respiratory syndrome coronavirus 2
RIRGGDGKMKDL

SEQ ID NO: 356 - protein, 11 aa, Severe acute respiratory syndrome coronavirus 2
IRGGDGKMKDL

SEQ ID NO: 357 - protein, 10 aa, Severe acute respiratory syndrome coronavirus 2
GGDGKMKDLS

SEQ ID NO: 358 - protein, 10 aa, Severe acute respiratory syndrome coronavirus 2
KDLSPRWYFY

SEQ ID NO: 359 - protein, 23 aa, Severe acute respiratory syndrome coronavirus 2
FYYLGTGPEAGLPYGANKDGIIW

SEQ ID NO: 360 - protein, 15 aa, Severe acute respiratory syndrome coronavirus 2
TGPEAGLPYGANKDG

SEQ ID NO: 361 - protein, 11 aa, Severe acute respiratory syndrome coronavirus 2
PYGANKDGIIW

SEQ ID NO: 362 - protein, 6 aa, Severe acute respiratory syndrome coronavirus 2
KDGIIW

SEQ ID NO: 363 - protein, 19 aa, Severe acute respiratory syndrome coronavirus 2
KDGIIWVATEGALNTPKDH

SEQ ID NO: 364 - protein, 11 aa, Severe acute respiratory syndrome coronavirus 2
PKDHIGTRNPA

SEQ ID NO: 365 - protein, 6 aa, Severe acute respiratory syndrome coronavirus 2
ANNAAI

SEQ ID NO: 366 - protein, 13 aa, Severe acute respiratory syndrome coronavirus 2
LQLPQGTTLPKGF

SEQ ID NO: 367 - protein, 9 aa, Severe acute respiratory syndrome coronavirus 2
QLPQGTTLP

SEQ ID NO: 368 - protein, 15 aa, Severe acute respiratory syndrome coronavirus 2
QLPQGTTLPKGFYAE

SEQ ID NO: 369 - protein, 14 aa, Severe acute respiratory syndrome coronavirus 2
LPQGTTLPKGFYAE

SEQ ID NO: 370 - protein, 11 aa, Severe acute respiratory syndrome coronavirus 2
SSRSSSRSRNS

SEQ ID NO: 371 - protein, 11 aa, Severe acute respiratory syndrome coronavirus 2
SSRSRNSSRNS

SEQ ID NO: 372 - protein, 11 aa, Severe acute respiratory syndrome coronavirus 2
SSRSRNSSRNS

SEQ ID NO: 373 - protein, 7 aa, Severe acute respiratory syndrome coronavirus 2
NSSRNST

SEQ ID NO: 374 - protein, 8 aa, Severe acute respiratory syndrome coronavirus 2
SRGTSPAR

SEQ ID NO: 375 - protein, 19 aa, Severe acute respiratory syndrome coronavirus 2
ARMAGNGGDAALALLLLDR

SEQ ID NO: 376 - protein, 10 aa, Severe acute respiratory syndrome coronavirus 2
AGNGGDAALA

SEQ ID NO: 377 - protein, 10 aa, Severe acute respiratory syndrome coronavirus 2
NGGDAALALL

SEQ ID NO: 378 - protein, 5 aa, Severe acute respiratory syndrome coronavirus 2
GGDAA

SEQ ID NO: 379 - protein, 13 aa, Severe acute respiratory syndrome coronavirus 2
GGDAALALLLLDR

SEQ ID NO: 380 - protein, 14 aa, Severe acute respiratory syndrome coronavirus 2
GGDAALALLLLDRL

SEQ ID NO: 381 - protein, 19 aa, Severe acute respiratory syndrome coronavirus 2
GGDAALALLLLDRLNQLES

SEQ ID NO: 382 - protein, 20 aa, Severe acute respiratory syndrome coronavirus 2
GGDAALALLLLDRLNQLESK

SEQ ID NO: 383 - protein, 30 aa, Severe acute respiratory syndrome coronavirus 2
GGDAALALLLLDRLNQLESKMSGKGQQQQG

SEQ ID NO: 384 - protein, 14 aa, Severe acute respiratory syndrome coronavirus 2
GDAALALLLLDRLN

SEQ ID NO: 385 - protein, 14 aa, Severe acute respiratory syndrome coronavirus 2
LALLLLDRLNQLES

SEQ ID NO: 386 - protein, 13 aa, Severe acute respiratory syndrome coronavirus 2
LLLLDRLNQLESK

SEQ ID NO: 387 - protein, 15 aa, Severe acute respiratory syndrome coronavirus 2
LLLLDRLNQLESKMS

SEQ ID NO: 388 - protein, 11 aa, Severe acute respiratory syndrome coronavirus 2
LLLDRLNQLES

SEQ ID NO: 389 - protein, 22 aa, Severe acute respiratory syndrome coronavirus 2
LLLDRLNQLESKMSGKGQQQQG

SEQ ID NO: 390 - protein, 13 aa, Severe acute respiratory syndrome coronavirus 2
LLDRLNQLESKMS

SEQ ID NO: 391 - protein, 19 aa, Severe acute respiratory syndrome coronavirus 2
LDRLNQLESKMSGKGQQQQ

SEQ ID NO: 392 - protein, 20 aa, Severe acute respiratory syndrome coronavirus 2
LDRLNQLESKMSGKGQQQQG

SEQ ID NO: 393 - protein, 10 aa, Severe acute respiratory syndrome coronavirus 2
DRLNQLESKM

SEQ ID NO: 394 - protein, 11 aa, Severe acute respiratory syndrome coronavirus 2
DRLNQLESKMS

SEQ ID NO: 395 - protein, 27 aa, Severe acute respiratory syndrome coronavirus 2
DRLNQLESKMSGKGQQQQGQTVTKKSA

SEQ ID NO: 396 - protein, 13 aa, Severe acute respiratory syndrome coronavirus 2
LESKMSGKGQQQQ

SEQ ID NO: 397 - protein, 14 aa, Severe acute respiratory syndrome coronavirus 2
LESKMSGKGQQQQG

SEQ ID NO: 398 - protein, 12 aa, Severe acute respiratory syndrome coronavirus 2
ESKMSGKGQQQQ

SEQ ID NO: 399 - protein, 14 aa, Severe acute respiratory syndrome coronavirus 2
ESKMSGKGQQQQGQ

SEQ ID NO: 400 - protein, 7 aa, Severe acute respiratory syndrome coronavirus 2
SKMSGKG

SEQ ID NO: 401 - protein, 12 aa, Severe acute respiratory syndrome coronavirus 2
GKGQQQQGQTVT

SEQ ID NO: 402 - protein, 13 aa, Severe acute respiratory syndrome coronavirus 2
QQQQGQTVTKKSA

SEQ ID NO: 403 - protein, 19 aa, Severe acute respiratory syndrome coronavirus 2
QQQQGQTVTKKSAAEASKK

SEQ ID NO: 404 - protein, 15 aa, Severe acute respiratory syndrome coronavirus 2
QQQGQTVTKKSAAEA

SEQ ID NO: 405 - protein, 9 aa, Severe acute respiratory syndrome coronavirus 2
QGQTVTKKS

SEQ ID NO: 406 - protein, 15 aa, Severe acute respiratory syndrome coronavirus 2
QTVTKKSAAEASKKP

SEQ ID NO: 407 - protein, 13 aa, Severe acute respiratory syndrome coronavirus 2
VTKKSAAEASKKP

SEQ ID NO: 408 - protein, 10 aa, Severe acute respiratory syndrome coronavirus 2
KSAAEASKKP

SEQ ID NO: 409 - protein, 10 aa, Severe acute respiratory syndrome coronavirus 2
ASKKPRQKRT

SEQ ID NO: 410 - protein, 11 aa, Severe acute respiratory syndrome coronavirus 2
KKPRQKRTATK

SEQ ID NO: 411 - protein, 11 aa, Severe acute respiratory syndrome coronavirus 2
RQKRTATKAYN

SEQ ID NO: 412 - protein, 15 aa, Severe acute respiratory syndrome coronavirus 2
QKRTATKAYNVTQAF

SEQ ID NO: 413 - protein, 5 aa, Severe acute respiratory syndrome coronavirus 2
KRTAT

SEQ ID NO: 414 - protein, 5 aa, Severe acute respiratory syndrome coronavirus 2
TATKA

SEQ ID NO: 415 - protein, 8 aa, Severe acute respiratory syndrome coronavirus 2
TKAYNVTQ

SEQ ID NO: 416 - protein, 11 aa, Severe acute respiratory syndrome coronavirus 2
YNVTQAFGRRG

SEQ ID NO: 417 - protein, 12 aa, Severe acute respiratory syndrome coronavirus 2
YNVTQAFGRRGP

SEQ ID NO: 418 - protein, 8 aa, Severe acute respiratory syndrome coronavirus 2
TQGNFGDQ

SEQ ID NO: 419 - protein, 9 aa, Severe acute respiratory syndrome coronavirus 2
IRQGTDYKH

SEQ ID NO: 420 - protein, 12 aa, Severe acute respiratory syndrome coronavirus 2
SAFFGMSRIGME

SEQ ID NO: 421 - protein, 12 aa, Severe acute respiratory syndrome coronavirus 2
LDDKDPNFKDQV

SEQ ID NO: 422 - protein, 23 aa, Severe acute respiratory syndrome coronavirus 2
LDDKDPNFKDQVILLNKHIDAYK

SEQ ID NO: 423 - protein, 13 aa, Severe acute respiratory syndrome coronavirus 2
NFKDQVILLNKHI

SEQ ID NO: 424 - protein, 8 aa, Severe acute respiratory syndrome coronavirus 2
DQVILLNK

SEQ ID NO: 425 - protein, 10 aa, Severe acute respiratory syndrome coronavirus 2
LLNKHIDAYK

SEQ ID NO: 426 - protein, 8 aa, Severe acute respiratory syndrome coronavirus 2
NKHIDAYK

SEQ ID NO: 427 - protein, 16 aa, Severe acute respiratory syndrome coronavirus 2
KHIDAYKTFPPTEPKK

SEQ ID NO: 428 - protein, 12 aa, Severe acute respiratory syndrome coronavirus 2
DAYKTFPPTEPK

SEQ ID NO: 429 - protein, 11 aa, Severe acute respiratory syndrome coronavirus 2
TFPPTEPKKDK

SEQ ID NO: 430 - protein, 8 aa, Severe acute respiratory syndrome coronavirus 2
FPPTEPKK

SEQ ID NO: 431 - protein, 7 aa, Severe acute respiratory syndrome coronavirus 2
PTEPKKD

SEQ ID NO: 432 - protein, 10 aa, Severe acute respiratory syndrome coronavirus 2
PTEPKKDKKK

SEQ ID NO: 433 - protein, 9 aa, Severe acute respiratory syndrome coronavirus 2
EPKKDKKKK

SEQ ID NO: 434 - protein, 11 aa, Severe acute respiratory syndrome coronavirus 2
EPKKDKKKKAD

SEQ ID NO: 435 - protein, 18 aa, Severe acute respiratory syndrome coronavirus 2
DKKKKADETQALPQRQKK

SEQ ID NO: 436 - protein, 11 aa, Severe acute respiratory syndrome coronavirus 2
KKKADETQALP

SEQ ID NO: 437 - protein, 16 aa, Severe acute respiratory syndrome coronavirus 2
KKADETQALPQRQKKQ

SEQ ID NO: 438 - protein, 5 aa, Severe acute respiratory syndrome coronavirus 2
DETQA

SEQ ID NO: 439 - protein, 12 aa, Severe acute respiratory syndrome coronavirus 2
DETQALPQRQKK

SEQ ID NO: 440 - protein, 13 aa, Severe acute respiratory syndrome coronavirus 2
DETQALPQRQKKQ

SEQ ID NO: 441 - protein, 22 aa, Severe acute respiratory syndrome coronavirus 2
DETQALPQRQKKQQTVTLLPAA

SEQ ID NO: 442 - protein, 11 aa, Severe acute respiratory syndrome coronavirus 2
ETQALPQRQKK

SEQ ID NO: 443 - protein, 13 aa, Severe acute respiratory syndrome coronavirus 2
LPQRQKKQQTVTL

SEQ ID NO: 444 - protein, 13 aa, Severe acute respiratory syndrome coronavirus 2
QKKQQTVTLLPAA

SEQ ID NO: 445 - protein, 8 aa, Severe acute respiratory syndrome coronavirus 2
KKQQTVTL

SEQ ID NO: 446 - protein, 10 aa, Severe acute respiratory syndrome coronavirus 2
KKQQTVTLLP

SEQ ID NO: 447 - protein, 5 aa, Severe acute respiratory syndrome coronavirus 2
TVTLL

SEQ ID NO: 448 - protein, 6 aa, Severe acute respiratory syndrome coronavirus 2
TVTLLP

SEQ ID NO: 449 - protein, 14 aa, Severe acute respiratory syndrome coronavirus 2
VTLLPAADLDDFSK

SEQ ID NO: 450 - protein, 15 aa, Severe acute respiratory syndrome coronavirus 2
VTLLPAADLDDFSKQ

SEQ ID NO: 451 - protein, 13 aa, Severe acute respiratory syndrome coronavirus 2
LLPAADLDDFSKQ

SEQ ID NO: 452 - protein, 12 aa, Severe acute respiratory syndrome coronavirus 2
PAADLDDFSKQL

SEQ ID NO: 453 - protein, 16 aa, Severe acute respiratory syndrome coronavirus 2
PAADLDDFSKQLQQSM

SEQ ID NO: 454 - protein, 16 aa, Severe acute respiratory syndrome coronavirus 2
DDFSKQLQQSMSSADS

SEQ ID NO: 455 - protein, 12 aa, Severe acute respiratory syndrome coronavirus 2
SKQLQQSMSSAD

SEQ ID NO: 456 - protein, 5 aa, Severe acute respiratory syndrome coronavirus 2
DSTQA

SEQ ID NO: 457 - protein, 9 aa, Severe acute respiratory syndrome coronavirus 2
ESLVPGFNE

SEQ ID NO: 458 - protein, 9 aa, Severe acute respiratory syndrome coronavirus 2
LVPGFNEKT

SEQ ID NO: 459 - protein, 13 aa, Severe acute respiratory syndrome coronavirus 2
RGFGDSVEEVLSE

SEQ ID NO: 460 - protein, 12 aa, Severe acute respiratory syndrome coronavirus 2
VFIKRSDARTAP

SEQ ID NO: 461 - protein, 11 aa, Severe acute respiratory syndrome coronavirus 2
LEGIQYGRSGE

SEQ ID NO: 462 - protein, 9 aa, Severe acute respiratory syndrome coronavirus 2
EIPVAYRKV

SEQ ID NO: 463 - protein, 14 aa, Severe acute respiratory syndrome coronavirus 2
PVAYRKVLLRKNGN

SEQ ID NO: 464 - protein, 5 aa, Severe acute respiratory syndrome coronavirus 2
RKVLL

SEQ ID NO: 465 - protein, 11 aa, Severe acute respiratory syndrome coronavirus 2
KVLLRKNGNKG

SEQ ID NO: 466 - protein, 10 aa, Severe acute respiratory syndrome coronavirus 2
LRKNGNKGAG

SEQ ID NO: 467 - protein, 8 aa, Severe acute respiratory syndrome coronavirus 2
DLGDELGT

SEQ ID NO: 468 - protein, 7 aa, Severe acute respiratory syndrome coronavirus 2
ELGTDPY

SEQ ID NO: 469 - protein, 5 aa, Severe acute respiratory syndrome coronavirus 2
PYEDF

SEQ ID NO: 470 - protein, 14 aa, Severe acute respiratory syndrome coronavirus 2
PYEDFQENWNTKHS

SEQ ID NO: 471 - protein, 14 aa, Severe acute respiratory syndrome coronavirus 2
PYEDFQENWNTKHS

SEQ ID NO: 472 - protein, 9 aa, Severe acute respiratory syndrome coronavirus 2
FQENWNTKH

SEQ ID NO: 473 - protein, 6 aa, Severe acute respiratory syndrome coronavirus 2
NTKHSS

SEQ ID NO: 474 - protein, 7 aa, Severe acute respiratory syndrome coronavirus 2
SGVTREL

SEQ ID NO: 475 - protein, 11 aa, Severe acute respiratory syndrome coronavirus 2
RELMRELNGGA

SEQ ID NO: 476 - protein, 6 aa, Severe acute respiratory syndrome coronavirus 2
ELMREL

SEQ ID NO: 477 - protein, 6 aa, Severe acute respiratory syndrome coronavirus 2
LMRELN

SEQ ID NO: 478 - protein, 8 aa, Severe acute respiratory syndrome coronavirus 2
GYPLECIK

SEQ ID NO: 479 - protein, 8 aa, Severe acute respiratory syndrome coronavirus 2
DLLARAGK

SEQ ID NO: 480 - protein, 17 aa, Severe acute respiratory syndrome coronavirus 2
KASCTLSEQLDFIDTKR

SEQ ID NO: 481 - protein, 5 aa, Severe acute respiratory syndrome coronavirus 2
SEKSY

SEQ ID NO: 482 - protein, 9 aa, Severe acute respiratory syndrome coronavirus 2
TPFEIKLAK

SEQ ID NO: 483 - protein, 13 aa, Severe acute respiratory syndrome coronavirus 2
TPFEIKLAKKFDT

SEQ ID NO: 484 - protein, 13 aa, Severe acute respiratory syndrome coronavirus 2
KKFDTFNGECPNF

SEQ ID NO: 485 - protein, 11 aa, Severe acute respiratory syndrome coronavirus 2
IKTIQPRVEKK

SEQ ID NO: 486 - protein, 11 aa, Severe acute respiratory syndrome coronavirus 2
TIQPRVEKKKL

SEQ ID NO: 487 - protein, 13 aa, Severe acute respiratory syndrome coronavirus 2
TIQPRVEKKKLDG

SEQ ID NO: 488 - protein, 17 aa, Severe acute respiratory syndrome coronavirus 2
PRVEKKKLDGFMGRIRS

SEQ ID NO: 489 - protein, 7 aa, Severe acute respiratory syndrome coronavirus 2
KKLDGFM

SEQ ID NO: 490 - protein, 11 aa, Severe acute respiratory syndrome coronavirus 2
RIRSVYPVASP

SEQ ID NO: 491 - protein, 15 aa, Severe acute respiratory syndrome coronavirus 2
ECNQMCLSTLMKCDH

SEQ ID NO: 492 - protein, 6 aa, Severe acute respiratory syndrome coronavirus 2
TKEGAT

SEQ ID NO: 493 - protein, 12 aa, Severe acute respiratory syndrome coronavirus 2
VVKIYCPACHNS

SEQ ID NO: 494 - protein, 9 aa, Severe acute respiratory syndrome coronavirus 2
CPACHNSEV

SEQ ID NO: 495 - protein, 8 aa, Severe acute respiratory syndrome coronavirus 2
KTILRKGG

SEQ ID NO: 496 - protein, 8 aa, Severe acute respiratory syndrome coronavirus 2
VPRASANI

SEQ ID NO: 497 - protein, 11 aa, Severe acute respiratory syndrome coronavirus 2
DNLLEILQKEK

SEQ ID NO: 498 - protein, 16 aa, Severe acute respiratory syndrome coronavirus 2
KEKVNINIVGDFKLNE

SEQ ID NO: 499 - protein, 8 aa, Severe acute respiratory syndrome coronavirus 2
VGDFKLNE

SEQ ID NO: 500 - protein, 6 aa, Severe acute respiratory syndrome coronavirus 2
KLNEEI

SEQ ID NO: 501 - protein, 6 aa, Severe acute respiratory syndrome coronavirus 2
NEEIAI

SEQ ID NO: 502 - protein, 9 aa, Severe acute respiratory syndrome coronavirus 2
EIAIILASF

SEQ ID NO: 503 - protein, 16 aa, Severe acute respiratory syndrome coronavirus 2
FSASTSAFVETVKGLD

SEQ ID NO: 504 - protein, 19 aa, Severe acute respiratory syndrome coronavirus 2
TVKGLDYKAFKQIVESCGN

SEQ ID NO: 505 - protein, 8 aa, Severe acute respiratory syndrome coronavirus 2
VKGLDYKA

SEQ ID NO: 506 - protein, 9 aa, Severe acute respiratory syndrome coronavirus 2
KGLDYKAFK

SEQ ID NO: 507 - protein, 15 aa, Severe acute respiratory syndrome coronavirus 2
LDYKAFKQIVESCGN

SEQ ID NO: 508 - protein, 6 aa, Severe acute respiratory syndrome coronavirus 2
DYKAFK

SEQ ID NO: 509 - protein, 11 aa, Severe acute respiratory syndrome coronavirus 2
SCGNFKVTKGK

SEQ ID NO: 510 - protein, 6 aa, Severe acute respiratory syndrome coronavirus 2
TKGKAK

SEQ ID NO: 511 - protein, 10 aa, Severe acute respiratory syndrome coronavirus 2
KKGAWNIGEQ

SEQ ID NO: 512 - protein, 7 aa, Severe acute respiratory syndrome coronavirus 2
IGEQKSI

SEQ ID NO: 513 - protein, 11 aa, Severe acute respiratory syndrome coronavirus 2
IVGGQIVTCAK

SEQ ID NO: 514 - protein, 11 aa, Severe acute respiratory syndrome coronavirus 2
NLGETFVTHSK

SEQ ID NO: 515 - protein, 14 aa, Severe acute respiratory syndrome coronavirus 2
GETFVTHSKGLYRK

SEQ ID NO: 516 - protein, 15 aa, Severe acute respiratory syndrome coronavirus 2
REETGLLMPLKAPKE

SEQ ID NO: 517 - protein, 11 aa, Severe acute respiratory syndrome coronavirus 2
MPLKAPKEIIF

SEQ ID NO: 518 - protein, 13 aa, Severe acute respiratory syndrome coronavirus 2
PTEVLTEEVVLKT

SEQ ID NO: 519 - protein, 18 aa, Severe acute respiratory syndrome coronavirus 2
TEVLTEEVVLKTGDLQPL

SEQ ID NO: 520 - protein, 16 aa, Severe acute respiratory syndrome coronavirus 2
TEEVVLKTGDLQPLEQ

SEQ ID NO: 521 - protein, 9 aa, Severe acute respiratory syndrome coronavirus 2
EEVVLKTGD

SEQ ID NO: 522 - protein, 10 aa, Severe acute respiratory syndrome coronavirus 2
EQPTSEAVEA

SEQ ID NO: 523 - protein, 11 aa, Severe acute respiratory syndrome coronavirus 2
TSEAVEAPLVG

SEQ ID NO: 524 - protein, 9 aa, Severe acute respiratory syndrome coronavirus 2
LMLLEIKDT

SEQ ID NO: 525 - protein, 9 aa, Severe acute respiratory syndrome coronavirus 2
LEIKDTEKY

SEQ ID NO: 526 - protein, 12 aa, Severe acute respiratory syndrome coronavirus 2
DTEKYCALAPNM

SEQ ID NO: 527 - protein, 14 aa, Severe acute respiratory syndrome coronavirus 2
PNMMVTNNTFTLKG

SEQ ID NO: 528 - protein, 10 aa, Severe acute respiratory syndrome coronavirus 2
NNTFTLKGGA

SEQ ID NO: 529 - protein, 7 aa, Severe acute respiratory syndrome coronavirus 2
TFGDDTV

SEQ ID NO: 530 - protein, 12 aa, Severe acute respiratory syndrome coronavirus 2
DDTVIEVQGYKS

SEQ ID NO: 531 - protein, 9 aa, Severe acute respiratory syndrome coronavirus 2
VQGYKSVNI

SEQ ID NO: 532 - protein, 10 aa, Severe acute respiratory syndrome coronavirus 2
ELDERIDKVL

SEQ ID NO: 533 - protein, 16 aa, Severe acute respiratory syndrome coronavirus 2
ELDERIDKVLNEKCSA

SEQ ID NO: 534 - protein, 6 aa, Severe acute respiratory syndrome coronavirus 2
KTLQPV

SEQ ID NO: 535 - protein, 10 aa, Severe acute respiratory syndrome coronavirus 2
TLQPVSELLT

SEQ ID NO: 536 - protein, 6 aa, Severe acute respiratory syndrome coronavirus 2
LQPVSE

SEQ ID NO: 537 - protein, 8 aa, Severe acute respiratory syndrome coronavirus 2
EDEEEGDC

SEQ ID NO: 538 - protein, 15 aa, Severe acute respiratory syndrome coronavirus 2
TQYEYGTEDDYQGKP

SEQ ID NO: 539 - protein, 13 aa, Severe acute respiratory syndrome coronavirus 2
KPLEFGATSAALQ

SEQ ID NO: 540 - protein, 8 aa, Severe acute respiratory syndrome coronavirus 2
DNQTTTIQ

SEQ ID NO: 541 - protein, 6 aa, Severe acute respiratory syndrome coronavirus 2
TTIQTI

SEQ ID NO: 542 - protein, 13 aa, Severe acute respiratory syndrome coronavirus 2
IVEVQPQLEMELT

SEQ ID NO: 543 - protein, 7 aa, Severe acute respiratory syndrome coronavirus 2
DNVYIKN

SEQ ID NO: 544 - protein, 11 aa, Severe acute respiratory syndrome coronavirus 2
DNVYIKNADIV

SEQ ID NO: 545 - protein, 15 aa, Severe acute respiratory syndrome coronavirus 2
DNVYIKNADIVEEAK

SEQ ID NO: 546 - protein, 9 aa, Severe acute respiratory syndrome coronavirus 2
EAKKVKPTV

SEQ ID NO: 547 - protein, 14 aa, Severe acute respiratory syndrome coronavirus 2
AKKVKPTVVVNAAN

SEQ ID NO: 548 - protein, 14 aa, Severe acute respiratory syndrome coronavirus 2
TVVVNAANVYLKHG

SEQ ID NO: 549 - protein, 13 aa, Severe acute respiratory syndrome coronavirus 2
HGGGVAGALNKAT

SEQ ID NO: 550 - protein, 10 aa, Severe acute respiratory syndrome coronavirus 2
GALNKATNNA

SEQ ID NO: 551 - protein, 9 aa, Severe acute respiratory syndrome coronavirus 2
NKATNNAMQ

SEQ ID NO: 552 - protein, 15 aa, Severe acute respiratory syndrome coronavirus 2
NKATNNAMQVESDDY

SEQ ID NO: 553 - protein, 12 aa, Severe acute respiratory syndrome coronavirus 2
PLKVGGSCVLSG

SEQ ID NO: 554 - protein, 8 aa, Severe acute respiratory syndrome coronavirus 2
GHNLAKHC

SEQ ID NO: 555 - protein, 15 aa, Severe acute respiratory syndrome coronavirus 2
HCLHVVGPNVNKGED

SEQ ID NO: 556 - protein, 12 aa, Severe acute respiratory syndrome coronavirus 2
GPNVNKGEDIQL

SEQ ID NO: 557 - protein, 12 aa, Severe acute respiratory syndrome coronavirus 2
NKGEDIQLLKSA

SEQ ID NO: 558 - protein, 20 aa, Severe acute respiratory syndrome coronavirus 2
EDIQLLKSAYENFNQHEVLL

SEQ ID NO: 559 - protein, 9 aa, Severe acute respiratory syndrome coronavirus 2
NFNQHEVLL

SEQ ID NO: 560 - protein, 11 aa, Severe acute respiratory syndrome coronavirus 2
LLSAGIFGADP

SEQ ID NO: 561 - protein, 9 aa, Severe acute respiratory syndrome coronavirus 2
DKNLYDKLV

SEQ ID NO: 562 - protein, 16 aa, Severe acute respiratory syndrome coronavirus 2
NLYDKLVSSFLEMKSE

SEQ ID NO: 563 - protein, 15 aa, Severe acute respiratory syndrome coronavirus 2
LYDKLVSSFLEMKSE

SEQ ID NO: 564 - protein, 11 aa, Severe acute respiratory syndrome coronavirus 2
EMKSEKQVEQK

SEQ ID NO: 565 - protein, 12 aa, Severe acute respiratory syndrome coronavirus 2
KSEKQVEQKIAE

SEQ ID NO: 566 - protein, 5 aa, Severe acute respiratory syndrome coronavirus 2
EKQVE

SEQ ID NO: 567 - protein, 9 aa, Severe acute respiratory syndrome coronavirus 2
VEQKIAEIP

SEQ ID NO: 568 - protein, 14 aa, Severe acute respiratory syndrome coronavirus 2
VEQKIAEIPKEEVK

SEQ ID NO: 569 - protein, 12 aa, Severe acute respiratory syndrome coronavirus 2
IAEIPKEEVKPF

SEQ ID NO: 570 - protein, 14 aa, Severe acute respiratory syndrome coronavirus 2
IPKEEVKPFITESK

SEQ ID NO: 571 - protein, 6 aa, Severe acute respiratory syndrome coronavirus 2
EEVKPF

SEQ ID NO: 572 - protein, 14 aa, Severe acute respiratory syndrome coronavirus 2
EEVKPFITESKPSV

SEQ ID NO: 573 - protein, 14 aa, Severe acute respiratory syndrome coronavirus 2
EEVKPFITESKPSV

SEQ ID NO: 574 - protein, 7 aa, Severe acute respiratory syndrome coronavirus 2
ESKPSVE

SEQ ID NO: 575 - protein, 13 aa, Severe acute respiratory syndrome coronavirus 2
ESKPSVEQRKQDD

SEQ ID NO: 576 - protein, 16 aa, Severe acute respiratory syndrome coronavirus 2
SKPSVEQRKQDDKKIK

SEQ ID NO: 577 - protein, 11 aa, Severe acute respiratory syndrome coronavirus 2
KPSVEQRKQDD

SEQ ID NO: 578 - protein, 6 aa, Severe acute respiratory syndrome coronavirus 2
RKQDDK

SEQ ID NO: 579 - protein, 10 aa, Severe acute respiratory syndrome coronavirus 2
YIDINGNLHP

SEQ ID NO: 580 - protein, 9 aa, Severe acute respiratory syndrome coronavirus 2
TLVSDIDIT

SEQ ID NO: 581 - protein, 11 aa, Severe acute respiratory syndrome coronavirus 2
DIDITFLKKDA

SEQ ID NO: 582 - protein, 15 aa, Severe acute respiratory syndrome coronavirus 2
KKDAPYIVGDVVQEG

SEQ ID NO: 583 - protein, 14 aa, Severe acute respiratory syndrome coronavirus 2
QEGVLTAVVIPTKK

SEQ ID NO: 584 - protein, 5 aa, Severe acute respiratory syndrome coronavirus 2
IPTKK

SEQ ID NO: 585 - protein, 11 aa, Severe acute respiratory syndrome coronavirus 2
PTKKAGGTTEM

SEQ ID NO: 586 - protein, 23 aa, Severe acute respiratory syndrome coronavirus 2
PTKKAGGTTEMLAKALRKVPTDN

SEQ ID NO: 587 - protein, 11 aa, Severe acute respiratory syndrome coronavirus 2
TKKAGGTTEML

SEQ ID NO: 588 - protein, 7 aa, Severe acute respiratory syndrome coronavirus 2
GGTTEML

SEQ ID NO: 589 - protein, 9 aa, Severe acute respiratory syndrome coronavirus 2
GGTTEMLAK

SEQ ID NO: 590 - protein, 10 aa, Severe acute respiratory syndrome coronavirus 2
TTEMLAKALR

SEQ ID NO: 591 - protein, 12 aa, Severe acute respiratory syndrome coronavirus 2
MLAKALRKVPTD

SEQ ID NO: 592 - protein, 12 aa, Severe acute respiratory syndrome coronavirus 2
ALRKVPTDNYIT

SEQ ID NO: 593 - protein, 8 aa, Severe acute respiratory syndrome coronavirus 2
RKVPTDNY

SEQ ID NO: 594 - protein, 13 aa, Severe acute respiratory syndrome coronavirus 2
RKVPTDNYITTYP

SEQ ID NO: 595 - protein, 9 aa, Severe acute respiratory syndrome coronavirus 2
GLNGYTVEE

SEQ ID NO: 596 - protein, 10 aa, Severe acute respiratory syndrome coronavirus 2
PSIISNEKQE

SEQ ID NO: 597 - protein, 7 aa, Severe acute respiratory syndrome coronavirus 2
SNEKQEI

SEQ ID NO: 598 - protein, 10 aa, Severe acute respiratory syndrome coronavirus 2
VCVETKAIVS

SEQ ID NO: 599 - protein, 11 aa, Severe acute respiratory syndrome coronavirus 2
RKYKGIKIQEG

SEQ ID NO: 600 - protein, 10 aa, Severe acute respiratory syndrome coronavirus 2
IKIQEGVVDY

SEQ ID NO: 601 - protein, 11 aa, Severe acute respiratory syndrome coronavirus 2
SLINTLNDLNE

SEQ ID NO: 602 - protein, 16 aa, Severe acute respiratory syndrome coronavirus 2
GLNLEEAARYMRSLKV

SEQ ID NO: 603 - protein, 18 aa, Severe acute respiratory syndrome coronavirus 2
RYMRSLKVPATVSVSSPD

SEQ ID NO: 604 - protein, 17 aa, Severe acute respiratory syndrome coronavirus 2
DAVTAYNGYLTSSSKTP

SEQ ID NO: 605 - protein, 13 aa, Severe acute respiratory syndrome coronavirus 2
NGYLTSSSKTPEE

SEQ ID NO: 606 - protein, 13 aa, Severe acute respiratory syndrome coronavirus 2
GYLTSSSKTPEEH

SEQ ID NO: 607 - protein, 10 aa, Severe acute respiratory syndrome coronavirus 2
SSKTPEEHFI

SEQ ID NO: 608 - protein, 5 aa, Severe acute respiratory syndrome coronavirus 2
YSGQS

SEQ ID NO: 609 - protein, 9 aa, Severe acute respiratory syndrome coronavirus 2
YSGQSTQLG

SEQ ID NO: 610 - protein, 10 aa, Severe acute respiratory syndrome coronavirus 2
FLKRGDKSVY

SEQ ID NO: 611 - protein, 11 aa, Severe acute respiratory syndrome coronavirus 2
RGDKSVYYTSN

SEQ ID NO: 612 - protein, 6 aa, Severe acute respiratory syndrome coronavirus 2
HLDGEV

SEQ ID NO: 613 - protein, 7 aa, Severe acute respiratory syndrome coronavirus 2
LLSLREV

SEQ ID NO: 614 - protein, 6 aa, Severe acute respiratory syndrome coronavirus 2
LREVRT

SEQ ID NO: 615 - protein, 9 aa, Severe acute respiratory syndrome coronavirus 2
TTVDNINLH

SEQ ID NO: 616 - protein, 11 aa, Severe acute respiratory syndrome coronavirus 2
QFGPTYLDGAD

SEQ ID NO: 617 - protein, 9 aa, Severe acute respiratory syndrome coronavirus 2
DGADVTKIK

SEQ ID NO: 618 - protein, 6 aa, Severe acute respiratory syndrome coronavirus 2
DVTKIK

SEQ ID NO: 619 - protein, 11 aa, Severe acute respiratory syndrome coronavirus 2
DPSFLGRYMSA

SEQ ID NO: 620 - protein, 10 aa, Severe acute respiratory syndrome coronavirus 2
RYMSALNHTK

SEQ ID NO: 621 - protein, 5 aa, Severe acute respiratory syndrome coronavirus 2
KWKYP

SEQ ID NO: 622 - protein, 16 aa, Severe acute respiratory syndrome coronavirus 2
YPQVNGLTSIKWADNN

SEQ ID NO: 623 - protein, 15 aa, Severe acute respiratory syndrome coronavirus 2
NPPALQDAYYRARAG

SEQ ID NO: 624 - protein, 11 aa, Severe acute respiratory syndrome coronavirus 2
LQDAYYRARAG

SEQ ID NO: 625 - protein, 9 aa, Severe acute respiratory syndrome coronavirus 2
YCNKTVGEL

SEQ ID NO: 626 - protein, 6 aa, Severe acute respiratory syndrome coronavirus 2
DVRETM

SEQ ID NO: 627 - protein, 5 aa, Severe acute respiratory syndrome coronavirus 2
RETMS

SEQ ID NO: 628 - protein, 7 aa, Severe acute respiratory syndrome coronavirus 2
ETMSYLF

SEQ ID NO: 629 - protein, 8 aa, Severe acute respiratory syndrome coronavirus 2
NLDSCKRV

SEQ ID NO: 630 - protein, 12 aa, Severe acute respiratory syndrome coronavirus 2
KTCGQQQTTLKG

SEQ ID NO: 631 - protein, 16 aa, Severe acute respiratory syndrome coronavirus 2
EAVMYMGTLSYEQFKK

SEQ ID NO: 632 - protein, 12 aa, Severe acute respiratory syndrome coronavirus 2
TLSYEQFKKGVQ

SEQ ID NO: 633 - protein, 9 aa, Severe acute respiratory syndrome coronavirus 2
YLVQQESPF

SEQ ID NO: 634 - protein, 13 aa, Severe acute respiratory syndrome coronavirus 2
VQQESPFVMMSAP

SEQ ID NO: 635 - protein, 19 aa, Severe acute respiratory syndrome coronavirus 2
QESPFVMMSAPPAQYELKH

SEQ ID NO: 636 - protein, 11 aa, Severe acute respiratory syndrome coronavirus 2
LKHGTFTCASE

SEQ ID NO: 637 - protein, 11 aa, Severe acute respiratory syndrome coronavirus 2
CGHYKHITSKE

SEQ ID NO: 638 - protein, 8 aa, Severe acute respiratory syndrome coronavirus 2
DGALLTKS

SEQ ID NO: 639 - protein, 12 aa, Severe acute respiratory syndrome coronavirus 2
TKSSEYKGPITD

SEQ ID NO: 640 - protein, 15 aa, Severe acute respiratory syndrome coronavirus 2
SSEYKGPITDVFYKE

SEQ ID NO: 641 - protein, 13 aa, Severe acute respiratory syndrome coronavirus 2
YKGPITDVFYKEN

SEQ ID NO: 642 - protein, 13 aa, Severe acute respiratory syndrome coronavirus 2
YKENSYTTTIKPV

SEQ ID NO: 643 - protein, 5 aa, Severe acute respiratory syndrome coronavirus 2
TTIKP

SEQ ID NO: 644 - protein, 7 aa, Severe acute respiratory syndrome coronavirus 2
IKPVTYK

SEQ ID NO: 645 - protein, 7 aa, Severe acute respiratory syndrome coronavirus 2
PVTYKLD

SEQ ID NO: 646 - protein, 7 aa, Severe acute respiratory syndrome coronavirus 2
YKLDGVV

SEQ ID NO: 647 - protein, 12 aa, Severe acute respiratory syndrome coronavirus 2
YKLDGVVCTEID

SEQ ID NO: 648 - protein, 13 aa, Severe acute respiratory syndrome coronavirus 2
CTEIDPKLDNYYK

SEQ ID NO: 649 - protein, 12 aa, Severe acute respiratory syndrome coronavirus 2
EIDPKLDNYYKK

SEQ ID NO: 650 - protein, 11 aa, Severe acute respiratory syndrome coronavirus 2
DPKLDNYYKKD

SEQ ID NO: 651 - protein, 5 aa, Severe acute respiratory syndrome coronavirus 2
PKLDN

SEQ ID NO: 652 - protein, 13 aa, Severe acute respiratory syndrome coronavirus 2
NYYKKDNSYFTEQ

SEQ ID NO: 653 - protein, 5 aa, Severe acute respiratory syndrome coronavirus 2
QPIDL

SEQ ID NO: 654 - protein, 10 aa, Severe acute respiratory syndrome coronavirus 2
NQPYPNASFD

SEQ ID NO: 655 - protein, 8 aa, Severe acute respiratory syndrome coronavirus 2
ASFDNFKF

SEQ ID NO: 656 - protein, 10 aa, Severe acute respiratory syndrome coronavirus 2
DNIKFADDLN

SEQ ID NO: 657 - protein, 14 aa, Severe acute respiratory syndrome coronavirus 2
NIKFADDLNQLTGY

SEQ ID NO: 658 - protein, 10 aa, Severe acute respiratory syndrome coronavirus 2
DLNQLTGYKK

SEQ ID NO: 659 - protein, 9 aa, Severe acute respiratory syndrome coronavirus 2
QLTGYKKPA

SEQ ID NO: 660 - protein, 19 aa, Severe acute respiratory syndrome coronavirus 2
HYTPSFKKGAKLLHKPIVW

SEQ ID NO: 661 - protein, 11 aa, Severe acute respiratory syndrome coronavirus 2
YTPSFKKGAKL

SEQ ID NO: 662 - protein, 10 aa, Severe acute respiratory syndrome coronavirus 2
FKKGAKLLHK

SEQ ID NO: 663 - protein, 14 aa, Severe acute respiratory syndrome coronavirus 2
KPIVWHVNNATNKA

SEQ ID NO: 664 - protein, 9 aa, Severe acute respiratory syndrome coronavirus 2
ATNKATYKP

SEQ ID NO: 665 - protein, 12 aa, Severe acute respiratory syndrome coronavirus 2
WCIRCLWSTKPV

SEQ ID NO: 666 - protein, 7 aa, Severe acute respiratory syndrome coronavirus 2
KPVETSN

SEQ ID NO: 667 - protein, 10 aa, Severe acute respiratory syndrome coronavirus 2
ETSNSFDVLK

SEQ ID NO: 668 - protein, 10 aa, Severe acute respiratory syndrome coronavirus 2
VLKSEDAQGM

SEQ ID NO: 669 - protein, 16 aa, Severe acute respiratory syndrome coronavirus 2
LKSEDAQGMDNLACED

SEQ ID NO: 670 - protein, 10 aa, Severe acute respiratory syndrome coronavirus 2
EEVVENPTIQ

SEQ ID NO: 671 - protein, 11 aa, Severe acute respiratory syndrome coronavirus 2
EVVENPTIQKD

SEQ ID NO: 672 - protein, 12 aa, Severe acute respiratory syndrome coronavirus 2
IQKDVLECNVKT

SEQ ID NO: 673 - protein, 15 aa, Severe acute respiratory syndrome coronavirus 2
VKTTEVVGDIILKPA

SEQ ID NO: 674 - protein, 10 aa, Severe acute respiratory syndrome coronavirus 2
VGDIILKPAN

SEQ ID NO: 675 - protein, 15 aa, Severe acute respiratory syndrome coronavirus 2
DIILKPANNSLKITE

SEQ ID NO: 676 - protein, 12 aa, Severe acute respiratory syndrome coronavirus 2
ILKPANNSLKIT

SEQ ID NO: 677 - protein, 14 aa, Severe acute respiratory syndrome coronavirus 2
ILKPANNSLKITEE

SEQ ID NO: 678 - protein, 10 aa, Severe acute respiratory syndrome coronavirus 2
SLKITEEVGH

SEQ ID NO: 679 - protein, 8 aa, Severe acute respiratory syndrome coronavirus 2
EEVGHTDL

SEQ ID NO: 680 - protein, 12 aa, Severe acute respiratory syndrome coronavirus 2
DLMAAYVDNSSL

SEQ ID NO: 681 - protein, 12 aa, Severe acute respiratory syndrome coronavirus 2
VDNSSLTIKKPN

SEQ ID NO: 682 - protein, 9 aa, Severe acute respiratory syndrome coronavirus 2
IKKPNELSR

SEQ ID NO: 683 - protein, 11 aa, Severe acute respiratory syndrome coronavirus 2
NELSRVLGLKT

SEQ ID NO: 684 - protein, 9 aa, Severe acute respiratory syndrome coronavirus 2
DTIANYAKP

SEQ ID NO: 685 - protein, 10 aa, Severe acute respiratory syndrome coronavirus 2
YAKPFLNKVV

SEQ ID NO: 686 - protein, 7 aa, Severe acute respiratory syndrome coronavirus 2
VCTNYMP

SEQ ID NO: 687 - protein, 5 aa, Severe acute respiratory syndrome coronavirus 2
SRIKA

SEQ ID NO: 688 - protein, 11 aa, Severe acute respiratory syndrome coronavirus 2
RIKASMPTTIA

SEQ ID NO: 689 - protein, 10 aa, Severe acute respiratory syndrome coronavirus 2
IKASMPTTIA

SEQ ID NO: 690 - protein, 13 aa, Severe acute respiratory syndrome coronavirus 2
IKASMPTTIAKNT

SEQ ID NO: 691 - protein, 13 aa, Severe acute respiratory syndrome coronavirus 2
MPTTIAKNTVKSV

SEQ ID NO: 692 - protein, 6 aa, Severe acute respiratory syndrome coronavirus 2
NLGMPS

SEQ ID NO: 693 - protein, 12 aa, Severe acute respiratory syndrome coronavirus 2
FISDEVARDLSL

SEQ ID NO: 694 - protein, 12 aa, Severe acute respiratory syndrome coronavirus 2
DEVARDLSLQFK

SEQ ID NO: 695 - protein, 10 aa, Severe acute respiratory syndrome coronavirus 2
FKRPINPTDQ

SEQ ID NO: 696 - protein, 9 aa, Severe acute respiratory syndrome coronavirus 2
VDSVTVKNG

SEQ ID NO: 697 - protein, 6 aa, Severe acute respiratory syndrome coronavirus 2
VKNGSI

SEQ ID NO: 698 - protein, 11 aa, Severe acute respiratory syndrome coronavirus 2
VKNGSIHLYFD

SEQ ID NO: 699 - protein, 8 aa, Severe acute respiratory syndrome coronavirus 2
FDKAGQKT

SEQ ID NO: 700 - protein, 9 aa, Severe acute respiratory syndrome coronavirus 2
NLDNLRANN

SEQ ID NO: 701 - protein, 10 aa, Severe acute respiratory syndrome coronavirus 2
DNLRANNTKG

SEQ ID NO: 702 - protein, 8 aa, Severe acute respiratory syndrome coronavirus 2
IVFDGKSK

SEQ ID NO: 703 - protein, 22 aa, Severe acute respiratory syndrome coronavirus 2
SKCEESSAKSASVYYSQLMCQP

SEQ ID NO: 704 - protein, 11 aa, Severe acute respiratory syndrome coronavirus 2
SSAKSASVYYS

SEQ ID NO: 705 - protein, 9 aa, Severe acute respiratory syndrome coronavirus 2
DSAEVAVKM

SEQ ID NO: 706 - protein, 6 aa, Severe acute respiratory syndrome coronavirus 2
MEKLKT

SEQ ID NO: 707 - protein, 9 aa, Severe acute respiratory syndrome coronavirus 2
AKNVSLDNV

SEQ ID NO: 708 - protein, 6 aa, Severe acute respiratory syndrome coronavirus 2
AARQGF

SEQ ID NO: 709 - protein, 14 aa, Severe acute respiratory syndrome coronavirus 2
RQGFVDSDVETKDV

SEQ ID NO: 710 - protein, 12 aa, Severe acute respiratory syndrome coronavirus 2
ETKDVVECLKLS

SEQ ID NO: 711 - protein, 12 aa, Severe acute respiratory syndrome coronavirus 2
NNYMLTYNKVEN

SEQ ID NO: 712 - protein, 15 aa, Severe acute respiratory syndrome coronavirus 2
NNYMLTYNKVENMTP

SEQ ID NO: 713 - protein, 18 aa, Severe acute respiratory syndrome coronavirus 2
ACIDCSARHINAQVAKSH

SEQ ID NO: 714 - protein, 5 aa, Severe acute respiratory syndrome coronavirus 2
AKSHN

SEQ ID NO: 715 - protein, 12 aa, Severe acute respiratory syndrome coronavirus 2
SLSEQLRKQIRS

SEQ ID NO: 716 - protein, 15 aa, Severe acute respiratory syndrome coronavirus 2
SLSEQLRKQIRSAAK

SEQ ID NO: 717 - protein, 16 aa, Severe acute respiratory syndrome coronavirus 2
SEQLRKQIRSAAKKNN

SEQ ID NO: 718 - protein, 8 aa, Severe acute respiratory syndrome coronavirus 2
KKNNLPFK

SEQ ID NO: 719 - protein, 13 aa, Severe acute respiratory syndrome coronavirus 2
TTKIALKGGKIVN

SEQ ID NO: 720 - protein, 8 aa, Severe acute respiratory syndrome coronavirus 2
SSEIIGYK

SEQ ID NO: 721 - protein, 12 aa, Severe acute respiratory syndrome coronavirus 2
SEIIGYKAIDGG

SEQ ID NO: 722 - protein, 14 aa, Severe acute respiratory syndrome coronavirus 2
DIASTDTCFANKHA

SEQ ID NO: 723 - protein, 13 aa, Severe acute respiratory syndrome coronavirus 2
WFSQRGGSYTNDK

SEQ ID NO: 724 - protein, 7 aa, Severe acute respiratory syndrome coronavirus 2
IEYTDFA

SEQ ID NO: 725 - protein, 21 aa, Severe acute respiratory syndrome coronavirus 2
AECTIFKDASGKPVPYCYDTN

SEQ ID NO: 726 - protein, 13 aa, Severe acute respiratory syndrome coronavirus 2
CTIFKDASGKPVP

SEQ ID NO: 727 - protein, 19 aa, Severe acute respiratory syndrome coronavirus 2
SLRPDTRYVLMDGSIIQFP

SEQ ID NO: 728 - protein, 8 aa, Severe acute respiratory syndrome coronavirus 2
RPDTRYVL

SEQ ID NO: 729 - protein, 8 aa, Severe acute respiratory syndrome coronavirus 2
SNYLKRRV

SEQ ID NO: 730 - protein, 9 aa, Severe acute respiratory syndrome coronavirus 2
LKRRVVFNG

SEQ ID NO: 731 - protein, 18 aa, Severe acute respiratory syndrome coronavirus 2
LKRRVVFNGVSFSTFEEA

SEQ ID NO: 732 - protein, 5 aa, Severe acute respiratory syndrome coronavirus 2
SFSTF

SEQ ID NO: 733 - protein, 5 aa, Severe acute respiratory syndrome coronavirus 2
FSTFE

SEQ ID NO: 734 - protein, 17 aa, Severe acute respiratory syndrome coronavirus 2
LLPLTQYNRYLALYNKY

SEQ ID NO: 735 - protein, 17 aa, Severe acute respiratory syndrome coronavirus 2
TQYNRYLALYNKYKYFS

SEQ ID NO: 736 - protein, 10 aa, Severe acute respiratory syndrome coronavirus 2
CCHLAKALND

SEQ ID NO: 737 - protein, 6 aa, Severe acute respiratory syndrome coronavirus 2
AKALND

SEQ ID NO: 738 - protein, 7 aa, Severe acute respiratory syndrome coronavirus 2
SAVLQSG

SEQ ID NO: 739 - protein, 7 aa, Severe acute respiratory syndrome coronavirus 2
QSGFRKM

SEQ ID NO: 740 - protein, 9 aa, Severe acute respiratory syndrome coronavirus 2
GFRKMAFPS

SEQ ID NO: 741 - protein, 11 aa, Severe acute respiratory syndrome coronavirus 2
LIRKSNHNFLV

SEQ ID NO: 742 - protein, 9 aa, Severe acute respiratory syndrome coronavirus 2
KLKVDTANP

SEQ ID NO: 743 - protein, 8 aa, Severe acute respiratory syndrome coronavirus 2
TANPKTPK

SEQ ID NO: 744 - protein, 8 aa, Severe acute respiratory syndrome coronavirus 2
YKFVRIQP

SEQ ID NO: 745 - protein, 9 aa, Severe acute respiratory syndrome coronavirus 2
MRPNFTIKG

SEQ ID NO: 746 - protein, 8 aa, Severe acute respiratory syndrome coronavirus 2
KGSFLNGS

SEQ ID NO: 747 - protein, 15 aa, Severe acute respiratory syndrome coronavirus 2
HMELPTGVHAGTDLE

SEQ ID NO: 748 - protein, 8 aa, Severe acute respiratory syndrome coronavirus 2
GDRWFLNR

SEQ ID NO: 749 - protein, 6 aa, Severe acute respiratory syndrome coronavirus 2
KELLQN

SEQ ID NO: 750 - protein, 7 aa, Severe acute respiratory syndrome coronavirus 2
LQNGMNG

SEQ ID NO: 751 - protein, 6 aa, Severe acute respiratory syndrome coronavirus 2
NGMNGR

SEQ ID NO: 752 - protein, 8 aa, Severe acute respiratory syndrome coronavirus 2
RTILGSAL

SEQ ID NO: 753 - protein, 5 aa, Severe acute respiratory syndrome coronavirus 2
GNALD

SEQ ID NO: 754 - protein, 17 aa, Severe acute respiratory syndrome coronavirus 2
SQGLLPPKNSIDAFKLN

SEQ ID NO: 755 - protein, 10 aa, Severe acute respiratory syndrome coronavirus 2
PPKNSIDAFK

SEQ ID NO: 756 - protein, 6 aa, Severe acute respiratory syndrome coronavirus 2
KNSIDA

SEQ ID NO: 757 - protein, 15 aa, Severe acute respiratory syndrome coronavirus 2
NIKLLGVGGKPCIKV

SEQ ID NO: 758 - protein, 15 aa, Severe acute respiratory syndrome coronavirus 2
GVGGKPCIKVATVQS

SEQ ID NO: 759 - protein, 9 aa, Severe acute respiratory syndrome coronavirus 2
GKPCIKVAT

SEQ ID NO: 760 - protein, 8 aa, Severe acute respiratory syndrome coronavirus 2
ILLAKDTT

SEQ ID NO: 761 - protein, 7 aa, Severe acute respiratory syndrome coronavirus 2
MLDNRAT

SEQ ID NO: 762 - protein, 17 aa, Severe acute respiratory syndrome coronavirus 2
DNRATLQAIASEFSSLP

SEQ ID NO: 763 - protein, 19 aa, Severe acute respiratory syndrome coronavirus 2
LPSYAAFATAQEAYEQAVA

SEQ ID NO: 764 - protein, 12 aa, Severe acute respiratory syndrome coronavirus 2
AFATAQEAYEQA

SEQ ID NO: 765 - protein, 19 aa, Severe acute respiratory syndrome coronavirus 2
TAQEAYEQAVANGDSEVVL

SEQ ID NO: 766 - protein, 6 aa, Severe acute respiratory syndrome coronavirus 2
EAYEQA

SEQ ID NO: 767 - protein, 12 aa, Severe acute respiratory syndrome coronavirus 2
EVVLKKLKKSLN

SEQ ID NO: 768 - protein, 8 aa, Severe acute respiratory syndrome coronavirus 2
LKKSLNVA

SEQ ID NO: 769 - protein, 13 aa, Severe acute respiratory syndrome coronavirus 2
LKKSLNVAKSEFD

SEQ ID NO: 770 - protein, 6 aa, Severe acute respiratory syndrome coronavirus 2
AKSEFD

SEQ ID NO: 771 - protein, 12 aa, Severe acute respiratory syndrome coronavirus 2
QMYKQARSEDKR

SEQ ID NO: 772 - protein, 12 aa, Severe acute respiratory syndrome coronavirus 2
MLRKLDNDALNN

SEQ ID NO: 773 - protein, 7 aa, Severe acute respiratory syndrome coronavirus 2
PDYNTYK

SEQ ID NO: 774 - protein, 11 aa, Severe acute respiratory syndrome coronavirus 2
DADSKIVQLSE

SEQ ID NO: 775 - protein, 7 aa, Severe acute respiratory syndrome coronavirus 2
SPNLAWP

SEQ ID NO: 776 - protein, 11 aa, Severe acute respiratory syndrome coronavirus 2
SPNLAWPLIVT

SEQ ID NO: 777 - protein, 11 aa, Severe acute respiratory syndrome coronavirus 2
ALRANSAVKLQ

SEQ ID NO: 778 - protein, 6 aa, Severe acute respiratory syndrome coronavirus 2
CAAGTT

SEQ ID NO: 779 - protein, 15 aa, Severe acute respiratory syndrome coronavirus 2
VLALLSDLQDLKWAR

SEQ ID NO: 780 - protein, 10 aa, Severe acute respiratory syndrome coronavirus 2
QDLKWARFPK

SEQ ID NO: 781 - protein, 9 aa, Severe acute respiratory syndrome coronavirus 2
IKGLNNLNR

SEQ ID NO: 782 - protein, 6 aa, Severe acute respiratory syndrome coronavirus 2
TEVPAN

SEQ ID NO: 783 - protein, 19 aa, Severe acute respiratory syndrome coronavirus 2
DAAKAYKDYLASGGQPITN

SEQ ID NO: 784 - protein, 13 aa, Severe acute respiratory syndrome coronavirus 2
KAYKDYLASGGQP

SEQ ID NO: 785 - protein, 12 aa, Severe acute respiratory syndrome coronavirus 2
NCVKMLCTHTGT

SEQ ID NO: 786 - protein, 6 aa, Severe acute respiratory syndrome coronavirus 2
MDQESF

SEQ ID NO: 787 - protein, 14 aa, Severe acute respiratory syndrome coronavirus 2
PKGFCDLKGKYVQI

SEQ ID NO: 788 - protein, 15 aa, Severe acute respiratory syndrome coronavirus 2
FLNRVCGVSAARLTP

SEQ ID NO: 789 - protein, 18 aa, Severe acute respiratory syndrome coronavirus 2
GTSTDVVYRAFDIYNDKV

SEQ ID NO: 790 - protein, 10 aa, Severe acute respiratory syndrome coronavirus 2
EKDEDDNLID

SEQ ID NO: 791 - protein, 11 aa, Severe acute respiratory syndrome coronavirus 2
DSYFVVKRHTF

SEQ ID NO: 792 - protein, 13 aa, Severe acute respiratory syndrome coronavirus 2
EETIYNLLKDCPA

SEQ ID NO: 793 - protein, 13 aa, Severe acute respiratory syndrome coronavirus 2
FKFRIDGDMVPHI

SEQ ID NO: 794 - protein, 14 aa, Severe acute respiratory syndrome coronavirus 2
RQRLTKYTMADLVY

SEQ ID NO: 795 - protein, 9 aa, Severe acute respiratory syndrome coronavirus 2
LTKYTMADL

SEQ ID NO: 796 - protein, 19 aa, Severe acute respiratory syndrome coronavirus 2
KYTMADLVYALRHFDEGNC

SEQ ID NO: 797 - protein, 6 aa, Severe acute respiratory syndrome coronavirus 2
DNQDLN

SEQ ID NO: 798 - protein, 9 aa, Severe acute respiratory syndrome coronavirus 2
LNGNWYDFG

SEQ ID NO: 799 - protein, 6 aa, Severe acute respiratory syndrome coronavirus 2
DLTKPY

SEQ ID NO: 800 - protein, 10 aa, Severe acute respiratory syndrome coronavirus 2
DLTKPYIKWD

SEQ ID NO: 801 - protein, 16 aa, Severe acute respiratory syndrome coronavirus 2
NQDVNLHSSRLSFKEL

SEQ ID NO: 802 - protein, 9 aa, Severe acute respiratory syndrome coronavirus 2
SRLSFKELL

SEQ ID NO: 803 - protein, 6 aa, Severe acute respiratory syndrome coronavirus 2
ALTNNV

SEQ ID NO: 804 - protein, 14 aa, Severe acute respiratory syndrome coronavirus 2
TNNVAFQTVKPGNF

SEQ ID NO: 805 - protein, 9 aa, Severe acute respiratory syndrome coronavirus 2
VKPGNFNKD

SEQ ID NO: 806 - protein, 13 aa, Severe acute respiratory syndrome coronavirus 2
PGNFNKDFYDFAV

SEQ ID NO: 807 - protein, 15 aa, Severe acute respiratory syndrome coronavirus 2
FNKDFYDFAVSKGFF

SEQ ID NO: 808 - protein, 9 aa, Severe acute respiratory syndrome coronavirus 2
KEGSSVELK

SEQ ID NO: 809 - protein, 10 aa, Severe acute respiratory syndrome coronavirus 2
VELKHFFFAQ

SEQ ID NO: 810 - protein, 9 aa, Severe acute respiratory syndrome coronavirus 2
YRYNLPTMC

SEQ ID NO: 811 - protein, 14 aa, Severe acute respiratory syndrome coronavirus 2
INANQVIVNNLDKS

SEQ ID NO: 812 - protein, 6 aa, Severe acute respiratory syndrome coronavirus 2
PFNKWG

SEQ ID NO: 813 - protein, 5 aa, Severe acute respiratory syndrome coronavirus 2
KARLY

SEQ ID NO: 814 - protein, 13 aa, Severe acute respiratory syndrome coronavirus 2
ARLYYDSMSYEDQ

SEQ ID NO: 815 - protein, 14 aa, Severe acute respiratory syndrome coronavirus 2
YYDSMSYEDQDALF

SEQ ID NO: 816 - protein, 13 aa, Severe acute respiratory syndrome coronavirus 2
EDQDALFAYTKRN

SEQ ID NO: 817 - protein, 9 aa, Severe acute respiratory syndrome coronavirus 2
QMNLKYAIS

SEQ ID NO: 818 - protein, 13 aa, Severe acute respiratory syndrome coronavirus 2
NLKYAISAKNRAR

SEQ ID NO: 819 - protein, 8 aa, Severe acute respiratory syndrome coronavirus 2
ISAKNRAR

SEQ ID NO: 820 - protein, 10 aa, Severe acute respiratory syndrome coronavirus 2
NRQFHQKLLK

SEQ ID NO: 821 - protein, 12 aa, Severe acute respiratory syndrome coronavirus 2
ICQAVTANVNAL

SEQ ID NO: 822 - protein, 8 aa, Severe acute respiratory syndrome coronavirus 2
STDGNKIA

SEQ ID NO: 823 - protein, 6 aa, Severe acute respiratory syndrome coronavirus 2
DFVNEF

SEQ ID NO: 824 - protein, 8 aa, Severe acute respiratory syndrome coronavirus 2
YASQGLVA

SEQ ID NO: 825 - protein, 10 aa, Severe acute respiratory syndrome coronavirus 2
ASIKNFKSVL

SEQ ID NO: 826 - protein, 12 aa, Severe acute respiratory syndrome coronavirus 2
KNFKSVLYYQNN

SEQ ID NO: 827 - protein, 11 aa, Severe acute respiratory syndrome coronavirus 2
YYQNNVFMSEA

SEQ ID NO: 828 - protein, 9 aa, Severe acute respiratory syndrome coronavirus 2
VFMSEAKCW

SEQ ID NO: 829 - protein, 6 aa, Severe acute respiratory syndrome coronavirus 2
KGPHEF

SEQ ID NO: 830 - protein, 14 aa, Severe acute respiratory syndrome coronavirus 2
TMLVKQGDDYVYLP

SEQ ID NO: 831 - protein, 7 aa, Severe acute respiratory syndrome coronavirus 2
KTDGTLM

SEQ ID NO: 832 - protein, 6 aa, Severe acute respiratory syndrome coronavirus 2
LAIDAY

SEQ ID NO: 833 - protein, 11 aa, Severe acute respiratory syndrome coronavirus 2
NQEYADVFHLY

SEQ ID NO: 834 - protein, 10 aa, Severe acute respiratory syndrome coronavirus 2
IATCDWTNAG

SEQ ID NO: 835 - protein, 11 aa, Severe acute respiratory syndrome coronavirus 2
ETLKATEETFK

SEQ ID NO: 836 - protein, 14 aa, Severe acute respiratory syndrome coronavirus 2
KPRPPLNRNYVFTG

SEQ ID NO: 837 - protein, 9 aa, Severe acute respiratory syndrome coronavirus 2
RVTKNSKVQ

SEQ ID NO: 838 - protein, 10 aa, Severe acute respiratory syndrome coronavirus 2
IGEYTFEKGD

SEQ ID NO: 839 - protein, 8 aa, Severe acute respiratory syndrome coronavirus 2
KGDYGDAV

SEQ ID NO: 840 - protein, 10 aa, Severe acute respiratory syndrome coronavirus 2
VYRGTTTYKL

SEQ ID NO: 841 - protein, 10 aa, Severe acute respiratory syndrome coronavirus 2
YQKVGMQKYS

SEQ ID NO: 842 - protein, 15 aa, Severe acute respiratory syndrome coronavirus 2
KVGMQKYSTLQGPPG

SEQ ID NO: 843 - protein, 12 aa, Severe acute respiratory syndrome coronavirus 2
MQKYSTLQGPPG

SEQ ID NO: 844 - protein, 16 aa, Severe acute respiratory syndrome coronavirus 2
DKFKVNSTLEQYVFCT

SEQ ID NO: 845 - protein, 16 aa, Severe acute respiratory syndrome coronavirus 2
ATNYDLSVVNARLRAK

SEQ ID NO: 846 - protein, 8 aa, Severe acute respiratory syndrome coronavirus 2
VNARLRAK

SEQ ID NO: 847 - protein, 8 aa, Severe acute respiratory syndrome coronavirus 2
RLRAKHYV

SEQ ID NO: 848 - protein, 11 aa, Severe acute respiratory syndrome coronavirus 2
AKHYVYIGDPA

SEQ ID NO: 849 - protein, 19 aa, Severe acute respiratory syndrome coronavirus 2
DPAQLPAPRTLLTKGTLEP

SEQ ID NO: 850 - protein, 8 aa, Severe acute respiratory syndrome coronavirus 2
APRTLLTK

SEQ ID NO: 851 - protein, 6 aa, Severe acute respiratory syndrome coronavirus 2
FNSVCR

SEQ ID NO: 852 - protein, 15 aa, Severe acute respiratory syndrome coronavirus 2
EIVDTVSALVYDNKL

SEQ ID NO: 853 - protein, 11 aa, Severe acute respiratory syndrome coronavirus 2
IVDTVSALVYD

SEQ ID NO: 854 - protein, 14 aa, Severe acute respiratory syndrome coronavirus 2
YDNKLKAHKDKSAQ

SEQ ID NO: 855 - protein, 9 aa, Severe acute respiratory syndrome coronavirus 2
AQCFKMFYK

SEQ ID NO: 856 - protein, 10 aa, Severe acute respiratory syndrome coronavirus 2
YKGVITHDVS

SEQ ID NO: 857 - protein, 13 aa, Severe acute respiratory syndrome coronavirus 2
THDVSSAINRPQI

SEQ ID NO: 858 - protein, 9 aa, Severe acute respiratory syndrome coronavirus 2
NPAWRKAVF

SEQ ID NO: 859 - protein, 5 aa, Severe acute respiratory syndrome coronavirus 2
RKAVF

SEQ ID NO: 860 - protein, 10 aa, Severe acute respiratory syndrome coronavirus 2
VFISPYNSQN

SEQ ID NO: 861 - protein, 12 aa, Severe acute respiratory syndrome coronavirus 2
PYNSQNAVASKI

SEQ ID NO: 862 - protein, 11 aa, Severe acute respiratory syndrome coronavirus 2
YNSQNAVASKI

SEQ ID NO: 863 - protein, 9 aa, Severe acute respiratory syndrome coronavirus 2
IFTQTTETA

SEQ ID NO: 864 - protein, 9 aa, Severe acute respiratory syndrome coronavirus 2
VGILCIMSD

SEQ ID NO: 865 - protein, 8 aa, Severe acute respiratory syndrome coronavirus 2
GILCIMSD

SEQ ID NO: 866 - protein, 8 aa, Severe acute respiratory syndrome coronavirus 2
GILCIMSD

SEQ ID NO: 867 - protein, 10 aa, Severe acute respiratory syndrome coronavirus 2
GILCIMSDRD

SEQ ID NO: 868 - protein, 12 aa, Severe acute respiratory syndrome coronavirus 2
LCIMSDRDLYDK

SEQ ID NO: 869 - protein, 10 aa, Severe acute respiratory syndrome coronavirus 2
FTSLEIPRRN

SEQ ID NO: 870 - protein, 8 aa, Severe acute respiratory syndrome coronavirus 2
EIPRRNVA

SEQ ID NO: 871 - protein, 8 aa, Severe acute respiratory syndrome coronavirus 2
EIPRRNVA

SEQ ID NO: 872 - protein, 13 aa, Severe acute respiratory syndrome coronavirus 2
IPRRNVATLQAEN

SEQ ID NO: 873 - protein, 10 aa, Severe acute respiratory syndrome coronavirus 2
QAENVTGLFK

SEQ ID NO: 874 - protein, 18 aa, Severe acute respiratory syndrome coronavirus 2
TGLFKDCSKVITGLHPTQ

SEQ ID NO: 875 - protein, 11 aa, Severe acute respiratory syndrome coronavirus 2
EGCHATREAVG

SEQ ID NO: 876 - protein, 16 aa, Severe acute respiratory syndrome coronavirus 2
GCHATREAVGTNLPLQ

SEQ ID NO: 877 - protein, 9 aa, Severe acute respiratory syndrome coronavirus 2
STGVNLVAV

SEQ ID NO: 878 - protein, 13 aa, Severe acute respiratory syndrome coronavirus 2
NNTDFSRVSAKPP

SEQ ID NO: 879 - protein, 8 aa, Severe acute respiratory syndrome coronavirus 2
VSAKPPPG

SEQ ID NO: 880 - protein, 11 aa, Severe acute respiratory syndrome coronavirus 2
AKPPPGDQFKH

SEQ ID NO: 881 - protein, 13 aa, Severe acute respiratory syndrome coronavirus 2
SDRVVFVLWAHGF

SEQ ID NO: 882 - protein, 9 aa, Severe acute respiratory syndrome coronavirus 2
LWAHGFELT

SEQ ID NO: 883 - protein, 9 aa, Severe acute respiratory syndrome coronavirus 2
DRRATCFST

SEQ ID NO: 884 - protein, 6 aa, Severe acute respiratory syndrome coronavirus 2
LQSNHD

SEQ ID NO: 885 - protein, 9 aa, Severe acute respiratory syndrome coronavirus 2
LAVHECFVK

SEQ ID NO: 886 - protein, 11 aa, Severe acute respiratory syndrome coronavirus 2
EYPIIGDELKI

SEQ ID NO: 887 - protein, 17 aa, Severe acute respiratory syndrome coronavirus 2
VQHMVVKAALLADKFPV

SEQ ID NO: 888 - protein, 7 aa, Severe acute respiratory syndrome coronavirus 2
ADKFPVL

SEQ ID NO: 889 - protein, 9 aa, Severe acute respiratory syndrome coronavirus 2
PQADVEWKF

SEQ ID NO: 890 - protein, 9 aa, Severe acute respiratory syndrome coronavirus 2
KFYDAQPCS

SEQ ID NO: 891 - protein, 9 aa, Severe acute respiratory syndrome coronavirus 2
KIEELFYSY

SEQ ID NO: 892 - protein, 9 aa, Severe acute respiratory syndrome coronavirus 2
CDGGSLYVN

SEQ ID NO: 893 - protein, 8 aa, Severe acute respiratory syndrome coronavirus 2
FDKSAFVN

SEQ ID NO: 894 - protein, 12 aa, Severe acute respiratory syndrome coronavirus 2
DKSAFVNLKQLP

SEQ ID NO: 895 - protein, 7 aa, Severe acute respiratory syndrome coronavirus 2
VNLKQLP

SEQ ID NO: 896 - protein, 9 aa, Severe acute respiratory syndrome coronavirus 2
NLKQLPFFY

SEQ ID NO: 897 - protein, 12 aa, Severe acute respiratory syndrome coronavirus 2
NLKQLPFFYYSD

SEQ ID NO: 898 - protein, 12 aa, Severe acute respiratory syndrome coronavirus 2
HGKQVVSDIDYV

SEQ ID NO: 899 - protein, 9 aa, Severe acute respiratory syndrome coronavirus 2
IDYVPLKSA

SEQ ID NO: 900 - protein, 10 aa, Severe acute respiratory syndrome coronavirus 2
ENVAFNVVNK

SEQ ID NO: 901 - protein, 16 aa, Severe acute respiratory syndrome coronavirus 2
HFDGQQGEVPVSIINN

SEQ ID NO: 902 - protein, 10 aa, Severe acute respiratory syndrome coronavirus 2
GEVPVSIINN

SEQ ID NO: 903 - protein, 9 aa, Severe acute respiratory syndrome coronavirus 2
TKVDGVDVE

SEQ ID NO: 904 - protein, 5 aa, Severe acute respiratory syndrome coronavirus 2
ENKTT

SEQ ID NO: 905 - protein, 16 aa, Severe acute respiratory syndrome coronavirus 2
NKTTLPVNVAFELWAK

SEQ ID NO: 906 - protein, 10 aa, Severe acute respiratory syndrome coronavirus 2
VNVAFELWAK

SEQ ID NO: 907 - protein, 15 aa, Severe acute respiratory syndrome coronavirus 2
AFELWAKRNIKPVPE

SEQ ID NO: 908 - protein, 17 aa, Severe acute respiratory syndrome coronavirus 2
WAKRNIKPVPEVKILNN

SEQ ID NO: 909 - protein, 10 aa, Severe acute respiratory syndrome coronavirus 2
AKRNIKPVPE

SEQ ID NO: 910 - protein, 10 aa, Severe acute respiratory syndrome coronavirus 2
KRNIKPVPEV

SEQ ID NO: 911 - protein, 15 aa, Severe acute respiratory syndrome coronavirus 2
KRNIKPVPEVKILNN

SEQ ID NO: 912 - protein, 7 aa, Severe acute respiratory syndrome coronavirus 2
KILNNLG

SEQ ID NO: 913 - protein, 10 aa, Severe acute respiratory syndrome coronavirus 2
STIGVCSMTD

SEQ ID NO: 914 - protein, 9 aa, Severe acute respiratory syndrome coronavirus 2
TDIAKKPTE

SEQ ID NO: 915 - protein, 11 aa, Severe acute respiratory syndrome coronavirus 2
TICAPLTVFFD

SEQ ID NO: 916 - protein, 15 aa, Severe acute respiratory syndrome coronavirus 2
ARNGVLITEGSVKGL

SEQ ID NO: 917 - protein, 7 aa, Severe acute respiratory syndrome coronavirus 2
SVKGLQP

SEQ ID NO: 918 - protein, 12 aa, Severe acute respiratory syndrome coronavirus 2
KGLQPSVGPKQA

SEQ ID NO: 919 - protein, 8 aa, Severe acute respiratory syndrome coronavirus 2
PKQASLNG

SEQ ID NO: 920 - protein, 12 aa, Severe acute respiratory syndrome coronavirus 2
PKQASLNGVTLI

SEQ ID NO: 921 - protein, 15 aa, Severe acute respiratory syndrome coronavirus 2
IGEAVKTQFNYYKKV

SEQ ID NO: 922 - protein, 7 aa, Severe acute respiratory syndrome coronavirus 2
GEAVKTQ

SEQ ID NO: 923 - protein, 12 aa, Severe acute respiratory syndrome coronavirus 2
VKTQFNYYKKVD

SEQ ID NO: 924 - protein, 9 aa, Severe acute respiratory syndrome coronavirus 2
TQFNYYKKV

SEQ ID NO: 925 - protein, 13 aa, Severe acute respiratory syndrome coronavirus 2
TQFNYYKKVDGVV

SEQ ID NO: 926 - protein, 12 aa, Severe acute respiratory syndrome coronavirus 2
KKVDGVVQQLPE

SEQ ID NO: 927 - protein, 12 aa, Severe acute respiratory syndrome coronavirus 2
PETYFTQSRNLQ

SEQ ID NO: 928 - protein, 11 aa, Severe acute respiratory syndrome coronavirus 2
LQEFKPRSQME

SEQ ID NO: 929 - protein, 5 aa, Severe acute respiratory syndrome coronavirus 2
EHIVY

SEQ ID NO: 930 - protein, 8 aa, Severe acute respiratory syndrome coronavirus 2
AKRFKESP

SEQ ID NO: 931 - protein, 10 aa, Severe acute respiratory syndrome coronavirus 2
KESPFELEDF

SEQ ID NO: 932 - protein, 8 aa, Severe acute respiratory syndrome coronavirus 2
MDSTVKNY

SEQ ID NO: 933 - protein, 6 aa, Severe acute respiratory syndrome coronavirus 2
KCVCSV

SEQ ID NO: 934 - protein, 10 aa, Severe acute respiratory syndrome coronavirus 2
VIDLLLDDFV

SEQ ID NO: 935 - protein, 8 aa, Severe acute respiratory syndrome coronavirus 2
ETFYPKLQ

SEQ ID NO: 936 - protein, 16 aa, Severe acute respiratory syndrome coronavirus 2
KCDLQNYGDSATLPKG

SEQ ID NO: 937 - protein, 12 aa, Severe acute respiratory syndrome coronavirus 2
RVIHFGAGSDKG

SEQ ID NO: 938 - protein, 8 aa, Severe acute respiratory syndrome coronavirus 2
VAPGTAVL

SEQ ID NO: 939 - protein, 12 aa, Severe acute respiratory syndrome coronavirus 2
LLVDSDLNDFVS

SEQ ID NO: 940 - protein, 7 aa, Severe acute respiratory syndrome coronavirus 2
NDFVSDA

SEQ ID NO: 941 - protein, 21 aa, Severe acute respiratory syndrome coronavirus 2
VHTANKWDLIISDMYDPKTKN

SEQ ID NO: 942 - protein, 20 aa, Severe acute respiratory syndrome coronavirus 2
ANKWDLIISDMYDPKTKNVT

SEQ ID NO: 943 - protein, 11 aa, Severe acute respiratory syndrome coronavirus 2
KWDLIISDMYD

SEQ ID NO: 944 - protein, 14 aa, Severe acute respiratory syndrome coronavirus 2
SDMYDPKTKNVTKE

SEQ ID NO: 945 - protein, 15 aa, Severe acute respiratory syndrome coronavirus 2
SDMYDPKTKNVTKEN

SEQ ID NO: 946 - protein, 10 aa, Severe acute respiratory syndrome coronavirus 2
KTKNVTKEND

SEQ ID NO: 947 - protein, 12 aa, Severe acute respiratory syndrome coronavirus 2
KTKNVTKENDSK

SEQ ID NO: 948 - protein, 7 aa, Severe acute respiratory syndrome coronavirus 2
NVTKEND

SEQ ID NO: 949 - protein, 5 aa, Severe acute respiratory syndrome coronavirus 2
ADLYK

SEQ ID NO: 950 - protein, 6 aa, Severe acute respiratory syndrome coronavirus 2
SVTSSI

SEQ ID NO: 951 - protein, 16 aa, Severe acute respiratory syndrome coronavirus 2
VITSGDGTTSPISEHD

SEQ ID NO: 952 - protein, 9 aa, Severe acute respiratory syndrome coronavirus 2
YQIGGYTEK

SEQ ID NO: 953 - protein, 9 aa, Severe acute respiratory syndrome coronavirus 2
FIYNKIVDE

SEQ ID NO: 954 - protein, 14 aa, Severe acute respiratory syndrome coronavirus 2
VDEPEEHVQIHTID

SEQ ID NO: 955 - protein, 5 aa, Severe acute respiratory syndrome coronavirus 2
NPVME

SEQ ID NO: 956 - protein, 9 aa, Severe acute respiratory syndrome coronavirus 2
VMEPIYDEP

SEQ ID NO: 957 - protein, 12 aa, Severe acute respiratory syndrome coronavirus 2
YDEPTTTTSVPL

SEQ ID NO: 958 - protein, 6 aa, Severe acute respiratory syndrome coronavirus 2
FHLVDF

SEQ ID NO: 959 - protein, 7 aa, Severe acute respiratory syndrome coronavirus 2
HLVDFQV

SEQ ID NO: 960 - protein, 7 aa, Severe acute respiratory syndrome coronavirus 2
VDFQVTI

SEQ ID NO: 961 - protein, 7 aa, Severe acute respiratory syndrome coronavirus 2
DFQVTIA

SEQ ID NO: 962 - protein, 6 aa, Severe acute respiratory syndrome coronavirus 2
FQVTIA

SEQ ID NO: 963 - protein, 10 aa, Severe acute respiratory syndrome coronavirus 2
IAEILLIIMR

SEQ ID NO: 964 - protein, 6 aa, Severe acute respiratory syndrome coronavirus 2
AEILLI

SEQ ID NO: 965 - protein, 7 aa, Severe acute respiratory syndrome coronavirus 2
IIMRTFK

SEQ ID NO: 966 - protein, 10 aa, Severe acute respiratory syndrome coronavirus 2
IIMRTFKVSI

SEQ ID NO: 967 - protein, 11 aa, Severe acute respiratory syndrome coronavirus 2
MRTFKVSIWNL

SEQ ID NO: 968 - protein, 7 aa, Severe acute respiratory syndrome coronavirus 2
TFKVSIW

SEQ ID NO: 969 - protein, 17 aa, Severe acute respiratory syndrome coronavirus 2
NLIIKNLSKSLTENKYS

SEQ ID NO: 970 - protein, 20 aa, Severe acute respiratory syndrome coronavirus 2
NLIIKNLSKSLTENKYSQLD

SEQ ID NO: 971 - protein, 5 aa, Severe acute respiratory syndrome coronavirus 2
IIKNL

SEQ ID NO: 972 - protein, 6 aa, Severe acute respiratory syndrome coronavirus 2
IIKNLS

SEQ ID NO: 973 - protein, 9 aa, Severe acute respiratory syndrome coronavirus 2
IKNLSKSLT

SEQ ID NO: 974 - protein, 15 aa, Severe acute respiratory syndrome coronavirus 2
IKNLSKSLTENKYSQ

SEQ ID NO: 975 - protein, 8 aa, Severe acute respiratory syndrome coronavirus 2
NLSKSLTE

SEQ ID NO: 976 - protein, 12 aa, Severe acute respiratory syndrome coronavirus 2
NLSKSLTENKYS

SEQ ID NO: 977 - protein, 12 aa, Severe acute respiratory syndrome coronavirus 2
NLSKSLTENKYS

SEQ ID NO: 978 - protein, 12 aa, Severe acute respiratory syndrome coronavirus 2
NLSKSLTENKYS

SEQ ID NO: 979 - protein, 12 aa, Severe acute respiratory syndrome coronavirus 2
NLSKSLTENKYS

SEQ ID NO: 980 - protein, 18 aa, Severe acute respiratory syndrome coronavirus 2
NLSKSLTENKYSQLDEEQ

SEQ ID NO: 981 - protein, 9 aa, Severe acute respiratory syndrome coronavirus 2
LSKSLTENK

SEQ ID NO: 982 - protein, 14 aa, Severe acute respiratory syndrome coronavirus 2
LSKSLTENKYSQLD

SEQ ID NO: 983 - protein, 10 aa, Severe acute respiratory syndrome coronavirus 2
SKSLTENKYS

SEQ ID NO: 984 - protein, 15 aa, Severe acute respiratory syndrome coronavirus 2
SKSLTENKYSQLDEE

SEQ ID NO: 985 - protein, 21 aa, Severe acute respiratory syndrome coronavirus 2
SKSLTENKYSQLDEEQPMEID

SEQ ID NO: 986 - protein, 6 aa, Severe acute respiratory syndrome coronavirus 2
KSLTEN

SEQ ID NO: 987 - protein, 10 aa, Severe acute respiratory syndrome coronavirus 2
KSLTENKYSQ

SEQ ID NO: 988 - protein, 12 aa, Severe acute respiratory syndrome coronavirus 2
KSLTENKYSQLD

SEQ ID NO: 989 - protein, 20 aa, Severe acute respiratory syndrome coronavirus 2
KSLTENKYSQLDEEQPMEID

SEQ ID NO: 990 - protein, 8 aa, Severe acute respiratory syndrome coronavirus 2
SLTENKYS

SEQ ID NO: 991 - protein, 9 aa, Severe acute respiratory syndrome coronavirus 2
SLTENKYSQ

SEQ ID NO: 992 - protein, 18 aa, Severe acute respiratory syndrome coronavirus 2
LTENKYSQLDEEQPMEID

SEQ ID NO: 993 - protein, 10 aa, Severe acute respiratory syndrome coronavirus 2
NKYSQLDEEQ

SEQ ID NO: 994 - protein, 11 aa, Severe acute respiratory syndrome coronavirus 2
QLDEEQPMEID

SEQ ID NO: 995 - protein, 9 aa, Severe acute respiratory syndrome coronavirus 2
DEEQPMEID

SEQ ID NO: 996 - protein, 8 aa, Severe acute respiratory syndrome coronavirus 2
EEQPMEID

SEQ ID NO: 997 - protein, 5 aa, Severe acute respiratory syndrome coronavirus 2
PMEID

SEQ ID NO: 998 - protein, 5 aa, Severe acute respiratory syndrome coronavirus 2
PMEID

SEQ ID NO: 999 - protein, 12 aa, Severe acute respiratory syndrome coronavirus 2
EGNSPFHPLADN

SEQ ID NO: 1000 - protein, 9 aa, Severe acute respiratory syndrome coronavirus 2
YVVDDPCPI

SEQ ID NO: 1001 - protein, 8 aa, Severe acute respiratory syndrome coronavirus 2
DEAGSKSP

SEQ ID NO: 1002 - protein, 6 aa, Severe acute respiratory syndrome coronavirus 2
VVLDFI

SEQ ID NO: 1003 - protein, 10 aa, Severe acute respiratory syndrome coronavirus 2
DPKISEMHPA

SEQ ID NO: 1004 - protein, 9 aa, Severe acute respiratory syndrome coronavirus 2
PIILRLGSP

SEQ ID NO: 1005 - protein, 16 aa, Severe acute respiratory syndrome coronavirus 2
PLSLNMARKTLNSLED

SEQ ID NO: 1006 - protein, 11 aa, Severe acute respiratory syndrome coronavirus 2
RKTLNSLEDKA

SEQ ID NO: 1007 - protein, 16 aa, Severe acute respiratory syndrome coronavirus 2
RKTLNSLEDKAFQLTP

SEQ ID NO: 1008 - protein, 17 aa, Severe acute respiratory syndrome coronavirus 2
EDKAFQLTPIAVQMTKL

SEQ ID NO: 1009 - protein, 6 aa, Severe acute respiratory syndrome coronavirus 2
TEELPD

SEQ ID NO: 1010 - protein, 10 aa, Severe acute respiratory syndrome coronavirus 2
QKASTQKGAE

SEQ ID NO: 1011 - protein, 16 aa, Severe acute respiratory syndrome coronavirus 2
PAYTNSFTRGVYYPDK

SEQ ID NO: 1012 - protein, 12 aa, Severe acute respiratory syndrome coronavirus 2
SFTRGVYYPDKV

SEQ ID NO: 1013 - protein, 8 aa, Severe acute respiratory syndrome coronavirus 2
NDGVYFAS

SEQ ID NO: 1014 - protein, 8 aa, Severe acute respiratory syndrome coronavirus 2
SLLIVNNA

SEQ ID NO: 1015 - protein, 6 aa, Severe acute respiratory syndrome coronavirus 2
CNDPFL

SEQ ID NO: 1016 - protein, 15 aa, Severe acute respiratory syndrome coronavirus 2
NDPFLGVYYHKNNKS

SEQ ID NO: 1017 - protein, 18 aa, Severe acute respiratory syndrome coronavirus 2
NDPFLGVYYHKNNKSWME

SEQ ID NO: 1018 - protein, 13 aa, Severe acute respiratory syndrome coronavirus 2
VYYHKNNKSWMES

SEQ ID NO: 1019 - protein, 7 aa, Severe acute respiratory syndrome coronavirus 2
YYHKNNK

SEQ ID NO: 1020 - protein, 11 aa, Severe acute respiratory syndrome coronavirus 2
YYHKNNKSWME

SEQ ID NO: 1021 - protein, 12 aa, Severe acute respiratory syndrome coronavirus 2
YYHKNNKSWMES

SEQ ID NO: 1022 - protein, 12 aa, Severe acute respiratory syndrome coronavirus 2
YYHKNNKSWMES

SEQ ID NO: 1023 - protein, 6 aa, Severe acute respiratory syndrome coronavirus 2
SSANNC

SEQ ID NO: 1024 - protein, 11 aa, Severe acute respiratory syndrome coronavirus 2
DLEGKQGNFKN

SEQ ID NO: 1025 - protein, 17 aa, Severe acute respiratory syndrome coronavirus 2
EGKQGNFKNLREFVFKN

SEQ ID NO: 1026 - protein, 9 aa, Severe acute respiratory syndrome coronavirus 2
GNFKNLREF

SEQ ID NO: 1027 - protein, 12 aa, Severe acute respiratory syndrome coronavirus 2
NIDGYFKIYSKH

SEQ ID NO: 1028 - protein, 13 aa, Severe acute respiratory syndrome coronavirus 2
DLPIGINITRFQT

SEQ ID NO: 1029 - protein, 13 aa, Severe acute respiratory syndrome coronavirus 2
GAAAYYVGYLQPR

SEQ ID NO: 1030 - protein, 19 aa, Severe acute respiratory syndrome coronavirus 2
QPRTFLLKYNENGTITDAV

SEQ ID NO: 1031 - protein, 7 aa, Severe acute respiratory syndrome coronavirus 2
YNENGTI

SEQ ID NO: 1032 - protein, 19 aa, Severe acute respiratory syndrome coronavirus 2
DAVDCALDPLSETKCTLKS

SEQ ID NO: 1033 - protein, 6 aa, Severe acute respiratory syndrome coronavirus 2
KCTLKS

SEQ ID NO: 1034 - protein, 11 aa, Severe acute respiratory syndrome coronavirus 2
CTLKSFTVEKG

SEQ ID NO: 1035 - protein, 17 aa, Severe acute respiratory syndrome coronavirus 2
VEKGIYQTSNFRVQPTE

SEQ ID NO: 1036 - protein, 7 aa, Severe acute respiratory syndrome coronavirus 2
VRFPNIT

SEQ ID NO: 1037 - protein, 9 aa, Severe acute respiratory syndrome coronavirus 2
PNITNLCPF

SEQ ID NO: 1038 - protein, 10 aa, Severe acute respiratory syndrome coronavirus 2
SVYAWNRKRI

SEQ ID NO: 1039 - protein, 11 aa, Severe acute respiratory syndrome coronavirus 2
YAWNRKRISNC

SEQ ID NO: 1040 - protein, 8 aa, Severe acute respiratory syndrome coronavirus 2
RKRISNCV

SEQ ID NO: 1041 - protein, 14 aa, Severe acute respiratory syndrome coronavirus 2
GDEVRQIAPGQTGK

SEQ ID NO: 1042 - protein, 14 aa, Severe acute respiratory syndrome coronavirus 2
QTGKIADYNYKLPD

SEQ ID NO: 1043 - protein, 13 aa, Severe acute respiratory syndrome coronavirus 2
IADYNYKLPDDFT

SEQ ID NO: 1044 - protein, 5 aa, Severe acute respiratory syndrome coronavirus 2
KLPDD

SEQ ID NO: 1045 - protein, 11 aa, Severe acute respiratory syndrome coronavirus 2
SNNLDSKVGGN

SEQ ID NO: 1046 - protein, 11 aa, Severe acute respiratory syndrome coronavirus 2
YRLFRKSNLKP

SEQ ID NO: 1047 - protein, 9 aa, Severe acute respiratory syndrome coronavirus 2
LFRKSNLKP

SEQ ID NO: 1048 - protein, 9 aa, Severe acute respiratory syndrome coronavirus 2
SNLKPFERD

SEQ ID NO: 1049 - protein, 11 aa, Severe acute respiratory syndrome coronavirus 2
TPCNGVEGFNC

SEQ ID NO: 1050 - protein, 12 aa, Severe acute respiratory syndrome coronavirus 2
NKCVNFNFNGLT

SEQ ID NO: 1051 - protein, 6 aa, Severe acute respiratory syndrome coronavirus 2
NFNGLT

SEQ ID NO: 1052 - protein, 12 aa, Severe acute respiratory syndrome coronavirus 2
GTGVLTESNKKF

SEQ ID NO: 1053 - protein, 6 aa, Severe acute respiratory syndrome coronavirus 2
VLTESN

SEQ ID NO: 1054 - protein, 13 aa, Severe acute respiratory syndrome coronavirus 2
ESNKKFLPFQQFG

SEQ ID NO: 1055 - protein, 11 aa, Severe acute respiratory syndrome coronavirus 2
SNKKFLPFQQF

SEQ ID NO: 1056 - protein, 12 aa, Severe acute respiratory syndrome coronavirus 2
SNKKFLPFQQFG

SEQ ID NO: 1057 - protein, 15 aa, Severe acute respiratory syndrome coronavirus 2
SNKKFLPFQQFGRDI

SEQ ID NO: 1058 - protein, 15 aa, Severe acute respiratory syndrome coronavirus 2
SNKKFLPFQQFGRDI

SEQ ID NO: 1059 - protein, 15 aa, Severe acute respiratory syndrome coronavirus 2
NKKFLPFQQFGRDIA

SEQ ID NO: 1060 - protein, 10 aa, Severe acute respiratory syndrome coronavirus 2
LPFQQFGRDI

SEQ ID NO: 1061 - protein, 10 aa, Severe acute respiratory syndrome coronavirus 2
TTDAVRDPQT

SEQ ID NO: 1062 - protein, 16 aa, Severe acute respiratory syndrome coronavirus 2
TDAVRDPQTLEILDIT

SEQ ID NO: 1063 - protein, 10 aa, Severe acute respiratory syndrome coronavirus 2
AVRDPQTLEI

SEQ ID NO: 1064 - protein, 11 aa, Severe acute respiratory syndrome coronavirus 2
AVRDPQTLEIL

SEQ ID NO: 1065 - protein, 11 aa, Severe acute respiratory syndrome coronavirus 2
AVRDPQTLEIL

SEQ ID NO: 1066 - protein, 5 aa, Severe acute respiratory syndrome coronavirus 2
TPGTN

SEQ ID NO: 1067 - protein, 8 aa, Severe acute respiratory syndrome coronavirus 2
PGTNTSNQ

SEQ ID NO: 1068 - protein, 16 aa, Severe acute respiratory syndrome coronavirus 2
PVAIHADQLTPTWRVY

SEQ ID NO: 1069 - protein, 14 aa, Severe acute respiratory syndrome coronavirus 2
DQLTPTWRVYSTGS

SEQ ID NO: 1070 - protein, 14 aa, Severe acute respiratory syndrome coronavirus 2
LTPTWRVYSTGSNV

SEQ ID NO: 1071 - protein, 10 aa, Severe acute respiratory syndrome coronavirus 2
TPTWRVYSTG

SEQ ID NO: 1072 - protein, 6 aa, Severe acute respiratory syndrome coronavirus 2
YSTGSN

SEQ ID NO: 1073 - protein, 8 aa, Severe acute respiratory syndrome coronavirus 2
IGAEHVNN

SEQ ID NO: 1074 - protein, 6 aa, Severe acute respiratory syndrome coronavirus 2
IGAGIC

SEQ ID NO: 1075 - protein, 15 aa, Severe acute respiratory syndrome coronavirus 2
QTQTNSPRRARSVAS

SEQ ID NO: 1076 - protein, 15 aa, Severe acute respiratory syndrome coronavirus 2
TQTNSPRRARSVASQ

SEQ ID NO: 1077 - protein, 18 aa, Severe acute respiratory syndrome coronavirus 2
SVASQSIIAYTMSLGAEN

SEQ ID NO: 1078 - protein, 5 aa, Severe acute respiratory syndrome coronavirus 2
SQSII

SEQ ID NO: 1079 - protein, 9 aa, Severe acute respiratory syndrome coronavirus 2
SQSIIAYTM

SEQ ID NO: 1080 - protein, 8 aa, Severe acute respiratory syndrome coronavirus 2
AYTMSLGA

SEQ ID NO: 1081 - protein, 14 aa, Severe acute respiratory syndrome coronavirus 2
YTMSLGAENSVAYS

SEQ ID NO: 1082 - protein, 17 aa, Severe acute respiratory syndrome coronavirus 2
ENSVAYSNNSIAIPTNF

SEQ ID NO: 1083 - protein, 7 aa, Severe acute respiratory syndrome coronavirus 2
SVAYSNN

SEQ ID NO: 1084 - protein, 7 aa, Severe acute respiratory syndrome coronavirus 2
NNSIAIP

SEQ ID NO: 1085 - protein, 6 aa, Severe acute respiratory syndrome coronavirus 2
TKTSVD

SEQ ID NO: 1086 - protein, 14 aa, Severe acute respiratory syndrome coronavirus 2
LTGIAVEQDKNTQE

SEQ ID NO: 1087 - protein, 15 aa, Severe acute respiratory syndrome coronavirus 2
LTGIAVEQDKNTQEV

SEQ ID NO: 1088 - protein, 14 aa, Severe acute respiratory syndrome coronavirus 2
GIAVEQDKNTQEVF

SEQ ID NO: 1089 - protein, 11 aa, Severe acute respiratory syndrome coronavirus 2
IAVEQDKNTQE

SEQ ID NO: 1090 - protein, 12 aa, Severe acute respiratory syndrome coronavirus 2
IAVEQDKNTQEV

SEQ ID NO: 1091 - protein, 20 aa, Severe acute respiratory syndrome coronavirus 2
VEQDKNTQEVFAQVKQIYKT

SEQ ID NO: 1092 - protein, 14 aa, Severe acute respiratory syndrome coronavirus 2
QIYKTPPIKDFGGF

SEQ ID NO: 1093 - protein, 10 aa, Severe acute respiratory syndrome coronavirus 2
IYKTPPIKDF

SEQ ID NO: 1094 - protein, 13 aa, Severe acute respiratory syndrome coronavirus 2
TPPIKDFGGFNFS

SEQ ID NO: 1095 - protein, 27 aa, Severe acute respiratory syndrome coronavirus 2
GFNFSQILPDPSKPSKRSFIEDLLFNK

SEQ ID NO: 1096 - protein, 25 aa, Severe acute respiratory syndrome coronavirus 2
FSQILPDPSKPSKRSFIEDLLFNKV

SEQ ID NO: 1097 - protein, 11 aa, Severe acute respiratory syndrome coronavirus 2
ILPDPSKPSKR

SEQ ID NO: 1098 - protein, 13 aa, Severe acute respiratory syndrome coronavirus 2
PSKPSKRSFIEDL

SEQ ID NO: 1099 - protein, 10 aa, Severe acute respiratory syndrome coronavirus 2
PSKRSFIEDL

SEQ ID NO: 1100 - protein, 15 aa, Severe acute respiratory syndrome coronavirus 2
PSKRSFIEDLLFNKV

SEQ ID NO: 1101 - protein, 15 aa, Severe acute respiratory syndrome coronavirus 2
PSKRSFIEDLLFNKV

SEQ ID NO: 1102 - protein, 14 aa, Severe acute respiratory syndrome coronavirus 2
SKRSFIEDLLFNKV

SEQ ID NO: 1103 - protein, 15 aa, Severe acute respiratory syndrome coronavirus 2
SKRSFIEDLLFNKVT

SEQ ID NO: 1104 - protein, 7 aa, Severe acute respiratory syndrome coronavirus 2
KRSFIED

SEQ ID NO: 1105 - protein, 13 aa, Severe acute respiratory syndrome coronavirus 2
KRSFIEDLLFNKV

SEQ ID NO: 1106 - protein, 13 aa, Severe acute respiratory syndrome coronavirus 2
KRSFIEDLLFNKV

SEQ ID NO: 1107 - protein, 13 aa, Severe acute respiratory syndrome coronavirus 2
KRSFIEDLLFNKV

SEQ ID NO: 1108 - protein, 13 aa, Severe acute respiratory syndrome coronavirus 2
RSFIEDLLFNKVT

SEQ ID NO: 1109 - protein, 9 aa, Severe acute respiratory syndrome coronavirus 2
FIEDLLFNK

SEQ ID NO: 1110 - protein, 15 aa, Severe acute respiratory syndrome coronavirus 2
GDIAARDLICAQKFN

SEQ ID NO: 1111 - protein, 16 aa, Severe acute respiratory syndrome coronavirus 2
QKFNGLTVLPPLLTDE

SEQ ID NO: 1112 - protein, 7 aa, Severe acute respiratory syndrome coronavirus 2
LAGTITS

SEQ ID NO: 1113 - protein, 8 aa, Severe acute respiratory syndrome coronavirus 2
MQMAYRFN

SEQ ID NO: 1114 - protein, 11 aa, Severe acute respiratory syndrome coronavirus 2
ENQKLIANQFN

SEQ ID NO: 1115 - protein, 6 aa, Severe acute respiratory syndrome coronavirus 2
FNSAIG

SEQ ID NO: 1116 - protein, 11 aa, Severe acute respiratory syndrome coronavirus 2
GKIQDSLSSTA

SEQ ID NO: 1117 - protein, 6 aa, Severe acute respiratory syndrome coronavirus 2
QDSLSS

SEQ ID NO: 1118 - protein, 24 aa, Severe acute respiratory syndrome coronavirus 2
LSSNFGAISSVLNDILSRLDKVEA

SEQ ID NO: 1119 - protein, 18 aa, Severe acute respiratory syndrome coronavirus 2
ISSVLNDILSRLDKVEAE

SEQ ID NO: 1120 - protein, 17 aa, Severe acute respiratory syndrome coronavirus 2
SSVLNDILSRLDKVEAE

SEQ ID NO: 1121 - protein, 11 aa, Severe acute respiratory syndrome coronavirus 2
NDILSRLDKVE

SEQ ID NO: 1122 - protein, 15 aa, Severe acute respiratory syndrome coronavirus 2
NDILSRLDKVEAEVQ

SEQ ID NO: 1123 - protein, 12 aa, Severe acute respiratory syndrome coronavirus 2
DILSRLDKVEAE

SEQ ID NO: 1124 - protein, 13 aa, Severe acute respiratory syndrome coronavirus 2
LDKVEAEVQIDRL

SEQ ID NO: 1125 - protein, 22 aa, Severe acute respiratory syndrome coronavirus 2
AAEIRASANLAATKMSECVLGQ

SEQ ID NO: 1126 - protein, 16 aa, Severe acute respiratory syndrome coronavirus 2
EIRASANLAATKMSEC

SEQ ID NO: 1127 - protein, 9 aa, Severe acute respiratory syndrome coronavirus 2
FPQSAPHGV

SEQ ID NO: 1128 - protein, 11 aa, Severe acute respiratory syndrome coronavirus 2
KNFTTAPAICH

SEQ ID NO: 1129 - protein, 8 aa, Severe acute respiratory syndrome coronavirus 2
EGVFVSNG

SEQ ID NO: 1130 - protein, 9 aa, Severe acute respiratory syndrome coronavirus 2
TQRNFYEPQ

SEQ ID NO: 1131 - protein, 14 aa, Severe acute respiratory syndrome coronavirus 2
QPELDSFKEELDKY

SEQ ID NO: 1132 - protein, 16 aa, Severe acute respiratory syndrome coronavirus 2
QPELDSFKEELDKYFK

SEQ ID NO: 1133 - protein, 17 aa, Severe acute respiratory syndrome coronavirus 2
QPELDSFKEELDKYFKN

SEQ ID NO: 1134 - protein, 15 aa, Severe acute respiratory syndrome coronavirus 2
PELDSFKEELDKYFK

SEQ ID NO: 1135 - protein, 15 aa, Severe acute respiratory syndrome coronavirus 2
ELDSFKEELDKYFKN

SEQ ID NO: 1136 - protein, 15 aa, Severe acute respiratory syndrome coronavirus 2
ELDSFKEELDKYFKN

SEQ ID NO: 1137 - protein, 15 aa, Severe acute respiratory syndrome coronavirus 2
ELDSFKEELDKYFKN

SEQ ID NO: 1138 - protein, 13 aa, Severe acute respiratory syndrome coronavirus 2
DSFKEELDKYFKN

SEQ ID NO: 1139 - protein, 10 aa, Severe acute respiratory syndrome coronavirus 2
SFKEELDKYF

SEQ ID NO: 1140 - protein, 11 aa, Severe acute respiratory syndrome coronavirus 2
SFKEELDKYFK

SEQ ID NO: 1141 - protein, 11 aa, Severe acute respiratory syndrome coronavirus 2
SFKEELDKYFK

SEQ ID NO: 1142 - protein, 13 aa, Severe acute respiratory syndrome coronavirus 2
SFKEELDKYFKNH

SEQ ID NO: 1143 - protein, 15 aa, Severe acute respiratory syndrome coronavirus 2
SFKEELDKYFKNHTS

SEQ ID NO: 1144 - protein, 10 aa, Severe acute respiratory syndrome coronavirus 2
FKEELDKYFK

SEQ ID NO: 1145 - protein, 11 aa, Severe acute respiratory syndrome coronavirus 2
FKEELDKYFKN

SEQ ID NO: 1146 - protein, 8 aa, Severe acute respiratory syndrome coronavirus 2
KEELDKYF

SEQ ID NO: 1147 - protein, 15 aa, Severe acute respiratory syndrome coronavirus 2
EELDKYFKNHTSPDV

SEQ ID NO: 1148 - protein, 6 aa, Severe acute respiratory syndrome coronavirus 2
LDKYFK

SEQ ID NO: 1149 - protein, 8 aa, Severe acute respiratory syndrome coronavirus 2
DKYFKNHT

SEQ ID NO: 1150 - protein, 15 aa, Severe acute respiratory syndrome coronavirus 2
PDVDLGDISGINASV

SEQ ID NO: 1151 - protein, 13 aa, Severe acute respiratory syndrome coronavirus 2
NIQKEIDRLNEVA

SEQ ID NO: 1152 - protein, 11 aa, Severe acute respiratory syndrome coronavirus 2
QKEIDRLNEVA

SEQ ID NO: 1153 - protein, 12 aa, Severe acute respiratory syndrome coronavirus 2
QKEIDRLNEVAK

SEQ ID NO: 1154 - protein, 18 aa, Severe acute respiratory syndrome coronavirus 2
EIDRLNEVAKNLNESLID

SEQ ID NO: 1155 - protein, 5 aa, Severe acute respiratory syndrome coronavirus 2
LNESL

SEQ ID NO: 1156 - protein, 20 aa, Severe acute respiratory syndrome coronavirus 2
VLTESNKKFLPFQQFGRDIA

SEQ ID NO: 1157 - protein, 20 aa, Severe acute respiratory syndrome coronavirus 2
ALTGIAVEQDKNTQEVFAQV

SEQ ID NO: 1158 - protein, 20 aa, Severe acute respiratory syndrome coronavirus 2
KPSKRSFIEDLLFNKVTLAD

SEQ ID NO: 1159 - protein, 19 aa, Severe acute respiratory syndrome coronavirus 2
LDSFKEELDKYFKNHTSPD

SEQ ID NO: 1160 - protein, 70 aa, Artificial Sequence
  Description of Artificial Sequence Synthetic polypeptide
ALTGIAVEQDKNTQEVFAQVKQIYKTPPIKDFGGFNFSQILPDPSKPSKRSFIEDLLFNKVTLADAGFIK

SEQ ID NO: 1161 - protein, 115 aa, Severe acute respiratory syndrome coronavirus 2
EVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHA

SEQ ID NO: 1162 - protein, 20 aa, Severe acute respiratory syndrome coronavirus 2
PGQTGKIADYNYKLPDDFTG

SEQ ID NO: 1163 - protein, 20 aa, Severe acute respiratory syndrome coronavirus 2
CVIAWNSNNLDSKVGGNYNY

SEQ ID NO: 1164 - protein, 20 aa, Severe acute respiratory syndrome coronavirus 2
GGNYNYLYRLFRKSNLKPFE

SEQ ID NO: 1165 - protein, 20 aa, Severe acute respiratory syndrome coronavirus 2
AGSTPCNGVEGFNCYFPLQS

SEQ ID NO: 1166 - protein, 194 aa, Severe acute respiratory syndrome coronavirus 2
NLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGP

SEQ ID NO: 1167 - protein, 32 aa, Severe acute respiratory syndrome coronavirus 2
GSNQNGERSGARSKQRRPQGLPNNTASWFTAL

SEQ ID NO: 1168 - protein, 65 aa, Severe acute respiratory syndrome coronavirus 2
GSSRGTSPARMAGNGGDAALALLLLDRLNQLESKMSGKGQQQQGQTVTKKSAAEASKKPRQKRTA

SEQ ID NO: 1169 - protein, 25 aa, Severe acute respiratory syndrome coronavirus 2
PANNAAIVLQLPQGTTLPKGFYAEG

SEQ ID NO: 1170 - protein, 46 aa, Severe acute respiratory syndrome coronavirus 2
FPPTEPKKDKKKKADETQALPQRQKKQQTVTLLPAADLDDFSKQLQ

SEQ ID NO: 1171 - protein, 56 aa, Severe acute respiratory syndrome coronavirus 2
VKQIYKTPPIKDFGGFNFSQILPDPSKPSKRSFIEDLLFNKVTLADAGFIKQYGDC

SEQ ID NO: 1172 - protein, 56 aa, Artificial Sequence
  Description of Artificial Sequence Synthetic polypeptide
AAAIYKTPPIKDFGGFNFSQILPDPSKPSKRSFIEDLLFNKVTLADAGFIKQYGDC

SEQ ID NO: 1173 - protein, 56 aa, Artificial Sequence
  Description of Artificial Sequence Synthetic polypeptide
VAAAYKTPPIKDFGGFNFSQILPDPSKPSKRSFIEDLLFNKVTLADAGFIKQYGDC

SEQ ID NO: 1174 - protein, 56 aa, Artificial Sequence
  Description of Artificial Sequence Synthetic polypeptide
VKAAAKTPPIKDFGGFNFSQILPDPSKPSKRSFIEDLLFNKVTLADAGFIKQYGDC

SEQ ID NO: 1175 - protein, 56 aa, Artificial Sequence
  Description of Artificial Sequence Synthetic polypeptide
VKQIYKTPPIKDFGGFNFSQILPDPSKPSKAAAIEDLLFNKVTLADAGFIKQYGDC

SEQ ID NO: 1176 - protein, 56 aa, Artificial Sequence
  Description of Artificial Sequence Synthetic polypeptide
VKQIYKTPPIKDFGGFNFSQILPDPSKPSKRAAAEDLLFNKVTLADAGFIKQYGDC

SEQ ID NO: 1177 - protein, 56 aa, Artificial Sequence
  Description of Artificial Sequence Synthetic polypeptide
VKQIYKTPPIKDFGGFNFSQILPDPSKPSKRSAAADLLFNKVTLADAGFIKQYGDC

SEQ ID NO: 1178 - protein, 56 aa, Artificial Sequence
  Description of Artificial Sequence Synthetic polypeptide
VKQIYKTPPIKDFGGFNFSQILPDPSKAAARSFIEDLLFNKVTLADAGFIKQYGDC

SEQ ID NO: 1179 - protein, 56 aa, Artificial Sequence
  Description of Artificial Sequence Synthetic polypeptide
VKQIYKTPPIKDFGGFNFSQILPDPSKPAAASFIEDLLFNKVTLADAGFIKQYGDC

SEQ ID NO: 1180 - protein, 56 aa, Artificial Sequence
  Description of Artificial Sequence Synthetic polypeptide
VKQIYKTPPIKDFGGFNFSQILPDPSKPSAAAFIEDLLFNKVTLADAGFIKQYGDC

SEQ ID NO: 1181 - protein, 56 aa, Artificial Sequence; identical to SEQ ID NO: 1175
  Description of Artificial Sequence Synthetic polypeptide

SEQ ID NO: 1182 - protein, 56 aa, Artificial Sequence; identical to SEQ ID NO: 1176
  Description of Artificial Sequence Synthetic polypeptide

SEQ ID NO: 1183 - protein, 56 aa, Artificial Sequence; identical to SEQ ID NO: 1177
  Description of Artificial Sequence Synthetic polypeptide

SEQ ID NO: 1184 - protein, 56 aa, Artificial Sequence
  Description of Artificial Sequence Synthetic polypeptide
VKQIYKTPPIKDFGGFNFSQILPDPSKPSKRSFAAALLFNKVTLADAGFIKQYGDC

SEQ ID NO: 1185 - protein, 56 aa, Artificial Sequence
  Description of Artificial Sequence Synthetic polypeptide
VKQIYKTPPIKDFGGFNFSQILPDPSKPSKRSFIAAALFNKVTLADAGFIKQYGDC

SEQ ID NO: 1186 - protein, 56 aa, Artificial Sequence
  Description of Artificial Sequence Synthetic polypeptide
VKQIYKTPPIKDFGGFNFSQILPDPSKPSKRSFIEAAAFNKVTLADAGFIKQYGDC

SEQ ID NO: 1187 - protein, 56 aa, Artificial Sequence
  Description of Artificial Sequence Synthetic polypeptide
VKQIYKTPPIKDFGGFNFSQILPDPSKPSKRSFIEDAAANKVTLADAGFIKQYGDC

SEQ ID NO: 1188 - protein, 56 aa, Artificial Sequence
  Description of Artificial Sequence Synthetic polypeptide
VKQIYKTPPIKDFGGFNFSQILPDPSKPSKRSFIEDLAAAKVTLADAGFIKQYGDC

SEQ ID NO: 1189 - protein, 56 aa, Artificial Sequence
  Description of Artificial Sequence Synthetic polypeptide
VKQIYKTPPIKDFGGFNFSQILPDPSKPSKRSFIEDLLAAAVTLADAGFIKQYGDC

SEQ ID NO: 1190 - protein, 37 aa, Artificial Sequence
  Description of Artificial Sequence Synthetic polypeptide
  MOD_RES (1)..(1): Propargylgylcine
XKKGDGTTSPISEHDYQIGGYTEKWESGVKDCVVLHS

SEQ ID NO: 1191 - protein, 14 aa, Artificial Sequence
  Description of Artificial Sequence Synthetic peptide
IIRSSNQSLAGGIT

SEQ ID NO: 1192 - protein, 14 aa, Artificial Sequence
  Description of Artificial Sequence Synthetic peptide
VIRVSNDTFLNGIT

SEQ ID NO: 1193 - protein, 21 aa, Artificial Sequence
  Description of Artificial Sequence Synthetic peptide
IFKEVSAAYYNNWQNLLYDSN

SEQ ID NO: 1194 - protein, 21 aa, Artificial Sequence
  Description of Artificial Sequence Synthetic peptide
IFVEVNATYYNSWQNLLYDSN

SEQ ID NO: 1195 - protein, 20 aa, Artificial Sequence
  Description of Artificial Sequence Synthetic peptide
ALRLSAHLETNDVSSMLTFD

SEQ ID NO: 1196 - protein, 20 aa, Artificial Sequence
  Description of Artificial Sequence Synthetic peptide
ALRNSARLESADVSEMLTFD

SEQ ID NO: 1197 - protein, 20 aa, Artificial Sequence
  Description of Artificial Sequence Synthetic peptide
ILNEVNDLLDITQLQVANAL

SEQ ID NO: 1198 - protein, 20 aa, Artificial Sequence
  Description of Artificial Sequence Synthetic peptide
ILTEVNELLDTTQLQVANSL

SEQ ID NO: 1199 - protein, 20 aa, Artificial Sequence
  Description of Artificial Sequence Synthetic peptide
RIAGRSALEDLLFSKVVTSG

SEQ ID NO: 1200 - protein, 23 aa, Artificial Sequence
  Description of Artificial Sequence Synthetic peptide
SGSRVAGRSAIEDILFSKLVTSG

SEQ ID NO: 1201 - protein, 20 aa, Artificial Sequence
  Description of Artificial Sequence Synthetic peptide
GSSSRSLLEDLLFNKVKLSD

SEQ ID NO: 1202 - protein, 20 aa, Artificial Sequence
  Description of Artificial Sequence Synthetic peptide
KASSRSAIEDLLFDKVKLSD

SEQ ID NO: 1203 - protein, 20 aa, Severe acute respiratory syndrome coronavirus 2
ELDSFKEELDKYFKNHTSPD

SEQ ID NO: 1204 - protein, 16 aa, Artificial Sequence
  Description of Artificial Sequence Synthetic peptide
TLQEFAQNLPKYVKPN

SEQ ID NO: 1205 - protein, 23 aa, Artificial Sequence
  Description of Artificial Sequence Synthetic peptide
EYIDVNKTLQELSYKLPNYTVPD

SEQ ID NO: 1206 - protein, 20 aa, Artificial Sequence
  Description of Artificial Sequence Synthetic peptide
NLSDFEAEFSLWFKNHTSIA

SEQ ID NO: 1207 - protein, 20 aa, Artificial Sequence
  Description of Artificial Sequence Synthetic peptide
NLPDFKEELDQWFKNQTSVA

SEQ ID NO: 1208 - protein, 20 aa, Unknown
  Description of Unknown SARS-CoV sequence
PGQTGVIADYNYKLPDDFMG

SEQ ID NO: 1209 - protein, 20 aa, Unknown
  Description of Unknown SARS-CoV sequence
CVLAWNTRNIDATSTGNYNY

SEQ ID NO: 1210 - protein, 20 aa, Unknown
  Description of Unknown SARS-CoV sequence
TGNYNYKYRYLRHGKLRPFE

SEQ ID NO: 1211 - protein, 19 aa, Unknown
  Description of Unknown SARS-CoV sequence
PDGKPCTPPALNCYWPLND